(19) United States
(12) Patent Application Publication
(10) Pub. No.: US 2022/0257670 A1
(43) Pub. Date: Aug. 18, 2022

(54) METHODS AND PRODUCTS FOR TREATMENT OF GASTROINTESTINAL DISORDERS

(71) Applicant: Finch Therapeutics Holdings LLC, Somerville, MA (US)

(72) Inventors: Mark SMITH, Somerville, MA (US); Anh-Thu Elaine VO, Somerville, MA (US); Rotem SADOVSKY, Somerville, MA (US); John HENSKE, Somerville, MA (US); Ylaine GERARDIN, Somerville, MA (US); Sonia TIMBERLAKE, Somerville, MA (US)

(21) Appl. No.: 17/628,451

(22) PCT Filed: Jul. 17, 2020
(86) PCT No.: PCT/US20/42546

§ 371 Date: Jan. 19, 2022

Related U.S. Application Data
(60) Provisional application No. 62/876,358, filed on Jul. 19, 2019.

(51) Int. Cl. A61K 35/74; A61P 1/00; C12Q 1/02; G01N 33/50; G01N 33/68

(52) U.S. Cl. CPC A61K 35/74; A61P 1/00; C12Q 1/025; G01N 33/5047; G01N 2800/065; G01N 2333/5255; G01N 2333/5428; G01N 2333/5434; G01N 33/6863

(57) ABSTRACT

Described herein are compositions and methods for the delivery of microbial therapeutics useful for the treatment of disorders related to intestinal dysbiosis.

### PRIORITY

This application claims the benefit of, and priority to, U.S. Provisional Application No. 62/876,358, filed Jul. 19, 2019, the contents of which are hereby incorporated by reference in their entirety.

### FIELD OF THE INVENTION

The present invention relates to, in part, compositions and methods for the delivery of bacterial isolates and/or cocktails of bacterial isolates useful for the treatment of disorders related to intestinal dysbiosis.

### DESCRIPTION OF THE TEXT FILE SUBMITTED ELECTRONICALLY

This application contains a sequence listing. It has been submitted electronically via EFS-Web as an ASCII text file entitled “FIN-020PC_ST25”. The sequence listing is 61,430 bytes in size, and was prepared on or about Jul. 19, 2019. The sequence listing is hereby incorporated by reference in its entirety.

### BACKGROUND

The human GI tract harbors a diverse microbial community of over one thousand distinct bacterial species and an estimated excess of 1×10¹⁴ microorganisms. This microbial community, also referred to as the microbiota (and the genetic component being the microbiome), has proven to be critical for human health. For example, a healthy microbiome provides the human host with multiple benefits, including resistance to pathogen infection, nutrient biosynthesis and absorption, and immune stimulation. A dysbiosis or disruption of the microbiome results in increased susceptibility to pathogens, altered metabolic profiles, and systemic inflammation or autoimmunity. Indeed, dysbiosis of the microbiome (i.e., intestinal dysbiosis) can predispose the human host to a variety of pathological conditions including gastrointestinal disorders such as inflammatory bowel disease (IBD) and irritable bowel syndrome (IBS) as well as infections by pathogens including Clostridium difficile (C. diff, e.g. Clostridium difficile infection (CDI)).

IBD affects over 1.6 million Americans with as many as 70,000 new cases being diagnosed in the United States each year. IBD is characterized by chronic inflammation in the GI tract. The two most common forms of IBD include ulcerative colitis (UC) and Crohn's Disease (CD). UC occurs in the colon while CD may be present in the entire GI tract. The clinical symptoms are diarrhea, abdominal pain, occasional rectal bleeding, weight loss, tiredness and sometimes fever. For most patients, IBD is a chronic condition with symptoms lasting for months to years. Currently, there are no medical cures for IBD. Instead, current therapies are directed to controlling the GI symptoms by reducing inflammation. In severe cases, surgical procedures including colectomy, proctocolectomy, and ileostomy may be used.

Researchers have attempted to restore a healthy microbiome in such patients to alleviate or eliminate these diseases. For instance, one approach involves transplantation of a fecal microbiota derived from stool of a healthy human donor, to repopulate the gut (so-called Fecal Microbiota Transplant (FMT)). However, this treatment is not ideal and particularly unpalatable. Accordingly, a more recent approach is to develop a rationally selected, defined mix of bacteria, which could be taken by patients and replace fecal transplants. This “bugs as drugs” concept looks to convert the therapeutic benefits of FMT to a more standardized and drugable system. Unfortunately, but not surprisingly, this approach has been met with early stumbles. For example, a recent clinical trial with SER-109, a mix of bacterial spores designed to treat patients with CDI, failed to meet its main goal in a Phase 2 trial. This drug failed to reduce the relative risk of CDI recurrence, compared to a placebo, up to eight weeks after treatment.

Accordingly, there remains a need for effective therapeutics that can restore a healthy intestinal microbiota thereby providing effective treatments for a variety of disorders related to intestinal dysbiosis, e.g., gastrointestinal disorders.

### SUMMARY

Described herein are compositions and methods for protecting the GI microbiome of a subject. In various embodiments, provided herein is a pharmaceutical composition comprising an isolated or purified bacterial isolate and/or a cocktail of isolated or purified bacterial isolates (e.g. from a human, e.g. from stool of a healthy human).

An aspect of the present invention is a method of preparing a therapeutic bacterial isolate, the method comprising: (i) providing a preparation of bacteria from fecal material from (a) a group of healthy subjects, (b) a group of patients with a gastrointestinal disease or disorder, and (c) a group of patients having clinical remission of one or more symptoms of the gastrointestinal disease or disorder following treatment of each patient of the group of patients with a fecal microbiota transplant, (ii) analyzing 16S rRNA sequences from the preparation of bacteria, and (iii) isolating a bacterial isolate from stool of a human donor that comprises a 16S rRNA sequence that is at least 97% identical to a 16S rRNA sequence of a bacterial strain from the preparation of bacteria that is (a) enriched in a group of healthy subjects over a group of patients with the gastrointestinal disease or disorder; and/or (b) correlated with clinical remission of one or more symptoms of the gastrointestinal disease or disorder in a group of patients following treatment of each patient of the group of patients with the fecal microbiota transplant, wherein a cross-sectional combined p-value of the bacterial isolate is less than 1×10⁻¹⁴.

In embodiments, the bacterial strain enriched in a group of healthy subjects over a group of patients with the gastrointestinal disease or disorder is selected from Faecalibacterium prausnitzii, Odoribacter splanchnicus, Anaerostipes hadrus, Alistipes onderdonkii, Faecalibacterium prausnitzii, Eubacterium rectale, Blautia obeum, Bacteroides uniformis, Bacteroides vulgatus, Bacteroides cellulosilyticus, Alistipes finegoldii, Alistipes shahii, Akkermansia muciniphila, Phascolarctobacterium faecium, Subdoligranulum variabile, Subdoligranulum variabile, Blautia sp., Alistipes putredinis, Alistipes putredinis, Alistipes putredinis, and any two or more thereof. In embodiments, the bacterial strain enriched in a group of healthy subjects over a group of patients with the gastrointestinal disease or disorder comprises a 16S rRNA sequence that has at least about 98%, or at least about 99% sequence identity with a nucleotide sequence selected from SEQ ID NOs: 1, 2, 3, 4, 7, 8, 9, 11, 12, 14, 15, 16, 18, 20, 21, 22, 23, 34, 35, 36, and 37.

In embodiments, the bacterial isolate comprises at least one of Odoribacter splanchnicus and Alistipes shahii. In embodiments, the bacterial isolate comprises a 16S rRNA sequence that has at least about 98%, or at least about 99% sequence identity with a nucleotide sequence of SEQ ID NO: 2 and SEQ ID NO: 18.

In embodiments, the bacterial isolate comprises a 16S rRNA sequence that is at least 99% identical to a 16S rRNA sequence of the bacterial strain.

In embodiments, the bacterial strain correlated with clinical remission of one or more symptoms of the gastrointestinal disease or disorder in a group of patients following treatment of each patient of the group of patients with the fecal microbiota transplant comprises: Alistipes finegoldii, but does not comprise Alistipes shahii or Alistipes putredinis, or Alistipes putredinis, but does not comprise Alistipes shahii or Alistipes finegoldii. In embodiments, the bacterial strain correlated with clinical remission of one or more symptoms of the gastrointestinal disease or disorder in a group of patients following treatment of each patient of the group of patients with the fecal microbiota transplant comprises: a 16S rRNA sequence that has at least about 95%, or at least about 97%, or at least about 98%, or at least about 99% sequence identity with a nucleotide sequence of SEQ ID NO: 15, but not a nucleotide sequence selected from SEQ ID NO: 18, 35, 36 or 37; or a 16S rRNA sequence that has at least about 95%, or at least about 97%, or at least about 98%, or at least about 99% sequence identity with the nucleotide sequence selected from SEQ ID NOs: 35-37, but not a nucleotide sequence selected from SEQ ID NO: 15 and 18.

In embodiments, the method further comprises isolating a second therapeutic bacterial isolate by: (iv) providing multiple bacterial isolates from human fecal samples; and (v) performing a functional assay comprising: (a) contacting a population of eukaryotic cells with the human-derived bacterial isolates, (b) measuring a level of a cytokine in the population of eukaryotic cells, and (c) selecting the bacterial isolate capable of modulation of the level of the cytokine in the population of eukaryotic cells. In embodiments, the population of eukaryotic cells comprises a population of PBMCs. In embodiments, the cytokine is selected from IL-10, GM-CSF, IFN-gamma, TNF-alpha, IL-23, and IL-12.

In embodiments, the second bacterial isolate is selected from Faecalibacterium prausnitzii, Odoribacter splanchnicus, Anaerostipes hadrus, Alistipes shahii, Akkermansia muciniphila, Subdoligranulum variabile, Bacteroides uniformis, and any two or more thereof. In embodiments, the second bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 11, 16, 18, 20, 22, and 23.

An aspect of the present invention is a method of preparing a microbial cocktail comprising a first and second bacterial isolate, the method comprising: (i) isolating multiple bacterial isolates from human stool; (ii) performing a first functional assay comprising: (a) contacting a population of eukaryotic cells with the bacterial isolates, (b) measuring an IL-10:IL-12 ratio and an IL-10:TNF-alpha ratio, and (c) identifying as the first bacterial isolate a bacterial isolate capable of inducing an IL-10:IL-12 ratio of at least about 50:1 or an IL-10:TNF-alpha ratio of at least about 1:1 when incubated with a population of eukaryotic cells, (iii) performing a second functional assay comprising: (a) measuring a level of a short chain fatty acid (SCFA) produced by the bacterial isolates, and (b) identifying as the second bacterial isolate a bacterial isolate that produces the SCFA at a concentration of at least about 10 mM, and (iv) combining the first and second bacterial isolates to produce the microbial cocktail.

In embodiments, the first bacterial isolate is selected from Faecalibacterium prausnitzii, Odoribacter splanchnicus, Anaerostipes hadrus, Bacteroides uniformis, Bacteroides vulgatus, Coprococcus comes, Alistipes shahii, Akkermansia muciniphila, Subdoligranulum variabile, and any two or more thereof. In embodiments, the first bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 11, 12, 16, 17, 18, 20, 22 and 23. In embodiments, the population of eukaryotic cells comprises a population of PBMCs.

In embodiments, the SCFA is selected from acetic acid, butyric acid, caproic acid, formic acid, heptanoic acid, isobutyric acid, isocaproic acid, isovaleric acid, propionic acid, valeric acid, and any two or more thereof. In embodiments, the SCFA is butyric acid. In embodiments, the second bacterial isolate is selected from Odoribacter splanchnicus, Eubacterium rectale, Coprococcus comes, Faecalibacterium prausnitzii, Roseburia faecis, Anaerostipes hadrus, Subdogranulum variabile, and any two or more thereof. In embodiments, the second bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 8, 17, 19, 22, and 23.

In embodiments, the method further comprises isolating a third bacterial isolate for incorporation into the microbial cocktail by: (i) analyzing 16S rRNA sequences from preparations of bacteria from fecal material of: (a) a group of healthy subjects, (b) a group of patients with a gastrointestinal disease or disorder, and (c) a group of patients having clinical remission of one or more symptoms of the gastrointestinal disease or disorder following treatment of each patient of the group of patients with a fecal microbiota transplant, and (ii) isolating as the third bacterial isolate a bacterial isolate from stool of a human donor that comprises a 16S rRNA sequence that is at least 97% identical to a 16S rRNA sequence of a bacterial strain from the preparations of bacteria that is (a) enriched in a group of healthy subjects over a group of patients with the gastrointestinal disease or disorder; and/or (b) correlated with clinical remission of one or more symptoms of the gastrointestinal disease or disorder in a group of patients following treatment of each patient of the group of patients with the fecal microbiota transplant.

In embodiments, the third bacterial isolate comprises a 16S rRNA sequence that is at least 99% identical to a 16S rRNA sequence of the bacterial strain.

In any of the embodiments disclosed herein, the gastrointestinal disease or disorder may be selected from inflammatory bowel disease (IBD), irritable bowel syndrome (IBS), C. difficile infection (CDI), C. difficile-associated disease (CDAD), and an antibiotic-induced adverse effect. In embodiments, the IBD is selected from one or more of ulcerative colitis (UC), Crohn's disease (CD), and pouchitis. In embodiments, the gastrointestinal disease or disorder is selected from is selected from inflammatory bowel disease (IBD), irritable bowel syndrome (IBS), C. difficile infection (CDI), C. difficile-associated disease (CDAD), and an antibiotic-induced adverse effect. In embodiments, the IBD is selected from one or more of ulcerative colitis (UC), Crohn's disease (CD), and pouchitis

Disclosed herein is a pharmaceutical composition comprising a cocktail of bacterial isolates, wherein at least one of the bacterial isolates comprises a 16S rRNA sequence that is at least 95% identical to a 16S rRNA sequence of a bacterial isolate provided in Table 1. In embodiments, at least two of the bacterial isolates comprise a 16S rRNA sequence that is at least 95% identical to a 16S rRNA sequence of a bacterial isolate provided in Table 1. In embodiments, at least three of the bacterial isolates comprise a 16S rRNA sequence that is at least 95% identical to a 16S rRNA sequence of a bacterial isolate provided in Table 1. In embodiments, at least four of the bacterial isolates comprise a 16S rRNA sequence that is at least 95% identical to a 16S rRNA sequence of a bacterial isolate provided in Table 1. In embodiments, at least five of the bacterial isolates comprise a 16S rRNA sequence that is at least 95% identical to a 16S rRNA sequence of a bacterial isolate provided in Table 1. In embodiments, at least six of the bacterial isolates comprise a 16S rRNA sequence that is at least 95% identical to a 16S rRNA sequence of a bacterial isolate provided in Table 1. In embodiments, at least seven of the bacterial isolates comprise a 16S rRNA sequence that is at least 95% identical to a 16S rRNA sequence of a bacterial isolate provided in Table 1. In embodiments, at least eight of the bacterial isolates comprise a 16S rRNA sequence that is at least 95% identical to a 16S rRNA sequence of a bacterial isolate provided in Table 1. In embodiments, at least nine of the bacterial isolates comprise a 16S rRNA sequence that is at least 95% identical to a 16S rRNA sequence of a bacterial isolate provided in Table 1. In embodiments, at least ten of the bacterial isolates comprise a 16S rRNA sequence that is at least 95% identical to a 16S rRNA sequence of a bacterial isolate provided in Table 1.

Disclosed herein is a pharmaceutical composition comprising a plurality of bacterial isolates, wherein the plurality of bacterial isolates comprises at least two bacterial isolates selected from the group consisting of Eubacterium rectale, Odoribacter splanchnicus and Subdoligranulum variabile. In embodiments, the Subdoligranulum variabile comprises a 16S rRNA sequence that is at least 95% identical to at least one of SEQ ID NO: 22 or SEQ ID NO: 23. In embodiments, the Odoribacter comprises a 16S rRNA sequence that is at least 95% identical to SEQ ID NO: 2. In embodiments, the Eubacterium rectale comprises a 16S rRNA sequence that is at least 95% identical to SEQ ID NO: 8. In embodiments, the at least two bacterial isolates do not include Eubacterium rectale. In embodiments, the at least two bacterial isolates do not include Odoribacter splanchnicus. In embodiments, the at least two bacterial isolates do not include Subdoligranulum variabile.

In embodiments, the at least two bacterial isolates comprise Odoribacter splanchnicus and Eubacterium rectale. In embodiments, the plurality of bacterial isolates comprises Odoribacter splanchnicus and Eubacterium rectale, and further comprises at least one of Bacteroides cellulosilyticus, Faecalibacterium prausnitzii, Alistipes shahii, and Blautia obeum. In embodiments, the plurality of bacterial isolates comprises Odoribacter splanchnicus and Eubacterium rectale, and further comprises each of Bacteroides cellulosilyticus, Faecalibacterium prausnitzii, Alistipes shahii, and Blautia obeum. In embodiments, the Bacteroides cellulosilyticus comprises a 16S rRNA sequence that is at least 95% identical to SEQ ID NO: 14. In embodiments, the Faecalibacterium prausnitzii comprises a 16S rRNA sequence that is at least 95% identical to at least one of SEQ ID NO: 1 or SEQ ID NO: 7. In embodiments, the Alistipes shahii comprises a 16S rRNA sequence that is at least 95% identical to SEQ ID NO: 18. In embodiments, the Blautia obeum comprises a 16S rRNA sequence that is at least 95% identical to SEQ ID NO: 9.

In embodiments, the plurality of bacterial isolates comprises Odoribacter splanchnicus and Eubacterium rectale, and further comprises at least one of Bacteroides cellulosilyticus, Faecalibacterium prausnitzii, Alistipes shahii, Anaerostipes hadrus, Roseburia faecis, and Blautia obeum. In embodiments, the plurality of bacterial isolates does not comprise at least one of Blautia obeum and Anaerostipes hadrus. In embodiments, the plurality of bacterial isolates comprises Odoribacter splanchnicus and Eubacterium rectale, and further comprises each of Bacteroides cellulosilyticus, Faecalibacterium prausnitzii, Alistipes shahii, Anaerostipes hadrus, and Roseburia faecis In embodiments, the Bacteroides cellulosilyticus comprises a 16S rRNA sequence that is at least 95% identical to SEQ ID NO: 14. In embodiments, the Faecalibacterium prausnitzii comprises a 16S rRNA sequence that is at least 95% identical to at least one of SEQ ID NO: 1 or SEQ ID NO: 7. In embodiments, the Alistipes shahii comprises a 16S rRNA sequence that is at least 95% identical to SEQ ID NO: 18. In embodiments, the Anaerostipes hadrus comprises a 16S rRNA sequence that is at least 95% identical to SEQ ID NO: 3. In embodiments, the Roseburia faecis comprises a 16S rRNA sequence that is at least 95% identical to SEQ ID NO: 19.

In embodiments, the plurality of bacterial isolates comprises Odoribacter splanchnicus and Eubacterium rectale, and further comprises each of Bacteroides cellulosilyticus, Faecalibacterium prausnitzii, Alistipes shahii, Blautia obeum, and Roseburia faecis. In embodiments, the Bacteroides cellulosilyticus comprises a 16S rRNA sequence that is at least 95% identical to SEQ ID NO: 14. In embodiments, the Faecalibacterium prausnitzii comprises a 16S rRNA sequence that is at least 95% identical to at least one of SEQ ID NO: 1 or SEQ ID NO: 7. In embodiments, the Alistipes shahii comprises a 16S rRNA sequence that is at least 95% identical to SEQ ID NO: 18. In embodiments, the Blautia obeum comprises a 16S rRNA sequence that is at least 95% identical to SEQ ID NO: 9. In embodiments, the Roseburia faecis comprises a 16S rRNA sequence that is at least 95% identical to SEQ ID NO: 19.

In embodiments, the plurality of bacterial isolates comprises Odoribacter splanchnicus and Eubacterium rectale, and further comprises at least one of Bacteroides cellulosilyticus, Alistipes shahii, and Roseburia faecis. In embodiments, the plurality of bacterial isolates comprises Odoribacter splanchnicus and Eubacterium rectale, and further comprises each of Bacteroides cellulosilyticus, Alistipes shahii, and Roseburia faecis. In embodiments, the Bacteroides cellulosilyticus comprises a 16S rRNA sequence that is at least 95% identical to SEQ ID NO: 14. In embodiments, the Roseburia faecis comprises a 16S rRNA sequence that is at least 95% identical to SEQ ID NO: 19. In embodiments, the Alistipes shahii comprises a 16S rRNA sequence that is at least 95% identical to SEQ ID NO: 18.

In embodiments, the plurality of bacterial isolates comprises Odoribacter splanchnicus and Eubacterium rectale, and further comprises at least one of Faecalibacterium prausnitzii, Roseburia faecis, and Anaerostipes hadrus. In embodiments, the plurality of bacterial isolates comprises Odoribacter splanchnicus and Eubacterium rectale, and further comprises each of Faecalibacterium prausnitzii, Roseburia faecis, and Anaerostipes hadrus. In embodiments, the Faecalibacterium prausnitzii comprises a 16S rRNA sequence that is at least 95% identical to at least one of SEQ ID NO: 1 or SEQ ID NO: 7. In embodiments, the Roseburia faecis comprises a 16S rRNA sequence that is at least 95% identical to SEQ ID NO: 19. In embodiments, the Anaerostipes hadrus comprises a 16S rRNA sequence that is at least 95% identical to SEQ ID NO: 3.

In embodiments, the plurality of bacterial isolates comprises Odoribacter splanchnicus and Eubacterium rectale, and further comprises at least one of Bacteroides cellulosilyticus, Blautia obeum, and Alistipes shahii. In embodiments, the plurality of bacterial isolates comprises Odoribacter splanchnicus and Eubacterium rectale, and further comprises each of Bacteroides cellulosilyticus, Blautia obeum, and Alistipes shahii. In embodiments, the Bacteroides cellulosilyticus comprises a 16S rRNA sequence that is at least 95% identical to SEQ ID NO: 14. In embodiments, the Blautia obeum comprises a 16S rRNA sequence that is at least 95% identical to SEQ ID NO: 9. In embodiments, the Alistipes shahii comprises a 16S rRNA sequence that is at least 95% identical to SEQ ID NO: 18.

In embodiments, the plurality of bacterial isolates comprises Odoribacter splanchnicus and Eubacterium rectale, and further comprises at least one of Faecalibacterium prausnitzii, Alistipes shahii, and Roseburia faecis. In embodiments, the plurality of bacterial isolates comprises Odoribacter splanchnicus and Eubacterium rectale, and further comprises each of Faecalibacterium prausnitzii, Alistipes shahii, and Roseburia faecis. In embodiments, the Faecalibacterium prausnitzii comprises a 16S rRNA sequence that is at least 95% identical to at least one of SEQ ID NO: 1 or SEQ ID NO: 7. In embodiments, the Alistipes shahii comprises a 16S rRNA sequence that is at least 95% identical to SEQ ID NO: 18. In embodiments, the Roseburia faecis comprises a 16S rRNA sequence that is at least 95% identical to SEQ ID NO: 19.

In embodiments, the plurality of bacterial isolates comprises Odoribacter splanchnicus and Eubacterium rectale, and further comprises at least one of Faecalibacterium prausnitzii, Blautia obeum, and Roseburia faecis. In embodiments, the plurality of bacterial isolates comprises Odoribacter splanchnicus and Eubacterium rectale, and further comprises each of Faecalibacterium prausnitzii, Blautia obeum, and Roseburia faecis. In embodiments, the Faecalibacterium prausnitzii comprises a 16S rRNA sequence that is at least 95% identical to at least one of SEQ ID NO: 1 or SEQ ID NO: 7. In embodiments, the Blautia obeum comprises a 16S rRNA sequence that is at least 95% identical to SEQ ID NO: 9. In embodiments, the Roseburia faecis comprises a 16S rRNA sequence that is at least 95% identical to SEQ ID NO: 19.

In embodiments, the at least two bacterial isolates comprise Eubacterium rectale and Subdoligranulum variabile. In embodiments, the plurality of bacterial isolates comprises Eubacterium rectale and Subdoligranulum variabile, and further comprises at least one of Faecalibacterium prausnitzii, Coprococcus comes, Anaerostipes hadrus, and Roseburia faecis. In embodiments, the plurality of bacterial isolates comprises Eubacterium rectale and Subdoligranulum variabile, and further comprises each of Faecalibacterium prausnitzii, Coprococcus comes, Anaerostipes hadrus, and Roseburia faecis. In embodiments, the Faecalibacterium prausnitzii comprises a 16S rRNA sequence that is at least 95% identical to at least one of SEQ ID NO: 1 or SEQ ID NO: 7. In embodiments, the Anaerostipes hadrus comprises a 16S rRNA sequence that is at least 95% identical to SEQ ID NO: 3. In embodiments, the Roseburia faecis comprises a 16S rRNA sequence that is at least 95% identical to SEQ ID NO: 19. In embodiments, the Coprococcus comes comprises a 16S rRNA sequence that is at least 95% identical to SEQ ID NO: 17.

In embodiments, the plurality of bacterial strains does not include at least one of Faecalibacterium prausnitzii, Roseburia faecis, Bacteroides cellulosilyticus, Alistipes shahii, or Blautia obeum.

Disclosed herein is a pharmaceutical composition comprising a plurality of bacterial isolates, wherein the plurality of bacterial isolates comprises Roseburia faecis and Bacteroides cellulosilyticus, and at least one of Faecalibacterium prausnitzii and Alistipes shahii. In embodiments, the plurality of bacterial isolates further comprises at least one of Eubacterium rectale, Anaerostipes hadrus, and Blautia obeum. In embodiments, the plurality of bacterial isolates does not comprise at least one of Anaerostipes hadrus and Blautia obeum. In embodiments, the plurality of bacterial isolates comprises each of Roseburia faecis, Bacteroides cellulosilyticus, Faecalibacterium prausnitzii, Alistipes shahii, Eubacterium rectale, and Anaerostipes hadrus. In embodiments, the Roseburia faecis comprises a 16S rRNA sequence that is at least 95% identical to SEQ ID NO: 19. In embodiments, the Bacteroides cellulosilyticus comprises a 16S rRNA sequence that is at least 95% identical to SEQ ID NO: 14. In embodiments, the Faecalibacterium prausnitzii comprises a 16S rRNA sequence that is at least 95% identical to at least one of SEQ ID NO: 1 or SEQ ID NO: 7. In embodiments, the Alistipes shahii comprises a 16S rRNA sequence that is at least 95% identical to SEQ ID NO: 18. In embodiments, the Eubacterium rectale comprises a 16S rRNA sequence that is at least 95% identical to SEQ ID NO: 8. In embodiments, the Anaerostipes hadrus comprises a 16S rRNA sequence that is at least 95% identical to SEQ ID NO: 3.

In embodiments, the plurality of bacterial isolates comprises each of Roseburia faecis, Bacteroides cellulosilyticus, Faecalibacterium prausnitzii, Alistipes shahii, Eubacterium rectale, and Blautia obeum. In embodiments, the Roseburia faecis comprises a 16S rRNA sequence that is at least 95% identical to SEQ ID NO: 19. In embodiments, the Bacteroides cellulosilyticus comprises a 16S rRNA sequence that is at least 95% identical to SEQ ID NO: 14. In embodiments, the Faecalibacterium prausnitzii comprises a 16S rRNA sequence that is at least 95% identical to at least one of SEQ ID NO: 1 or SEQ ID NO: 7. In embodiments, the Alistipes shahii comprises a 16S rRNA sequence that is at least 95% identical to SEQ ID NO: 18. In embodiments, the Eubacterium rectale comprises a 16S rRNA sequence that is at least 95% identical to SEQ ID NO: 8. In embodiments, the Blautia obeum comprises a 16S rRNA sequence that is at least 95% identical to SEQ ID NO: 9.

In embodiments, one or more of the plurality of bacterial isolates in the above pharmaceutical compositions secretes a short-chain fatty acid (SCFA) in an intestine of a subject administered the composition. In embodiments, the SCFA is selected from the group consisting of acetic acid, butyric acid, caproic acid, formic acid, heptanoic acid, isobutyric acid, isocaproic acid, isovaleric acid, propionic acid, valeric acid, and a combination thereof. In embodiments, the SCFA is butyric acid. In embodiments, a level of the butyric acid produced by the one or more bacterial isolates over a period of 24 hours is at least 5 mM. In embodiments, a level of the butyric acid produced by the one or more bacterial isolates over a period of 24 hours is at least 10 mM. In embodiments, a level of the butyric acid produced by the one or more bacterial isolates over a period of 24 hours is at least 15 mM. In embodiments, a level of the butyric acid produced by the one or more bacterial isolates over a period of 24 hours is at least 20 mM. In embodiments, a level of the butyric acid produced by the one or more bacterial isolates over a period of 24 hours is at least 25 mM. In embodiments, a level of the butyric acid produced by the one or more bacterial isolates over a period of 24 hours is at least 30 mM. In embodiments, a level of the butyric acid produced by the one or more bacterial isolates over a period of 24 hours is at least 35 mM. In embodiments, a level of the butyric acid produced by the one or more bacterial isolates over a period of 24 hours is at least 40 mM. In embodiments, the one or more bacterial isolates producing at least one SCFA comprises a 16S rRNA sequence that is at least 95% identical to a 16S rRNA sequence of a bacterial isolate provided in Table 2.

Disclosed herein is a pharmaceutical composition comprising a plurality of bacterial isolates, wherein the plurality of bacterial isolates comprises a bacterial isolate comprising Parabacteroides merdae and at least one of Alistipes finegoldii and Alistipes onderdonkii. In embodiments, the plurality of bacterial isolates comprises both Alistipes finegoldii and Alistipes onderdonkii. In embodiments, the plurality of bacterial isolates does not include one of Alistipes finegoldii and Alistipes onderdonkii. In embodiments, the plurality of bacterial isolates further comprises at least one of Akkermansia muciniphila, Dorea longicatena, Blautia obeum, Blautia sp., Bacteroides uniformis or Bacteroides vulgatus. In embodiments, the plurality of bacterial isolates comprises Parabacteroides merdae and each of Akkermansia muciniphila, Alistipes finegoldii, Dorea longicatena, Alistipes onderdonkii, Blautia sp., Bacteroides uniformis and Bacteroides vulgatus. In embodiments, the Parabacteroides merdae comprises a 16S rRNA sequence that is at least 95% identical to SEQ ID NO: 5. In embodiments, the Akkermansia muciniphila comprises a 16S rRNA sequence that is at least 95% identical to SEQ ID NO: 20. In embodiments, the Alistipes finegoldii comprises a 16S rRNA sequence that is at least 95% identical to SEQ ID NO: 15. In embodiments, the Dorea longicatena comprises a 16S rRNA sequence that is at least 95% identical to SEQ ID NO: 6. In embodiments, the Alistipes onderdonkii comprises a 16S rRNA sequence that is at least 95% identical to SEQ ID NO: 4. In embodiments, the Blautia sp. comprises a 16S rRNA sequence that is at least 95% identical to SEQ ID NO: 34. In embodiments, the Bacteroides uniformis comprises a 16S rRNA sequence that is at least 95% identical to at least one of SEQ ID NO: 11 and SEQ ID NO: 16. In embodiments, the Bacteroides vulgatus comprises a 16S rRNA sequence that is at least 95% identical to SEQ ID NO: 12.

In embodiments, the plurality of bacterial isolates comprises Parabacteroides merdae and each of Akkermansia muciniphila, Alistipes finegoldii, Dorea longicatena, Blautia obeum, Blautia sp., Bacteroides uniformis and Bacteroides vulgatus. In embodiments, the Parabacteroides merdae comprises a 16S rRNA sequence that is at least 95% identical to SEQ ID NO: 5. In embodiments, the Akkermansia muciniphila comprises a 16S rRNA sequence that is at least 95% identical to SEQ ID NO: 20. In embodiments, the Alistipes finegoldii comprises a 16S rRNA sequence that is at least 95% identical to SEQ ID NO: 15. In embodiments, the Dorea longicatena comprises a 16S rRNA sequence that is at least 95% identical to SEQ ID NO: 6. In embodiments, the Blautia obeum comprises a 16S rRNA sequence that is at least 95% identical to SEQ ID NO: 9. In embodiments, the Blautia sp. comprises a 16S rRNA sequence that is at least 95% identical to SEQ ID NO: 34. In embodiments, the Bacteroides uniformis comprises a 16S rRNA sequence that is at least 95% identical to at least one of SEQ ID NO: 11 and SEQ ID NO: 16. In embodiments, the Bacteroides vulgatus comprises a 16S rRNA sequence that is at least 95% identical to SEQ ID NO: 12.

Disclosed herein is a pharmaceutical composition comprising a plurality of bacterial isolates, wherein the plurality of bacterial isolates comprises Alistipes finegoldii and at least one of Bacteroides unformis and Dorea longicatena. In embodiments, the plurality of bacterial isolates does not comprise one of Bacteroides uniformis or Dorea longicatena. In embodiments, the plurality of bacterial isolates further comprises at least one of Akkermansia muciniphila, Bacteroides vulgatus, and Blautia sp. In embodiments, the plurality of bacterial isolates comprises each of Alistipes finegoldii, Bacteroides uniformis, Dorea longicatena, Akkermansia muciniphila, Bacteroides vulgatus, and Blautia sp. In embodiments, the Alistipes finegoldii comprises a 16S rRNA sequence that is at least 95% identical to SEQ ID NO: 15. In embodiments, the Bacteroides uniformis comprises a 16S rRNA sequence that is at least 95% identical to SEQ ID NO: 11. In embodiments, the Dorea longicatena comprises a 16S rRNA sequence that is at least 95% identical to SEQ ID NO: 6. In embodiments, the Akkermansia muciniphila comprises a 16S rRNA sequence that is at least 95% identical to SEQ ID NO: 20. In embodiments, the Bacteroides vulgatus comprises a 16S rRNA sequence that is at least 95% identical to SEQ ID NO: 12. In embodiments, the Blautia sp. comprises a 16S rRNA sequence that is at least 95% identical to SEQ ID NO: 34.

In embodiments, the plurality of bacterial isolates of any of the above pharmaceutical compositions comprises at least one bacterial isolate provided in Table 3. In embodiments, the at least one bacterial isolate modulates cytokine production or release by a eukaryotic cell. In embodiments, the at least one bacterial isolate decreases production or release of a pro-inflammatory cytokine by the eukaryotic cell. In embodiments, the pro-inflammatory cytokine is selected from the group consisting of: IFNγ, IL-12p70, IL-1 (e.g., IL-1α, IL-1β), IL-6, IL-8, IL-12, IL-17, IL-18, IL-23, MCP1, MIP1α, MIP1β, TNFα, TNF-γ, and a combination thereof. In embodiments, the at least one bacterial isolate increases production or release of an anti-inflammatory cytokine by the eukaryotic cell. In embodiments, the anti-inflammatory cytokine is selected from the group consisting of: IL-10, IL-13, IL-4, IL-5, TGF-β, and a combination thereof. In embodiments, the anti-inflammatory cytokine is IL-10. In embodiments, the at least one bacterial isolate induces the eukaryotic cell to produce or release at least 500 pg/ml of IL-10. In embodiments, the at least one bacterial isolate induces the eukaryotic cell to produce or release at least 1000 pg/ml of IL-10. In embodiments, the at least one bacterial isolate induces the eukaryotic cell to produce or release at least 1500 pg/ml of IL-10. In embodiments, the at least one bacterial isolate induces the eukaryotic cell to produce or release at least 2000 pg/ml of IL-10. In embodiments, the at least one bacterial isolate induces the eukaryotic cell to produce or release at least 2500 pg/ml of IL-10. In embodiments, the at least one bacterial isolate induces the eukaryotic cell to produce or release at least 3000 pg/ml of IL-10. In embodiments, the eukaryotic cell is a cultured cell. In embodiments, the cultured cell is a peripheral blood mononuclear cell (PBMC). In embodiments, the eukaryotic cell is a cell of a subject administered the composition. In embodiments, the cell of the subject is selected from the group consisting of: an epithelial cell, an intestinal lamina propria cell, an endothelial cell, a fibroblast, a stromal cell, a macrophage, a B lymphocyte, a T lymphocyte, a mast cell, and a peripheral blood mononuclear cell (PBMC). In embodiments, the cell of the subject is an epithelial cell and the epithelial cell is an intestinal epithelial cell.

In embodiments, the pharmaceutical composition is formulated as a capsule for oral administration. In embodiments, the capsule comprises a delayed-release coating. In embodiments, the capsule comprises a hydrophobic coating. In embodiments, the pharmaceutical composition is formulated for delivery of the microbial cocktail to the intestine. In embodiments, the pharmaceutical composition is formulated for delivery of the microbial cocktail to the small intestine. In embodiments, the composition is formulated for delivery of the microbial cocktail to the large intestine. In embodiments, the microbial cocktail is lyophilized. In embodiments, the pharmaceutical composition further comprises at least one of a pharmaceutically acceptable antioxidant, cryoprotectant, lyoprotectant, binder, disintegrant, excipient, filler, preservative, acid suppressant, antacid, H2 antagonist, and/or proton pump inhibitor.

In embodiments, the pharmaceutical composition is for administration to a subject having a disorder related to an intestinal dysbiosis. In embodiments, the disorder is selected from the group consisting of inflammatory bowel disease (IBD), irritable bowel syndrome (IBS), C. difficile infection (CDI), C. difficile-associated disease (CDAD), an antibiotic-induced adverse effect, and a combination thereof.

Disclosed herein is a method of treating or preventing irritable bowel syndrome in a subject in need thereof, comprising administering to the subject a bacterial isolate comprising Bacteroides cellulosilyticus. In embodiments, the Bacteroides cellulosilyticus comprises a 16S rRNA sequence that is at least 95% identical to SEQ ID NO: 26. In embodiments, the method further comprises administering to the subject a bacterial isolate comprising Odoribacter splanchnicus and/or a bacterial isolate comprising Subdoligranulum sp.

Disclosed herein is a method of manufacturing a pharmaceutical composition containing a cocktail of bacterial isolates, the method comprising selecting a first bacterial isolate based on a level of a short-chain fatty acid (SCFA) produced by the first bacterial isolate; selecting a second bacterial isolate based on a relative abundance of a corresponding bacterial strain in a healthy human subject versus a subject having inflammatory bowel disease, wherein the second bacterial isolate comprises a 16S rRNA sequence at least 97% identical to a 16S rRNA sequence of the corresponding bacterial strain; and combining the first and second bacterial isolates to produce the cocktail of bacterial isolates. In embodiments, the first bacterial isolate comprises a 16S rRNA sequence at least 95% identical to a 16S rRNA sequence of a bacterial isolate provided in Table 2. In embodiments, the second bacterial isolate comprises a 16S rRNA sequence at least 95% identical to a 16S rRNA sequence of a bacterial isolate provided in Table 4. In embodiments, the method further comprises selecting a third bacterial isolate based on a modulation of a level of a cytokine by the bacterial isolate. In embodiments, the third bacterial isolate comprises a 16S rRNA sequence at least 95% identical to a 16S rRNA sequence of a bacterial isolate provided in Table 3.

Disclosed herein is a pharmaceutical composition, comprising: a first human-derived bacterial isolate which induces at least one of an IL-10:IL-12 ratio of at least 50 or an IL-10:TNF-alpha ratio of at least 1 when incubated with a population of eukaryotic cells in a first functional assay; and a second human-derived bacterial isolate which produces a short chain fatty acid (SCFA) at a concentration of at least 10 mM as measured by a second functional assay; wherein the first and second bacterial isolates are capable of engrafting into the intestine of a subject following administration of the pharmaceutical composition to the subject.

In an aspect, the IL-10:IL-12 ratio is at least 100. In an aspect, the IL-10:IL-12 ratio is at least 500. In an aspect, the IL-10:IL-12 ratio is at least 1000. In an aspect, the IL-10:IL-12 ratio is at least 2000. In an aspect, the IL-10:TNF-alpha ratio is at least 2. In an aspect, the IL-10:TNF-alpha ratio is at least 5. In an aspect, the IL-10:TNF-alpha ratio is at least 10. In an aspect, the IL-10:TNF-alpha ratio is at least 20. In an aspect, the population of eukaryotic cells comprises a population of PBMCs. In an aspect, the first human-derived bacterial isolate is incubated with the population of eukaryotic cells for about 24 hours. In an aspect, the SCFA is butyrate. In an aspect, the SCFA is produced at a concentration of at least 20 mM. In an aspect, the SCFA is produced at a concentration of at least 25 mM. In an aspect, the SCFA is produced at a concentration of at least 30 mM. In an aspect, the SCFA is produced at a concentration of at least 35 mM. In an aspect, the SCFA is butyrate.

In an aspect, the second functional assay comprises incubating the second bacterial isolate with a substrate. In an aspect, the substrate comprises at least one of an oligosaccharide, sunfiber, or barley malt. In an aspect, the oligosaccharide comprises at least one of a fructooligosaccharide (FOS) and an xylooligosaccharide (XOS). In an aspect, the substrate comprises both of a fructooligosaccharide (FOS) and an xylooligosaccharide (XOS). In an aspect, the second bacterial isolate comprises a 16S rRNA sequence at least 95% identical to a 16S rRNA sequence of a bacterial isolate provided in Table 2. In an aspect, the second bacterial isolate comprises Anaerostipes sp. In an aspect, the Anaerostipes sp. is Anaerostipes hadrus. In an aspect, the second bacterial isolate comprises a 16S rRNA sequence at least 95% identical to the sequence corresponding to SEQ ID NO: 3. In an aspect, the second bacterial isolate comprises Roseburia sp. In an aspect, the Roseburia sp. is Roseburia faecis. In an aspect, the second bacterial isolate comprises a 16S rRNA sequence at least 95% identical to the sequence corresponding to SEQ ID NO: 19. In an aspect, the second bacterial isolate comprises Eubacterium sp. In an aspect, the Eubacterium sp. is Eubacterium rectale. In an aspect, the second bacterial isolate comprises a 16S rRNA sequence at least 95% identical to the sequence corresponding to SEQ ID NO: 8. In an aspect, the second bacterial isolate comprises Coprococcus sp. In an aspect, the Coprococcus sp. is Coprococcus comes. In an aspect, the second bacterial isolate comprises a 16S rRNA sequence at least 95% identical to the sequence corresponding to SEQ ID NO: 17. In an aspect, the first bacterial isolate comprises a 16S sequence at least 95% identical to a 16S rRNA sequence of a bacterial isolate provided in Table 3.

Disclosed herein is a method of treating inflammatory bowel disease comprising administering the pharmaceutical composition comprising the first and second human-derived bacterial isolates to a subject in need thereof. In an aspect, the dosage of at least one of the first and second bacterial isolates is less than 10¹⁰ cells/ml.

In an aspect, there is provided a pharmaceutical composition comprising a plurality of bacterial isolates, wherein the plurality of bacterial isolates comprises Bacteroides cellulosilyticus and at least one of Odoribacter splanchnicus, Roseburia faecis, Faecalibacterium prausnitzii, Akkermansia muciniphila, Alistipes shahii, Subdoligranulum variabile, and Eubacterium rectale, wherein at least two of the plurality of bacterial isolates are isolated from stool samples of different donors.

In embodiments, the plurality of bacterial isolates comprises Odoribacter splanchnicus. In embodiments, the plurality of bacterial isolates comprises Faecalibacterium prausnitzii. In embodiments, the plurality of bacterial isolates comprises two different bacterial isolates that are each members of the species Faecalibacterium prausnitzii. In embodiments, the plurality of bacterial isolates comprises Subdoligranulum variabile. In embodiments, the plurality of bacterial isolates comprises Roseburia faecis, Akkermansia muciniphila, Alistipes shahii, and Eubacterium rectale. In embodiments, the plurality of bacterial isolates comprises Roseburia faecis, Akkermansia muciniphila, Alistipes shahii, and Eubacterium rectale.

In an aspect, there is provided a pharmaceutical composition comprising a plurality of bacterial isolates, wherein the plurality of bacterial isolates comprises two different bacterial isolates that are each members of the species Faecalibacterium prausnitzii.

In embodiments, the two different bacterial isolates are isolated from stool samples of different human donors. In embodiments, the plurality of bacterial isolates further comprises at least one of Bacteroides cellulosilyticus, Odoribacter splanchnicus, Roseburia faecis, Akkermansia muciniphila, Alistipes shahii, and Eubacterium rectale. In embodiments, the plurality of bacterial isolates further comprises each of Bacteroides cellulosilyticus, Odoribacter splanchnicus, Roseburia faecis, Akkermansia muciniphila, Alistipes shahii, and Eubacterium rectale.

In an aspect, there is provided a method of treating or preventing irritable bowel syndrome in a subject in need thereof, comprising administering to the subject a plurality of bacterial isolates, wherein one of the bacterial isolates comprises Bacteroides cellulosilyticus, and wherein at least two of the plurality of bacterial isolates are administered to the subject in different pharmaceutical compositions.

In embodiments, the plurality of bacterial isolates further comprises at least one of Faecalibacterium prausnitzii, Odoribacter splanchnicus, Roseburia faecis, Akkermansia muciniphila, Alistipes shahii, Subdoligranulum variabile and Eubacterium rectale. In embodiments, the plurality of bacterial isolates further comprises each of Faecalibacterium prausnitzii, Odoribacter splanchnicus, Roseburia faecis, Akkermansia muciniphila, Alistipes shahii, Subdoligranulum variabile and Eubacterium rectale. In embodiments, the plurality of bacterial isolates further comprises each of Odoribacter splanchnicus, Roseburia faecis, Akkermansia muciniphila, Alistipes shahii, Eubacterium rectale, and two different bacterial isolates that are each members of the species Faecalibacterium prausnitzii.

In an aspect, there is provided a method of manufacture, the method comprising culturing Bacteroides cellulosilyticus as a pure culture; and lyophilizing bacteria from the pure culture of B. cellulosilyticus to produce a B. cellulosilyticus lyophilate.

In embodiments, the method further comprises combining the B. cellulosilyticus lyophilate with a second lyophilate, wherein the second lyophilate is produced by lyophilizing bacteria from a pure culture of at least one of Faecalibacterium prausnitzii, Odoribacter splanchnicus, Roseburia faecis, Akkermansia muciniphila, Alistipes shahii, Subdoligranulum variabile and Eubacterium rectale.

In an aspect, there is provided a pharmaceutical composition comprising a plurality of bacterial isolates, wherein an amount of cells of a first bacterial isolate of the plurality of bacterial isolates is at least 1% greater than an amount of cells of a second bacterial isolate of the plurality of bacterial isolates, wherein the plurality of bacterial isolates are selected from the group consisting of: Bacteroides cellulosilyticus, Faecalibacterium prausnitzii, Subdogranulum variabile, Eubacterium rectale, Odoribacter splanchnicus, Alistipes shahii, and Akkermansia muciniphila.

In embodiments, the amount of cells of the first bacterial isolate is at least 10% greater than the amount of cells of the second bacterial isolate. In embodiments, the first bacterial isolate comprises one of Eubacterium rectale and Faecalibacterium prausnitzii. In embodiments, the second bacterial isolate comprises one of Alistipes shahii, and Akkermansia muciniphila. In embodiments, the first and second bacterial isolates comprises Faecalibacterium prausnitzii. In embodiments, the first bacterial isolate comprises a 16S rRNA sequence that is at least 95% identical to SEQ ID NO: 7. In embodiments, the second bacterial isolate comprises a 16S rRNA sequence that is at least 95% identical to SEQ ID NO: 1.

In an aspect, there is provided a pharmaceutical composition comprising a plurality of bacterial isolates, wherein an amount of cells of a first bacterial isolate of the plurality of bacterial isolates is at least 1% greater than an amount of cells of a second bacterial isolate of the plurality of bacterial isolates, wherein each of the first and second bacterial isolates comprises Faecalibacterium prausnitzii.

In embodiments, the amount of cells of the first bacterial isolate is at least 10% greater than the amount of cells of the second bacterial isolate. In embodiments, the first bacterial isolate comprises a 16S rRNA sequence that is at least 95% identical to SEQ ID NO: 7. In embodiments, the second bacterial isolate comprises a 16S rRNA sequence that is at least 95% identical to SEQ ID NO: 1.

In an aspect, there is provided a pharmaceutical composition comprising a bacterial isolate, wherein the bacterial isolate comprises a 16S rRNA sequence that is at least 97% identical to a 16S rRNA sequence of a bacterial strain that is either (i) enriched in a group of healthy subjects over a group of patients with ulcerative colitis (UC) and/or (ii) correlated with clinical remission of one or more UC symptoms in a group of patients following treatment of each patient of the group of patients with a fecal microbiota transplant, wherein a cross-sectional combined p-value of the bacterial strain is less than 1×10⁻¹⁰.

In embodiments, the bacterial isolate comprises a 16S rRNA sequence that is at least 99% identical to a 16S rRNA sequence of the bacterial strain. In embodiments, the bacterial isolate comprises at least one of Odoribacter splanchnicus, Eubacterium rectale, Bacteroides cellulosilyticus and Alistipes shahii. In embodiments, the cross-sectional combined p-value of the bacterial strain is less than 1×10⁻¹⁴. In embodiments, the bacterial isolate comprises at least one of Odoribacter splanchnicus and Alistipes shahii. In embodiments, the cross-sectional combined p-value of the bacterial strain is less than 1×10⁻²⁰. In embodiments, the bacterial isolate comprises Alistipes shahii. In embodiments, the the Alistipes shahii comprises a 16S rRNA sequence that is at least 97% identical to SEQ ID NO: 18.

In aspect, there is provided a method of treating inflammatory bowel disease comprising administering the pharmaceutical composition described herein to a subject in need thereof.

Any aspect or embodiment described herein can be combined with any other aspect or embodiment as disclosed herein.

### BRIEF DESCRIPTION OF THE FIGURES

FIG. 1A is a graph showing the results of a short-chain fatty acid (SCFA) quantification assay measuring butyrate concentration (in mM) secreted by various bacterial isolates after a twenty-four incubation in substrate buffer. FIG. 1B shows the percentage of converted butyrate normalized to carbon count.

FIG. 2 shows the relationship between the dosage of bacterial strains corresponding to various bacterial isolates and engraftment of the strains following the administration of uncultured fecal bacteria containing the strains to a subject.

FIG. 3A-E shows the anti-inflammatory effects of an 8-strain bacterial cocktail when incubated with PBMCs treated with inflammation-inducing E. coli on IL-23 (FIG. 3A), TNF-α (FIG. 3B), IL-10 (FIG. 3C), IFN-γ (FIG. 3D) and IL-12p70 (FIG. 3E).

FIG. 4A-E shows the anti-inflammatory effects of a 7-strain bacterial cocktail when incubated with PBMCs treated with inflammation-inducing E. coli on IL-23 (FIG. 4A), TNF-α (FIG. 4B), IL-10 (FIG. 4C), IFN-γ (FIG. 4D) and IL-12p70 (FIG. 4E).

FIG. 5 shows the engraftment of four bacterial isolates following administration to germ free mice of a pharmaceutical composition comprising the isolates.

### DETAILED DESCRIPTION

Described herein are bacterial isolates and cocktails of bacterial isolates that can be utilized for the effective prevention and/or treatment of various disorders related to an intestinal dysbiosis.

Unless defined otherwise herein, terms are to be understood according to conventional usage by those of ordinary skill in the relevant art.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a percentage, density, volume and the like, is meant to encompass variations of 20%, 19%, 18%, 17%, 16%, 15%, 14%, 13%, 12%, 11% 10%, 9%, 8%, 7%, 6%, 5%, 4%, 3%, 2%, 1%, 0.5%, or even 0.1% of the specified amount.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 34%, between 4-5%, or greater than 5%.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, “microbiota,” and “flora” refer to a community of microbes that live in or on a subject's body, both sustainably and transiently, including eukaryotes, archaea, bacteria, and viruses (including bacterial viruses (i.e., phage)). A non-selected fecal microbiota refers to a community or mixture of fecal microbes derived from a donor's fecal sample without selection and substantially resembling microbial constituents and population structure found in such fecal sample.

As used herein, a “sterile fecal filtrate” or a “non-cellular fecal filtrate” refers to a liquid component of a fecal material, where the liquid component is free or substantially free of cell-based living organisms (e.g., bacteria, fungi, or their spores), but retains bacteriophages and non-cellular biological materials. In embodiments, a non-cellular or sterile fecal filtrate is also free of viruses for eukaryotic host cells.

As used herein, “eukaryotic” refers to belonging to a cell that contains a nucleus and membrane-bound organelles.

As used herein, “bacteria,” “bacterium,” and “archaea” refer to single-celled prokaryotes that lack membrane bound nuclei and organelles.

As used herein, “colony forming units” (cfu) refers to an estimate of the number of viable microorganism cells in a given sample.

As used herein, “viable” means possessing the ability to multiply. In one embodiment, a bacterial spore is viable. In another embodiment, a vegetative bacterial cell is viable.

As used herein, “fecal bacteria” refers to bacteria that can be found in fecal matter.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, a “combination” of two or more bacteria includes the physical co-existence of the two bacteria, either in the same material or product or in physically connected products, as well as the temporal co-administration or co-localization of the two bacteria.

As used herein, a “bacterial isolate” refers to a population of substantially genetically identical bacterial cells generated by proliferation via binary fission from a single predecessor bacterial cell (e.g., by culturing the bacteria). Typically, a bacterial isolate is originally isolated as a genetically pure cell or population of cells, for example, as a single colony on solid culture media or via serial dilutions in liquid culture, and thereafter archived (e.g. as a frozen stock) to provide a consistent and stable source for the isolate. Once isolated, in some embodiments, bacterial isolate can be grown as a pure population of cells; in other embodiments, multiple isolates of bacteria can be grown simultaneously in the same vessel as a mixed culture. The term “substantially genetically identical” refers to the very high (>99%) genetic identity shared by different cells in uncontaminated mixtures, owing to their proliferation from a common predecessor, but accounts for minor genetic dissimilarity within the population due to accumulations of relatively rare mutations. Generally, a bacterial isolate is synonymous with a cultured population of cells. Typically, a bacterial isolate consists of non-pathogenic bacteria.

As used herein, the term “microbial cocktail”, sometimes called a “microbial consortium” or “synthetic bacterial mixture” or “bacterial mixture”, refers to an engineered composition (e.g. pharmaceutical composition) comprising a defined consortium of multiple bacterial isolates. The term “defined consortium of multiple bacterial isolates” means that the microbial cocktail contains two or more bacterial isolates, and that the number and identity of each bacterial isolate in the cocktail is known, and thus the cocktail can be consistently produced (e.g. by combining isolated bacterial strains) as a pharmaceutical composition having stable properties across separate batches. Herein “identity” of a bacterial isolate can refer to any characteristic of the isolate that uniquely identifies the isolate as different from one or more other isolates or bacterial strains. Examples of identifying characteristics include DNA sequences such as 16S rRNA sequence, the sequence of one or more coding or non-coding regions and entire genome sequences, levels of gene expression, physiological or metabolic traits, or anatomical traits such as staining pattern or cell wall characteristics.

A microbial cocktail or consortium described herein (e.g. derived from bacterial strains of fecal origin) can be distinguished from a composition (e.g. pharmaceutical composition) comprising an “uncultured fecal microbiota”, which refers to a mixture of multiple bacterial strains that have been at least partially extracted or purified from a stool sample without culturing the strains in culture medium. Steps taken to extract a microbiota or population of fecal bacteria from a stool sample can include, for example, homogenization and filtering of the stool sample to separate the fecal bacterial strains from non-cellular stool material such as fiber and rough particulate matter, as well as, for example, eukaryotic host cells and viruses. Preparation of a pharmaceutical composition comprising an uncultured fecal microbiota can in some embodiments involve removal of certain types (e.g. species) of bacteria from the microbiota and/or addition of one or more bacterial strains to the microbiota. In certain embodiments, a pharmaceutical composition can comprise one or more cultured bacterial strains (e.g. bacterial isolates) combined with an uncultured fecal microbiota.

Herein “uncultured fecal bacteria” or a “preparation of uncultured fecal bacteria” refer to a preparation comprising multiple non-pathogenic viable bacterial strains that have been harvested, extracted or purified from one or more stool samples, without culturing the strains (e.g. in culturing medium). Such a preparation of uncultured fecal bacteria can also be referred to as a collection of uncultured fecal bacteria or a population of uncultured fecal bacteria. In certain embodiments, an uncultured fecal microbiota comprises a preparation of uncultured fecal bacteria.

The present disclosure contemplates compositions (e.g. pharmaceutical compositions) that comprise both a bacterial isolate (e.g., single microbial isolate or microbial cocktail) and an uncultured fecal microbiota or preparation of uncultured fecal bacteria. For example, in certain embodiments, a composition can comprise a substantially complete fecal microbiota or a preparation of uncultured fecal bacteria extracted or purified from a stool sample of a healthy individual supplemented or “spiked” with one or more bacterial isolates.

Herein the terms “microbial mixture” and “microbial therapeutic” are meant to broadly encompass any composition or treatment that incorporates a bacterial isolate, microbial cocktail, preparation of uncultured fecal bacteria and/or uncultured fecal microbiota.

Herein “at least 95% identical”, when used with reference to a 16S rRNA sequence, refers to a subject DNA sequence that shares identity to a reference DNA sequence of at least 95%, at least 95.5%, at least 96%, at least 96.5%, at least 97%, at least 97.5%, at least 98%, at least 98.5%, at least 99%, at least 99.5% or 100%.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. Preferred subjects are human subjects. The human subject may be a pediatric, adult or a geriatric subject. In some embodiments, the terms “patient” and “subject” are used interchangeably.

As used herein, “Shannon Diversity Index” refers to a diversity index that accounts for abundance and evenness of species present in a given community using the formula

$$H = - {\sum }_{i = 1}^{R} {p}_{i} ln {p}_{i}$$

where H is Shannon Diversity Index, R is the total number of species in the community, and pi is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

As used herein, an “intermittent dosing schedule” refers to a dosing schedule where a pharmaceutical composition is administered for a period of time (initial treatment period) which is then followed by a second period of time where treatment with such pharmaceutical composition is withheld (a rest period). The rest period can optionally be followed by a third period wherein the treatment is again administered (either at the same or different dosage as in the initial treatment period), which can be followed by further rest and treatment periods as needed. Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day, as the case may be.

As used herein, a “continuous dosing schedule” refers to a dosing schedule where a pharmaceutical composition is administered during a treatment period without a rest period. Throughout the treatment period of a continuous dosing schedule, a pharmaceutical composition can be administered, for example, daily, or every other day, or every third day. On a day when a pharmaceutical composition is administered, it can be administered in a single dose, or in multiple doses throughout the day.

As used herein, “dosing frequency” refers to the frequency of administering doses of a pharmaceutical composition in a given time. Dosing frequency can be indicated as the number of doses per a given time, for example, once per day, once a week, or once in two weeks.

As used herein, “dosing interval” refers to the amount of time that elapses between consecutive doses of a pharmaceutical composition being administered to a subject.

As used herein, “a disorder related to an intestinal dysbiosis” or “a disorder related to an GI dysbiosis” or “dysbiosis” refers to a disorder or disease caused by an atypical or unhealthy microbiome, e.g., which comprises certain undesirable bacterial strains and/or lacks certain desirable bacterial strains. Examples include but are not limited to inflammatory bowel disease (IBD), irritable bowel syndrome (IBS), C. difficile infection (CDI), C. difficile-associated disease (CDAD), and antibiotic-induced adverse effect. Examples of IBD include ulcerative colitis (UC), Crohn's disease (CD), and pouchitis.

In one aspect, the subject has been diagnosed with a disorder related to an intestinal dysbiosis. In another aspect, a subject being treated is at risk for or is predisposed to having a disorder related to an intestinal dysbiosis is to be prevented. In aspects, a subject being treated is a subject in which a disorder related to an intestinal dysbiosis is to be prevented.

### Bacterial Isolates and Microbial Cocktails

Described herein are pharmaceutical compositions, formulations, methods of manufacture, and uses of bacterial isolates and microbial cocktails of bacterial isolates in the treatment of various disorders related to an intestinal dysbiosis, e.g., gastrointestinal disorders.

In aspects, a pharmaceutical composition comprises one or more bacterial isolates that comprised a 16S rRNA sequence at least 95% identical (e.g., at least 95% identical, at least 95.5% identical, at least 96% identical, at least 96.5% identical, at least 97% identical, at least 97.5% identical, at least 98% identical, at least 98.5% identical, at least 99% identical, at least 99.5% identical, or 100% identical) to the 16S rRNA sequence of one of the bacterial isolates provided in Table 1. In embodiments, the composition comprises a single bacterial isolate. In embodiments, the composition is a microbial cocktail that comprises at least two bacterial isolates, at least three bacterial isolates, at least four bacterial isolates, at least five bacterial isolates, at least six bacterial isolates, at least seven bacterial isolates, at least eight bacterial isolates, at least nine bacterial isolates, at least ten bacterial isolates, or a greater number of bacterial isolates, e.g., fifteen, twenty, twenty-five, thirty, or more bacterial isolates. In embodiments, each of the, for example, three, four, five, six, seven, eight, nine, or ten bacterial isolates comprises a 16S rRNA sequence that is at least 95% identical to the 16S rRNA sequence of one of the bacterial isolates provided in Table 1.

In one aspect, a pharmaceutical composition administered herein comprises fecal bacteria. In one aspect, a pharmaceutical composition administered herein comprises one or more bacterial isolates extracted, isolated and/or cultured from a stool sample of a healthy human donor. In one aspect, a pharmaceutical composition administered herein comprises one or more, two or more, three or more, four or more, or five or more isolated, purified, or cultured microorganisms selected from the group consisting of Akkermansia, Alistipes, Anaerostipes, Bacillus, Bacteroides, Blautia, Clostridium, Collinsella, Coprococcus, Dorea, Eubacterium, Faecalibacterium, Fusobacterium, Odoribacter, Parabacteroides, Phascolarctobacterium, Propionibacterium, Roseburia, Subdoligranulum, Lactobacillus, Ruminococcus, Escherichia, Gemmiger, Desulfomonas, Peptostreptococcus, and a combination thereof.

In one aspect, a pharmaceutical composition administered herein comprises at least one, at least two, at least three, at least four, at least five, at least six, or at least seven, at least eight, at least nine, at least ten, at least eleven, or at least twelve fecal microorganisms (e.g., bacterial isolates) selected from the group consisting of a Faecalibacterium prausnitzii, Odoribacter splanchnicus, Anaerostipes hadrus, Alistipes onderdonkii, Alistipes putredinis, Parabacteroides merdae, Dorea longicatena, Eubacterium rectale, Blautia obeum, Blautia sp., Bacteroides uniformis, Bacteroides vulgatus, Bacteroides cellulosilyticus, Alistipes finegoldii, Coprococcus comes, Alistipes shahii, Roseburia faecis, Akkermansia muciniphila, Phascolarctobacterium faecium, Subdoligranulum variabile, and a combination thereof. In embodiments, a bacterial isolate comprises Odoribacter splanchnicus. In embodiments, a bacterial isolate comprising Odoribacter sp. comprises Odoribacter splanchnicus. In embodiments, a bacterial isolate comprising Odoribacter sp. comprises Odoribacter laneus. In embodiments, a bacterial isolate comprising Blautia sp. comprises Blautia obeum. In embodiments, a bacterial isolate comprising Blautia sp. comprises Blautia massiliensis. In embodiments, a bacterial isolate comprising Blautia sp. comprises Blautia coccoides. In embodiments, a bacterial isolate comprising Blautia sp. comprises Blautia producta. In embodiments, a bacterial isolate comprising Blautia sp. comprises Blautia schinkii. In embodiments, a bacterial isolate comprising Blautia sp. comprises Blautia hydrogenotrophica. In embodiments, a bacterial isolate comprising Blautia sp. comprises Blautia luti. In embodiments, a bacterial isolate comprising Blautia sp. comprises Blautia hansenii. In embodiments, a bacterial isolate comprising Blautia sp. comprises Blautia faecis. In embodiments, a bacterial isolate comprising Blautia sp. comprises Blautia stercoris. In embodiments, a bacterial isolate comprising Blautia sp. comprises Blautia wexlerae.

In one aspect, a pharmaceutical composition administered herein comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof. In another aspect, a pharmaceutical composition administered herein comprises no viable Bacteroides fragilis sp. vulgatus, Collinsella aerofaciens, Bacteroides fragilis sp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis sp. A, Eubacterium biforme, Bifidobacterium infantis, Eubacterium rectale III-F, Coprococcus comes, Pseudoflavonifractor capillosus, Ruminococcus albus, Dorea formicigenerans, Eubacterium halihi, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis sp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis sp. ovatus, -sp. d, -sp. f, Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, —CC; Eubacterium tenue, Eubacterium ramulus, Bacteroides clostridiiformis sp. clostridliformis, Bacteroides coagulans, Prevotella oralis, Prevotella ruminicola, Odoribacter sp. (e.g. Odoribacter splanchnicus and/or Odoriacter laneus), Desuifomonas pigra, Lactobacillus G, Succinivibrio A, or a combination thereof.

In various embodiments, the bacterial isolates described herein comprise bacteria isolated or purified from a human. In various embodiments, all or a subset of bacterial isolates incorporated into a microbial cocktail described herein are isolated or purified from a human. For instance, one or more bacterial isolates can be purified or isolated from a stool sample of a one or more healthy human donor pre-screened for infectious agents. In other examples, a bacterial isolate can be isolated or purified from aspirates of the fluid in the GI tract or mucosal biopsies from a site in the GI tract.

In embodiments, a pharmaceutical composition or microbial cocktail comprises a plurality of bacterial isolates isolated or purified from stool samples of multiple human donors. In embodiments, a pharmaceutical composition or microbial cocktail comprises a plurality of bacterial isolates isolated or purified from a stool sample or stool samples of only a single human donor.

In some embodiments, a bacterial isolate incorporated into a pharmaceutical composition described herein comprises live, vegetative cells. In some embodiments, the bacterial isolate comprises bacteria capable of forming spores. In some embodiments, the bacterial isolate comprises bacteria in the form of spores, e.g. viable spores. In some embodiments, the bacterial isolate comprises bacteria in the form of live, vegetative cells and spores. In some embodiments, a bacterial isolate is substantially free of live, vegetative cells. In some embodiments, an entire microbial cocktail is substantially free of live vegetative cells. In some embodiments, a bacterial isolate is substantially free of spores. In some embodiments, an entire microbial cocktail is substantially free of spores.

In aspects, a pharmaceutical composition comprises at least one bacterial isolate provided in Table 1, or a bacterial isolate comprising a 165 rRNA sequence that is at least 95% identical to the 16S rRNA sequence ofone or more ofthe bacterial isolates provided in Table 1. In certain embodiments, a pharmaceutical composition comprises a microbial cocktail comprising at least two bacterial isolates provided in Table 1, or at least two bacterial isolates comprising a 165 rRNA sequence that is at least 95% identical to the 165 rRNA sequence of one or more ofthe bacterial isolates provided in Table 1. Each bacterial isolate in Table 1 is identified by Latin name, an Identification Number (ID number), and the Sequence Identifier (SEQ ID NO) for its 16S rRNA sequence.

TABLE 1

| | | SEQ ID NO for 16S |
|---|---|---|
| Isolate Latin Name | ID Number | rRNA Sequence |
| Faecalibacterium prausnitzii | PI00000329 | 1 |
| Odoribacter splanchnicus | PI00000072 | 2 |
| Anaerostipes hadrus | PI00000094 | 3 |
| Alistipes onderdonkii | IS00004389 | 4 |
| Parabacteroides merdae | IS00006167 | 5 |
| Dorea longicatena | IS00006618 | 6 |
| Faecalibacterium prausnitzii | IS00006632 | 7 |
| Eubacterium rectale | IS00006864 | 8 |
| Blautia obeum | PI00000053 | 9 |
| Bacteroides uniformis | PI00000137 | 11 |
| Bacteroides vulgatus | PI00000138 | 12 |
| Bacteroides cellulosilyticus | PI00000316 | 14 |
| Alistipes finegoldii | PI00000340 | 15 |
| Bacteroides uniformis | PI00000352 | 16 |
| Coprococcus comes | PI00000370 | 17 |
| Alistipes shahii | PI00000395 | 18 |
| Roseburia faecis | PI00000404 | 19 |
| Akkermansia muciniphila | IS00007180 | 20 |
| Phascolarctobacterium faecium | PI00000289 | 21 |
| Subdoligranulum variabile | IS00007359 | 22 |
| Subdoligranulum variabile | IS00007357 | 23 |
| Blautia sp. | IS00002788 | 34 |
| Alistipes putredinis | IS00008139 | 35 |
| Alistipes putredinis | IS00008142 | 36 |
| Alistipes putredinis | IS00008177 | 37 |

In embodiments, the pharmaceutical composition comprises a microbial cocktail comprising about 2 to about 50 bacterial isolates, about 3 to about 50 bacterial isolates, about 3 to about 45 bacterial isolates, about 3 to about 40 bacterial isolates, about 3 to about 35 bacterial isolates, about 3 to about 30 bacterial isolates, about 3 to about 20 bacterial isolates, about 3 to about 15 bacterial isolates, about 3 to about 10 bacterial isolates, and about 3 to about 9 bacterial isolates. In embodiments, the pharmaceutical composition comprises a microbial cocktail comprising about 30 bacterial isolates, or about 29 bacterial isolates, or about 28 bacterial isolates, or about 27 bacterial isolates, or about 26 bacterial isolates, or about 25 bacterial isolates, or about 24 bacterial isolates, or about 23 bacterial isolates, or about 22 bacterial isolates, or about 21 bacterial isolates, or about 19 bacterial isolates, or about 18 bacterial isolates, or about 17 bacterial isolates, or about 16 bacterial isolates, or about 15 bacterial isolates, or about 14 bacterial isolates, or about 13 bacterial isolates, or about 12 bacterial isolates, or about 11 bacterial isolates. In embodiments, the pharmaceutical composition comprises a microbial cocktail comprising 10 bacterial isolates. In embodiments, the pharmaceutical composition comprises a microbial cocktail comprising 9 bacterial isolates. In embodiments, the pharmaceutical composition comprises a microbial cocktail comprising 8 bacterial isolates. In embodiments, the pharmaceutical composition comprises a microbial cocktail comprising 7 bacterial isolates. In embodiments, the pharmaceutical composition comprises a microbial cocktail comprising 6 bacterial isolates. In embodiments, the pharmaceutical composition comprises a microbial cocktail comprising 5 bacterial isolates. In embodiments, the pharmaceutical composition comprises a microbial cocktail comprising 4 bacterial isolates. In embodiments, the pharmaceutical composition comprises a microbial cocktail comprising 3 bacterial isolates. In embodiments, the pharmaceutical composition comprises a microbial cocktail comprising 2 bacterial isolates.

In embodiments, the microbial cocktail comprises at least two bacterial isolates, at least three bacterial isolates, at least four bacterial isolates, at least five bacterial isolates, at least six bacterial isolates, at least seven bacterial isolates, at least eight bacterial isolates, at least nine bacterial isolates, at least ten bacterial isolates, or a greater number of bacterial isolates, e.g., fifteen, twenty, twenty-five, thirty, or more bacterial isolates. In embodiments, each of the three, four, five, six, seven, eight, nine, or ten bacterial isolates comprises a 16S rRNA sequence that is at least 95% identical to the 16S rRNA sequence of at least one of the bacterial isolates provided in Table 1.

In embodiments, the pharmaceutical composition comprises a microbial cocktail comprising about 6 to about 20 bacterial isolates provided in Table 1, or having a 16S rRNA sequence that is at least 95% identical to the 16S rRNA sequence of at least one of the bacterial isolates provided in Table 1, or e.g., about 6 to about 15 bacterial isolates, about 6 to about 10 bacterial isolates, about 6 to about 9 bacterial isolates. In embodiments, the pharmaceutical composition comprises a microbial cocktail comprising 30 bacterial isolates, or 29 bacterial isolates, or 28 bacterial isolates, or 27 bacterial isolates, or 26 bacterial isolates, or 25 bacterial isolates, or 24 bacterial isolates, or 23 bacterial isolates, or 22 bacterial isolates, or 21 bacterial isolates, or 20 bacterial isolates, or 19 bacterial isolates, or 18 bacterial isolates, or 17 bacterial isolates, or 16 bacterial isolates, or 15 bacterial isolates, or 14 bacterial isolates, or 13 bacterial isolates, or 12 bacterial isolates, or 11 bacterial isolates, or 10 bacterial isolates, or 9 bacterial isolates, or 8 bacterial isolates, or 7 bacterial isolates, or 6 bacterial isolates, or 5 bacterial isolates, or 4 bacterial isolates, or 3 bacterial isolates, or 2 bacterial isolates, or 1 bacterial isolate provided in Table 1, or having a 16S rRNA sequence that is at least 95% identical to a 16S rRNA sequence of one or more bacterial isolates provided in Table 1. In embodiments, the pharmaceutical composition comprises a microbial cocktail comprising 8 bacterial isolates provided in Table 1, or having a 16S rRNA sequence that is at least 95% identical to a 16S rRNA sequence of one or more bacterial isolates provided in Table 1. In embodiments, the pharmaceutical composition comprises a microbial cocktail comprising 7 bacterial isolates provided in Table 1, or having a 16S rRNA sequence that is at least 95% identical to a 16S rRNA sequence of one or more bacterial isolates provided in Table 1. In embodiments, the pharmaceutical composition comprises a microbial cocktail comprising 6 bacterial isolates provided in Table 1, or having a 16S rRNA sequence that is at least 95% identical to a 16S rRNA sequence of one or more bacterial isolates provided in Table 1. In embodiments, the pharmaceutical composition comprises one bacterial isolate provided in Table 1, or having a 16S rRNA sequence that is at least 95% identical to a 16S rRNA sequence of one or more bacterial isolates provided in Table 1.

In embodiments, a bacterial isolate incorporated into a pharmaceutical composition described herein is capable of improving the health of a subject administered the composition. In embodiments, the bacterial isolate impacts the health of a subject by inducing or influencing one or more biological mechanisms that act in the subject to affect the health of the subject. Exemplary mechanisms include the production of SCFA by the bacterial isolate in the gut of the subject or the modulation by the bacterial isolate of cytokine production and/or release by a cell of the subject (e.g., intestinal cell). In another example, a bacterial isolate is more abundant in the gut or fecal microbiota of a healthy human subject relative to a patient with an intestinal dysbiosis, or a more abundant in a human subject in remission from an intestinal dysbiosis relative to a patient having the intestinal dysbiosis. The increased abundance of the bacterial isolate in the intestine of a healthy subject can be indicative of a positive impact of the bacterial isolate on the health of the subject, even though the precise mechanism of action by which the bacterial isolate produces its effect may not be understood (i.e., the bacterial isolate has an impact on the health of the subject that is mechanism agnostic).

In embodiments, a pharmaceutical composition comprises one or more bacterial isolates having the ability to produce one or more SCFAs, or to enhance SCFA production by one or more bacterial strains. As used herein, an ‘SCFA’ refers to fatty acids with an aliphatic tail of one to six carbon atoms. SCFAs can be produced by bacteria during bacterial metabolism, such as during fermentation of, for example, carbohydrates, proteins, peptides and glycoprotein precursors. Illustrative SCFAs include, but are not limited to, acetic acid, butyric acid, caproic acid, formic acid, heptanoic acid, isobutyric acid, isocaproic acid, isovaleric acid, propionic acid, and valeric acid. Without wishing to be bound by theory, SCFAs are thought to play an essential role in maintaining the health of colonic mucosa, and the presence of gut SCFA-producing bacteria are associated with sustained clinical remission of certain gut dysbioses, such as UC. Accordingly, in some embodiments, a bacterial isolate incorporated into a pharmaceutical composition described herein can produce one or more SCFAs. For example, a bacterial isolate can produce one or more SCFAs in an intestine of a subject after the composition (e.g., comprising a microbial cocktail) is administered to the subject (e.g., a subject having UC). In another example, a bacterial isolate can produce one or more SCFAs in an in vitro assay capable of detecting and/or measuring SCFA production by bacteria.

In embodiments, a bacterial isolate described herein produces an SCFA selected from the group consisting of: acetic acid, butyric acid, caproic acid, formic acid, heptanoic acid, isobutyric acid, isocaproic acid, isovaleric acid, propionic acid, valeric acid, and a combination thereof.

In embodiments, a pharmaceutical composition comprises one or more bacterial isolates that produce an SCFA (e.g., butyrate) at a concentration of at least 5 mM, at least 10 mM, at least 15 mM, at least 20 mM, at least 25 mM, at least 30 mM, at least 35 mM, at least 40 mM, at least 45 mM at least 50 mM, at least 60 mM, at least 70 mM, at least 80 mM, at least 90 mM, at least 100 mM, at least 110 mM, at least 120 mM, at least 130 mM, at least 140 mM, at least 150 mM, or greater than 150 mM during a period of 24 hours. In an embodiment, the SCFA is measured in a functional assay (i.e., an assay conducted ex vivo and designed to measure the concentration or amount of an SCFA produced by a bacterial isolate, microbial cocktail, a preparation of uncultured fecal bacteria, or an uncultured fecal microbiota during a period of time (e.g., 24 hours)). For example, a functional assay can comprise incubating one or more bacterial isolates with a substrate (e.g., for 24 hours); and measuring the level of SCFA (e.g., butyrate) produced by the one or more bacterial isolates after metabolism of the substrate. In embodiments, the substrate can comprise at least one of an oligosaccharide (e.g., a fructooligosaccharide (FOS) or an xylooligosaccharide (XOS)), sunfiber/partially hydrolyzed guar gum (PHGG), or barley malt.

In an embodiment, the SCFA is produced in the intestine of a subject administered a composition described herein.

In an embodiment, a pharmaceutical composition comprises Odoribacter splanchnicus, wherein the Odoribacter splanchnicus produces an SCFA (e.g., butyrate) at a concentration of at least 20 mM, at least 21 mM, at least 22 mM, at least 23 mM, at least 24 mM, at least 25 mM, at least 26 mM, at least 27 mM, at least 28 mM, at least 29 mM, at least 30 mM, at least 31 mM, at least 32 mM, at least 33 mM, at least 34 mM, at least 35 mM, at least 36 mM, at least 37 mM, at least 38 mM, at least 39 mM, at least 40 mM, at least 41 mM, at least 42 mM, at least 43 mM, at least 44 mM, at least 45 mM, at least 46 mM, at least 47 mM, at least 48 mM, at least 49 mM, at least 50 mM, or greater than 50 mM over a period of 24 hours. In an embodiment, the Odoribacter splanchnicus comprises a 16S rRNA sequence having at least 95% sequence identity to SEQ ID NO: 2.

In an embodiment, a pharmaceutical composition comprises Roseburia sp. (e.g., Roseburia faecis), wherein the Roseburia sp. produces an SCFA (e.g., butyrate) at a concentration of at least 20 mM, at least 21 mM, at least 22 mM, at least 23 mM, at least 24 mM, at least 25 mM, at least 26 mM, at least 27 mM, at least 28 mM, at least 29 mM, at least 30 mM, at least 31 mM, at least 32 mM, at least 33 mM, at least 34 mM, at least 35 mM, at least 36 mM, at least 37 mM, at least 38 mM, at least 39 mM, at least 40 mM, at least 41 mM, at least 42 mM, at least 43 mM, at least 44 mM, at least 45 mM, at least 46 mM, at least 47 mM, at least 48 mM, at least 49 mM, at least 50 mM, or greater than 50 mM over a period of 24 hours. In an embodiment, the Roseburia sp. comprises a 16S rRNA sequence having at least 95% sequence identity to SEQ ID NO: 19.

In an embodiment, a pharmaceutical composition comprises Eubacteria sp. (e.g., Eubacteria rectale), wherein the Eubacteria sp. produces an SCFA (e.g., butyrate) at a concentration of at least 20 mM, at least 21 mM, at least 22 mM, at least 23 mM, at least 24 mM, at least 25 mM, at least 26 mM, at least 27 mM, at least 28 mM, at least 29 mM, at least 30 mM, at least 31 mM, at least 32 mM, at least 33 mM, at least 34 mM, at least 35 mM, at least 36 mM, at least 37 mM, at least 38 mM, at least 39 mM, at least 40 mM, at least 41 mM, at least 42 mM, at least 43 mM, at least 44 mM, at least 45 mM, at least 46 mM, at least 47 mM, at least 48 mM, at least 49 mM, at least 50 mM, or greater than 50 mM over a period of 24 hours. In an embodiment, the Eubacteria sp. comprises a 16S rRNA sequence having at least 95% sequence identity to SEQ ID NO: 8.

In an embodiment, a pharmaceutical composition comprises Coprococcus sp. (e.g., Coprococcus comes), wherein the Coprococcus sp. produces an SCFA (e.g., butyrate) at a concentration of at least 20 mM, at least 21 mM, at least 22 mM, at least 23 mM, at least 24 mM, at least 25 mM, at least 26 mM, at least 27 mM, at least 28 mM, at least 29 mM, at least 30 mM, at least 31 mM, at least 32 mM, at least 33 mM, at least 34 mM, at least 35 mM, at least 36 mM, at least 37 mM, at least 38 mM, at least 39 mM, at least 40 mM, at least 41 mM, at least 42 mM, at least 43 mM, at least 44 mM, at least 45 mM, at least 46 mM, at least 47 mM, at least 48 mM, at least 49 mM, at least 50 mM, or greater than 50 mM over a period of 24 hours. In an embodiment, the Coprococcus sp. comprises a 16S rRNA sequence having at least 95% sequence identity to SEQ ID NO: 17.

In an embodiment, a pharmaceutical composition comprises a microbial cocktail comprising two or more bacterial isolates comprising Odoribacter splanchnicus, Roseburia sp. (e.g., Roseburia faecis), Eubacteria sp. (e.g., Eubacteria rectale), or Coprococcus sp. (e.g., Coprococcus comes), wherein the microbial cocktail produces an SCFA (e.g., butyrate) at a concentration of at least 20 mM, at least 21 mM, at least 22 mM, at least 23 mM, at least 24 mM, at least 25 mM, at least 26 mM, at least 27 mM, at least 28 mM, at least 29 mM, at least 30 mM, at least 31 mM, at least 32 mM, at least 33 mM, at least 34 mM, at least 35 mM, at least 36 mM, at least 37 mM, at least 38 mM, at least 39 mM, at least 40 mM, at least 41 mM, at least 42 mM, at least 43 mM, at least 44 mM, at least 45 mM, at least 46 mM, at least 47 mM, at least 48 mM, at least 49 mM, at least 50 mM, at least 60 mM, at least 65 mM, at least 70 mM, at least 75 mM, at least 80 mM, at least 85 mM, at least 90 mM, at least 95 mM, at least 100 mM, at least 105 mM, at least 110 mM, at least 115 mM, at least 120 mM, at least 125 mM, at least 130 mM, at least 135 mM, at least 140 mM, at least 145 mM, at least 150 mM, or greater than 150 mM over a period of 24 hours. In an embodiment, the Odoribacter splanchnicus comprises a 16S rRNA sequence having at least 95% sequence identity to SEQ ID NO: 2. In an embodiment, the Roseburia sp. comprises a 16S rRNA sequence having at least 95% sequence identity to SEQ ID NO: 19. In an embodiment, the Eubacteria sp. comprises a 16S rRNA sequence having at least 95% sequence identity to SEQ ID NO: 8. In an embodiment, the Coprococcus sp. comprises a 16S rRNA sequence having at least 95% sequence identity to SEQ ID NO: 17.

Additionally, in some embodiments, one or more bacterial isolates administered in a composition described herein can produce levels of SCFAs comparable to that of a healthy individual in a subject previously having a functionally deficient microbial community (e.g., the microbial community of an IBD patient) who exhibited SCFA production levels lower than that of the healthy individual.

In certain embodiments, a bacterial isolate described herein can induce one or more bacterial strains to produce one or more SCFAs when the bacterial isolate and the bacterial strain(s) are present together in a common microbiota. For example, a bacterial isolate, upon administration to a subject in a composition described herein, can induce one or more bacterial strains endogenous to a gut microbiota of the subject (i.e., present in the gut of the subject prior to administration of the microbial cocktail) to produce one or more SCFAs. In another example, a bacterial isolate administered to a subject in a composition described herein can induce a second bacterial isolate administered to the subject (either in the same or a different composition) to produce one or more SCFAs. Without wishing to be bound by theory, induction of SCFA production by a second bacterial isolate can involve, for example, the production and release (e.g., by secretion) by the first bacterial isolate of one or more compounds that can be utilized by the second bacterial isolate to generate an SCFA. In one example, the compound produced and released by the first bacterial isolate to be used by the second bacterial strain to generate an SCFA is succinate, lactic acid or a lactic acid derivative. In one embodiment, the SCFA produced by the second bacterial isolate in response to exposure to the compound (e.g., lactic acid or lactic acid derivative) produced by the first bacterial isolate is butyric acid. Non-limiting examples of lactic acid derivatives include sodium isostearoyl lactate, sodium lactate, calcium lactate, aluminum lactate, ammonium lactate, potassium lactate, cetyl lactate, myristyl lactate, sodium stearoyl lactate, lactide, butyl lactate, and ethyl lactate.

In embodiments, a lactic acid-producing bacterial isolate (i.e., first bacterial isolate) included in a pharmaceutical composition described herein belongs to the Bifidobacterium genus. For example, the lactic acid-producing bacterial isolate can belong to Bifidobacterium longum or Bifidobacterium adolescentis. In embodiments, a second bacterial isolate (e.g., included with a first bacterial isolate in a microbial cocktail described herein or in a separate composition) is capable of using lactic acid produced by the Bifidobacterium to generate one or more SCFAs. In certain examples the second bacterial isolate comprises a 16S rRNA sequence that is at least 95% identical to the 16S rRNA sequence of one of the bacterial isolates provided in Table 2.

In embodiments, a bacterial isolate incorporated into a pharmaceutical composition is capable of using lactic acid produced by the gut microbiota following administration of the cocktail to generate one or more SCFAs. In certain examples the bacterial isolate comprises a 16S rRNA sequence that is at least 95% identical to the 16S rRNA sequence of one of the bacterial isolates provided in Table 2.

In embodiments, upon administration of a first and a second bacterial isolate to a subject (e.g., in the form of a microbial cocktail or in separate compositions), the first bacterial isolate produces and secretes lactic acid, and the second bacterial isolate utilizes the lactic acid to generate one or more SCFAs (e.g., butyrate). Thus, multiple bacterial isolates administered to a subject can interact synergistically within the gut of the subject to produce therapeutic effects (e.g., generation of one or more SCFAs) for treatment of an intestinal dysbiosis (e.g., IBD or ulcerative colitis) of the subject.

In embodiments, one or more bacterial isolates described herein can be administered with a probiotic that includes one or more bacterial strains that when administered to a subject produce and release a compound that can be used by the bacterial isolate to produce one or more SCFAs. For example, the probiotic can include one or more bacterial strains belonging to the genus Bifidobacterium (e.g., Bifdobacterium adolescentis or Bifidobacterium longum). In embodiments, the probiotic can be administered to a subject at the same time as a bacterial isolate, before administration of the bacterial isolate, or after administration of the bacterial isolate. In embodiments, a pharmaceutical composition comprises the probiotic and the bacterial isolate. In other embodiments, the probiotic and the bacterial isolate are in separate compositions. In some embodiments, the composition comprises a microbial cocktail of bacterial isolates.

In embodiments, one or more bacterial isolates described herein can be administered together with a prebiotic (e.g., comprising lactic acid and/or high fiber) that provides a nutrient that when utilized (e.g., metabolized) by the bacterial isolate facilitates a therapeutic effect in the subject (e.g., production of an SCFA). In embodiments, the prebiotic can be administered to a subject at the same time as a bacterial isolate, before administration of the bacterial isolate, or after administration of the bacterial isolate. In embodiments, a pharmaceutical composition comprises the prebiotic and the bacterial isolate. In other embodiments, the prebiotic and the bacterial isolate are in separate compositions. In some embodiments, the composition comprises a microbial cocktail of bacterial isolates. In some embodiments, the prebiotic is selected from the group consisting of an amino acid, lactic acid, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, nitrogen, oligodextrose, oligofructoses, oligofructose-enriched inulin, an oligosaccharide, pectin, phosphate salts, phosphorus, a polydextrose, a polyol, potash, potassium, sodium nitrate, starch, sucrose, sulfur, sun fiber, tagatose, thiamine, trans-galactooligosaccharides, trehalose, a vitamin, a water-soluble carbohydrate, a xylooligosaccharide (XOS), and a combination thereof.

In embodiments, a bacterial isolate having the ability to produce one or more SCFAs (e.g., bacterial isolates listed in Table 2) refers to a bacterial isolate actually demonstrated (e.g., by a laboratory assay) to produce one or more SCFAs. Any method can be used to detect an SCFA produced by a bacterial strain, including chromatography (e.g., liquid or gas) and/or mass spectrometry. In other embodiments, a bacterial isolate having the ability to produce one or more SCFAs refers to a bacterial isolate predicted to produce butyrate. A prediction that a bacterial isolate can produce an SCFA can be based, for example, on the isolate's taxonomy (e.g., genus and/or species) and/or a sequence of a gene or polypeptide of the bacterial isolate known to mediate SCFA production. For example, an example of a gene known to mediate butyrate production is butyrate kinase. In an embodiment, a bacterial isolate is predicted to produce butyrate based on the identification of a butyrate kinase gene in the genome of the bacterial isolate. In an embodiment, the butyrate kinase gene when translated into a protein is predicted to generate a functional (i.e., enzymatically active) butyrate kinase enzyme.

Examples of bacterial isolates having the ability to produce SCFA are provided in Table 2. Each bacterial isolate is identified by Latin name, an Identification Number (ID number), and the Sequence Identifier (SEQ ID NO) for its 16S rRNA sequence. In aspects, a pharmaceutical composition (e.g., a microbial cocktail) comprises at least one bacterial isolate provided in Table 2, or at least one bacterial isolate comprising a 16S rRNA sequence at least 95% identical to the 16S rRNA sequence of one or more of the bacterial isolates provided in Table 2. In embodiments, the pharmaceutical composition comprises at least two bacterial isolates, at least three bacterial isolates, at least four bacterial isolates, at least five bacterial isolates, at least six bacterial isolates, at least seven bacterial isolates, at least eight bacterial isolates, or at least nine bacterial isolates that each comprise a 16S rRNA sequence at least 95% identical to a 16S rRNA sequence of at least one of the bacterial isolates provided in Table 2.

TABLE 2

| | | SEQ ID NO |
|---|---|---|
| Isolate Latin Name | ID Number | for 16S rRNA Sequence |
| Faecalibacterium prausnitzii | PI00000329 | 1 |
| Odoribacter splanchnicus | PI00000072 | 2 |
| Anaerostipes hadrus | PI00000094 | 3 |
| Faecalibacterium prausnitzii | IS00006632 | 7 |
| Eubacterium rectale | IS00006864 | 8 |
| Coprococcus comes | PI00000370 | 17 |
| Roseburia faecis | PI00000404 | 19 |
| Subdoligranulum variabile | IS00007359 | 22 |
| Subdoligranulum variabile | IS00007357 | 23 |

In embodiments, a pharmaceutical composition (e.g., comprising a microbial cocktail) comprises one or more bacterial isolates having the ability to modulate production of a cytokine (e.g., IL-10, GM-CSF, IFN-gamma, TNF-alpha, IL-23, and IL-12) by a eukaryotic cell. Herein “eukaryotic cell” refers to both a cell (e.g. intestinal cell) positioned ‘in situ’ within a body of a subject administered a composition described herein, as well as a cell grown or growing “ex vivo” outside of an organism, for example, in culture medium.

In one example, one or more bacterial isolates in a pharmaceutical composition described herein, once administered to a subject, can modulate production of a cytokine in a cell of the subject (referred to herein as a “host cell”). In embodiments, one or more bacterial isolates modulate production and/or secretion of a cytokine from a host cell of the subject, wherein the cytokine tends to exert anti-inflammatory effects on a tissue of the subject (e.g., intestinal tissue). Examples of such anti-inflammatory cytokines that can be produced and/or secreted from a host cell in response to the presence of a bacterial isolate administered in a composition described herein include IL-10, IL-13, IL-4, IL-5, TGF-β, and a combination thereof. In other embodiments, one or more bacterial isolates administered in a composition described herein inhibit production and/or secretion of a cytokine from a host cell of a subject, wherein the cytokine tends to exert pro-inflammatory effects on a tissue of the subject (e.g., intestinal tissue). Examples of such pro-inflammatory cytokines include IFNγ, IL-12p70, IL-1 (e.g., IL-1α, IL-1β), IL-6, IL-8, IL-12, IL-17, IL-18, IL-23, MCP1, MIP1α, MIP1β, TNFα, TNF-γ, and a combination thereof. By providing a composition containing one or more bacterial isolates that can induce a host cell to produce or secrete an anti-inflammatory cytokine and/or inhibit production and/or secretion by the host cell of a pro-inflammatory cytokine, the compositions (e.g., microbial cocktails) described herein can treat, alleviate, inhibit, and/or prevent inflammation associated with an intestinal dysbiosis of a subject, for example, Inflammatory Bowel Disease. Herein a bacterial isolate capable of modulating cytokine production and/or secretion by a host cell is referred to as an “immunomodulatory” bacterial isolate.

In embodiments, a bacterial isolate can directly and/or indirectly modulate production and/or release of a cytokine from a cell of a subject administered a pharmaceutical composition. In one embodiment an immunomodulatory bacterial isolate can act directly on a host cell of a subject via, for example, microbe-associated molecular patterns (MAMPS) secreted by the bacterial isolate or displayed on the surface of the bacterial isolate. Such MAMPS play a major role in host immune responses to particular bacterial species. MAMPS are sensed by pattern recognition receptors (PRRs) expressed on most host cell types that are in contact with bacteria. Examples of MAMPS of a bacterial isolate described herein include unmethylated 2′-deoxyribo(cytidine-phosphate-guanine) (CpG) dinucleotides, bacterial peptidogylcans, bacterial lipopolysaccharides (LPS, which interacts with co-receptors MD-2, CD14, and LPB to facilitate high affinity binding to TLR-4 and subsequent host cell activation), bacterial lipoproteins (LPs), lipoteichoic acid, flagellin, membrane vesicles, and exopolysaccharides. Examples of PRRs expressed by host cells in the gut and resident intestinal immune cells that can mediate modulation of cytokine production via interaction with MAMPS include Toll-like receptors (TLRs), nucleotide-binding oligomerization domains (Nods), NOD like receptors and C-type lectins. The interaction of intestinal cell PRRs and microbial ligands trigger signaling pathways associated with the innate and adaptive immune systems that are required to maintain immune tolerance and intestinal health.

In another embodiment, an immunomodulatory bacterial isolate can act indirectly on a cell (e.g., immune cell) of a subject administered a pharmaceutical composition by, for example, secreting a metabolite that modulates the activity of the host cell of the subject, for example, by inducing the cell to express a cytokine.

Examples of host cells whose production and/or release of cytokines can be modulated by a bacterial isolate described herein include an intestinal cell, an epithelial cell, an intestinal mucosal cell, an intestinal epithelial cell, an intestinal lamina propria cell, an endothelial cell, fibroblast, a stromal cell, a macrophage, a B lymphocyte, a T lymphocyte, a mast cell, and a peripheral blood mononuclear cell (PBMC).

In another embodiment, a bacterial isolate described herein can modulate cytokine production and/or release (e.g., increase cytokine production) by a eukaryotic cell (e.g., PBMC) situated or growing in culture medium, when the bacterial isolate is co-cultured with the eukaryotic cell.

In certain embodiments, a bacterial isolate described herein can induce production and/or release of a cytokine (e.g., IL-10) by a eukaryotic cell at a level of at least 500 pg/ml, at least 1000 pg/ml, at least 1500 pg/ml, at least 2000 pg/ml, at least 2500 pg/ml, or at least 3000 pg/ml. In an embodiment, the cytokine is measured in a functional assay (i.e., an assay conducted ex vivo and designed to measure the concentration or amount of a cytokine produced by a eukaryotic cell (e.g., PBMC) in contact with a bacterial isolate, microbial cocktail, a preparation of uncultured fecal bacteria, or an uncultured fecal microbiota during a period of time (e.g., 24 hours)). In an embodiment, the cytokine is produced in the intestine of a subject administered a composition described herein.

In certain embodiments, a bacterial isolate described herein can induce an anti-inflammatory cytokine profile. In an embodiment, a bacterial isolate exhibits an anti-inflammatory cytokine profile when it produces a level of IL-10 that is increased relative to that of a control strain. In an embodiment, a bacterial isolate exhibits an anti-inflammatory cytokine profile when it produces a level of IL-12 that is decreased relative to that of a control strain. In an embodiment, a bacterial isolate exhibits an anti-inflammatory cytokine profile when it produces a level of GM-CSF that is decreased relative to that of a control strain. In an embodiment, a bacterial isolate exhibits an anti-inflammatory cytokine profile when it produces a level of IFN-gamma that is decreased relative to that of a control strain. In an embodiment, a bacterial isolate exhibits an anti-inflammatory cytokine profile when it produces a level of TNF-alpha that is decreased relative to that of a control strain. In an embodiment, a bacterial isolate exhibits an anti-inflammatory cytokine profile when it produces a level of IL-23 that is decreased relative to that of a control strain. In an embodiment, a bacterial isolate exhibits an anti-inflammatory cytokine profile when it produces a level of IL-12 that is decreased relative to that of a control strain. In an embodiment, a bacterial isolate exhibits an anti-inflammatory cytokine profile when it produces a ration of IL-10:IL-12 that is increased relative to that of a control strain. In an embodiment, a bacterial isolate exhibits an anti-inflammatory cytokine profile when it produces a ration of IL-10:TNF-alpha that is increased relative to that of a control strain.

In embodiments, a pharmaceutical composition comprises one or more bacterial isolates which, when in contact with a eukaryotic cell (e.g., population of PBMCs) during a period of time (e.g., 24 hours), induce production of IL-10 at a concentration of at least 1000 pg/ml, at least 1500 pg/ml, at least 2000 pg/ml, at least 2500 pg/ml, at least 3000 pg/ml, at least 3500 pg/ml, at least 4000 pg/ml, at least 5000 pg/ml, at least 6000 pg/ml, at least 7000 pg/ml, at least 8000 pg/ml, at least 9000 pg/ml, at least 10,000 pg/ml, or greater than 10,000 pg/ml. In an embodiment, the bacterial isolate is provided in Table 3, or comprises a 16S rRNA sequence at least 95% identical to a 16S rRNA sequence corresponding to a SEQ ID NO of a bacterial isolate provided in Table 3.

In embodiments, a pharmaceutical composition comprises one or more bacterial isolates which, when in contact with a eukaryotic cell (e.g., population of PBMCs) during a period of time (e.g., 24 hours), limit production of GM-CSF to a concentration of no more than 5 pg/ml, 10 pg/ml, 15 pg/ml, 20 pg/ml, 25 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, 80 pg/ml, 85 pg/ml, 90 pg/ml, 95 pg/ml, 100 pg/ml, 105 pg/ml, 110 pg/ml, 115 pg/ml, 120 pg/ml, 125 pg/ml, 130 pg/ml, 135 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, or 200 pg/ml. In an embodiment, the bacterial isolate is provided in Table 3, or comprises a 16S rRNA sequence at least 95% identical to a 16S rRNA sequence corresponding to a SEQ ID NO of a bacterial isolate provided in Table 3.

In embodiments, a pharmaceutical composition comprises one or more bacterial isolates which, when in contact with a eukaryotic cell (e.g., population of PBMCs) during a period of time (e.g., 24 hours), limit production of IL-12 (e.g., IL-12p70) to a concentration of no more than 5 pg/ml, 10 pg/ml, 15 pg/ml, 20 pg/ml, 25 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, 80 pg/ml, 85 pg/ml, 90 pg/ml, 95 pg/ml, 100 pg/ml, 105 pg/ml, 110 pg/ml, 115 pg/ml, 120 pg/ml, 125 pg/ml, 130 pg/ml, 135 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, or 200 pg/ml. In an embodiment, the bacterial isolate is provided in Table 3, or comprises a 16S rRNA sequence at least 95% identical to a 16S rRNA sequence corresponding to a SEQ ID NO of a bacterial isolate provided in Table 3.

In embodiments, a pharmaceutical composition comprises one or more bacterial isolates which, when in contact with a eukaryotic cell (e.g., population of PBMCs) during a period of time (e.g., 24 hours), limit production of IFN-gamma to a concentration of no more than 5 pg/ml, 10 pg/ml, 15 pg/ml, 20 pg/ml, 25 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, 80 pg/ml, 85 pg/ml, 90 pg/ml, 95 pg/ml, 100 pg/ml, 105 pg/ml, 110 pg/ml, 115 pg/ml, 120 pg/ml, 125 pg/ml, 130 pg/ml, 135 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, or 200 pg/ml. In an embodiment, the bacterial isolate is provided in Table 3, or comprises a 16S rRNA sequence at least 95% identical to a 16S rRNA sequence corresponding to a SEQ ID NO of a bacterial isolate provided in Table 3.

In embodiments, a pharmaceutical composition comprises one or more bacterial isolates which, when in contact with a eukaryotic cell (e.g., population of PBMCs) during a period of time (e.g., 24 hours), limit production of TNF-alpha to a concentration of no more than 20 pg/ml, 30 pg/ml, 40 pg/ml, 50 pg/ml, 75 pg/ml, 100 pg/ml, 150 pg/ml, 200 pg/ml, 250 pg/ml, 300 pg/ml, 350 pg/ml, 400 pg/ml, 450 pg/ml, 500 pg/ml, 550 pg/ml, 600 pg/ml, 650 pg/ml, 700 pg/ml, 750 pg/ml, 800 pg/ml, 850 pg/ml, 900 pg/ml, 950 pg/ml, 1000 pg/ml, 1100 pg/ml, 1200 pg/ml, 1300 pg/ml, 1400 pg/ml, 1500 pg/ml, 1600 pg/ml, 1700 pg/ml, 1800 pg/ml, 1900 pg/ml, 2000 pg/ml, 2100 pg/ml, 2200 pg/ml, 2300 pg/ml, 2400 pg/ml, or 2500 pg/ml. In an embodiment, the bacterial isolate is provided in Table 3, or comprises a 16S rRNA sequence at least 95% identical to a 16S rRNA sequence corresponding to a SEQ ID NO of a bacterial isolate provided in Table 3.

In embodiments, a pharmaceutical composition comprises one or more bacterial isolates which, when in contact with a eukaryotic cell (e.g., population of PBMCs) during a period of time (e.g., 24 hours), limit production of IL-23 to a concentration of no more than 5 pg/ml, 10 pg/ml, 15 pg/ml, 20 pg/ml, 25 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, 80 pg/ml, 85 pg/ml, 90 pg/ml, 95 pg/ml, 100 pg/ml, 105 pg/ml, 110 pg/ml, 115 pg/ml, 120 pg/ml, 125 pg/ml, 130 pg/ml, 135 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, 200 pg/ml, 250 pg/ml, or 300 pg/ml. In an embodiment, the bacterial isolate is provided in Table 3, or comprises a 16S rRNA sequence at least 95% identical to a 16S rRNA sequence corresponding to a SEQ ID NO of a bacterial isolate provided in Table 3.

In embodiments, a pharmaceutical composition comprises one or more bacterial isolates which, when in contact with a eukaryotic cell (e.g., population of PBMCs) during a period of time (e.g., 24 hours), induce a ratio of IL-10:IL-12 of at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 325, at least 350, at least 375, at least 400, at least 425, at least 450, at least 475, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, at least 2000, at least 2200, at least 2400, at least 2600, at least 2800, at least 3000, at least 3200, at least 3400, at least 3600, at least 3800, at least 4000, or greater than 4000. In an embodiment, the bacterial isolate is provided in Table 3, or comprises a 16S rRNA sequence at least 95% identical to a 16S rRNA sequence corresponding to a SEQ ID NO of a bacterial isolate provided in Table 3.

In embodiments, a pharmaceutical composition comprises one or more bacterial isolates which, when in contact with a eukaryotic cell (e.g., population of PBMCs) during a period of time (e.g., 24 hours), induce a ratio of IL-10:TNF-alpha of at least 0.5, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, at least 75, at least 80, at least 85, at least 90, at least 95, or at least 100. In an embodiment, the bacterial isolate is provided in Table 3, or comprises a 16S rRNA sequence at least 95% identical to a 16S rRNA sequence corresponding to a SEQ ID NO of a bacterial isolate provided in Table 3.

Examples of bacterial isolates having the ability to modulate cytokine production by a host cell are provided in Table 3. Each isolate is identified by Latin name, an Identification Number (ID number), and the Sequence Identifier (SEQ ID NO) for its 16S rRNA sequence. In aspects, a pharmaceutical composition (e.g., comprising a microbial cocktail) comprises at least one bacterial isolate provided in Table 3, or at least one bacterial isolate that comprises a 16S rRNA sequence at least 95% identical to the 16S rRNA sequence of one or more of the bacterial isolates provided in Table 3. In embodiments, the pharmaceutical composition comprises at least two bacterial isolates, at least three bacterial isolates, at least four bacterial isolates, at least five bacterial isolates, at least six bacterial isolates, at least seven bacterial isolates, at least eight bacterial isolates, at least nine bacterial isolates, at least ten bacterial isolates, at least eleven bacterial isolates, at least twelve bacterial isolates, at least thirteen bacterial isolates, or at least fourteen bacterial isolates that each comprise a 16S rRNA sequence at least 95% identical to a 16S rRNA sequence of at least one of the bacterial isolates provided in Table 3.

TABLE 3

| | | SEQ ID NO |
|---|---|---|
| Isolate Latin Name | ID Number | for 16S rRNA Sequence |
| Faecalibacterium prausnitzii | PI00000329 | 1 |
| Odoribacter splanchnicus | PI00000072 | 2 |
| Anaerostipes hadrus | PI00000094 | 3 |
| Faecalibacterium prausnitzii | IS00006632 | 7 |
| Bacteroides uniformis | PI00000137 | 11 |
| Bacteroides vulgatus | PI00000138 | 12 |
| Bacteroides uniformis | PI00000352 | 16 |
| Coprococcus comes | PI00000370 | 17 |
| Alistipes shahii | PI00000395 | 18 |
| Akkermansia mucimphila | IS00007180 | 20 |
| Subdoligranulum variabile | IS00007359 | 22 |
| Subdoligranulum variabile | IS00007357 | 23 |

In embodiments, a pharmaceutical composition comprises at least one bacterial isolate corresponding to a bacterial strain having a greater relative abundance in a healthy human subject relative to a patient with an intestinal dysbiosis (e.g., an IBD such as UC), or a greater relative abundance in a human subject in remission from an intestinal dysbiosis relative to a patient having the intestinal dysbiosis. Herein the term “greater relative abundance” refers to a higher number of viable cells of the bacterial strain (i.e., corresponding to the bacterial isolate) in a healthy human subject (compared to a patient with an intestinal dysbiosis) or in a human subject in remission from an intestinal dysbiosis (compared to a patient having the intestinal dysbiosis). In some embodiments, the term “higher number of viable cells” refers to the absolute number of viable cells of the bacterial strain in an intestinal microbiota or portion thereof (e.g., in a stool sample), while in other embodiments, the term refers to the proportional number of viable cells of the bacterial strain relative to the approximate entire number of viable cells in the intestinal microbiota or portion thereof (e.g., in a stool sample). In certain examples, a bacterial strain corresponding to a bacterial isolate included in a pharmaceutical composition can have a greater relative abundance across multiple tested human subjects (e.g., healthy individuals, or individuals in remission from an intestinal dysbiosis), for example, at least 5 human subjects, at least 10 human subjects, at least 20 human subjects, at least 30 human subjects, at least 40 human subjects, at least 50 human subjects, at least 75 human subjects, at least 100 human subjects, at least 200 human subjects, at least 300 human subjects, at least 400 human subjects, at least 500 human subjects, at least 750 human subjects, at least 1000 human subjects, or greater than 1000 human subjects. In addition or alternatively, a bacterial strain corresponding to a bacterial isolate can have a greater relative abundance in a proportion of tested human subjects (e.g., healthy individuals, or individuals in remission from an intestinal dysbiosis), for example, at least 50% of tested individuals, at least 55% of tested individuals, at least 60% of tested individuals, at least 65% of tested individuals, at least 70% of tested individuals, at least 75% of tested individuals, at least 80% of tested individuals, at least 80% of tested individuals, at least 85% of tested individuals, at least 90% of tested individuals, at least 95% of tested individuals, or 100% of tested individuals. Herein a bacterial strain “corresponding to a bacterial isolate” refers to a bacterial strain in a gut microbiota of a subject, wherein the bacterial strain has a 16S rRNA sequence that typically shares at least 97% identity (e.g., at least 97.5% identity, at least 98% identity, at least 98.5% identity, at least 99% identity, at least 99.5% identity, or greater than 99.5% identity) to a 16S rRNA sequence of the bacterial isolate. Likewise, a bacterial isolate “corresponding to a bacterial strain” refers to a bacterial isolate that typically shares at least 97% identity (e.g., at least 97.5% identity, at least 98% identity, at least 98.5% identity, at least 99% identity, at least 99.5% identity, or greater than 99.5% identity) to a 16S rRNA sequence of a bacterial strain in a gut microbiota of a subject.

In embodiments, a bacterial isolate can correspond to a bacterial strain having a greater relative abundance in a human subject in remission from an intestinal dysbiosis relative to a patient having the intestinal dysbiosis. The increased abundance of the bacterial strain in the subject in remission identifies the bacterial isolate corresponding to the bacterial strain as potentially advantageous for the treatment of a disorder related to an intestinal dysbiosis, such as UC. The remission of the intestinal dysbiosis in the human subject can be related to or caused by an intervention administered to the subject while the intestinal dysbiosis is active. For example, the remission can arise following treatment of the subject with a microbial therapeutic. Examples of a microbial therapeutic include compositions comprising a preparation of uncultured fecal bacteria, an uncultured fecal microbiota, a cultured fecal microbiota, and/or a bacterial isolate. In embodiments, the microbial therapeutic which induces remission from an intestinal dysbiosis is a substantially complete uncultured fecal microbiota. For example, a subject can be administered a fecal microbiota transplant (FMT) to induce remission of the intestinal dysbiosis. In certain embodiments, the intestinal dysbiosis of the subject is due to ulcerative colitis (UC).

In an embodiment, this mechanism agnostic approach to identifying a bacterial isolate reduces the dysbiosis associated with Ulcerative Colitis (UC). In an embodiment, 16S ribosomal DNA (rDNA) and shotgun metagenomic sequences can be incorporated from for example interventional (FMT), cross-sectional and time series datasets to develop predictive features associated with either a healthy status or clinical response to FMT. These features can be used to rank and select bacterial phylogenetic clades for (i) enrichment in healthy subjects over patients diagnosed with UC; and/or (ii) association/correlation with clinical remission or response of UC symptoms in UC patients following FMT treatment. Clades can be ranked based on a “cross-sectional combined p-value” which compares the presence and abundances of bacterial strains in fecal material between healthy subjects and patients with UC. The lower the p-value, the more likely the organisms in the clade are having an effect on the treatment, inhibition or prevention of UC based on: (i) depletion of the strain in UC patients and/or (ii) high abundance of the strain in healthy subjects. Isolated bacterial strains can then be selected from donor stool samples by 16S rDNA similarity to the ranked phylogenetic clades or by ranking their 16S rDNA directly according to the aforementioned criteria.

In an embodiment, a value of a cross-sectional combined p-value is less than 0.1, less than 0.01, less than 1×10⁻³, less than 1×10⁻⁴, less than 1×10⁻⁵, less than 1×10⁻⁶, less than 1×10⁻⁷, less than 1×10⁻⁸, less than 1×10⁻⁹, less than 1×10⁻¹⁰, less than 1×10⁻¹¹, less than 1×10⁻¹², less than 1×10⁻¹³, less than 1×10⁻¹⁴, less than 1×10⁻¹⁵, less than 1×10⁻¹⁶, less than 1×10⁻¹⁷, less than 1×10⁻¹⁸, less than 1×10⁻¹⁹, less than 1×10⁻²⁰, less than 1×10⁻²¹, less than 1×10⁻²², less than 1×10⁻²³, or less than 1×10⁻²⁴.

Table 4 lists examples of bacterial isolates which can be included in a pharmaceutical composition (e.g., comprising a microbial cocktail) that have corresponding bacterial strains present and/or more highly abundant in a healthy subject relative to a patient with UC. Each isolate is identified by Latin name, an Identification Number (ID number), and the Sequence Identifier (SEQ ID NO) for its 16S rRNA sequence. In aspects, a pharmaceutical composition comprises at least one bacterial isolate provided in Table 4, or at least one bacterial isolate that comprises a 16S rRNA sequence at least 95% identical to the 16S rRNA sequence of one or more of the bacterial isolates provided in Table 4. In embodiments, the pharmaceutical composition comprises at least two bacterial isolates, at least three bacterial isolates, at least four bacterial isolates, at least five bacterial isolates, at least six bacterial isolates, at least seven bacterial isolates, at least eight bacterial isolates, at least nine bacterial isolates, at least ten bacterial isolates, at least eleven bacterial isolates, at least twelve bacterial isolates, at least thirteen bacterial isolates, at least fourteen bacterial isolates, at least fifteen bacterial isolates, at least sixteen bacterial isolates, at least seventeen bacterial isolates, at least eighteen bacterial isolates, at least nineteen bacterial isolates, at least twenty bacterial isolates, at least twenty one bacterial isolates, at least twenty two bacterial isolates, or at least twenty three bacterial isolates that each comprise a 16S rRNA sequence at least 95% identical to a 16S rRNA sequence of at least one of the bacterial isolates provided in Table 4.

TABLE 4

| | | SEQ ID NO for 16S |
|---|---|---|
| Isolate Latin Name | ID Number | rRNA Sequence |
| Faecalibacterium prausnitzii | PI00000329 | 1 |
| Odoribacter splanchnicus | PI00000072 | 2 |
| Anaerostipes hadrus | PI00000094 | 3 |
| Alistipes onderdonkii | IS00004389 | 4 |
| Parabacteroides merdae | IS00006167 | 5 |
| Dorea longicatena | IS00006618 | 6 |
| Faecalibacterium prausnitzii | IS00006632 | 7 |
| Eubacterium rectale | IS00006864 | 8 |
| Blautia obeum | PI00000053 | 9 |
| Bacteroides uniformis | PI00000137 | 11 |
| Bacteroides vulgatus | PI00000138 | 12 |
| Bacteroides cellulosilyticus | PI00000316 | 14 |
| Alistipes finegoldii | PI00000340 | 15 |
| Bacteroides uniformis | PI00000352 | 16 |
| Alistipes shahii | PI00000395 | 18 |
| Akkermansia muciniphila | IS00007180 | 20 |
| Phascolarctobacterium faecium | PI00000289 | 21 |
| Subdoligranulum variabile | IS00007359 | 22 |
| Subdoligranulum variabile | IS00007357 | 23 |
| Blautia sp. | IS00002788 | 34 |
| Alistipes putredinis | IS00008139 | 35 |
| Alistipes putredinis | IS00008142 | 36 |
| Alistipes putredinis | IS00008177 | 37 |

In an embodiment, a bacterial isolate is identified as suitable for inclusion in a pharmaceutical composition described herein (e.g., comprising a microbial cocktail) based on its correspondence to a bacterial strain that is abundant in healthy versus UC patients in the data set reported in Morgan et al., Genome Biology 13 (2012), the entire contents of which are hereby incorporated by reference.

In an embodiment, a bacterial isolate is identified as suitable for inclusion in a pharmaceutical composition described herein (e.g., comprising a microbial cocktail) based on its correspondence to a bacterial strain that is abundant in healthy versus UC patients in the data set reported in Papa et al., PLOS ONE 7, no. 6 (Jun. 29, 2012), the entire contents of which are hereby incorporated by reference.

In embodiments, a microbial cocktail described herein can contain two or more bacterial isolates which are related bacterial isolates. Herein “related bacterial isolates” have 16S rRNA sequences which typically share at least 95% sequence identity. In certain embodiments, related bacterial isolates have 16S sequences which share at least 97% sequence identity, and thus the bacterial isolates are members of the same species. In embodiments, a microbial cocktail contains two or more related bacterial isolates. In other embodiments, a microbial cocktail does not contain two related bacterial isolates. In embodiments, a first version of a microbial cocktail contains a first bacterial isolate, and a second version of a microbial cocktail contains a second bacterial isolate that is a related bacterial isolate to the first bacterial isolate.

Examples of related bacterial isolates to those listed in Table 4 that can be included in a microbial cocktail described herein are shown in Table 5. Like the bacterial isolates in Table 4, those in Table 5 correspond to bacterial strains that have a greater abundance in a healthy human subject relative to a patient with UC. Table 5 lists the bacterial isolates' identification numbers, the ID number of the corresponding bacterial isolate in Table 4, the percent identity of the related bacterial strain's 16S sequences to the sequence of the corresponding bacterial isolate in Table 4, and the Sequence Identifier (SEQ ID NO) for its 16S rRNA sequence.

TABLE 5

| | Related to ID | % identity of 16S rRNA | SEQ ID NO for |
|---|---|---|---|
| | Number of | Sequence to Related ID | 16S rRNA |
| ID Number | Table 4 | Number of Table 4 | Sequence |
| PI00000070 | PI00000072 | 98.6 | 24 |
| PI00000092 | PI00000094 | 98.1 | 25 |
| PI00000339 | IS00004389 | 96.6 | 26 |
| PI00000327 | IS00006167 | 99.4 | 27 |
| PI00000056 | PI00000053 | 96.2 | 28 |
| PI00000152 | PI00000138 | 98.7 | 29 |
| PI00000043 | PI00000316 | 99.2 | 30 |
| IS00003009 | PI00000340 | 97.6 | 31 |
| PI00000052 | PI00000352 | 96.6 | 32 |
| PI00000330 | PI00000395 | 95.3 | 33 |

In embodiments, a pharmaceutical composition comprises all or a subset of the bacterial isolates in at least one of Tables 1-4, or a bacterial isolate having a 16S rRNA sequence that is at least 95% identical to a 16S rRNA sequence of one or more of the bacterial isolates in Tables 1-4. In embodiments, all bacterial isolates in a pharmaceutical composition (e.g., comprising a microbial cocktail) are found in at least one of Tables 2-4, or share at least 95% identity in a 16S rRNA sequence to that of a bacterial isolate provided in at least one of Tables 2-4. In embodiments, all bacterial isolates in a pharmaceutical composition are listed together in one of Tables 2-4, or share at least 95% identity in a 16S rRNA sequence to that of a bacterial isolate provided in at least one of Tables 2-4. In an aspect, a pharmaceutical composition does not contain any bacterial isolates from at least one of Tables 2-4, or does not contain a bacterial isolate that shares 95% identity in a 16S rRNA sequence to that of a bacterial isolate provided in at least one of Tables 2-4.

In an aspect, a pharmaceutical composition can comprise one or more bacterial isolates (e.g., at least 1, at least 2, at least 3, at least 4, at least 5, at least 6, at least 7, at least 8, or at least 9 bacterial isolates) from Table 2, or comprising a 16S rRNA sequence that is at least 95% identical to a 16S rRNA sequence of one or more of the bacterial isolates provided in Table 2. A microbial cocktail described herein can therefore comprise multiple bacterial isolates capable of generating one or more SCFAs such as butyrate. By providing a microbial cocktail containing multiple bacterial isolates that produce an SCFA such as butyrate in a gut of a subject administered the cocktail, a pharmaceutical composition can induce a spike or elevation or ‘burst’ of butyrate in the gut of a subject (e.g., which is depleted of SCFAs). Table 7, Table 8 and Table 9 illustrate exemplary microbial cocktails containing bacterial isolates that are all found in Table 2; each isolate is identified by Latin name, an Identification Number (ID number), and the Sequence Identifier (SEQ ID NO) for its 16S rRNA sequence. In certain embodiments, a composition comprises a microbial cocktail comprising each of the bacterial isolates in one of Tables 7-9, or bacterial isolates comprising 16S rRNA sequences at least 95% identical to the 16S rRNA sequence of each of the bacterial isolates in one of Tables 7-9. In embodiments, the pharmaceutical composition comprises at least eight bacterial isolates that each comprise a 16S rRNA sequence at least 95% identical to a 16S rRNA sequence of at least one of the bacterial isolates provided in Table 7. In embodiments, the pharmaceutical composition comprises at least six bacterial isolates that each comprise a 16S rRNA sequence at least 95% identical to a 16S rRNA sequence of at least one of the bacterial isolates provided in one of Tables 8-9. In certain embodiments, a composition comprises a microbial cocktail consisting essentially of the bacterial isolates listed in one of Tables 7-9, or bacterial isolates comprising 16S rRNA sequences at least 95% identical to the 16S rRNA sequence of each of the bacterial isolates in one of Tables 7-9. In certain embodiments, a composition comprises a microbial cocktail consisting of the bacterial isolates listed in one of Tables 7-9, or bacterial isolates comprising 16S rRNA sequences at least 95% identical to the 16S rRNA sequence of each of the bacterial isolates in one of Tables 7-9.

TABLE 7

| | | SEQ ID NO |
|---|---|---|
| Isolate Latin Name | ID Number | for 16S rRNA Sequence |
| Odoribacter splanchnicus | PI00000072 | 2 |
| Subdoligranulum variabile | IS00007359 | 22 |
| Subdoligranulum variabile | IS00007357 | 23 |
| Eubacterium rectale | IS00006864 | 8 |
| Roseburia faecis | PI00000404 | 19 |
| Faecalibacteriurn prausnitzii | PI00000329 | 1 |
| Faecalibacteriurn prausnitzii | IS00006632 | 7 |
| Anaerostipes hadrus | PI00000094 | 3 |

TABLE 8

| | | SEQ ID NO |
|---|---|---|
| Isolate Latin Name | ID Number | for 16S rRNA Sequence |
| Faecalibacterium prausnitzii | PI00000329 | 1 |
| Coprococcus comes | PI00000370 | 17 |
| Anaerostipes hadrus | PI00000094 | 3 |
| Eubacterium rectale | IS00006864 | 8 |
| Roseburia faecis | PI00000404 | 19 |
| Subdoligranulum variabile | IS00007359 | 22 OR |
| | OR | 23 |
| | IS00007357 | |

TABLE 9

| | | SEQ ID NO |
|---|---|---|
| Isolate Latin Name | ID Number | for 16S rRNA Sequence |
| Faecalibacterium prausnitzii | IS00006632 | 7 |
| Anaerostipes hadrus | PI00000094 | 3 |
| Eubacterium rectale | IS00006864 | 8 |
| Roseburia faecis | PI00000404 | 19 |
| Coprococcus comes | PI00000370 | 17 |
| Subdoligranulum variabile | IS00007359 | 22 OR |
| | OR | 23 |
| | IS00007357 | |

In embodiments, a pharmaceutical composition (e.g., comprising a microbial cocktail) can comprise one or more bacterial isolates (e.g., at least 1, at least 2, at least 3, at least 4, at least 5, at least 6, at least 7, at least 8, at least 9, at least 10, at least 11, at least 12, at least 13, at least 14, at least 15, or greater than 15 bacterial isolates) that are not in Table 2. In embodiments, a pharmaceutical composition lacks at least one of the bacterial isolates in Table 2, or lacks bacterial isolates comprising 16S rRNA sequences at least 95% identical to the 16S rRNA sequence of at least one of the bacterial isolates in Table 2. In embodiments, a pharmaceutical composition (e.g., comprising a microbial cocktail) does not contain any bacterial isolate from Table 2. A pharmaceutical composition described herein can therefore comprise or consist of one or more bacterial isolates which substantially do not produce an SCFA in the gut of a subject, and/or does not induce a spike of SCFA in the gut of the subject post-administration. Table 10, Table 11 and Table 12 illustrate exemplary microbial cocktails containing bacterial isolates that are not found in Table 2; each isolate is identified by Latin name, an Identification Number (ID number), and the Sequence Identifier (SEQ ID NO) for its 16S rRNA sequence. In certain embodiments, a composition comprises a microbial cocktail comprising each of the bacterial isolates in one of Tables 10-12, or bacterial isolates comprising 16S rRNA sequences at least 95% identical to the 16S rRNA sequence of each of the bacterial isolates in one of Tables 10-12. In embodiments, the pharmaceutical composition comprises at least eight bacterial isolates that each comprise a 16S rRNA sequence at least 95% identical to a 16S rRNA sequence of at least one of the bacterial isolates provided in one of Tables 10-11. In embodiments, the pharmaceutical composition comprises at least six bacterial isolates that each comprise a 16S rRNA sequence at least 95% identical to a 16S rRNA sequence of at least one of the bacterial isolates provided in Table 12. In certain embodiments, a composition comprises a microbial cocktail consisting essentially of the bacterial isolates listed in one of Tables 10-12, or bacterial isolates comprising 16S rRNA sequences at least 95% identical to the 16S rRNA sequence of each of the bacterial isolates in one of Tables 10-12. In certain embodiments, a composition comprises a microbial cocktail consisting of the bacterial isolates listed in one of Tables 10-12, or bacterial isolates comprising 16S rRNA sequences at least 95% identical to the 16S rRNA sequence of each of the bacterial isolates in one of Tables 10-12. In an embodiment, a microbial cocktail comprises bacterial isolates comprising 16S rRNA sequences at least 95% identical to the 16S rRNA sequence of each of the bacterial isolates in one of Tables 10-12, as well as an additional bacterial isolate. For example, the additional bacterial isolate can be a member of the genus Blautia.

TABLE 10

| | | SEQ ID NO |
|---|---|---|
| Isolate Latin Name | ID Number | for 16S rRNA Sequence |
| Akkermansia muciniphila | IS00007180 | 20 |
| Parabacteroides merdae | IS00006167 | 5 |
| Bacteroides uniformis | PI00000137 | 11 OR |
| | OR | 16 |
| | PI00000352 | |
| Alistipes finegoldii | PI00000340 | 15 |
| Bacteroides vulgatus | PI00000138 | 12 |
| Dorea longicatena | IS00006618 | 6 |
| Blautia obeum | PI00000053 | 9 |
| Blautia sp. | IS00002788 | 34 |

TABLE 11

| | | SEQ ID NO |
|---|---|---|
| Isolate Latin Name | ID Number | for 16S rRNA Sequence |
| Akkermansia muciniphila | IS00007180 | 20 |
| Parabacteroides merdae | IS00006167 | 5 |
| Bacteroides uniformis | PI00000137 | 11 |
| | OR | OR |
| | PI00000352 | 16 |
| Alistipes finegoldii | PI00000340 | 15 |
| Bacteroides vulgatus | PI00000138 | 12 |
| Dorea longicatena | IS00006618 | 6 |
| Alistipes onderdonkii | IS00004389 | 4 |
| Blautia sp. | IS00002788 | 34 |

TABLE 12

| | | SEQ ID NO |
|---|---|---|
| Isolate Latin Name | ID Number | for 16S rRNA Sequence |
| Akkermansia muciniphila | IS00007180 | 20 |
| Bacteroides uniformis | PI00000137 | 11 |
| | OR | OR |
| | PI00000352 | 16 |
| Alistipes finegoldii | PI00000340 | 15 |
| Bacteroides vulgatus | PI00000138 | 12 |
| Dorea longicatena | IS00006618 | 6 |
| Blautia sp. | IS00002788 | 34 |

In embodiments, a pharmaceutical composition (e.g., comprising a microbial cocktail) can comprise one or more bacterial isolates (e.g., at least 1, at least 2, at least 3, at least 4, at least 5, at least 6, at least 7, at least 8, at least 9, at least 10, or 11 bacterial isolates) from Table 3, and/or one or more bacterial isolates (e.g., at least 1, at least 2, at least 3, at least 4, at least 5, at least 6, at least 7, at least 8, at least 9, at least 10, at least 11, at least 12, at least 13, at least 14, at least 15, or greater than 15 bacterial isolates) comprising a 16S rRNA sequence that is at least 95% identical to a 16S rRNA sequence of one or more bacterial isolates provided in Table 3. A microbial cocktail described herein can therefore comprise multiple bacterial isolates capable of modulating production and/or release of one or more cytokines from a eukaryotic cell (e.g., a cell of a subject administered a composition comprising the microbial cocktail).

In embodiments, a pharmaceutical composition (e.g., comprising a microbial cocktail) can comprise one or more bacterial isolates (e.g., at least 1, at least 2, at least 3, at least 4, at least 5, at least 6, at least 7, at least 8, at least 9, at least 10, at least 11, at least 12, at least 13, at least 14, at least 15, or greater than 15 bacterial isolates) that are not in Table 3. In embodiments, a pharmaceutical composition lacks at least one of the bacterial isolates in Table 3, or lacks bacterial isolates comprising 16S rRNA sequences at least 95% identical to the 16S rRNA sequence of at least one of the bacterial isolates in Table 2. In embodiments, a pharmaceutical composition (e.g., comprising a microbial cocktail) does not contain any bacterial isolate from Table 3. A pharmaceutical composition described herein can therefore comprise or consist of one or more bacterial isolates which do not substantially modulate production and/or release of one or more cytokines from a eukaryotic cell (e.g., a cell of a subject administered a composition comprising the microbial cocktail).

In embodiments, a pharmaceutical composition can comprise one or more bacterial isolates (e.g., at least 1, at least 2, at least 3, at least 4, at least 5, at least 6, at least 7, at least 8, at least 9, at least 10, at least 11, at least 12, at least 13, at least 14, at least 15, or greater than 15 bacterial isolates) from Table 4 comprising a 16S rRNA sequence that is at least 95% identical to a 16S rRNA sequence of one or more bacterial isolates provided in Table 4. A microbial cocktail described herein can therefore comprise multiple bacterial isolates corresponding to bacterial strains more highly abundant in a healthy subject relative to a patient with UC.

In embodiments, a pharmaceutical composition (e.g., comprising a microbial cocktail) can comprise one or more bacterial isolates (e.g., at least 1, at least 2, at least 3, at least 4, at least 5, at least 6, at least 7, at least 8, at least 9, at least 10, at least 11, at least 12, at least 13, at least 14, at least 15, or greater than 15 bacterial isolates) that are not in Table 4. In embodiments, a pharmaceutical composition lacks at least one of the bacterial isolates in Table 4, or lacks bacterial isolates comprising 16S rRNA sequences at least 95% identical to the 16S rRNA sequence of at least one of the bacterial isolates in Table 4. In embodiments, a pharmaceutical composition (e.g., comprising a microbial cocktail) does not contain any bacterial isolate from Table 4. A pharmaceutical composition described herein can therefore comprise or consist of one or more bacterial isolates which do not correspond to bacterial strains more highly abundant in a healthy subject relative to a patient with UC.

In embodiments, a microbial cocktail can comprise bacterial isolates comprising 16S rRNA sequences at least 95% identical to 16S rRNA sequences of bacterial isolates provided in more than one of Tables 2-4. Such a microbial cocktail can therefore advantageously influence the health of a subject administered the cocktail (e.g., in the form of a pharmaceutical composition) via multiple mechanisms. For example, a microbial cocktail can comprise one or more bacterial isolates that comprise a 16S rRNA sequence at least 95% identical to a 16S rRNA sequence of one or more bacterial isolates provided in Table 2, and one or more bacterial isolates that comprise a 16S rRNA sequence at least 95% identical to a 16S rRNA sequence of one or more bacterial isolates provided in Table 3. In such cases at least one bacterial isolate in the composition can produce one or more SCFAs to increase alevel of SCFAs (e.g., butyrate) in the gut of the subject (i.e., ‘Table 2 bacterial isolate’) and at least one bacterial isolate administered to the subject can modulate production and/or release of a cytokine by a host cell of the subject (i.e., ‘Table 3 bacterial isolate’).

In another example, a microbial cocktail can comprise one or more bacterial isolates that comprise a 16S rRNA sequence at least 95% identical to a 16S rRNA sequence of one or more bacterial isolates provided in Table 2, and one or more bacterial isolates that comprise a 16S rRNA sequence at least 95% identical to a 16S rRNA sequence of one or more bacterial isolates provided in Table 4. In such cases, at least one bacterial isolate in the composition can produce one or more SCFAs to increase a level of SCFAs (e.g., butyrate) in the gut of the subject (i.e., ‘Table 2 bacterial isolate’) and at least one bacterial isolate in the composition corresponds to a bacterial strain more highly abundant in a healthy subject relative to a patient with UC (‘Table 4 bacterial isolate’).

In another example, a microbial cocktail can comprise one or more bacterial isolates that comprise a 16S rRNA sequence at least 95% identical to a 16S rRNA sequence of one or more bacterial isolates provided in Table 3, and one or more bacterial isolates that comprise a 16S rRNA sequence at least 95% identical to a 16S rRNA sequence of one or more bacterial isolates provided in Table 4. In such cases, at least one bacterial isolate in the composition can modulate production and/or release of a cytokine by a host cell of the subject (i.e., ‘Table 3 bacterial isolate’) and at least one bacterial isolate in the composition corresponds to a bacterial strain more highly abundant in a healthy subject relative to a patient with UC (‘Table 4 bacterial isolate’).

In another example, a microbial cocktail can comprise one or more bacterial isolates that comprise a 16S rRNA sequence at least 95% identical to a 16S rRNA sequence of one or more bacterial isolates provided in Table 2, one or more bacterial isolates that comprise a 16S rRNA sequence at least 95% identical to a 16S rRNA sequence of one or more bacterial isolates provided in Table 3, and one or more bacterial isolates that comprise a 16S rRNA sequence at least 95% identical to a 16S rRNA sequence of one or more bacterial isolates provided in Table 4. In such cases, at least one bacterial isolate in the composition can produce one or more SCFAs to increase a level of SCFAs in the gut of the subject (i.e., ‘Table 2 bacterial isolate’), at least one bacterial isolate in the composition can modulate production and/or release of a cytokine by a host cell of the subject (i.e., ‘Table 3 bacterial isolate’), and at least one bacterial isolate in the composition corresponds to a bacterial strain more highly abundant in a healthy subject relative to a patient with UC (‘Table 4 bacterial isolate’).

Tables 13-21 illustrate different exemplary microbial cocktails containing bacterial isolates found in Tables 2-4, such that collectively the bacterial isolates in the microbial cocktail, once administered to a subject, can act to (i) increase a level of SCFAs in the gut of the subject; (ii) modulate release of a cytokine by a host cell of the subject; and (iii) provide for bacterial isolates in the gut of the subject that correspond to bacterial strains more highly abundant in a healthy subject relative to a patient with UC. In each table, isolates are identified by Latin name, an Identification Number (ID number), the Sequence Identifier (SEQ ID NO) for its 16S rRNA sequence, and the above Table(s) (i.e., Table 2, 3 and/or 4) in which the isolate appears.

In certain embodiments, a composition comprises a microbial cocktail comprising each of the bacterial isolates provided in one of Tables 13-21, or bacterial isolates having 16S rRNA sequences at least 95% identical to the 16S rRNA sequence of each of the bacterial isolates provided in one of Tables 13-21. In embodiments, the pharmaceutical composition comprises at least eight bacterial isolates that each comprise a 16S rRNA sequence at least 95% identical to a 16S rRNA sequence of at least one of the bacterial isolates provided in one of Tables 13-15, 15a, and 15b. In embodiments, the pharmaceutical composition comprises at least seven bacterial isolates that each comprise a 16S rRNA sequence at least 95% identical to a 16S rRNA sequence of at least one of the bacterial isolates provided in one of Tables 16-18. In embodiments, the pharmaceutical composition comprises at least six bacterial isolates that each comprise a 16S rRNA sequence at least 95% identical to a 16S rRNA sequence of at least one of the bacterial isolates provided in one of Tables 19-21. In certain embodiments, a composition comprises a microbial cocktail consisting essentially of each of the bacterial isolates provided in one of Tables 13-21, or bacterial isolates having 16S rRNA sequences at least 95% identical to the 16S rRNA sequence of each of the bacterial isolates provided in one of Tables 13-21. In certain embodiments, a composition comprises a microbial cocktail consisting of each of the bacterial isolates provided in one of Tables 13-21, or bacterial isolates having 16S rRNA sequences at least 95% identical to the 16S rRNA sequence of each of the bacterial isolates provided in one of Tables 13-21.

TABLE 13

| | | SEQ ID NO for | |
|---|---|---|---|
| Isolate Latin Name | ID Number | 16S rRNA Sequence | Table(s)* |
| Odoribacter splanchnicus | PI00000072 | 2 | 2, 3, 4 |
| Subdoligranulum variabile | IS00007359 | 22 | 2, 4 |
| | OR | OR | |
| | IS00007357 | 23 | |
| Eubacterium rectale | IS00006864 | 8 | 2, 4 |
| Alistipes shahii | PI00000395 | 18 | 3, 4 |
| Phascolarctobacterium | PI00000289 | 21 | 4 |
| faecium | | | |
| Bacteroides cellulosilyticus | PI00000316 | 14 | 4 |
| Akkermansia muciniphila | IS00007180 | 20 | 4 |
| Anaerostipes hadrus | PI00000094 | 3 | 2, 3, 4 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

TABLE 14

| | | SEQ ID NO for | |
|---|---|---|---|
| Isolate Latin Name | ID Number | 16S rRNA Sequence | Table(s)* |
| Odoribacter splanchnicus | PI00000072 | 2 | 2, 3, 4 |
| Subdoligranulum variabile | IS00007359 | 22 | 2, 4 |
| | OR | OR | |
| | IS00007357 | 23 | |
| Eubacterium rectale | IS00006864 | 8 | 2, 4 |
| Alistipes onderdonkii | IS00004389 | 4 | 4 |
| Phascolarctobacterium | PI00000289 | 21 | 4 |
| faecium | | | |
| Bacteroides cellulosilyticus | PI00000316 | 14 | 4 |
| Akkermansia muciniphila | IS00007180 | 20 | 4 |
| Anaerostipes hadrus | PI00000094 | 3 | 2, 3, 4 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

TABLE 15

| | | SEQ ID NO for | |
|---|---|---|---|
| Isolate Latin Name | ID Number | 16S rRNA Sequence | Table(s)* |
| Odoribacter splanchnicus | PI00000072 | 2 | 2, 3, 4 |
| Subdoligranulum variabile | IS00007359 | 22 | 2, 4 |
| | OR | OR | |
| | IS00007357 | 23 | |
| Eubacterium rectale | IS00006864 | 8 | 2, 4 |
| Alistipes finegoldii | PI00000340 | 15 | 4 |
| Phascolarctobacterium | PI00000289 | 21 | 4 |
| faecium | | | |
| Bacteroides cellulosilyticus | PI00000316 | 14 | 4 |
| Akkermansia muciniphila | IS00007180 | 20 | 4 |
| Anaerostipes hadrus | PI00000094 | 3 | 2, 3, 4 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

TABLE 15a

| | | SEQ ID NO | |
|---|---|---|---|
| Isolate Latin Name | ID Number | for 16S rRNA Sequence | Table* |
| Odoribacter splanchnicus | PI00000072 | 2 | 2, 3, 4 |
| Subdoligranulum variabile | IS00007359 | 22 | 2, 4 |
| | OR | OR | |
| | IS00007357 | 23 | |
| Eubacterium rectale | IS00006864 | 8 | 2, 4 |
| Alistipes shahii | PI00000395 | 18 | 3, 4 |
| Faecalibacterium prausnitzii | PI00000329 | 1 | 2, 3, 4 |
| | OR | OR | |
| | IS00006632 | 7 | |
| Bacteroides cellulosilyticus | PI00000316 | 14 | 4 |
| Akkermansia muciniphila | IS00007180 | 20 | 4 |
| Roseburia faecis | PI00000404 | 19 | 2 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

TABLE 15b

| | | SEQ ID NO | |
|---|---|---|---|
| Isolate Latin Name | ID Number | for 16S rRNA Sequence | Table* |
| Odoribacter splanchnicus | PI00000072 | 2 | 2, 3, 4 |
| Faecalibacterium prausnitzii | IS00006632 | 7 | 2, 3, 4 |
| Eubacterium rectale | IS00006864 | 8 | 2, 4 |
| Alistipes shahii | PI00000395 | 18 | 3, 4 |
| Faeca/ibacterium prausnitzii | PI00000329 | 1 | 2, 3, 4 |
| Bacteroides cellulosilyticus | PI00000316 | 14 | 4 |
| Akkermansia muciniphila | IS00007180 | 20 | 4 |
| Roseburia faecis | PI00000404 | 19 | 2 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

TABLE 16

| | | SEQ ID NO | |
|---|---|---|---|
| Isolate Latin Name | ID Number | for 16S rRNA Sequence | Table* |
| Odoribacter splanchnicus | PI00000072 | 2 | 2, 3, 4 |
| Subdoligranulum variabile | IS00007359 | 22 | 2, 4 |
| | OR | OR | |
| | IS00007357 | 23 | |
| Eubacterium rectale | IS00006864 | 8 | 2, 4 |
| Alistipes shahii | PI00000395 | 18 | 3, 4 |
| Faeca/ibacterium prausnitzii | PI00000329 | 1 | 2, 3, 4 |
| | OR | OR | |
| | IS00006632 | 7 | |
| Bacteroides cellulosilyticus | PI00000316 | 14 | 4 |
| Anaerostipes hadrus | PI00000094 | 3 | 2, 3, 4 |

*Table 2: Bacterial isolat\es that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

TABLE 17

| | | SEQ ID NO for | |
|---|---|---|---|
| Isolate Latin Name | ID Number | 16S rRNA Sequence | Table* |
| Odoribacter splanchnicus | PI00000072 | 2 | 2, 3, 4 |
| Subdoligranulum variabile | IS00007359 | 22 | 2, 4 |
| | OR | OR | |
| | IS00007357 | 23 | |
| Eubacterium rectale | IS00006864 | 8 | 2, 4 |
| Alistipes onderdonkii | IS00004389 | 4 | 4 |
| Faecalibacterium | PI00000329 | 1 | 2, 3, 4 |
| prausnitzii | OR | OR | |
| | IS00006632 | 7 | |
| Bacteroides cellulosilyticus | PI00000316 | 14 | 4 |
| Anaerostipes hadrus | PI00000094 | 3 | 2, 3, 4 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

TABLE 18

| | | SEQ ID NO for | |
|---|---|---|---|
| Isolate Latin Name | ID Number | 16S rRNA Sequence | Table* |
| Odoribacter splanchnicus | PI00000072 | 2 | 2, 3, 4 |
| Subdoligranulum variabile | IS00007359 | 22 | 2, 4 |
| | OR | OR | |
| | IS00007357 | 23 | |
| Eubacterium rectale | IS00006864 | 8 | 2, 4 |
| Alisti pes finegoldii | PI00000340 | 15 | 4 |
| Faecalibacterium prausnitzii | PI00000329 | 1 | 2, 3, 4 |
| | OR | OR | |
| | IS00006632 | 7 | |
| Bacteroides cellulosilyticus | PI00000316 | 14 | 4 |
| Anaerostipes hadrus | PI00000094 | 3 | 2, 3, 4 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

TABLE 19

| | | SEQ ID NO for | |
|---|---|---|---|
| Isolate Latin Name | ID Number | 16S rRNA Sequence | Table* |
| Eubacterium rectale | IS00006864 | 8 | 2, 4 |
| Bacteroide cellulosilyticus | PI00000316 | 14 | 4 |
| Faecalibacterium | PI00000329 | 1 | 2, 3, 4 |
| prausnitzii | OR | OR | |
| | IS00006632 | 7 | |
| Alistipes shahii | PI00000395 | 18 | 3, 4 |
| Anaerostipes hadrus | PI00000094 | 3 | 2, 3, 4 |
| Roseburia faecis | PI00000404 | 19 | 2 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

TABLE 20

| | | SEQ ID NO for | |
|---|---|---|---|
| Isolate Latin Name | ID Number | 16S rRNA Sequence | Table* |
| Eubacterium rectale | IS00006864 | 8 | 2, 4 |
| Bacteroides cellulosilyticus | PI00000316 | 14 | 4 |
| Faecalibacterium | PI00000329 | 1 | 2, 3, 4 |
| prausnitzii | OR | OR | |
| | IS00006632 | 7 | |
| Alistipes shahii | PI00000395 | 18 | 3, 4 |
| Blautia obeum | PI00000053 | 9 | 4 |
| Roseburia faecis | PI00000404 | 19 | 2 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

TABLE 21

| | | SEQ ID NO for | |
|---|---|---|---|
| Isolate Latin Name | ID Number | 16S rRNA Sequence | Table* |
| Odoribacter splanchnicus | PI00000072 | 2 | 2, 3, 4 |
| Eubacterium rectale | IS00006864 | 8 | 2, 4 |
| Bacteroides cellulosilyticus | PI00000316 | 14 | 4 |
| Faecalibacterium | PI00000329 | 1 | 2, 3, 4 |
| prausnitzii | OR | OR | |
| | IS00006632 | 7 | |
| Alistipes shahii | PI00000395 | 18 | 3, 4 |
| Blautia obeum | PI00000053 | 9 | 4 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

In embodiments, a pharmaceutical composition described herein can comprise a bacterial mixture comprising multiple bacterial isolates that together provide for redundancy of one or more advantageous phenotypes in the bacterial mixture, which may include for example the ability to induce a particular mechanism or pathway in the intestine of the subject when the composition is administered to the subject. Such redundancy can increase the likelihood that a subject administered the composition will benefit from a particular advantageous trait or phenotype in common between the “redundant” isolates of bacteria (e.g. a propensity to increase a level of an SCFA in the gut of the subject). Without intending to be bound by theory, a reason for this is potential variation across subjects in the likelihood of a particular bacterial isolate to engraft in the intestine of each subject, which is typically necessary for the bacterial isolate to exert its beneficial effects. By formulating a composition comprising genetically distinct isolates that each exhibit the same phenotype of interest (e.g. the production of one or more SCFAs above a threshold level), the composition may exhibit enhanced efficacy across individuals (i.e., compared to a formulation without built-in redundancy), since the likelihood of at least one of the multiple redundant bacterial isolates engrafting is increased compared to the likelihood of engraftment of a single bacterial isolate without a redundant counterpart.

In an aspect, redundancy can be incorporated into the composition by including in the bacterial mixture multiple bacterial isolates that each possess at least one of the following phenotypes: (i) each of the multiple bacterial isolates produces a level of one or more SCFAs above a threshold level (e.g., by incorporating into the bacterial mixture or administering to the subject multiple bacterial isolates selected from Table 2); (ii) each of the multiple bacterial isolates induces release of a cytokine by a host cell of the subject (e.g., by incorporating into the bacterial mixture or administering to the subject multiple bacterial isolates selected from Table 3); or (iii) each of the multiple bacterial isolates corresponds to a bacterial strain that is more highly abundant in a healthy subject relative to a patient with UC (e.g., by incorporating into the bacterial mixture or administering to the subject multiple bacterial isolates selected from Table 4).

In an aspect, a bacterial mixture comprising multiple bacterial isolates that provide for redundancy of phenotypes is shown in Table 15b, which contains at least five bacterial isolates that produce significant levels of an SCFA (here butyrate; see Example 2) (i.e., Odoribacter splanchnicus, Eubacterium rectale, Roseburia faecis, and two isolates of Faecalibacterium prausnitzii), at least four bacterial isolates that induce release of pro-inflammatory cytokines by human cells (here PBMCs; see Example 3) (i.e., Odoribacter sphlanchnicus, Alistipes shahii, and two isolates of Faecalibacterium prausnitzii), and at least seven bacterial isolates that correspond to bacterial strains more highly abundant in a healthy subject relative to a patient with UC (see Example 4) (i.e., Bacteroides cellulosilyticus, Odoribacter splanchnicus, Akkermansia muciniphila, Alistipes shahii, Eubacterium rectale and two isolates of Faecalibacterium prausnitzii).

In embodiments, a pharmaceutical composition can comprise multiple distinct bacterial isolates that are each a member of a particular genus, or multiple bacterial isolates that are each a member of a particular species. This can be advantageous, for example, by providing redundancy where multiple bacterial isolates of the same genus or species are capable of optimally inducing or influencing a particular mechanism of interest (e.g. by producing SCFA or inducing an anti-inflammatory profile. By including multiple taxonomically related bacterial isolates that each exhibit a similar optimal trait or phenotype (e.g. ability to produce high levels of an SCFA) in the same pharmaceutical composition, redundancy is built into the composition such that there is a higher likelihood that a subject administered the composition will benefit from the trait or phenotype. A reason for this is potential variation across subjects in the ability of a particular bacterial isolate to engraft in the intestines of the subjects, which is typically necessary for the bacterial isolate to exert its beneficial effects. For example, a pharmaceutical composition can comprise multiple (e.g. two, three, four, five, or more than five) bacterial isolates that are each a member of the genus Faecalibacterium, or multiple (e.g. two, three, four, five, or more than five) bacterial isolates that are each a member of the species Faecalibacterium prausnitzii (see e.g. Table 15b). In another example, a pharmaceutical composition can comprise multiple (e.g. two, three, four, five, or more than five) bacterial isolates that are each a member of the genus Bacteroides.

In embodiments, a pharmaceutical composition can comprise only one bacterial isolate that is a member of a particular genus, or only one bacterial isolate that is a member of a particular species. This can be advantageous, for example, to minimize the total number of bacterial isolates in a pharmaceutical composition (e.g. to reduce costs and resources related to growing and formulating each bacterial isolate), while maintaining a diversity of taxa represented by the bacterial isolates, which can correspond to induction of multiple treatment-related mechanisms (e.g. SCFA production or anti-inflammatory cytokine induction) in a subject administered the composition. For example, a pharmaceutical composition can comprise only one bacterial isolate that is a member of the genus Bacteroides. In an embodiment, the single bacterial isolate from the genus Bacteroides is B. cellulosilyticus (see e.g. Tables 15a and 15b). In another example, a pharmaceutical composition can comprise only one bacterial isolate that is a member of the genus Eubacterium, or only one bacterial isolate that is a member of the species Eubacterium rectale (see e.g. Tables 15a and 15b). In another example, a pharmaceutical composition can comprise only one bacterial isolate that is a member of the genus Roseburia, or only one bacterial isolate that is a member of the species Roseburia faecis (see e.g. Tables 15a and 15b). In another example, a pharmaceutical composition can comprise only one bacterial isolate that is a member of the genus Coprococcus, or only one bacterial isolate that is a member of the species Coprococcus comes.

In another example, a pharmaceutical composition can comprise only one bacterial isolate that is a member of the genus Alistipes. In certain embodiments, a pharmaceutical composition comprises a bacterial isolate comprising Alistipes shahii, but does not comprise Alistipes finegoldii or Alistipes putredinis (see e.g. Tables 15a and 15b). The inclusion of A. shahii over A. finegoldii and A. putredinis is consistent with the Examples below, which show that A. shahii has a lower cross sectional combined p-value than A. finegoldii and A. putredinis for (i) enrichment in healthy subjects over patients diagnosed with UC; and/or (ii) association/correlation with clinical remission or response of UC symptoms in UC patients following FMT treatment. In other embodiments, a pharmaceutical composition comprises a bacterial isolate comprising Alistipes finegoldii, but does not comprise Alistipes shahii or Alistipes putredinis (see e.g. Table 15). In certain embodiments, a pharmaceutical composition comprises a bacterial isolate comprising Alistipes Alistipes putredinis, but does not comprise Alistipes shahii or Alistipes finegoldii.

In embodiments, a pharmaceutical composition can exclude or omit a bacterial genus, species or strain that may not be beneficial, or may be detrimental, for treating a condition related to a gut dysbiosis (e.g., IBD including ulcerative colitis). For example, a pharmaceutical composition can exclude or omit a strain or bacterial isolate from the genus Escherichia (e.g., Escherichia coli). The below Examples show that E. coli produces little or no butyrate and is characterized by a cytokine profile that is pro-inflammatory (i.e. low ratios of IL-10/IL-12 and/or IL-10/TNF-alpha).

In other examples, a pharmaceutical composition can omit or exclude one or more of the following bacterial taxa: a member of the genus Adlercreutzia, Adlercreutzia equolifaciens, a member of the genus Akkermansia, Akkermansia muciniphila, a member of the genus Alistipes, Alistipes finegoldii, Alistipes putredinis, Alistipes shahii, a member of the genus Bacteroides, Bacteroides capillosus, Bacteroides cellulosilyticus, Bacteroides eggerthii, Bacteroides ovatus, Bacteroides thetaiotaomicron, Bacteroides uniformis, a member of the genus Bacillus, Bacillus circulans, Bacillus simplex, a member of the genus Bifidobacterium, Bifidobacterium longum, a member of the genus Blautia, Blautia hydrogenotrophica, a member of the genus Brevibacillus, Brevibacillus parabrevis, a member of the genus Catabacter, Catabacter hongkongensis, a member of the genus Catenibacterium, Catenibacterium mitsuokai, a member of the genus Clostridium, Clostridium coccoides, Clostridium aldenense, Clostridium asparagiforme, Clostridium celerecrescens, Clostridium hathewayi, Clostridium hylemonae, Clostridium inocuum, Clostridium lavalense, Clostridium leptum, Clostridium scindens, Clostridium staminisolvens, Clostridium sulfatireducens, Clostridium symbiosum, Clostridium thermocellum, a member of the genus Collinsella, Collinsella aerofaciens, a member of the genus Coprococcus, Coprococcus catus, Coprococcus comes, Coprococcus eutactus, a member of the genus Dorea, Dorea formicigenerans, Dorea longicatena, a member of the genus Eubacterium, Eubacterium biforme, Eubacterium callanderi, Eubacterium dolichum, Eubacterium eligens, Eubacterium fissicatena, Eubacterium rectale, Eubacterium siraeum, Eubacterium ventriosum, Eubacterium xylanophilum, a member of the genus Faecalibacterium, Faecalibacterium prausnitzii, a member of the genus Holdemania, Holdemania filimormis, a member of the genus Subdoligranulum, Subdoligranulum variabile, a member of the genus Microbacterium, Microbacterium schleiferi, Micrococcus luteus, a member of the genus Odoribacter, Odoribacter splanchnicus, a member of the genus Oscillibacter, Oscillibacter valericigenes, a member of the genus Parabacteroides, Parabacteroides merdae, Parabacteroides gordonii, a member of the genus Parasutterella, Parasutterella excrementihominis, a member of the genus Phascolarctobacterium, Phascolarctobacterium faecium, a member of the genus Roseburia, Roseburia faecalis, Roseburia faecis, Roseburia hominis, Roseburia intestinalis, a member of the genus Ruminococcus, Ruminococcus albus, Ruminococcus bromii, Ruminococcus lactaris, Ruminococcus luti, Ruminococcus obeum, Ruminococcus torques, a member of the genus Staphylococcus, Staphylococcus epidermidis, a member of the genus Streptococcus, Streptococcus mitis, Streptococcus thermophilus, a member of the genus Synergistes, a member of the genus Turicibacter, and Turicibacter sanguinis.

In embodiments, a bacterial isolate incorporated into a pharmaceutical composition can act through multiple mechanisms to advantageously impact the health of a subject. Such a bacterial isolate can therefore advantageously influence the health of a subject administered the cocktail (e.g., in the form of a pharmaceutical composition) via multiple mechanisms. For example, a pharmaceutical composition can comprise a bacterial isolate that when administered to a subject can produce one or more SCFAs to increase a level of SCFAs (e.g., butyrate) in the gut of the subject (‘Table 2 bacterial isolate’), and can also modulate production and/or release of a cytokine by a host cell of the subject (‘Table 3 bacterial isolate’). In another example, a pharmaceutical composition can comprise a bacterial isolate that when administered to a subject can produce one or more SCFAs to increase a level of SCFAs (e.g., butyrate) in the gut of the subject (‘Table 2 bacterial isolate’), and also corresponds to a bacterial strain more highly abundant in a healthy subject relative to a patient with UC (‘Table 4 bacterial isolate’). In another example, a pharmaceutical composition can comprise a bacterial isolate that when administered to a subject can modulate production and/or release of a cytokine by a host cell of the subject (‘Table 3 bacterial isolate’), and also correspond to a bacterial strain more highly abundant in a healthy subject relative to a patient with UC (‘Table 4 bacterial isolate’). In another example, a pharmaceutical composition can comprise a bacterial isolate that when administered to a subject can produce one or more SCFAs to increase a level of SCFAs (e.g., butyrate) in the gut of the subject (‘Table 2 bacterial isolate’), can also modulate production and/or release of a cytokine by a host cell of the subject (‘Table 3 bacterial isolate’), and also corresponds to a bacterial strain more highly abundant in a healthy subject relative to a patient with UC (‘Table 4 bacterial isolate’).

Table 22 illustrates exemplary bacterial isolates capable of positively affecting the health of a subject via multiple mechanisms (e.g., production of SCFAs, induction of cytokine release by host cells, or providing bacteria in the gut that correspond to bacterial strains more highly abundant in a healthy subject relative to a patient with UC). In Table 22, isolates are identified by Latin name, an Identification Number (ID number), the Sequence Identifier (SEQ ID NO) for its 16S rRNA sequence, and the above Tables 2-4 in which the bacterial isolate appears (with Table 2 representing bacterial isolates that secrete SCFAs in the gut of a subject, Table 3 representing bacterial isolates capable of modulating cytokine production by a host cell, Table 4 representing bacterial isolates corresponding to bacterial strains more highly abundant in a healthy subject than a patient with UC). In certain embodiments, a pharmaceutical composition (e.g., a microbial cocktail) comprises one or more bacterial isolates provided in Table 22, and/or one or more bacterial isolates having a 16S rRNA sequence that is at least 95% identical to the 16S rRNA sequence of one or more of the bacterial isolates provided in Table 22.

TABLE 22

| | | SEQ ID NO for | |
|---|---|---|---|
| Isolate Latin Name | ID Number | 16S rRNA Sequence | Table* |
| Faecalibacterium prausnitzii | PI00000329 | 1 | 2, 3, 4 |
| Odoribacter splanchnicus | PI00000072 | 2 | 2, 3, 4 |
| Anaerostipes hadrus | PI00000094 | 3 | 2, 3, 4 |
| Faecalibacterium prausnitzii | IS00006632 | 7 | 2, 3, 4 |
| Eubacterium rectale | IS00006864 | 8 | 2, 4 |
| Bacteroides uniformis | PI00000137 | 11 | 3, 4 |
| Bacteroides vulgatus | PI00000138 | 12 | 3, 4 |
| Bacteroides uniformis | PI00000352 | 16 | 3, 4 |
| Coprococcus comes | PI00000370 | 17 | 2, 3 |
| Alistipes shahii | PI00000395 | 18 | 3, 4 |
| Subdoligranulum variabile | IS00007359 | 22 | 2, 4 |
| Subdoligranulum variabile | IS00007357 | 23 | 2, 4 |

*Table 2: Bacterial isolates that secrete SCFAs in the gut of a subject. Table 3: Bacterial isolates capable of modulating cytokine production by a host cell of a subject. Table 4: Bacterial isolates that correspond to a bacterial strain more highly abundant in a healthy subject relative to a patient with UC.

In embodiments, a bacterial isolate can comprise a 16S rRNA sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the 16S rRNA sequence of at least one of the bacterial isolates provided in Table 1 to Table 22. In embodiments, a bacterial isolate comprises a 16S rRNA sequence that is at least 97% identical to the 16S rRNA sequence of at least one of the bacterial isolates provided in Table 1 to Table 22.

In embodiments, a pharmaceutical composition (e.g., comprising a microbial cocktail) described herein can comprise at least one bacterial isolate that has been previously identified. For example, in certain embodiments a known type strain (e.g., from the ATCC or DSM archived collections) can comprise a 16S rRNA sequence that is at least 95% identical (e.g., at least 97% identical) to a 16S rRNA sequence of a bacterial isolate provided in Table 1 to Table 22. Exemplary publicly available strains that can be included in a microbial cocktail described herein are provided in Table 23. In an embodiment, for a particular pharmaceutical composition, one or more bacterial isolates provided in Table 1 to Table 22 can be substituted with the corresponding representative strain (i.e., having the same taxonomic designation) from Table 23.

TABLE 23

| Isolate Latin Name | Representative Strain* |
|---|---|
| Faecalibacterium prausnitzii | ATCC 27768 |
| Odoribacter splanchnicus | ATCC 29572 |
| Odoribacter laneus | DSM 22474 |
| Anaerostipes hadrus | DSM 3319 |
| Alistipes onderdonkii | ATCC BAA-1178 |
| Parabacteroides merdae | ATCC 43184 |
| Dorea longicatena | DSM 13814 |
| Eubacterium rectale | ATCC 33656 |
| Blautia obeum | DSM 25238 |
| Bacteroides uniformis | ATCC 8492 |
| Bacteroides vulgatus | DSM 1447 |
| Bacteroides cellulosilyticus | DSM 14838 |
| Alistipes finegoldii | DSM 17242 |
| Coprococcus comes | ATCC 27758 |
| Alistipes shahii | ATCC BAA-1179 |
| Roseburia faecis | DSM 16840 |
| Akkermansia muciniphila | DSM 22959 |
| Phascolarctobacterium faecium | DSM 14760 |
| Subdoligranulum variabile | DSM 15176 |
| Alistipes putredinis | ATCC 29800 |

*DSM: Leibniz Institute DSMZ-German Collection of Microorganisms and Cell Cultures ATCC: American Type Culture Collection

In embodiments, a pharmaceutical composition or microbial cocktail comprises a plurality of bacterial isolates isolated or purified from stool samples of multiple different human donors. An advantage of sourcing bacterial isolates from stool samples of multiple human donors, rather than a single donor, is that stool samples of multiple donors offer a larger pool of bacterial strains that can be subjected to functional screens to identify bacterial isolates that optimally induce a particular mechanism of interest. For example, while the stool of one donor may carry one or more bacterial strains that produce a high (optimal) level of an SCFA (e.g., butyrate), the same donor's stool may carry bacterial isolates that only moderately induce an anti-inflammatory profile (e.g. moderate IL-10:IL-12 ratio) when subjected to a functional screen. Alternatively, a different donor's stool may carry one or more bacterial strains that induce an optimal anti-inflammatory profile (e.g. high IL-10:IL-12 ratio), but not carry bacterial strains that produce high levels of an SCFA.

Another advantage of using stool from multiple different donors as a basis of selection when identifying bacterial isolates to include within a pharmaceutical composition described herein is that microbiota from multiple donors yields a larger pool of bacterial strains within which to identify a bacterial isolate corresponding to a bacterial strain having (i) a greater relative abundance in a healthy human subject relative to a patient with an intestinal dysbiosis, or (ii) a greater relative abundance in a human subject in remission from an intestinal dysbiosis relative to a patient having the intestinal dysbiosis (i.e., bacterial isolates provided in Table 4). In general, the match between a 16S rRNA sequence of a bacterial strain having (i) or (ii) above and the 16S rRNA sequence of a selected bacterial isolate should be as close as possible (e.g., at least 97%, at least 97.5%, at least 98%, at least 98.5%, at least 99%, at least 99.5%, or at least 100%). The likelihood of identifying the desired match increases with a greater pool of bacterial strains to select from, which is provided by sourcing stool from multiple donors.

Therefore, in general, the rational mechanism-based selection scheme set out herein to construct a microbial therapeutic favors increasing the pool of bacterial strains beyond those of a single donor to enable selection of optimal bacterial isolates for induction of each mechanism of interest in a subject administered the therapeutic. This multi-donor selection process is therefore advantageous over known approaches which attempt to recreate a synthetic version of a single donor's stool.

In other embodiments, a pharmaceutical composition or microbial cocktail comprises a plurality of bacterial isolates isolated or purified from a stool sample or stool samples of only a single human donor.

In various embodiments, a pharmaceutical composition (e.g., comprising a microbial cocktail) comprises one or more bacterial isolates capable of engrafting in a subject's GI tract following administration of the composition to the subject. Herein “engrafting” or “engraftment” refers to the stable presence over time of cells of a bacterial strain or bacterial isolate in the intestinal tract of a subject (e.g., after introducing the bacterial strain or isolate into the subject's intestinal tract by administering a composition described herein, for example, orally or rectally). Typically, engraftment of a bacterial isolate introduced into the intestine of a patient (e.g. by oral and/or rectal administration) is measured longitudinally, or over time, by comparing the abundance of the bacterial isolate in fecal samples of the subject before and after administration of the bacterial isolate to the subject. In an embodiment, the bacterial isolate introduced into the intestine of the subject was absent prior to the administration. In another embodiment, the bacterial isolate introduced into the intestine of the subject was present in the intestine prior to the administration, but is increased abundance following the administration. In certain embodiments, engraftment is determined by identifying an increase in abundance of a bacterial strain administered to an intestine of the subject after at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14, days, at least 1 week, at least 2 weeks, at least 3 weeks, at least 4 weeks, at least 5 weeks, at least 6 weeks, at least 7 weeks, at least 8 weeks, at least 1 month, at least 2 months, at least 3 months, at least 4 months, at least 5 months, at least 6 months, or greater than 6 months following administration of the bacterial strain to the subject.

In embodiments, engraftment of a bacterial isolate in an intestine of a subject occurs when the bacterial isolate is administered to the subject at or above a threshold dose. In embodiments, engraftment of a bacterial isolate in an intestine of a subject does not occur, or occurs with relative inefficiency (e.g., across patients), when the bacterial isolate is administered to the subject below the threshold dose. For example, engraftment of a bacterial isolate into the intestine of a subject can occur when the bacterial isolate is administered to the subject (e.g., orally or rectally in a pharmaceutical composition described herein) at a dose of at least 10⁶ cells, at least 10⁷ cells, at least 10⁸ cells, at least 10⁹ cells, at least 10¹⁰ cells, at least 10¹¹ cells, or at least 10¹² cells.

In embodiments, a dose of one or more bacterial isolates to a patient in need thereof can depend on the engraftment threshold of the bacterial isolate.

In embodiments, a bacterial isolate in a pharmaceutical composition administered to a subject engrafts in the duodenum of the subject. In embodiments, a bacterial isolate in a pharmaceutical composition administered to a subject engrafts in the jejunum of the subject. In embodiments, a bacterial isolate in a pharmaceutical composition administered to a subject engrafts in the ileum of the subject. In embodiments, a bacterial isolate in a pharmaceutical composition administered to a subject engrafts in the colon of the subject.

In embodiments, engraftment of a bacterial isolate in an intestine of a subject occurs when the bacterial isolate is administered to the subject at or below a threshold dose. In embodiments, engraftment of a bacterial isolate in an intestine of a subject does not occur, or occurs with relative inefficiency (e.g., across patients), when the bacterial isolate is administered to the subject above the threshold dose. For example, engraftment of a bacterial isolate into the intestine of a subject can occur when the bacterial isolate is administered to the subject (e.g., orally or rectally in a pharmaceutical composition described herein) at a dose of not more than 10⁸ cells, not more than 10⁹ cells, not more than 10¹⁰ cells, not more than 10¹¹ cells, or not more than 10¹² cells.

In various embodiments, the present pharmaceutical compositions (e.g., microbial cocktails) includes one or more bacterial isolates that interact, including synergistically, to have an inhibitory effect on the growth and/or survival of a pathogenic bacterium present in a gut of a subject administered the composition. For example, one or more bacterial isolates can have a cytotoxic or cytostatic effect on the pathogenic bacterium. In various embodiments, one or more bacterial isolates exert an inhibitory effect on a pathogenic bacterium present in or entering into the GI tract of a patient. In various embodiments, the one or more bacterial isolates augment growth of at least one type of bacteria not detectably present in a patient's GI tract prior to administration.

In various embodiments, a pharmaceutical composition (e.g., comprising a microbial cocktail) includes one or more isolated or purified bacterial strains that interact synergistically to have an inhibitory effect on the growth or survival of a pathogenic bacterium (e.g., via cytotoxic and/or cytostatic effects). Illustrative pathogenic bacteria that can be affected by administration of one or more bacterial isolates described herein include C. difficile, Salmonella sp., enteropathogenic E. coli, multi-drug resistant bacteria such as Klebsiella, and E. coli, Carbapenem-resistant Enterobacteriaceae (CRE), extended spectrum beta-lactam resistant Enterococci (ESBL), and vancomycin-resistant Enterococci (VRE). Further illustrative bacteria include Yersinia, Vibrio, Treponema, Streptococcus, Staphylococcus, Shigella, Salmonella, Rickettsia, Orientia, Pseudomonas, Neisseria, Mycoplasma, Mycobacterium, Listeria, Leptospira, Legionella, Klebsiella, Helicobacter, Haemophilus, Francisella, Escherichia, Ehrlichia, Enterococcus, Coxiella, Corynebacterium, Clostridium, Chlamydia, Chlamydophila, Campylobacter, Burkholderia, Brucella, Borrelia, Bordetella, Bifidobacterium, Bacillus, multi-drug resistant bacteria, extended spectrum beta-lactam resistant Enterococci (ESBL), Carbapenem-resistant Enterobacteriaceae (CRE), and vancomycin-resistant Enterococci (VRE). Illustrative pathogenic bacteria include Aeromonas hydrophila, Campylobacter fetus, Plesiomonas shigelloides, Bacillus cereus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enterotoxigenic Escherichia coli (such as, but not limited to, LT and/or ST), Escherichia coli O157:H7, Helicobacter pylori, Klebsiellia pneumonia, Lysteria monocytogenes, Plesiomonas shigelloides, Salmonella sp., Salmonella typhi, Salmonella paratyphi, Shigella sp., Staphylococcus sp., Staphylococcus aureus, vancomycin-resistant enterococcus sp., Vibrio sp., Vibrio cholerae, Vibrio parahaemolyticus, Vibrio vulnificus, and Yersinia enterocolitica.

In some embodiments, a bacterial isolate is a non-pathogenic bacterial strain. In embodiments, a non-pathogenic bacterial strain comprises a genome that lacks genes, or expression thereof, which cause virulence and/or toxicity. For instance, in some embodiments, a microbial cocktail comprising one or more bacterial isolates is substantially free of organisms or entities (e.g., substantially free of pathogenic bacteria) which are capable of causing a disease or disorder in a subject administered the microbial cocktail.

In embodiments, the microbial cocktail does not include bacterial cells other than the one or more bacterial isolates incorporated into the microbial cocktail during the manufacturing process. For example, the microbial cocktail can be free of particular species of bacteria, whether cultured or uncultured, including Bacteroides, Bifidobacterium, Desulfomonas, Clostridium, Escherichia coli, Eubacterium, Fusobacterium, Lactobacillus, Monilia, Peptostreptococcus, Propionibacterium, or Ruminococcus.

In embodiments, a bacterial isolate can be obtained from a laboratory stock or a bacterial cell bank of a bacterial strain originally obtained from a stool sample of a healthy human donor. For example, a fecal microbiota (e.g., purified from a stool sample using methods described herein) can be used as the source of a bacterial isolate incorporated into a pharmaceutical composition described herein. In certain embodiments, all or a portion of a fecal microbiota of a stool sample is cultured on a solid media substrate and one or more bacterial isolates are identified as single colonies. In other embodiments, all or a portion of a fecal microbiota can be inoculated into liquid culture to produce a mixed bacterial culture that is then serially diluted to produce a culture containing a single cell of a bacterial isolate. In embodiments, an identified bacterial isolate can then be cultured (e.g., in solid or liquid media) using known techniques and expanded. Methods for isolating, purifying, and/or culturing bacterial strains are described in Sadowsky et al., WO 2012/122478 and described in Borody et al., WO 2012/016287, each of which is incorporated herein by reference.

### Uncultured Fecal Microbiota or Preparations of Uncultured Fecal Bacteria

In one aspect, a pharmaceutical composition administered herein comprises an uncultured fecal microbiota or a preparation of uncultured fecal bacteria. For example, an uncultured fecal microbiota or a preparation of uncultured fecal bacteria can comprise a substantially complete fecal microbiota (e.g., purified from a healthy human donor).

In embodiments, the preparation of a fecal microbiota used herein, or the manufacture of a preparation of uncultured fecal bacteria, involves a treatment selected from the group consisting of ethanol treatment, detergent treatment, heat treatment, irradiation, and sonication. In another aspect, the preparation of a fecal microbiota used herein, or the manufacture of a preparation of uncultured fecal bacteria, involves no treatment selected from the group consisting of ethanol treatment, detergent treatment, heat treatment, irradiation, and sonication. In one aspect, the preparation of a fecal microbiota used herein, or the manufacture of a preparation of uncultured fecal bacteria, involves a separation step selected from the group consisting of density gradients, filtration (e.g., sieves, nylon mesh), and chromatography. In another aspect, the preparation of a fecal microbiota used herein, or the manufacture of a preparation of uncultured fecal bacteria, involves no separation step selected from the group consisting of density gradients, filtration (e.g., sieves, nylon mesh), and chromatography. In another aspect, a fecal microbiota or preparation of uncultured fecal bacteria used herein comprises a donor's entire fecal microbiota. In another aspect, a pharmaceutical composition administered herein comprises a fecal microbiota or preparation of uncultured fecal bacteria substantially free of eukaryotic cells from the fecal microbiota's donor.

In another aspect, a pharmaceutical composition administered herein comprises an uncultured fecal microbiota or a preparation of uncultured fecal bacteria further supplemented, spiked, or enhanced with one or more bacterial isolates described herein. In one aspect, an uncultured fecal microbiota or a preparation of uncultured fecal bacteria is spiked with a bacterial isolate provided in Table 1. In one aspect, an uncultured fecal microbiota or a preparation of uncultured fecal bacteria is supplemented with a non-pathogenic (or with attenuated pathogenicity) bacterium of Clostridium, Collinsella, Dorea, Ruminococcus, Coprococcus, Prevotella, Veillonella, Bacteroides, Baccillus, or a combination thereof. In another aspect, a pharmaceutical composition administered herein comprises an uncultured fecal microbiota or a preparation of uncultured fecal bacteria further supplemented, spiked, or enhanced with a species of Veillonellaceae, Firmicutes, Gammaproteobacteria, Bacteroidetes, or a combination thereof. In another aspect, a pharmaceutical composition administered herein comprises an uncultured fecal microbiota or a preparation of uncultured fecal bacteria further supplemented with fecal bacterial spores. In one aspect, fecal bacterial spores are Clostridium spores, Bacillus spores, or both.

In an aspect, a pharmaceutical composition comprises an uncultured fecal microbiota or a preparation of uncultured fecal bacteria from a subject selected from the group consisting of a human, a bovine, a dairy calf, a ruminant, an ovine, a caprine, or a cervine. In another aspect, a pharmaceutical composition can be administered to a subject selected from the group consisting of a human, a bovine, a dairy calf, a ruminant, an ovine, a caprine, or a cervine. In an aspect, a pharmaceutical composition is substantially or nearly odorless.

In an aspect, a pharmaceutical composition provided or administered herein comprises an uncultured fecal microbiota or a preparation of uncultured fecal bacteria comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a pharmaceutical composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a pharmaceutical composition comprises a preparation of flora in proportional content that resembles a normal healthy human fecal flora.

In a further aspect, a pharmaceutical composition comprises fecal bacteria from at least 1, 2, 3, 4, 5, 6, 7, 8, 9, or 10 different families. In another aspect, a pharmaceutical composition comprises fecal bacteria from at least 11, 12, 13, 14, 15, 16, 17, 18, 19, or 20 different families. In yet another aspect, a pharmaceutical composition comprises fecal bacteria from at least 21, 22, 23, 24, 25, 26, 27, 28, 29, or 30 different families. In a further aspect, a pharmaceutical composition comprises fecal bacteria from at least 31, 32, 33, 34, 35, 36, 37, 38, 39, or 40 different families. In another aspect, a pharmaceutical composition comprises fecal bacteria from at least 41, 42, 43, 44, 45, 46, 47, 48, 49, or 50 different families. In another aspect, a pharmaceutical composition comprises fecal bacteria from between 1 and 10, between 10 and 20, between 20 and 30, between 30 and 40, between 40 and 50 different families. In an aspect, a pharmaceutical composition provided or administered herein comprises an uncultured fecal microbiota or a preparation of uncultured fecal bacteria comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a pharmaceutical composition provided or administered herein comprises an uncultured fecal microbiota or a preparation of uncultured fecal bacteria comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a pharmaceutical composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.33 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.002 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one embodiment, “biological material” refers to the living material, e.g., the microbes, eukaryotic cells, and viruses, which are present in the colon of a normal healthy human. In an aspect, a pharmaceutical composition provided or administered herein comprises an extract of human stool, wherein the composition is substantially odorless. In an aspect, a pharmaceutical composition provided or administered herein comprises fecal material or a fecal floral preparation in a lyophilized, crude, semi-purified or purified formulation.

In an aspect, an uncultured fecal microbiota or a preparation of uncultured fecal bacteria in a pharmaceutical composition comprises highly refined or purified fecal flora, e.g., substantially free of non-floral fecal material. In an aspect, an uncultured fecal microbiota or a preparation of uncultured fecal bacteria can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, an uncultured fecal microbiota or a preparation of uncultured fecal bacteria in a pharmaceutical composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, an uncultured fecal microbiota or a preparation of uncultured fecal bacteria in a pharmaceutical composition comprises a donor's substantially entire or non-selected fecal microbiota. In another aspect, the fecal microbiota in a pharmaceutical composition comprises no antibiotic resistant population. In another aspect, a pharmaceutical composition comprises an uncultured fecal microbiota or a preparation of uncultured fecal bacteria and is largely free of extraneous matter (e.g., non-living matter including acellular matter such as residual fiber, DNA, RNA, viral coat material, non-viable material; and living matter such as eukaryotic cells from the fecal matter's donor).

In an aspect, an uncultured fecal microbiota or a preparation of uncultured fecal bacteria in a pharmaceutical composition used herein is derived from a disease-screened stool sample of a human donor. In an aspect, a stool sample does not include an antibiotic resistant population. For example, a fecal composition can comprise a preparation of viable flora which can in proportional content, resemble normal healthy human fecal flora which does not include antibiotic resistant populations. Suitable microorganisms in a flora can be selected from the following: Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a pharmaceutical composition used in a treatment disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, excreted or otherwise liquid components or a microbiota, e.g., biologically active molecules (BAMs), which can be antibiotics or anti-inflammatories, are preserved, retained or reconstituted in a flora extract.

In one aspect, an exemplary pharmaceutical composition comprising a fecal filtrate comprises starting material from a donor from a defined donor pool, where this donor contributes a stool that is centrifuged, then filtered with very high-level filtration using e.g., either metal sieving or Millipore filters, or equivalent, to ultimately permit only cells of bacterial origin to remain, e.g., often less than about 5 micrometers diameter. After the initial centrifugation, the solid material can be separated from the liquid, and the solid is then filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation can be filtered progressively in a filtering, e.g., a Millipore filtering or equivalent systems, to produce a liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a pharmaceutical composition used herein comprises a reconstituted fecal flora consisting essentially of a combination of a purified fecal microbiota and a non-cellular fecal filtrate. In another aspect, a pharmaceutical composition used herein comprises a purified fecal microbiota supplemented with one or more non-cellular non-particulate fecal components. In one aspect, a pharmaceutical composition used here comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a treatment method provided here comprises the use of both fecal bacterial cells, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a fecal bacteria-based pharmaceutical composition is used concurrently with a fecal non-cellular filtrate-based pharmaceutical composition. In another aspect, a patient is treated with a first fecal non-cellular filtrate-based pharmaceutical composition before being given a second fecal bacteria-based pharmaceutical composition, or vice versa. In a further aspect, a treatment method comprises three steps: first, antibiotic pretreatment to non-selectively remove infectious pathogen(s); second, a fecal non-cellular filtrate-based treatment step to further suppress selected infectious pathogen(s); and third, giving the patient a fecal bacteria-based pharmaceutical composition to re-establish a functional intestinal microbiome.

In an aspect, a treatment method effects a cure, reduction of the symptoms, or a percentage reduction of symptoms of a disorder related to an intestinal dysbiosis. The change of flora can be as “near-complete” as possible and the flora is replaced by viable organisms which will crowd out any remaining, original flora. Typically, the change in enteric flora comprises introduction of an array of predetermined flora into the gastro-intestinal system, and thus in an aspect the method of treatment comprises substantially or completely displacing pathogenic enteric flora in patients requiring such treatment.

In an aspect, uncultured fecal microbiota or a preparation of uncultured fecal bacteria for incorporation into a pharmaceutical composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordelihi, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In an aspect, uncultured fecal microbiota or a preparation of uncultured fecal bacteria for incorporation into a pharmaceutical composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a pharmaceutical composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, an uncultured fecal microbiota or a preparation of uncultured fecal bacteria for incorporation into a pharmaceutical composition comprises two or more genera selected from the group consisting of Collinsella, Coprococcus, Dorea, Eubacterium, and Ruminococcus. In another aspect, a pharmaceutical composition comprises two or more genera selected from the group consisting of Coprococcus, Dorea, Eubacterium, and Ruminococcus. In a further aspect, a pharmaceutical composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, an uncultured fecal microbiota or a preparation of uncultured fecal bacteria for incorporation into a pharmaceutical composition described herein comprises at least about 10⁵, 10⁶, 10⁷, 10⁸, 10⁹, 10¹⁰, 10¹¹, 10¹², or 10¹³ cfu or total cell count. In another aspect, a pharmaceutical composition comprises at most about 10⁵, 10⁶, 10⁷, 10⁸, 10⁹, 10¹⁰, 10¹¹, 10¹², 10¹³ or 10¹⁴ cfu or total cell count.

In another aspect, an uncultured fecal microbiota or a preparation of uncultured fecal bacteria comprises at least about 10⁵, 10⁶, 10⁷, 10⁸, 10⁹, 10¹⁰, 10¹¹, 10¹², or 10¹³ cells or total cell count. In another aspect, a pharmaceutical composition comprises at most about 10⁵, 10⁶, 10⁷, 10⁸, 10⁹, 10¹⁰, 10¹¹, 10¹², 10¹³ or 10¹⁴ cells or total cell count.

Extraction and Purification of Bacteria from Stool

The pharmaceutical compositions described here can comprise microbes, e.g. bacteria, derived from a stool sample of a donor, e.g. a healthy human donor. In an aspect, a composition incorporates all or a portion of a fecal microbiota of a stool sample. For example, a composition can incorporate a substantially complete fecal microbiota of a stool sample of a healthy human donor. In an aspect, a composition incorporates a bacterial isolate of a fecal microbiota, wherein the bacterial isolate has been purified and/or cultured from all or a portion of the fecal microbiota. The extraction and/or purification of a fecal microbiota from a stool sample can thus be performed to prepare a composition comprising at least one of a fecal microbiota (e.g., a substantially complete fecal microbiota) or a bacterial isolate.

In one aspect, an exemplary fecal microbiota for use in preparing a composition described herein comprises starting material from a human donor. In another aspect, an exemplary fecal microbiota comprises material from one or more healthy human donors. In yet another aspect, an exemplary fecal microbiota comprises starting material from a pool of known, defined donors. In another aspect, a donor is an adult male. In a further aspect, a donor is an adult female. In yet another aspect, a donor is an adolescent male. In another aspect, a donor is an adolescent female. In another aspect, a donor is a female toddler. In another aspect, a donor is a male toddler. In another aspect, a donor is healthy. In one aspect, a human donor is a child below about 18, 15, 12, 10, 8, 6, 4, 3, 2, or 1-year-old. In another aspect, a human donor is an elderly individual. In a further aspect, a human donor is an individual above about 30, 35, 40, 45, 50, 55, 60, 65, 70, 75, 80, 85, 90, or 95 years old. In another aspect, a donor is between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a donor is a young old individual (65-74 years). In one aspect, a donor is a middle old individual (75-84 years). In one aspect, a donor is an old individual (>85 years). In yet another aspect, a donor is a carefully screened, healthy, neurotypical human.

In an aspect, a carefully screened donor undergoes a complete medical history and physical exam. Donors are excluded if they have a risk of infectious agents. Additional exclusion criteria comprise the following:
1. Known viral infection with Hepatitis B, C or HIV
2. Known exposure to HIV or viral hepatitis at any time
3. High risk behaviors including sex for drugs or money, men who have sex with men, more than one sexual partner in the preceding 12 months, any past use of intravenous drugs or intranasal cocaine, history of incarceration.
4. Tattoo or body piercing within 12 months.
5. Travel to areas of the world where risk of traveler's diarrhea is higher than the US.
6. Current communicable disease, e.g., upper respiratory viral infection.
7. History of irritable bowel syndrome. Specific symptoms can include frequent abdominal cramps, excessive gas, bloating, abdominal distension, fecal urgency, diarrhea, constipation.
8. History of inflammatory bowel disease such as Crohn's disease, ulcerative colitis, microscopic colitis.
9. Chronic diarrhea.
10. Chronic constipation or use of laxatives.
11. History of gastrointestinal malignancy or known colon polyposis.
12. History of any abdominal surgery, e.g., gastric bypass, intestinal resection, appendectomy, cholecystectomy, etc.
13. Use of Probiotics or any other over the counter aids used by the potential donor for purpose of regulating digestion. Yogurt and kefir products are allowed if taken merely as food rather than nutritional supplements.
14. Antibiotics for any indication within the preceding 6 months.
15. Any prescribed immunosuppressive or anti-neoplastic medications.
16. Metabolic Syndrome, established or emerging. Criteria used for definition here are stricter than any established criteria. These include history of increased blood pressure, history of diabetes or glucose intolerance.
17. Known systemic autoimmunity, e.g., connective tissue disease, multiple sclerosis.
18. Known atopic diseases including asthma or eczema.
19. Chronic pain syndromes including fibromyalgia, chronic fatigue syndrome.
20. Ongoing (even if intermittent) use of any prescribed medications, including inhalers or topical creams and ointments.
21. Neurologic, neurodevelopmental, and neurodegenerative disorders including autism, Parkinson's disease.
22. General. Body mass index >26 kg/m2, central obesity defined by waste:hip ratio >0.85 (male) and >0.80 (female).
23. Blood pressure >135 mmHg systolic and >85 mmHg diastolic.
24. Skin—presence of a rash, tattoos or body piercing placed within a year, orjaundice
25. Enlarged lymph nodes.
26. Wheezing on auscultation.
27. Hepatomegaly or stigmata of liver disease.
28. Swollen or tender joints. Muscle weakness.
29. Abnormal neurologic examination.
30. Positive stool Clostridium difficile toxin B tested by PCR.
31. Positive stool cultures for any of the routine pathogens including Salmonella, Shigella, Yersinia, Campylobacter, E. coli O157:H7.
32. Abnormal ova and parasites examination.
33. Positive Giardia, Cryptosporidium, or Helicobacter pylori antigens.
34. Positive screening for any viral illnesses, including HIV 1 and 2, Viral Hepatitis A IgM, Hepatitis surface antigen and core Ab.
35. Abnormal RPR (screen for syphilis).
36. Any abnormal liver function tests including alkaline phosphatase, aspartate aminotransaminase, alanine aminotransferase.
37. Raised serum triglycerides >150 mg/Dl
38. HDL cholesterol <40 mg/dL (males) and <50 mg/dL (females)
39. High sensitivity CRP >2.4 mg/L
40. Raised fasting plasma glucose (>100 mg/dL)

An aspect of the present invention is a method of preparing a therapeutic bacterial isolate, the method comprising: (i) providing a preparation of bacteria from fecal material from (a) a group of healthy subjects, (b) a group of patients with a gastrointestinal disease or disorder, and (c) a group of patients having clinical remission of one or more symptoms of the gastrointestinal disease or disorder following treatment of each patient of the group of patients with a fecal microbiota transplant, (ii) analyzing 16S rRNA sequences from the preparation of bacteria, and (iii) isolating a bacterial isolate from stool of a human donor that comprises a 16S rRNA sequence that is at least 97% identical to a 16S rRNA sequence of a bacterial strain from the preparation of bacteria that is (a) enriched in a group of healthy subjects over a group of patients with the gastrointestinal disease or disorder; and/or (b) correlated with clinical remission of one or more symptoms of the gastrointestinal disease or disorder in a group of patients following treatment of each patient of the group of patients with the fecal microbiota transplant, wherein a cross-sectional combined p-value of the bacterial isolate is less than 1×10⁻¹⁴.

In embodiments, the bacterial strain enriched in a group of healthy subjects over a group of patients with the gastrointestinal disease or disorder is selected from Faecalibacterium prausnitzii, Odoribacter splanchnicus, Anaerostipes hadrus, Alistipes onderdonkii, Faecalibacterium prausnitzii, Eubacterium rectale, Blautia obeum, Bacteroides uniformis, Bacteroides vulgatus, Bacteroides cellulosilyticus, Alistipes finegoldii, Alistipes shahii, Akkermansia muciniphila, Phascolarctobacterium faecium, Subdoligranulum variabile, Subdoligranulum variabile, Blautia sp., Alistipes putredinis, Alistipes putredinis, Alistipes putredinis, and any two or more thereof. In embodiments, the bacterial strain enriched in a group of healthy subjects over a group of patients with the gastrointestinal disease or disorder comprises a 16S rRNA sequence that has at least about 98%, or at least about 99% sequence identity with a nucleotide sequence selected from SEQ ID NOs: 1, 2, 3, 4, 7, 8, 9, 11, 12, 14, 15, 16, 18, 20, 21, 22, 23, 34, 35, 36, and 37.

In embodiments, the bacterial isolate comprises at least one of Odoribacter splanchnicus and Alistipes shahii. In embodiments, the bacterial isolate comprises a 16S rRNA sequence that has at least about 98%, or at least about 99% sequence identity with a nucleotide sequence of SEQ ID NO: 2 and SEQ ID NO: 18.

In embodiments, the bacterial isolate comprises a 16S rRNA sequence that is at least 99% identical to a 16S rRNA sequence of the bacterial strain.

In embodiments, the bacterial strain correlated with clinical remission of one or more symptoms of the gastrointestinal disease or disorder in a group of patients following treatment of each patient of the group of patients with the fecal microbiota transplant comprises: Alistipes finegoldii, but does not comprise Alistipes shahii or Alistipes putredinis, or Alistipes putredinis, but does not comprise Alistipes shahii or Alistipes finegoldii. In embodiments, the bacterial strain correlated with clinical remission of one or more symptoms of the gastrointestinal disease or disorder in a group of patients following treatment of each patient of the group of patients with the fecal microbiota transplant comprises: a 16S rRNA sequence that has at least about 95%, or at least about 97%, or at least about 98%, or at least about 99% sequence identity with a nucleotide sequence of SEQ ID NO: 15, but not a nucleotide sequence selected from SEQ ID NO: 18, 35, 36 or 37; or a 16S rRNA sequence that has at least about 95%, or at least about 97%, or at least about 98%, or at least about 99% sequence identity with the nucleotide sequence selected from SEQ ID NOs: 35-37, but not a nucleotide sequence selected from SEQ ID NO: 15 and 18.

In embodiments, the method further comprises isolating a second therapeutic bacterial isolate by: (iv) providing multiple bacterial isolates from human fecal samples; and (v) performing a functional assay comprising: (a) contacting a population of eukaryotic cells with the human-derived bacterial isolates, (b) measuring a level of a cytokine in the population of eukaryotic cells, and (c) selecting the bacterial isolate capable of modulation of the level of the cytokine in the population of eukaryotic cells. In embodiments, the population of eukaryotic cells comprises a population of PBMCs. In embodiments, the cytokine is selected from IL-10, GM-CSF, IFN-gamma, TNF-alpha, IL-23, and IL-12.

In embodiments, the second bacterial isolate is selected from Faecalibacterium prausnitzii, Odoribacter splanchnicus, Anaerostipes hadrus, Alistipes shahii, Akkermansia muciniphila, Subdoligranulum variabile, Bacteroides uniformis, and any two or more thereof.

In embodiments, the second bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 11, 16, 18, 20, 22, and 23.

An aspect of the present invention is a method of preparing a microbial cocktail comprising a first and second bacterial isolate, the method comprising: (i) isolating multiple bacterial isolates from human stool; (ii) performing a first functional assay comprising: (a) contacting a population of eukaryotic cells with the bacterial isolates, (b) measuring an IL-10:IL-12 ratio and an IL-10:TNF-alpha ratio, and (c) identifying as the first bacterial isolate a bacterial isolate capable of inducing an IL-10:IL-12 ratio of at least about 50:1 or an IL-10:TNF-alpha ratio of at least about 1:1 when incubated with a population of eukaryotic cells, (iii) performing a second functional assay comprising: (a) measuring a level of a short chain fatty acid (SCFA) produced by the bacterial isolates, and (b) identifying as the second bacterial isolate a bacterial isolate that produces the SCFA at a concentration of at least about 10 mM, and (iv) combining the first and second bacterial isolates to produce the microbial cocktail.

In embodiments, the first bacterial isolate is selected from Faecalibacterium prausnitzii, Odoribacter splanchnicus, Anaerostipes hadrus, Bacteroides uniformis, Bacteroides vulgatus, Coprococcus comes, Alistipes shahii, Akkermansia muciniphila, Subdoligranulum variabile, and any two or more thereof. In embodiments, the first bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 11, 12, 16, 17, 18, 20, 22 and 23. In embodiments, the population of eukaryotic cells comprises a population of PBMCs.

In embodiments, the SCFA is selected from acetic acid, butyric acid, caproic acid, formic acid, heptanoic acid, isobutyric acid, isocaproic acid, isovaleric acid, propionic acid, valeric acid, and any two or more thereof. In embodiments, the SCFA is butyric acid. In embodiments, the second bacterial isolate is selected from Odoribacter splanchnicus, Eubacterium rectale, Coprococcus comes, Faecalibacterium prausnitzii, Roseburia faecis, Anaerostipes hadrus, Subdogranulum variabile, and any two or more thereof. In embodiments, the second bacterial isolate comprises a 16S rRNA sequence that has at least about 95%, or at least about 97%, or at least about 98%, or at least about 99% sequence identity with anucleotide sequence selected from SEQ ID NOs: 1, 2, 3, 7, 8, 17, 19, 22, and 23.

In embodiments, the method further comprises isolating a third bacterial isolate for incorporation into the microbial cocktail by: (i) analyzing 16S rRNA sequences from preparations of bacteria from fecal material of: (a) a group of healthy subjects, (b) a group of patients with a gastrointestinal disease or disorder, and (c) a group of patients having clinical remission of one or more symptoms of the gastrointestinal disease or disorder following treatment of each patient of the group of patients with a fecal microbiota transplant, and (ii) isolating as the third bacterial isolate a bacterial isolate from stool of a human donor that comprises a 16S rRNA sequence that is at least 97% identical to a 16S rRNA sequence of a bacterial strain from the preparations of bacteria that is (a) enriched in a group of healthy subjects over a group of patients with the gastrointestinal disease or disorder; and/or (b) correlated with clinical remission of one or more symptoms of the gastrointestinal disease or disorder in a group of patients following treatment of each patient of the group of patients with the fecal microbiota transplant.

In embodiments, the third bacterial isolate comprises a 16S rRNA sequence that is at least 99% identical to a 16S rRNA sequence of the bacterial strain.

In any of the embodiments disclosed herein, the gastrointestinal disease or disorder may be selected from inflammatory bowel disease (IBD), irritable bowel syndrome (IBS), C. difficile infection (CDI), C. difficile-associated disease (CDAD), and an antibiotic-induced adverse effect. In embodiments, the IBD is selected from one or more of ulcerative colitis (UC), Crohn's disease (CD), and pouchitis. In embodiments, the gastrointestinal disease or disorder is selected from is selected from inflammatory bowel disease (IBD), irritable bowel syndrome (IBS), C. difficile infection (CDI), C. difficile-associated disease (CDAD), and an antibiotic-induced adverse effect. In embodiments, the IBD is selected from one or more of ulcerative colitis (UC), Crohn's disease (CD), and pouchitis

In one aspect, provided herein is a process for preparing fecal flora (e.g., an entire (or substantially entire) microbiota), first comprising a collection from one or more healthy (e.g., screened) donor(s). In one aspect, a fresh stool is transported via a stool collection device, which can provide or comprises a suitably oxygen free (or substantially oxygen free) appropriate container. In one aspect, the container can be made oxygen free by e.g., incorporating into the container a built in or clipped-on oxygen-scavenging mechanism, e.g., oxygen scavenging pellets as described e.g., in U.S. Pat. No. 7,541,091. In another aspect, the container itself is made of an oxygen scavenging material, e.g., oxygen scavenging iron, e.g., as described by O2BLOCK®, or equivalents, which uses a purified and modified layered clay as a performance-enhancing carrier of oxygen-scavenging iron; the active iron is dispersed directly in the polymer. In one aspect, oxygen-scavenging polymers are used to make the container itself or to coat the container, or as pellets to be added; e.g., as described in U.S. Pat. App. Pub. 20110045222, describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one aspect, oxygen-scavenging polymers are used to make the container itself or to coat the container, or as pellets to be added; e.g., as described in U.S. Pat. App. Pub. 20110008554, describing compositions comprising a polyester, a copolyester ether and an oxidation catalyst, wherein the copolyester ether comprises a polyether segment comprising poly(tetramethylene-co-alkylene ether). In one aspect, oxygen-scavenging polymers are used to make the container itself or to coat the container, or as pellets to be added; e.g., as described in U.S. Pat. App. Pub. 201000255231, describing a dispersed iron/salt particle in a polymer matrix, and an oxygen scavenging film with oxygen scavenging particulates.

Alternatively, in addition to or in place of the oxygen-scavenging mechanism, the air in the container can be replaced (completely or substantially) with nitrogen and/or other inert non-reactive gas or gases. In one aspect, the container simulates (creates) partially, substantially or completely an anaerobic environment.

In one aspect, the stool (e.g., fecal sample) is held in an aesthetically acceptable container that will not leak nor smell yet maintain an anaerobic environment. In one aspect, the container is sterile before receiving the fecal flora.

In one aspect, a stool sample provided herein is maintained at room temperature during most or all of its transportation and/or storage at e.g., a “stool bank”. For example, once delivered to a “processing stool bank” it is stored at ambient temperature, e.g., room temperature. In one aspect, stabilizing agents such as glycerol are added to the harvested and/or stored material.

In one aspect, the stool is tested for various pathogens, as noted above. In one aspect, once cleared of infective agents, a stool sample is homogenized and filtered to remove large particles of matter. In one aspect, the stool is subdivided into desired volumes, e.g., which can be between 5 cc and 3 or more liters. For example, in one aspect, a container comprises a 50 gram (g) stool, which can be held in an appropriate oxygen resistant plastic, e.g., a metallized polyethylene terephthalate polyester film, or a metallized MYLAR™.

In one aspect, the stool is subject to homogenization by for example, mixing, agitating, stirring or shaking. In certain aspects, a stool sample is diluted with a homogenization buffer prior to homogenization. A homogenization buffer can, for example, contain a cryoprotectant (e.g., trehalose), an antioxidant or reducing agent (e.g., cysteine), and a buffer (e.g., 0.25×PBS at pH 7.4).

In one aspect, to separate the non-bacterial components from the fecal microbiota, the stool can be homogenized and filtered from rough particulate matter. In one aspect, the microscopic fiber/nonliving matter is then separated from the bacteria. Several methods can be used, including e.g., recurrent filtration with filter sizes, e.g., progressively coming down to the size of a typical bacterium.

In one aspect, different filters are used to isolate bacterial sp., or a technique as used by Williams in WO 2011/033310A1, which uses a crude technique of filtration with a gauze.

In one aspect, a filtration procedure for filtering whole stool is suitably used to reach the highest concentration of almost 100% bacteria. In one aspect, the filtering procedure is a two-step procedure suitably using glass fibre depth filters for initial clarification. In one aspect, the stool is filtered under positive pressure. In one aspect, this would be using a combination or sandwich configuration with a 30 micron PVDF filter. In one aspect, this sandwich procedure will be filtering the product under positive pressure. Later, membrane concentration can, in one aspect, be used as another step to reduce the volume of the filtrate. In one aspect, this can be done prior to freeze drying or spray drying under nitrogen cover.

Alternative membranes that can be used for filtration include, but not limited to, nylon filters, cellulose nitrate filters, polyethersulfone (PES) filters, polytetrafluorethylene (PTFE) filters, TEFLON™ filters, mixed cellulose Ester filters, polycarbonate filters, polypropylene filters, Polyvinylchloride (PVC) filters or quartz filters. Various combinations of these can be used to achieve a high purity of bacteria with solids and liquid removed.

In one aspect, a subject in need thereof is administered a pharmaceutical composition comprising fecal microbiota of multiple carefully screened, healthy donors. In an aspect, a subject is administered a pharmaceutical composition over a dosing period wherein a first dose comprises at least one pharmaceutical composition comprises fecal microbiota of a single donor, and a second dose of a pharmaceutical composition comprises fecal microbiota of a single donor different from the donor of the first dose. In another aspect, a first dose comprises a pharmaceutical composition comprising fecal microbiota of a single donor and a second dose comprises fecal microbiota of a donor pool. The first and second dose do not indicate the order of administration to a subject, but rather that fecal microbiota from separate donors can be used in a non-blended form.

In another aspect, the present disclosure provides for methods for treating a subject in need thereof with capsules containing a pharmaceutical composition comprising a fecal microbiota from a single donor. In another aspect, a capsule comprises a pharmaceutical composition comprising fecal microbiota from multiple donors. In one aspect a subject is administered two or more pills comprising fecal microbiota from a single but different donor.

In one aspect, the present disclosure provides for methods for treating a subject in need thereof comprising administering a pharmaceutical composition orally or by infusions through a colonoscope, an enema or via a nasojejunal tube. In another aspect, each administration comprises a pharmaceutical composition comprising fecal microbiota of a single donor similar to or different from a prior administration in a treatment period. In another aspect, a treatment period includes administration of a first dose comprising a pharmaceutical composition comprising fecal microbiota of a single donor and administration of a second dose comprising a pharmaceutical composition comprising fecal microbiota of multiple donors.

### Pharmaceutical Compositions, Formulations, and Administration

Described herein are pharmaceutical compositions comprising one or more bacterial isolates (and/or additional therapeutic agents, e.g. a substantially complete fecal microbiota) in various formulations. Any pharmaceutical composition (and/or additional therapeutic agents) described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the pharmaceutical compositions can conveniently be presented in unit dosage forms. For example, the dosage forms can be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting).

In another aspect, a pharmaceutical composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and a combination thereof. In another aspect, a pharmaceutical composition can contain auxiliary substances such as wetting or emulsifying agents, stabilizing or pH buffering agents. In an aspect, a pharmaceutical composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a pharmaceutical composition contains about 2%-70%, 5%-60%, 10%-50%, 15%-40%, 20%-30%, 25%-60%, 30%-60%, or 35%-60% of active ingredient.

In an aspect, a pharmaceutical composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, for preparing solid compositions such as tablets, an active ingredient is mixed with a pharmaceutical carrier, e.g., conventional tableting ingredients such as cornstarch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition described herein. When referring to these preformulation compositions as homogeneous, it is meant that the active ingredient is dispersed evenly throughout the composition so that the composition can be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A pharmaceutical composition used herein can be flavored.

In one embodiment, a pharmaceutical composition comprising one or more bacterial isolates (and/or additional therapeutic agents) described herein is formulated as a composition adapted for a mode of administration described herein.

In various embodiments, the administration of the pharmaceutical compositions is any one of oral, intravenous, intraperitoneal, and parenteral. For example, routes of administration include, but are not limited to, oral, intraperitoneal, intravenous, intramuscular, or rectally. In various embodiments, the administration of the pharmaceutical compositions is oral, naso-gastric, antegrade gastrointestinal, retrograde gastrointestinal, endoscopic, or enemic.

In one embodiment, the pharmaceutical compositions described herein are formulated as a composition adapted for oral administration. Compositions for oral delivery can be in the form of tablets, lozenges, aqueous or oily suspensions, granules, powders, sprinkles, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration to provide a sustained action over an extended period of time. Selectively permeable membranes surrounding an osmotically active agent driving any bacterial isolate (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material, such as glycerol monostearate or glycerol stearate, can also be useful. Oral compositions can include standard excipients such as mannitol, lactose, starch, magnesium stearate, sodium saccharin, cellulose, ethacrylic acid and derivative polymers thereof, and magnesium carbonate. In one embodiment, the excipients are of pharmaceutical grade. Suspensions, in addition to the active compounds, can contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In various embodiments, the pharmaceutical compositions are formulated as solid dosage forms such as tablets, dispersible powders, granules, and capsules. In one embodiment, the pharmaceutical compositions are formulated as a capsule. In another embodiment, the pharmaceutical compositions are formulated as a tablet. In yet another embodiment, the pharmaceutical compositions are formulated as a soft-gel capsule. In a further embodiment, the pharmaceutical compositions are formulated as a gelatin capsule.

In an aspect, a pharmaceutical composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In various embodiments, formulations can additionally comprise a pharmaceutically acceptable carrier or excipient. As one skilled in the art will recognize, the formulations can be in any suitable form appropriate for the desired use and route of administration.

In some dosage forms, the pharmaceutical compositions (e.g., microbial cocktails) comprising one or more bacterial isolates described herein are mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients can have two or more functions in the oral dosage form. In the case of an oral dosage form, for example, a capsule or a tablet, the dosage form can also comprise buffering agents.

In embodiments, a pharmaceutical composition comprising one or more bacterial isolates is combined with one or more pharmaceutically acceptable cryoprotectants, lyoprotectants, binders, disintegrants, excipients, fillers, and/or preservatives, acid suppressants, antacids, H2 antagonists, and proton pump inhibitors, or combinations thereof.

In an aspect, a pharmaceutical composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a pharmaceutical composition.

In one aspect, a pharmaceutical composition administered herein further comprises an acid suppressant, an antacid, an H2 antagonist, a proton pump inhibitor or a combination thereof. In one aspect, a pharmaceutical composition administered herein substantially free of non-living matter. In another aspect, a pharmaceutical composition administered herein substantially free of acellular material selected from the group consisting of residual fiber, DNA, viral coat material, and non-viable material. In another aspect, a pharmaceutical composition administered does not comprise an acid suppressant, an antacid, an H2 antagonist, a proton pump inhibitor or a combination thereof. In yet another aspect, a pharmaceutical composition administered does not comprise an acid suppressant. In another aspect, a pharmaceutical composition administered does not comprise an antacid. In another aspect, a pharmaceutical composition administered does not comprise an H2 antagonist. In another aspect, a pharmaceutical composition administered does not comprise a proton pump inhibitor.

In another aspect, a pharmaceutical composition administered does not comprise metoclopramide.

In embodiments, the bacterial mixture is dry, e.g., when it includes lyophilized bacterial cells/spores or comprises dry binders, fillers, and dispersants. Alternately, the microbial cocktail comprising bacterial isolates is aqueous, e.g., when it comprises non-dry binders, fillers, and dispersants.

In embodiments, a bacterial mixture described herein can be subject to lyophilization. As used herein, “lyophilization” or “freeze drying” refers to the process of drying a material by first freezing it and then encouraging the ice within it to sublimate in a vacuum environment. Disclosed herein is a method of manufacturing a microbial cocktail, the method comprising culturing a first bacterial isolate as a pure culture; culturing a second bacterial isolate as a pure culture; lyophilizing the first bacterial isolate; lyophilizing the second bacterial isolate; and combining the first and second lyophilized bacterial isolates into the microbial cocktail. In another embodiment, disclosed herein is a method of manufacturing a microbial cocktail, the method comprising culturing a first bacterial isolate as a pure culture; culturing a second bacterial isolate as a pure culture; and combining the first bacterial isolate with the second bacterial isolate to form the microbial cocktail. Optionally, the microbial cocktail comprising the first and second bacterial isolates can be lyophilized. It will therefore be understood that a microbial cocktail can be lyophilized or non-lyophilized, and that a lyophilized microbial cocktail can be produced by lyophilizing bacterial isolates that form the cocktail before or after combining the bacterial isolates.

In one aspect, a bacterial mixture comprises a lyophilized formulation further comprising a reducing agent and/or antioxidant. In certain embodiments, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, glutathione, methionine, thioglycerol, and alpha tocopherol.

In one aspect, cysteine is at a concentration of at least about 0.005%, at least about 0.010%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 10%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In embodiments, a pharmaceutical composition comprising one or more bacterial isolates comprises a cryoprotectant or mixture of cryoprotectants. As used herein, a “cryoprotectant” refers to a substance that is added to a formulation in order to protect an active ingredient during freezing. For example, a cryoprotectant can comprise, consist essentially of, or consist of polyethylene glycol, skim milk, erythritol, arabitol, sorbitol, glucose, fructose, alanine, glycine, proline, sucrose, lactose, ribose, trehalose, dimethyl sulfoxide (DMSO) or equivalent, a glycerol, a polyethylene glycol (PEG) or equivalent, or an amino acid (e.g., alanine, glycine, proline). In embodiments of the present disclosure, a cryoprotectant can be selected from the group comprising 5% Sucrose; 10% Sucrose; 10% Skim milk; 10% Trehalose with 2.5% sucrose; 5% Trehalose with 2.5% sucrose; 5% Mannitol; 5% Mannitol with 0.1% Polysorbate 80; 10% Mannitol; 10% Mannitol with 0.1% Polysorbate 80; 5% Trehalose; 5% Trehalose with 0.1% Polysorbate 80; 10% Trehalose; and 10% Trehalose with 0.1% Polysorbate 80.

In embodiments, a pharmaceutical composition comprising one or more bacterial isolates comprises a lyoprotectant. As used herein, a “lyoprotectant” refers to a substance that is added to a formulation in order to protect an active ingredient during the stage of a lyophilization (also known as freeze-drying). In embodiments, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and a combination thereof. In embodiments, a lyoprotectant is a non-reducing sugar, such as trehalose or sucrose. In embodiments, a cryoprotectant or a lyoprotectant consists essentially of, or consists of, one or more substances mentioned in this paragraph and the paragraph above.

In embodiments, a cryoprotectant or a lyoprotectant comprise an intracellular agent, e.g., DMSO, Glycerol, or PEG, which penetrates inside the cell preventing the formation of ice crystals that could result in membrane rupture. In embodiments, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, the present disclosure provides a pharmaceutical composition comprising a lyophilized fecal microbe preparation comprising a lyophilization formulation comprising at least about 12.5% trehalose.

In embodiments, a lyophilized formulation comprises trehalose. In embodiments, a lyophilized formulation comprises 2% to 30%, 3% to 25%, 4% to 20%, 5% to 15%, 6% to 10%, 2% to 30%, 2% to 25%, 2% to 20%, 2% to 15%, or 2% to 10% trehalose. In embodiments, a lyophilized formulation comprises at least 2%, 3%, 4%, 5%, 6%, 7%, 8%, 9%, 10%, or 15% trehalose. In embodiments, a lyophilized formulation comprises at most 2%, 3%, 4%, 5%, 6%, 7%, 8%, 9%, 10%, or 15% trehalose. In embodiments, a lyophilized formulation comprises about 5% trehalose. In embodiments, a lyophilized formulation comprises trehalose and sucrose. In embodiments, a lyophilized formulation comprises between about 8% and 12% trehalose with between about 1.5% and 3.5% sucrose and between about 0.5% and 1.5% NaCl.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In embodiments, a pharmaceutical composition provided here, after at least 12 weeks of storage at ambient temperature or lower, is effective for treating one or more disorders selected from the group consisting of recurrent or primary C. diff infection, ulcerative colitis, Crohn's disease, and irritable bowel syndrome. In embodiments, a pharmaceutical composition remains effective after at least 4, 8, 10, 16, 20, 24, 30, 40, 50, 60, 70, 80 or 100 weeks of storage at ambient temperature or lower.

In embodiments, a pharmaceutical composition (e.g., comprising a microbial cocktail) described herein can be lyophilized or freeze dried and stored at ambient temperatures (e.g., room temperature), at a freezing temperature, or at between about 2° C. and 8° C. In embodiments, freeze-drying allows the majority of cells to remain viable, and produces a powdered form of the product that can be gently pulverized into a powder. The powder, or lyophilized or freeze-dried composition, then can be encapsulated into a carrier, e.g., a tablet, geltab, pill or capsule, e.g., an enteric-coated capsule, or placed into oil-filled capsules for ingestion. Alternatively, the freeze-dried or lyophilized product, or powder, can be reconstituted at ambient temperatures before delivery to an individual in e.g., a fluid, e.g., a sterile fluid, such as saline, a buffer or a media such as a fluid-glucose-cellobiose agar (RGCA) media.

For freeze-drying, in embodiments, bacterial isolates are held in a liquid that will prevent bursting of cells on thawing. This can include various stabilizers, e.g., glycerol and appropriate buffers, and/or ethylene glycol. In embodiments, the cryoprotecting process uses final concentrations of stabilizer(s) of between about 10% and 80%, 20% and 70%, 30% and 60%, or 40% and 50%, depending on the stabilizer(s) used; in embodiments, this helps stabilize proteins by preventing formation of ice crystals that would otherwise destroy protein structures.

In embodiments, stabilizers that help reduce destruction of living bacteria include skim milk, erythritol, arabitol, sorbitol, glucose, fructose and other polyols. Polymers such as dextran and polyethylene glycol can also be used to stabilize bacterial cells.

In embodiments, manufacturing a pharmaceutical composition can comprise steps of: (1) coating the exterior of a dissociated capsule (i.e., comprising separate capsule body and capsule cap) with the exterior enteric coating, (2) filling the capsule body with a pharmaceutical composition (e.g., comprising a microbial cocktail) comprising one or more bacterial isolates, and (3) closing the capsule cap over the capsule body, thereby encapsulating the composition comprising in the exterior, enteric-coated capsule.

Optionally, manufacturing a pharmaceutical composition can comprise steps of: (1) coating the exterior of a dissociated capsule (i.e., comprising separate capsule body and capsule cap) with the exterior enteric coating, (2) coating the interior of the dissociated capsule with an interior coating, (3) filling the capsule body with a pharmaceutical composition (e.g., comprising a microbial cocktail) comprising one or more bacterial isolates, and (4) closing the capsule cap over the capsule body, thereby encapsulating the composition in the dual-coated capsule.

Alternately, manufacturing a pharmaceutical composition can comprise step of: (1) coating the interior of the dissociated capsule (i.e., comprising separate capsule body and capsule cap) with an interior coating, (2) coating the exterior of a dissociated capsule with the exterior enteric coating, (3) filling the capsule body with a pharmaceutical composition (e.g., comprising a microbial cocktail) comprising bacterial isolates, and (4) closing the capsule cap over the capsule body, thereby encapsulating the composition in the dual-coated capsule.

In embodiments, one or more additional therapeutic agents can be combined with a pharmaceutical composition (e.g., comprising a microbial cocktail) and encapsulated by the capsule.

In embodiments, the bodies and caps of gelatin capsules (e.g., size #00) are separated. An exterior enteric coating suspension is prepared by dispersing one or more enteric coating polymers along with other components in a solution. The exterior enteric coating suspension is applied to the exterior of separated capsule bodies and caps, e.g., using a fluid bed Wurster column coater, Fluid Bed Coater, or an equivalent). The capsules are fluidized in the product bowl and the exterior enteric coating suspension is sprayed to produce the outer coating to a target of between about 2 mg/cm² and 6 mg/cm², e.g., 3 mg/cm². After completion of this step, the capsules are set to dry, e.g., between about 8 hours and 24 hours. After drying, exemplary capsules are weighed to calculate weight gain from the exterior enteric coating. Capsules can be inspected for irregularities.

In an embodiment, EUDRAGIT® S100 (poly(methacrylic acid, methylmethacrylate)), starch, triethyl citrate, and PlasACRYL™ T20 are dissolved in a solution of water, ethanol, and n-butanol, mixed, and then charged to a suitable spraying device. The solution is then spray coated on the outer surface of the capsule bodies and capsule caps to a target weight gain. The capsule bodies and capsule caps are allowed to dry for about 8 hours to about 24 hours, or longer, e.g., for a week, a month, or more, before further procession, e.g., filling with a microbial cocktail comprising bacterial isolates.

In embodiments, it may be desirable to provide an amount of the pharmaceutical composition (e.g., comprising a microbial cocktail) to a capsule's cap in addition to providing the composition in the capsule's body. In this embodiment, more of the composition will be included in a capsule and/or less air will be contained in a closed capsule.

In embodiments, the interior surface of a capsule is provided an internal coating.

Any of the above-described compositions (e.g., microbial cocktails), inner coatings, capsules, and outer coatings can be combined into a pharmaceutical composition described herein. A skilled artisan would know how to select an inner coating; capsule, and outer coating according to his/her present need, which could be based, for example, on the specific bacterial isolate(s) and/or the location in a subject (e.g., in the colon) where the bacterial isolate(s) should be delivered.

Additional relevant teachings are disclosed in WO 2007122374, which is hereby incorporated herein by reference in its entirety.

In embodiments, during the manufacture of a pharmaceutical composition, a pharmaceutically-acceptable cryoprotectant, lyoprotectant, binder, disintegrant, filler, preservative, acid suppressant, antacid, H2 antagonist, and proton pump inhibitor, or combination thereof can be mixed into the pharmaceutical composition (e.g., comprising a microbial cocktail) to promote desirable properties.

The pharmaceutical composition can additionally include a surface active agent. Surface active agents suitable for use include, but are not limited to, any pharmaceutically acceptable, non-toxic surfactant. Classes of surfactants suitable for use include, but are not limited to, polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some embodiments, compositions can comprise one or more surfactants including, but not limited to, sodium lauryl sulfate, polysorbate 20, polysorbate 40, polysorbate 60, polysorbate 80, and triethyl citrate.

The pharmaceutical composition can also contain pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

The pharmaceutical composition can also include one or more application solvents. Some of the more common solvents that can be used to apply, for example, a delayed-release coating composition include isopropyl alcohol, acetone, methylene chloride and the like.

The pharmaceutical composition can also include one or more alkaline materials. Alkaline material suitable for use in compositions include, but are not limited to, sodium, potassium, calcium, magnesium and aluminum salts of acids such as phosphoric acid, carbonic acid, citric acid and other aluminum/magnesium compounds. In addition, the alkaline material can be selected from antacid materials such as aluminum hydroxides, calcium hydroxides, magnesium hydroxides and magnesium oxide.

Besides inert diluents, the oral compositions can also include adjuvants such as sweetening, flavoring, and perfuming agents.

In various embodiments, the pharmaceutical compositions are formulated for systemic or local delivery. In an embodiment, administration is systemic. In another embodiment, it may be desirable to administer locally to the area in need of treatment.

Various methods can be used to formulate and/or deliver a pharmaceutical composition (e.g., comprising a microbial cocktail) described herein to a location of interest. For example, the pharmaceutical compositions can be formulated for delivery to the GI tract. The GI tract includes organs of the digestive system such as mouth, esophagus, stomach, duodenum, small intestine, large intestine and rectum and includes all subsections thereof (e.g. the small intestine may include the duodenum, jejunum and ileum; the large intestine may include the colon transversum, colon descendens, colon ascendens, colon sigmoidenum and cecum). For example, the compositions can be formulated for delivery to one or more of the stomach, small intestine, large intestine and rectum and includes all subsections thereof (e.g. duodenum, jejunum and ileum, colon transversum, colon descendens, colon ascendens, colon sigmoidenum and cecum). In some embodiments, the compositions described herein can be formulated to deliver to the upper or lower GI tract. In an embodiment, the compositions can be administered to a subject, by, for example, directly or indirectly contacting the mucosal tissues of the GI tract.

In various embodiments, the administration of the pharmaceutical compositions is into the GI tract via, for example, oral delivery, nasogastral tube, intestinal intubation (e.g. an enteral tube or feeding tube such as, for example, a jejunal tube or gastro-jejunal tube, etc.), direct infusion (e.g., duodenal infusion), endoscopy, colonoscopy, or enema.

In one aspect, a method comprises administering a pharmaceutical composition orally, by enema, or via rectal suppository. In one aspect, a pharmaceutical composition administered herein is formulated as an enteric coated (and/or acid-resistant) capsule or microcapsule, or formulated as part of or administered together with a food, a food additive, a dairy-based product, a soy-based product or a derivative thereof, a jelly, flavored liquid, ice block, ice cream, or a yogurt. In another aspect, a pharmaceutical composition administered herein is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

In an aspect, a pharmaceutical composition comprises a liquid culture. In another aspect, a pharmaceutical composition is homogenized, lyophilized, pulverized and powdered. It can then be infused, dissolved such as in saline, as an enema. Alternatively, the powder can be encapsulated as enteric-coated and/or acid-resistant delayed release capsules for oral administration. In an aspect, the powder can be double encapsulated with acid-resistant/delayed release capsules for oral administration. These capsules can take the form of enteric-coated and/or acid-resistant delayed release microcapsules. A powder can be provided in a palatable form for reconstitution for drinking or for reconstitution as a food additive. In a further aspect, a food is yogurt. In one aspect, a powder can be reconstituted to be infused via naso-duodenal infusion.

In another aspect, a pharmaceutical composition administered herein is in a liquid, frozen, freeze-dried, spray-dried, foam-dried, lyophilized, or powder form. In a further aspect, a pharmaceutical composition administered herein is formulated as a delayed or gradual enteric release form. In another aspect, a pharmaceutical composition administered herein comprises an excipient, a saline, a buffer, a buffering agent, or a fluid-glucose-cellobiose agar (RGCA) media. In another aspect, a pharmaceutical composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In various embodiments, provided herein are modified-release formulations comprising one or more bacterial isolates (and/or additional therapeutic agents), wherein the formulation releases a substantial amount of the bacterial isolates (and/or additional therapeutic agents) into one or more regions of the GI tract. For example, the formulation can release at least about 60% of the bacterial isolates after the stomach and into one or more regions of the GI tract.

In various embodiments, the modified-release formulation can release at least 60% of the bacterial isolates (and/or additional therapeutic agents) after the stomach into one or more regions of the intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial isolates (and/or additional therapeutic agents) in the intestines.

In various embodiments, the modified-release formulation can release at least 60% of the bacterial isolates (and/or additional therapeutic agents) in the small intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial isolates (and/or additional therapeutic agents) in the small intestine (e.g., one or more of duodenum, jejunum, ileum, and ileocecal junction).

In various embodiments, the modified-release formulation can release at least 60% of the bacterial isolates (and/or additional therapeutic agents) in the large intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial isolates (and/or additional therapeutic agents) in the large intestine (e.g., one or more of cecum, ascending, transverse, descending or sigmoid portions of the colon, and rectum).

In some embodiments, the pharmaceutical composition is formulated for release in the stomach. In other embodiments, the pharmaceutical composition is formulated so as to not substantially release the bacterial isolates in the stomach.

In certain embodiments, the modified-release formulation releases the bacterial isolates (and/or additional therapeutic agents) at a specific pH. For example, in some embodiments, the modified-release formulation is substantially stable in an acidic environment and substantially unstable (e.g., dissolves rapidly or is physically unstable) in a near neutral to alkaline environment. In some embodiments, stability is indicative of not substantially releasing while instability is indicative of substantially releasing. For example, in some embodiments, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some embodiments, the present formulations are stable in lower pH areas and therefore do not substantially release in, for example, the stomach. In some embodiments, modified-release formulation is substantially stable at a pH of about 1 to about 4 or lower and substantially unstable at pH values that are greater. In these embodiments, the modified-release formulation does not substantially release in the stomach. In these embodiments, the modified-release formulation substantially releases in the small intestine (e.g. one or more of the duodenum, jejunum, and ileum) and/or large intestine (e.g. one or more of the cecum, ascending colon, transverse colon, descending colon, and sigmoid colon). In some embodiments, modified-release formulation is substantially stable at a pH of about 4 to about 5 or lower and consequentially is substantially unstable at pH values that are greater and therefore is not substantially released in the stomach and/or small intestine (e.g. one or more of the duodenum, jejunum, and ileum). In these embodiments, the modified-release formulation substantially releases in the large intestine (e.g. one or more of the cecum, ascending colon, transverse colon, descending colon, and sigmoid colon). In various embodiments, the pH values recited herein can be adjusted as known in the art to account for the state of the subject, e.g. whether in a fasting or postprandial state.

In some embodiments, the modified-release formulation is substantially stable in gastric fluid and substantially unstable in intestinal fluid and, accordingly, is substantially released in the small intestine (e.g. one or more of the duodenum, jejunum, and ileum) and/or large intestine (e.g. one or more of the cecum, ascending colon, transverse colon, descending colon, and sigmoid colon).

In some embodiments, the modified-release formulation is stable in gastric fluid or stable in acidic environments. These modified-release formulations release about 30% or less by weight of the pharmaceutical composition (e.g., comprising bacterial isolates of a microbial cocktail) in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of can release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the composition in the modified-release formulation in gastric fluid with a pH of 4-5, or less or simulated gastric fluid with a pH of 4-5 or less, in about 15, or about 30, or about 45, or about 60, or about 90 minutes. Modified-release formulations can release about 1%, about 2%, about 3%, about 4%, about 5%, about 6%, about 7%, about 8%, about 9%, or about 10% by weight of the total composition in the modified-release formulation in gastric fluid with a pH of 5 or less, or simulated gastric fluid with a pH of 5 or less, in about 15, or about 30, or about 45, or about 60, or about 90 minutes.

In some embodiments, the modified-release formulation is unstable in intestinal fluid. These modified-release formulations release about 70% or more by weight of the bacterial isolates and/or additional therapeutic agent in the modified-release formulation in intestinal fluid or simulated intestinal fluid in about 15, or about 30, or about 45, or about 60, or about 90 minutes. In some embodiments, the modified-release formulation is unstable in near neutral to alkaline environments. These modified-release formulations release about 70% or more by weight of the bacterial isolates and/or additional therapeutic agent in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments can release 70% or more by weight of the pharmaceutical composition (e.g., comprising a microbial cocktail) in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In various embodiments, the modified-release formulation can be substantially stable in chyme. For example, there is, in some embodiments, a loss of less about 50% or about 40%, or about 30%, or about 20%, or about 10% of bacterial isolates activity in about 10, or 9, or 8, or 7, or 6, or 5, or 4, or 3, or 2, or 1 hour from administration.

In various embodiments, the modified-release formulations can be designed for immediate release (e.g. upon ingestion). In various embodiments, the modified-release formulations can have sustained-release profiles, i.e. slow release of the active ingredient(s) in the body (e.g., GI tract) over an extended period of time. In various embodiments, the modified-release formulations can have a delayed-release profile, i.e. not immediately release the active ingredient(s) upon ingestion; rather, postponement of the release of the active ingredient(s) until the composition is lower in the GI tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine.

In various embodiments, the modified-release formulations can utilize one or more modified-release coatings such as delayed-release coatings to provide for effective, delayed yet substantial delivery of the bacterial isolates to the GI tract together with, optionally, additional therapeutic agents.

In one embodiment, the delayed-release coating includes an enteric agent that is substantially stable in acidic environments and substantially unstable in near neutral to alkaline environments. In an embodiment, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymers include, for example, EUDRAGIT® FS 30D, L 30 D-55, L 100-55, L 100, L 12, 5, L 12, 5 P, RL 30 D, RL PO, RL 100, RL 12, 5, RS 30 D, RS PO, RS 100, RS 12, 5, NE 30 D, NE 40 D, NM 30 D, S 100, S 12, 5, and S 12, 5 P. Similar polymers include Kollicoat® MAE 30 DP and Kollicoat® MAE 100 P. In some embodiments, one or more of EUDRAGIT® FS 30D, L 30 D-55, L 100-55, L 100, L 12, 5, L 12, 5 P RL 30 D, RL PO, RL 100, RL 12, 5, RS 30 D, RS PO, RS 100, RS 12, 5, NE 30 D, NE 40 D, NM 30 D, S 100, S 12, 5 S 12, 5 P, Kollicoat® MAE 30 DP and Kollicoat® MAE 100 P is used. In various embodiments, the enteric agent can be a combination of the foregoing solutions or dispersions.

In certain embodiments, one or more coating system additives are used with the enteric agent. For example, one or more PlasACRYL™ additives can be used as an anti-tacking agent coating additive. Illustrative PlasACRYL™ additives include, but are not limited to, PlasACRYL™ HTP20 and PlasACRYL™ T20.

In another embodiment, the delayed-release coating can degrade as a function of time when in aqueous solution without regard to the pH and/or presence of enzymes in the solution. Such a coating can comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings can be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers can include polyvinyl esters, polyvinyl acetals, polyacrylic acid esters, butadiene styrene copolymers, and the like. In one embodiment, colonic delivery is achieved by use of a slowly eroding wax plug (e.g., various PEGS, including for example, PEG6000).

In a further embodiment, the delayed-release coating can be degraded by a microbial enzyme present in the gut flora. In one embodiment, the delayed-release coating can be degraded by bacteria present in the small intestine. In another embodiment, the delayed-release coating can be degraded by bacteria present in the large intestine.

In various embodiments, the modified release formulation can be designed for release in the colon. Various colon-specific delivery approaches can be utilized. For example, the modified release formulation can be formulated using a colon-specific drug delivery system (CODES) as described for example, in Li et al., AAPS PharmSciTech (2002), 3(4): 1-9, the entire contents of which are incorporated herein by reference. Drug release in such a system is triggered by colonic microflora coupled with pH-sensitive polymer coatings. For example, the formulation can be designed as a core tablet with three layers of polymer. The first coating is an acid-soluble polymer (e.g., EUDRAGIT E), the outer coating is enteric, along with a hydroxypropyl methylcellulose barrier layer interposed in between. In another embodiment, colon delivery can be achieved by formulating the pharmaceutical composition (e.g., comprising a microbial cocktail) with specific polymers that degrade in the colon such as, for example, pectin. The pectin can be further gelled or crosslinked with a cation such as a zinc cation. In an embodiment, the formulation is in the form of ionically crosslinked pectin beads which are further coated with a polymer (e.g., EUDRAGIT polymer). Additional colon specific formulations include, but are not limited to, pressure-controlled drug delivery systems (prepared with, for example, ethylcellulose) and osmotic controlled drug delivery systems (i.e., ORDS-CT).

Formulations for colon specific delivery of the bacterial isolates (and/or additional therapeutic agents), as described herein, can be evaluated using, for example, in vitro dissolution tests. For example, parallel dissolution studies in different buffers can be undertaken to characterize the behavior of the formulations at different pH levels. Alternatively, in vitro enzymatic tests can be carried out. For example, the formulations can be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In a further embodiment, in vivo evaluations can be carried out using animal models such as dogs, guinea pigs, rats, and pigs. Further, clinical evaluation of colon specific drug delivery formulations can be evaluated by calculating drug delivery index (DDI) which considers the relative ratio of RCE (relative colonic tissue exposure to the drug) to RSC (relative amount of drug in blood i.e. that is relative systemic exposure to the drug). Higher drug DDI indicates better colon drug delivery. Absorption of drugs from the colon can be monitored by colonoscopy and intubation.

In various embodiments, the present formulation provides for substantial uniform delivery of the bacterial isolates (and/or additional therapeutic agent) in the area of release in the GI tract. In an embodiment, the present formulation minimizes patchy or heterogeneous release of the bacterial isolates.

In various embodiments, the present formulations provide for release of multiple doses of the bacterial isolates along the GI tract. For example, the composition and/or formulation can release multiple doses of the bacterial isolates at different locations along the intestines, at different times, and/or at different pH. The overall release profile of such a formulation can be adjusted using, for example, multiple particle types or multiple layers. For example, in one embodiment, the first dose of the bacterial isolates can be formulated for release in, for example, the small intestine (e.g., one or more of duodenum, jejunum, ileum), whereas the second dose is formulated for delayed release in, for example, the large intestines (e.g., one or more of cecum, ascending, transverse, descending or sigmoid portions of the colon, and rectum). In another example, the first dose of the bacterial isolates can be formulated for release in, for example, the small intestine (e.g., one or more of duodenum, jejunum, ileum), whereas the second dose is formulated for delayed release in, for example, another part of the small intestine (e.g., one or more of duodenum, jejunum, ileum). In another embodiment, the first dose of the bacterial isolates can be formulated for release in, for example, the large intestine (e.g., one or more of cecum, ascending, transverse, descending or sigmoid portions of the colon, and rectum), whereas the second dose is formulated for delayed release in, for example, another part of the large intestine (e.g., one or more of cecum, ascending, transverse, descending or sigmoid portions of the colon, and rectum). In various embodiments, the composition and/or formulation can release at least one dose, at least two doses, at least three doses, at least four doses, or at least five doses of the bacterial isolates at different locations along the intestines, at different times, and/or at different pH.

In some embodiments, the bacterial isolates described herein are in the form of live, vegetative cells. In some embodiments, the bacterial isolates described herein are in the form of spores. In some embodiments, the bacterial isolates described herewith are lyophilized. By way of non-limiting example, lyophilization can be via methods known in the art, including those described in U.S. Pat. No. 7,799,328, the contents of which are hereby incorporated by reference in their entirety. In some embodiments, lyophilized bacterial isolates described herein are placed in an enterically coated soft gel or capsule.

In various embodiments, formulations can take the form of those described in one or more of U.S. Pat. Nos. 8,535,713 and 8,9117,77 and US Patent Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In various embodiments, formulations can take the form of those as described in International Patent Publication No. WO 2008/135090, the contents of which are hereby incorporated by reference in their entirety.

In various embodiments, formulations can take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and US Patent Publication Nos. 2014/0302132; 2014/0227357; 20140088202; 20130287842; 2013/0295188; 2013/0307962; and 20130184290, the contents of which are hereby incorporated by reference in their entirety.

### Administration and Dosage

It will be appreciated that the actual dose of a pharmaceutical composition described herein (e.g., a microbial cocktail comprising bacterial isolates and/or additional therapeutic agents) will vary according to, for example, the particular dosage form and the mode of administration to a subject. Many factors that may modify the action of the bacterial isolates (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In various embodiments, the dose of the pharmaceutical composition (e.g., comprising a microbial cocktail) is effective to modulate a patient's microbiome to favor an ecological balance, i.e. treating or preventing a disorder related to intestinal dysbiosis described herein (including a gastrointestinal disorder).

In one aspect, a pharmaceutically active or therapeutically effective dose of a bacterial isolate administered to a subject (i.e., in single or multiple administrations) to treat at least one symptom of a disorder related to a gut dysbiosis comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ CFUs of the bacterial isolate. In another aspect, a pharmaceutically active or therapeutically effective dose of a bacterial isolate administered to a subject (i.e., in single or multiple administrations) to treat at least one symptom of a disorder related to a gut dysbiosis comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ CFUs of the bacterial isolate. In a further aspect, a pharmacologically active or therapeutically effective dose of a bacterial isolate administered to a subject (i.e., in single or multiple administrations) to treat at least one symptom of a disorder related to a gut dysbiosis is selected from the group consisting of: from 10⁸ CFUs to 10¹⁴ CFUs, from 10⁹ CFUs to 10¹³ CFUs, from 10¹⁰ CFUs to 10¹² CFUs, from 10¹⁰ CFUs to 10¹¹ CFUs, from 10⁹ CFUs to 10¹⁴ CFUs, from 10⁹ CFUs to 10¹² CFUs, from 10⁹ CFUs to 10¹¹ CFUs, from 10⁹ CFUs to 10¹⁰ CFUs, from 10¹⁰ CFUs to 10¹⁴ CFUs, from 10¹⁰ CFUs to 10¹³ CFUs, from 10¹¹ CFUs to 10¹⁴ CFUs, from 10¹¹ CFUs to 10¹³ CFUs, from 10¹² CFUs to 10¹⁴ CFUs, and from 10¹³ CFUs to 10¹⁴ CFUs of the bacterial isolate.

In an aspect, a pharmaceutical composition comprises one or more bacterial isolates, with each bacterial isolate present in each unit dose at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutically effective dose of a bacterial isolate administered to a subject (i.e., in single or multiple administrations) to treat at least one symptom of a disorder related to a gut dysbiosis comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of the bacterial isolate. In another aspect, a pharmaceutically active or therapeutically effective dose of a bacterial isolate administered to a subject i.e. in single or multiple administrations) to treat at least one symptom of a disorder related to a gut dysbiosis comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ total cells or spores of the bacterial isolate. In a further aspect, a pharmacologically active or therapeutically effective dose of a bacterial isolate administered to a subject (i.e., in single or multiple administrations) to treat at least one symptom of a disorder related to a gut dysbiosis is selected from the group consisting of: from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores of the bacterial isolate.

In an aspect, the pharmaceutically active or therapeutically effective dose cell count of a bacterial isolate is directed to live cells. In one aspect, a pharmaceutical composition comprises one or more bacterial isolates, with each bacterial isolates present in each dosage unit at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In an aspect, a pharmaceutical composition described herein is in the form of a capsule, and each capsule comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores.

In an aspect, a pharmaceutical composition described herein is in the form of a capsule, and each capsule comprises from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, or from 10¹³ to 10¹⁴ cells or spores of a bacterial isolate.

In embodiments, a pharmaceutical composition comprises a microbial cocktail that comprises multiple bacterial isolates. In embodiments, at least two bacterial isolates are present in the microbial cocktail at about the same amounts (e.g., about the same number of total cells and/or about the same number of live cells). In embodiments, at least three bacterial isolates, at least four bacterial isolates, at least five bacterial isolates, at least six bacterial isolates, at least seven bacterial isolates, at least eight bacterial isolates, at least nine bacterial isolates, at least ten bacterial isolates, or more than ten bacterial isolates are present in the pharmaceutical composition at about the same amounts (e.g., about the same number of total cells and/or about the same number of live cells). In embodiments, all of the bacterial isolates of a microbial cocktail are present in about the same amounts.

In embodiments, at least two bacterial isolates of a microbial cocktail described herein are present in the microbial cocktail at different amounts (e.g., different numbers of total cells and/or different numbers of live cells). In embodiments, at least three, at least four, at least five, at least six, at least seven, at least eight, at least nine, at least ten, or more than ten bacterial isolates are present in the microbial cocktail at different amounts.

In embodiments, first and second bacterial isolates of a microbial cocktail are present in the microbial cocktail at different amounts, and a relative proportion of the first and second bacterial isolates to each other in the microbial cocktail is about the same as the relative proportion of first and second bacterial strains corresponding to the first and second bacterial isolates in a microbiota of a fecal sample of a human (e.g., a fecal microbiota of a donor of one or more of the first and second bacterial isolates or a fecal microbiota of a subject administered the first and second bacterial isolates).

In embodiments, first and second bacterial isolates of a microbial cocktail are present in the microbial cocktail at different amounts, and a relative proportion of the first and second bacterial isolates to each other in the microbial cocktail is different than or does not resemble a proportion of first and second bacterial strains corresponding to the first and second bacterial isolates in a microbiota of a fecal sample of a human (e.g., a fecal microbiota of a donor of one or more of the first and second bacterial isolates or a fecal microbiota of a subject administered the first and second bacterial isolates).

In embodiments, a pharmaceutical composition comprises one or more bacterial isolates at an amount or dosage which is at or above the minimum amount or dosage of the bacterial isolate required to be administered to a subject for engraftment of the bacterial isolate to occur in the intestine of the subject. For example, a minimum dosage of the bacterial isolate required for engraftment of the bacterial isolate into the intestine of the subject can be at least 10⁶ cells, at least 10⁷ cells, at least 10⁸ cells, at least 10⁹ cells, at least 10¹⁰ cells, at least 10¹¹ cells, or at least 10¹² cells. In embodiments a first and second bacterial isolate of a microbial cocktail engraft in the intestine of a subject at different minimal dosages or amounts, and a dosage or amount of each of the first and second bacterial isolate in the microbial cocktail varies corresponding to the respective minimal dosage or amount required for engraftment of the respective bacterial isolate.

In embodiments, a microbial cocktail comprises Odoribacter splanchnicus and Faecalibacterium prausnitzii, and the O. splanchnicus cells are more abundant in the microbial cocktail than the F. prausnitzii cells. In embodiments, a microbial cocktail comprises O. splanchnicus and Faecalibacterium prausnitzii, and the O. splanchnicus cells are less abundant in the microbial cocktail than the F. prausnitzii cells. In embodiments, a microbial cocktail comprises O. splanchnicus and Faecalibacterium prausnitzii, and the O. splanchnicus cells are more abundant in the microbial cocktail than the F. prausnitzii cells. In embodiments, a microbial cocktail comprises O. splanchnicus and Eubacterium rectale, and the O. splanchnicus cells are less abundant in the microbial cocktail than the E. rectale cells. In embodiments, a microbial cocktail comprises O. splanchnicus and Akkermansia muciniphila, and the O. splanchnicus cells are more abundant in the microbial cocktail than the A. muciniphila cells. In embodiments, a microbial cocktail comprises O. splanchnicus and Bacteroides cellulosilyticus, and the O. splanchnicus cells are more abundant in the microbial cocktail than the B. cellulosilyticus cells. In embodiments, a microbial cocktail comprises O. splanchnicus and Roseburia faecis, and the O. splanchnicus cells are less abundant in the microbial cocktail than the R. faecis cells. In embodiments, a microbial cocktail comprises O. splanchnicus and Alistipes shahii, and the O. splanchnicus cells are more abundant in the microbial cocktail than the A. shahii cells.

In embodiments, a microbial cocktail comprises Eubacterium rectale, the cells of which are more abundant in the microbial cocktail than the cells of any other single bacterial isolate of the microbial cocktail (i.e., E. rectale is the most abundant bacterial isolate in the microbial cocktail). In embodiments, a microbial cocktail comprises E. rectale and F. prausnitzii, and the E. rectale cells are more abundant in the microbial cocktail than the F. prausnitzii cells. In embodiments, a microbial cocktail comprises E. rectale and A. shahii, and the E. rectale cells are more abundant in the microbial cocktail than the A. shahii cells. In embodiments, a microbial cocktail comprises E. rectale and R. faecis, and the E. rectale cells are more abundant in the microbial cocktail than the R. faecis cells. In embodiments, a microbial cocktail comprises E. rectale and A. muciniphila, and the E. rectale cells are more abundant in the microbial cocktail than the A. muciniphila cells.

In embodiments, a microbial cocktail comprises R. faecis and F. prausnitzii, and the R. faecis cells are more abundant than the F. prausnitzii cells. In embodiments, a microbial cocktail comprises R. faecis and F. prausnitzii, and the R. faecis cells are more abundant than the F. prausnitzii cells. In embodiments, a microbial cocktail comprises R. faecis and A. muciniphila, and the R. faecis cells are more abundant than the A. muciniphila cells. In embodiments, a microbial cocktail comprises R. faecis and A. shahii, and the R. faecis cells are more abundant than the A. shahii cells. In embodiments, a microbial cocktail comprises R. faecis and B. cellusloilyticus, and the R. faecis cells are more abundant than the B. cellulosilyticus cells.

In embodiments, a microbial cocktail comprises B. cellulosilyticus, the cells of which are less abundant in the microbial cocktail than the cells of any other single bacterial isolate of the microbial cocktail (i.e., B. cellulosilyticus is the least abundant bacterial isolate in the microbial cocktail). In embodiments, a microbial cocktail comprises B. cellulosilyticus and A. shahii, and the B. cellulosilyticus cells are less abundant than the A. shahii cells. In embodiments, a microbial cocktail comprises B. cellulosilyticus and A. muciniphila, and the B. cellulosilyticus cells are less abundant than the A. muciniphila cells. In embodiments, a microbial cocktail comprises B. cellulosilyticus and F. prausnitzii, and the B. cellulosilyticus cells are less abundant than the F. prausnitzii cells.

In embodiments, a microbial cocktail comprises A. shahii and A. muciniphila, and the A. shahii cells are less abundant than the A. muciniphila cells. In embodiments, a microbial cocktail comprises A. shahii and F. prausnitzii, and the A. shahii cells are less abundant than the F. prausnitzii cells.

In embodiments, a microbial cocktail comprises multiple bacterial isolates belonging to the species F. prausnitzii, and the cells of one of the bacterial isolates are more abundant than the cells of at least one of the other bacterial isolates in the microbial cocktail. In embodiments, a microbial cocktail comprises two bacterial isolates belonging to the species F. prausnitzii, and the cells of one of the bacterial isolates are more abundant than the cells of the other bacterial isolate of the microbial cocktail.

In embodiments, a pharmaceutical composition comprises O. splanchnicus at a dosage of at least 10⁸ cells. In embodiments, a pharmaceutical composition comprises O. splanchnicus at a dosage of between 10⁸ to 10⁹ cells. In embodiments, a pharmaceutical composition comprises O. splanchnicus at a dosage of not more than 10⁹ cells. In embodiments, a pharmaceutical composition comprises O. splanchnicus at a dosage of at least 10⁹ cells.

In embodiments, a pharmaceutical composition comprises B. cellulosilyticus at a dosage of at least 10⁷ cells. In embodiments, a pharmaceutical composition comprises B. cellulosilyticus at a dosage of between 10⁷ to 10⁸ cells. In embodiments, a pharmaceutical composition comprises B. cellulosilyticus at a dosage of at least 10⁸ cells. In embodiments, a pharmaceutical composition comprises B. cellulosilyticus at a dosage of between 10⁸ to 10⁹ cells. In embodiments, a pharmaceutical composition comprises B. cellulosilyticus at a dosage of not more than 10⁹ cells. In embodiments, a pharmaceutical composition comprises B. cellulosilyticus at a dosage of at least 10⁹ cells.

In embodiments, a pharmaceutical composition comprises A. shahii at a dosage of at least 10⁸ cells. In embodiments, a pharmaceutical composition comprises A. shahii at a dosage of between 10⁸ to 10⁹ cells. In embodiments, a pharmaceutical composition comprises A. shahii at a dosage of not more than 10⁹ cells. In embodiments, a pharmaceutical composition comprises A. shahii at a dosage of at least 10⁹ cells.

In embodiments, a pharmaceutical composition comprises A. muciniphila at a dosage of at least 10⁸ cells. In embodiments, a pharmaceutical composition comprises A. muciniphila at a dosage of between 10⁸ to 10⁹ cells. In embodiments, a pharmaceutical composition comprises A. muciniphila at a dosage of not more than 10⁹ cells. In embodiments, a pharmaceutical composition comprises A. muciniphila at a dosage of at least 10⁹ cells.

In embodiments, a pharmaceutical composition comprises R. faecis at a dosage of at least 10⁸ cells. In embodiments, a pharmaceutical composition comprises R. faecis at a dosage of between 10⁸ to 10⁹ cells. In embodiments, a pharmaceutical composition comprises A. muciniphila at a dosage of not more than 10⁹ cells. In embodiments, a pharmaceutical composition comprises A. muciniphila at a dosage of at least 10⁹ cells. In embodiments, a pharmaceutical composition comprises R. faecis at a dosage of between 10⁹ to 10¹⁰ cells. In embodiments, a pharmaceutical composition comprises A. muciniphila at a dosage of not more than 10¹⁰ cells.

In embodiments, a pharmaceutical composition comprises F. prausnitzii at a dosage of at least 10⁸ cells. In embodiments, a pharmaceutical composition comprises F. prausnitzii at a dosage of between 10⁸ to 10⁹ cells. In embodiments, a pharmaceutical composition comprises F. prausnitzii at a dosage of not more than 10⁹ cells. In embodiments, a pharmaceutical composition comprises F. prausnitzii at a dosage of at least 10⁹ cells. In embodiments, a pharmaceutical composition comprises R. faecis at a dosage of between 10⁹ to 10¹⁰ cells. In embodiments, a pharmaceutical composition comprises F. prausnitzii at a dosage of not more than 10¹⁰ cells. In embodiments, a pharmaceutical composition comprises F. prausnitzii at a dosage of at least 10¹⁰ cells. In embodiments, a pharmaceutical composition comprises F. prausnitzii at a dosage of not more than 10¹¹ cells.

In embodiments, a pharmaceutical composition comprises E. rectale at a dosage of at least 10⁸ cells. In embodiments, a pharmaceutical composition comprises E. rectale at a dosage of between 10⁸ to 10⁹ cells. In embodiments, a pharmaceutical composition comprises E. rectale at a dosage of not more than 10⁹ cells. In embodiments, a pharmaceutical composition comprises E. rectale at a dosage of at least 10⁹ cells. In embodiments, a pharmaceutical composition comprises E. rectale at a dosage of between 10⁹ to 10¹⁰ cells. In embodiments, a pharmaceutical composition comprises E. rectale at a dosage of not more than 10¹⁰ cells. In embodiments, a pharmaceutical composition comprises E. rectale at a dosage of at least 10¹⁰ cells. In embodiments, a pharmaceutical composition comprises E. rectale at a dosage of between 10¹⁰ to 10¹¹ cells. In embodiments, a pharmaceutical composition comprises E. rectale at a dosage of not more than 10¹¹ cells. In embodiments, a pharmaceutical composition comprises E. rectale at a dosage of at least 10¹¹ cells.

In embodiments, every about 200 mg of a pharmaceutical composition comprises a pharmacologically active dose. In embodiments, every about 75, 100, 125, 150, 175, 200, 250, 300, 350, 400, 450, 500, 750, 1000, 1500, or 2000 mg of a pharmaceutical composition comprises a pharmacologically active dose.

Individual doses of the pharmaceutical composition (e.g., comprising a microbial cocktail) can be administered in unit dosage forms (e.g., tablets or capsules) containing, for example, from about 0.01 mg to about 5,000 mg, from about 0.01 mg to about 4,000 mg, from about 0.01 mg to about 3,000 mg, from about 0.01 mg to about 2,000 mg, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg of the active ingredient per unit dosage form, or from about 5 mg to about 80 mg per unit dosage form. For example, a unit dosage form can include about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg of the active ingredient, inclusive of all values and ranges therebetween.

In one embodiment, the pharmaceutical composition (e.g., comprising a microbial cocktail) is administered at an amount of from about 0.01 mg to about 100 mg daily, an amount of from about 0.01 mg to about 5,000 mg daily, about 0.01 mg to about 4,000 mg daily, about 0.01 mg to about 3,000 mg daily, about 0.01 mg to about 2,000 mg daily, about 0.01 mg to about 1,000 mg daily, from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various embodiments, the bacterial isolates (and/or additional therapeutic agents) is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg inclusive of all values and ranges therebetween.

In some embodiments, a suitable dosage of the pharmaceutical composition (e.g., comprising a microbial cocktail) is in a range of about 0.01 mg/kg to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other embodiments, a suitable dosage of the composition in a range of about 0.01 mg/kg to about 100 mg/kg of body weight, in a range of about 0.01 mg/kg to about 90 mg/kg of body weight, in a range of about 0.01 mg/kg to about 80 mg/kg of body weight, in a range of about 0.01 mg/kg to about 70 mg/kg of body weight, in a range of about 0.01 mg/kg to about 60 mg/kg of body weight, in a range of about 0.01 mg/kg to about 50 mg/kg of body weight, in a range of about 0.01 mg/kg to about 40 mg/kg of body weight, in a range of about 0.01 mg/kg to about 30 mg/kg of body weight, in a range of about 0.01 mg/kg to about 20 mg/kg of body weight, in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain embodiments, the pharmaceutical composition (e.g., comprising a microbial cocktail) can be administered, for example, more than once daily, about once per day, about every other day, about every third day, about once a week, about once every two weeks, about once every month, about once every two months, about once every three months, about once every six months, or about once every year.

In embodiments, a pharmaceutical composition can be administered to a patient in need thereof at least once daily for at least two consecutive days. In embodiments, a pharmaceutical composition is administered at least once daily for at least 3, 4, 5, 6, 7, 8, 9, 10, 11, 12, 13, 14, or 15 consecutive days. In embodiments, a pharmaceutical composition is administered at least once daily for at least 1, 2, 3, 4, 5, 6, 7, 8, 9, 10, 11, or 12 consecutive weeks. In embodiments, a pharmaceutical composition is administered at least twice, three times, four times, or five times per week for at least 1, 2, 3, 4, 5, 6, 7, 8, 9, 10, 11, or 12 consecutive weeks. In embodiments, a pharmaceutical composition is administered at least once daily for at most 4, 5, 6, 7, 8, 9, 10, 11, 12, 13, 14, 15, 16, 17, 18, 19, or 20 consecutive days or weeks. In embodiments, a pharmaceutical composition is administered at least once daily for at most 1, 2, 3, 4, 5, 6, 7, 8, 9, 10, 11, or 12 consecutive weeks or months. In embodiments, a pharmaceutical composition is administered at least once for at least 1, 2, 3, 4, 5, 6, 7, 8, 9, 10, 11, or 12 consecutive months or years, chronically for a subject's entire life span, or an indefinite period of time.

In embodiments, a pharmaceutical composition can be administered to a patient in need thereof at least twice daily for at least two consecutive days. In embodiments, a pharmaceutical composition is administered at least twice daily for at least 3, 4, 5, 6, 7, 8, 9, 10, 11, 12, 13, 14, or 15 consecutive days. In embodiments, a pharmaceutical composition is administered at least twice daily for at least 1, 2, 3, 4, 5, 6, 7, 8, 9, 10, 11, or 12 consecutive weeks. In embodiments, a pharmaceutical composition is administered at least twice daily for at most 4, 5, 6, 7, 8, 9, 10, 11, 12, 13, 14, 15, 16, 17, 18, 19, or 20 consecutive days or week. In embodiments, a pharmaceutical composition is administered at least twice daily for at most 1, 2, 3, 4, 5, 6, 7, 8, 9, 10, 11, or 12 consecutive weeks or months. In embodiments, a pharmaceutical composition is administered at least twice for at least 1, 2, 3, 4, 5, 6, 7, 8, 9, 10, 11, or 12 consecutive months or years, chronically for a subject's entire life span, or an indefinite period of time.

In embodiments, a pharmaceutical composition can be administered to a patient in need thereof at least three times daily for at least two consecutive days. In embodiments, a pharmaceutical composition is administered at least three times daily for at least 3, 4, 5, 6, 7, 8, 9, 10, 11, 12, 13, 14, or 15 consecutive days. In embodiments, a pharmaceutical composition is administered at least three times daily for at least 1, 2, 3, 4, 5, 6, 7, 8, 9, 10, 11, or 12 consecutive weeks. In embodiments, a pharmaceutical composition is administered at least three times daily for at most 4, 5, 6, 7, 8, 9, 10, 11, 12, 13, 14, 15, 16, 17, 18, 19, or 20 consecutive days or weeks. In embodiments, a pharmaceutical composition is administered at least three times daily for at most 1, 2, 3, 4, 5, 6, 7, 8, 9, 10, 11, or 12 consecutive weeks or months. In embodiments, a pharmaceutical composition is administered at least three times for at least 1, 2, 3, 4, 5, 6, 7, 8, 9, 10, 11, or 12 consecutive months or years, chronically for a subject's entire life span, or an indefinite period of time.

In embodiments, a pharmaceutical composition can be administered to a patient in need thereof at a dosing schedule of at least once or twice daily for at least three consecutive days or weeks. In embodiments, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In embodiments, a pharmaceutical composition can be administered to a patient in need thereof at a dosing schedule of once-a-week, twice-a-week, or thrice-a-week. The term “once-a-week” means that a dose is administered typically only once in a week, for example, on the same day of each week. “Twice-a-week” means that a dose is administered typically only two times in a week, for example, on the same two days of each weekly period. “Thrice-a-week” means that a dose is administered typically only three times in a week, for example, on the same three days of each weekly period.

In embodiments, a pharmaceutical composition can be administered to a patient in need thereof, wherein the administration comprises a first dosing schedule followed by a second dosing schedule. In embodiments, a first dosing schedule comprises a treatment or induction dose. In embodiments, a second dosing schedule comprises a maintenance dose. For example, a pharmaceutically active maintenance dose of a second dosage schedule can be lower than or equal to a pharmaceutically active induction dose of a first dosing schedule. In other examples, a maintenance dose of a second dosing schedule can be higher than an induction dose of a first dosing schedule.

At least one of a first and second dosing schedule for administering a pharmaceutical composition can comprise administration of the composition at least once daily for at least one day. In embodiments, at least one of a first or second dosing schedule comprises administration of the composition at least once daily for at least 2, 3, 4, 5, 6, 7, 8, 9, 10, 11, 12, 13, 14, or 15 consecutive days. In embodiments, at least one of a first or second dosing schedule comprises administration of the composition at least once daily for at least 1, 2, 3, 4, 5, 6, 7, 8, 9, 10, 11, or 12 consecutive weeks. In embodiments, at least one of a first or second dosing schedule comprises administration of the composition for at most 4, 5, 6, 7, 8, 9, 10, 11, 12, 13, 14, 15, 16, 17, 18, 19, or 20 consecutive days or weeks. In embodiments, at least one of a first or second dosing schedule comprises administration of the composition for at most 1, 2, 3, 4, 5, 6, 7, 8, 9, 10, 11, or 12 consecutive weeks or months. In embodiments, at least one of a first or second dosing schedule comprises administration of the composition for at least 1, 2, 3, 4, 5, 6, 7, 8, 9, 10, 11, or 12 consecutive months or years, chronically for a subject's entire life span, or an indefinite period of time.

In embodiments, at least one of a first or second dosing schedule used in a method can be once-a-week, twice-a-week, or thrice-a-week.

In embodiments, at least one of a first and second dosing schedule can last for at least about 2, 4, 6, 8, 10, 12, 18, 24, 36, 48, 72, or 96 months. In embodiments, a second dosing schedule lasts permanently, for a treated subject's entire life span, or an indefinite period of time. In embodiments, at least one of a first and second dosing schedule is a continuous dosing schedule. In embodiments, at least one of a first and second dosing schedule is an intermittent dosing schedule. In embodiments, at least one of a first and second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In embodiments, at least one of a first and second dosing schedule comprises administering a dose every other day, every two days, or every 3, 4, 5, 6, 7, 8 days. In embodiments, a dose is administered for an extended period of time with or without titration (or otherwise changing the dosage or dosing schedule).

In embodiments, the interval between a first and a second dosing schedule is at least about 1, 2, 3, 4, 5, 6, or 7 days, or at least about 1, 2, 3, 4, 5, 6, 7, 8, 9, 10, 11, or 12 weeks, or at least about 1, 2, 3, 4, 6, 7, 8, 9, 10, 11, or 12 months.

In embodiments, a second dosing schedule (e.g., a maintenance dose) comprises a dosage about 2, 3, 4, 5, 6, 7, 8, 9, 10, 20, 30, 40, 50, 75, 100, 200, 400, 800, 1000, 5000 or more fold lower than the dosage used in a first dosing schedule (e.g., an initial induction dose). In embodiments, a second dosing schedule (e.g., a maintenance dosing schedule) has an equal or lower dosing frequency than a first dosing schedule (e.g., an initial treatment dosing schedule). In embodiments, a second dosing schedule (e.g., a maintenance dosing schedule) has a higher dosing interval than a first dosing schedule (e.g., an initial treatment dosing schedule).

In some embodiments, the terms “patient” and “subject” are used interchangeably. In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish.

In various embodiments, methods described herein are useful in treatment of a human subject. In some embodiments, the human is a pediatric human. In other embodiments, the human is an adult human. In other embodiments, the human is a geriatric human. In other embodiments, the human may be referred to as a patient. In some embodiments, the human is a female. In some embodiments, the human is a male.

In certain embodiments, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1-year-old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

### Additional Therapeutic Agents and Combination Therapy or Co-Formulation

The pharmaceutical compositions described herein can include one or more additional therapeutic agents, which can be administered to a subject in need thereof in a method described herein. The additional therapeutic agent can be administered simultaneous or sequential with a microbial therapeutic (e.g., microbial cocktail) described herein. Further, the present compositions and formulations can comprise the additional therapeutic agent (e.g. via co-formulation). For example, the additional therapeutic agent and one or more bacterial isolates can be combined into a single formulation.

In one embodiment, the additional therapeutic agent and a microbial therapeutic (e.g., one or more bacterial isolates) are administered to a subject simultaneously. The term “simultaneously” as used herein, means that the additional therapeutic agent and the microbial therapeutic are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the microbial therapeutic can be by simultaneous administration of a single formulation (e.g., a formulation comprising the additional therapeutic agent and one or more bacterial isolates) or of separate formulations (e.g., a first formulation including the additional therapeutic agent and a second formulation including one or more bacterial isolates).

Co-administration does not require an additional therapeutic agent to be administered simultaneously, if the timing of its administration is such that the pharmacological activities of the additional therapeutic agent and the microbial therapeutic (e.g., one or more bacterial isolates) overlap in time. For example, the additional therapeutic agent and the microbial therapeutic can be administered sequentially. The term “sequentially” as used herein means that the additional therapeutic agent and the microbial therapeutic are administered with a time separation of more than about 60 minutes. For example, the time between the sequential administration of the additional therapeutic agent and the microbial therapeutic can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent and the microbial therapeutic being administered. Either of the additional therapeutic agent or the microbial therapeutic (e.g., one or more bacterial isolates) can be administered first.

In a further embodiment, the additional therapeutic agent and the microbial therapeutic (e.g., one or more bacterial isolates) are administered to a subject simultaneously but the release of additional therapeutic agent and the microbial therapeutic from their respective dosage forms (or single unit dosage form if co-formulated) in the GI tract occurs sequentially.

Co-administration also does not require multiple additional therapeutic agents to be administered to the subject by the same route of administration. Rather, each additional therapeutic agent can be administered by any appropriate route, for example, parenterally or non-parenterally.

In some embodiments, the additional therapeutic agent is an agent used in the current standard-of-care induction therapies for IBD such as UC. Such agents include but are not limited to 5-aminosalicylic acid (5-ASA), corticosteroids, or biologics (e.g., Infliximab, Adalimumab or Vedolizumab).

In some embodiments, the additional therapeutic agent is an anti-inflammatory agent such as steroidal anti-inflammatory agents or non-steroidal anti-inflammatory agents (NSAIDS). Steroids, particularly the adrenal corticosteroids and their synthetic analogues, are well known in the art. Examples of corticosteroids include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that can be used, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. Additional anti-inflammatory agents are described, for example, in U.S. Pat. No. 4,537,776, the entire contents of which is incorporated by reference herein.

In some embodiments, the additional therapeutic agent is a probiotic. Probiotics suitable for use include, but are not limited to, Saccharomyces boulardii; Lactobacillus rhamnosus GG; Lactobacillus plantarum 299v; Clostridium butyricum M588; Clostridium difficile VP20621 (non-toxigenic C. difficile strain); combination of Lactobacillus casei, Lactobacillus acidophilus (Bio-K+CL1285); combination of Lactobacillus casei, Lactobacillus bulgaricus, Streptococcus thermophilus (Actimel); combination of Lactobacillus acidophilus, Bifidobacterium bifidum (Florajen3); combination of Lactobacillus acidophilus, Lactobacillus bulgaricus delbrueckii subsp. bulgaricus, Lactobacillus bulgaricus casei, Lactobacillus bulgaricus plantarum, Bifidobacterium longum, Bifidobacterium infantis, Bifidobacterium breve, and Streptococcus salivarius subsp. thermophilus (VSL #3)).

In an aspect, the present disclosure provides for administering a probiotic prior to administering a pharmaceutical composition comprising a non-selected fecal microbiota. In another aspect, the disclosure provides for co-administering a probiotic and a pharmaceutical composition comprising a non-selected fecal microbiota. In another aspect, the probiotic is selected from the group consisting of one or more Akkermansia or Parabacteroides species. In another aspect, the probiotic is selected from the group consisting of two or more Akkermansia or Parabacteroides species. In another aspect, the probiotic is selected from the group consisting of three or more Akkermansia or Parabacteroides species. In another aspect, the probiotic is selected fromthe group consisting of four or more Akkermansia orParabacteroides species. In another aspect, the probiotic is selected from the group consisting of five or more Akkermansia or Parabacteroides species. In another aspect, the probiotic is selected from the group consisting of six or more Akkermansia or Parabacteroides species. In another aspect, the probiotic is selected from the group consisting of 1 to 3, 3 to 5, 5 to 8, or 8 to 10 Akkermansia or Parabacteroides species. A probiotic can be provided in a single dose or in multiple doses. When provided as a single composition, the single composition can comprise a single probiotic or a mixture of probiotics. When provided in multiple compositions, each composition can comprise a single probiotic or a mixture of probiotics.

In some embodiments, the additional therapeutic agent is a prebiotic. A prebiotic is a compound or compounds (e.g., comprising one or more nutrients) administered to a subject to promote the growth, proliferation, or activity of one or more microorganisms (e.g., bacteria) in the intestine of the subject (e.g., by providing a substrate to be metabolized by the one or more microorganisms). Without wishing to be bound by theory, prebiotics can be added to a pharmaceutical composition to nutritionally supplement bacteria in the endogenous microbiome of the subject and/or in the pharmaceutical composition itself, e.g., to stimulate the growth or activity of one or more strains of an uncultured population of fecal bacteria and/or one or more bacterial isolates. Additionally, one or more prebiotics can be added to a composition to buffer against “shock” to bacteria cells when transitioning those cells to a new environment, for example, subsequent to the isolation and/or purification of an uncultured population of fecal bacteria, or before or after freezing, freeze-drying, spray-drying, reconstitution in solution and the like.

Non-limiting examples of prebiotics that can be added to a pharmaceutical composition or administered to a subject include amino acids, lactic acid, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, and/or xylooligosaccharides (XOSs), and a combination thereof. Illustrative prebiotics include complex carbohydrates, amino acids, peptides, or other essential nutritional components for the survival of the bacterial composition.

In an aspect, a subject is not pretreated with a prebiotic nutrient prior to treatment with a pharmaceutical composition. In another aspect, the pharmaceutical composition is not supplemented with a prebiotic nutrient.

In embodiments, a prebiotic can be added (e.g., in dry or liquid forms) in a pharmaceutical composition described herein, for example, a composition comprising one or more bacterial isolates.

Alternately, or additionally, a prebiotic can be included (e.g., in dry or liquid forms) in a distinct pharmaceutical composition lacking a microbial therapeutic.

A prebiotic can be provided to a subject before, contemporaneously with, and/or after administration of a pharmaceutical composition comprising a microbial therapeutic (e.g., one or more bacterial isolates), either in the same pharmaceutical composition or in a separate pharmaceutical composition.

A prebiotic can be provided in a single dose or in multiple doses. When provided as a single composition, the single composition can comprise a single prebiotic or a mixture of prebiotics. When provided in multiple compositions, each composition can comprise a single prebiotic or a mixture of prebiotics.

As examples, when multiple doses are provided, a first composition comprising a prebiotic can include one specific prebiotic, e.g., inulin, and a second composition can include a second specific prebiotic, e.g., pectin. Alternately, a first composition can include a mixture of prebiotics, e.g., inulin and pectin and a second composition can include different mixture of prebiotics, e.g., inulin and a FOS. A first composition can include a mixture of prebiotics and a second composition can include one specific prebiotic.

The amount of prebiotic provided to a subject/patient and/or included in a composition depends on the specific prebiotic, the specific bacterial strain of beneficial bacteria, and/or the disease state of the subject/patient.

In some embodiments, the additional therapeutic agent is an antidiarrheal agent.

Antidiarrheal agents suitable for use include, but are not limited to, DPP-IV inhibitors, natural opioids, such as tincture of opium, paregoric, and codeine, synthetic opioids, such as diphenoxylate, difenoxin and loperamide, bismuth subsalicylate, lanreotide, vapreotide and octreotide, motiln antagonists, COX2 inhibitors like celecoxib, glutamine, thalidomide and traditional antidiarrheal remedies, such as kaolin, pectin, berberine and muscarinic agents.

In some embodiments, the additional therapeutic agent can be an analgesic. Analgesics useful in the compositions and methods described herein include, without limitation, morphine, codeine, heroine, methadone and related compounds, thebaine, orpiavine, and their derivatives, buprenorphine, the piperidines, morphinans, benzomorphans, tetrahydroisoquinolines, thiambutanes, benzylamines, tilidine, viminol, nefopam, capsaicin(8-methyl-N-vanillyl-6E-nonenamide), “synthetic” capsaicin(N-vanillylnonamide), and related compounds.

In some embodiments, the additional therapeutic agent is an anti-bacterial agent, which includes, but is not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In some embodiments, the anti-bacterial agent can be any of the penicillin, cephalosporin, monobactam, and carbapenem antibiotics.

In one aspect, a method further comprises pretreating a subject with an antibiotic composition prior to administering a therapeutic bacterial or microbiota composition. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In another aspect, a subject is not pretreated with an antibiotic composition prior to administering a therapeutic bacterial or microbiota composition. In another aspect, the pharmaceutical composition is not supplemented with an antibiotic composition. In a further aspect, a method further comprises pretreating a subject with an anti-inflammatory drug prior to administration of a therapeutic bacterial or microbiota composition. In yet another aspect, a subject is not pretreated with an anti-inflammatory drug prior to administering a therapeutic bacterial or microbiota composition. In another aspect, a therapeutic bacterial or microbiota composition is not supplemented with an anti-inflammatory.

For all additional therapeutic agent compositions and methods, targeting to various parts of the GI tract can be employed as described herein.

### Methods of Treatment

Disclosed herein are methods of preventing or treating a disorder related to an intestinal dysbiosis in a subject in need thereof, comprising administering to the subject an effective amount of any herein-disclosed pharmaceutical composition. In embodiments, the disorder is selected from inflammatory bowel disease (IBD), irritable bowel syndrome (IBS), C. difficile infection (CDI), C. difficile-associated disease (CDAD), and antibiotic-induced adverse effect. In embodiments, the disorder is IBD selected from ulcerative colitis (UC), Crohn's disease (CD), and pouchitis. In embodiments, the method treats or prevents a nosocomial infection and/or a secondary emergent infection and/or treats and/or prevents the overgrowth of one or more pathogenic microorganisms in the GI tract of the subject. In embodiments, the method comprises maintenance of a normal intestinal microbiota. In embodiments, the method further comprises administering at least one prebiotic to the subject.

In various embodiments, provided herein is a method of modulating a microbiome of a subject in need thereof to provide or restore an ecological balance, comprising administering to the subject a composition described herein. For instance, in various embodiments, there is provided methods of diminishing or inhibiting one or more pathogenic bacteria by administering a composition described herein. In various embodiments, admistration of one or more bacterial isolates described herein augments growth of at least one type of bacteria not detectably present in a patient's GI tract prior to administration and, in various embodiments, which is non-pathogenic.

In various embodiments, provided herein is a method of restoring or enhancing ecological control over gut pathogens or pathobionts in a subject in need thereof, comprising administering to the subject a composition described herein.

In various embodiments, a method comprises administering a composition described herein to treat a disease or condition associated with GI dysbiosis in a subject in need thereof. In some embodiments, the subject has inflammatory bowel diseases (IBD), for example, Crohn's disease, colitis (e.g., UC or microscopic colitis), or pouchitis. IBD is a group of inflammatory conditions of the large intestine and, in some cases, the small intestine. Examples of IBD that can be treated by the compositions, formulations and methods described herein include, but are not limited to, Crohn's disease, UC, pouchitis, collagenous colitis, lymphocytic colitis, ischemic colitis, diversion colitis, Behçet's syndrome, infective colitis, and indeterminate colitis. In an embodiment, provided herein is a method of treating UC comprising administering a composition described herein to a subject in need thereof. In another embodiment, provided herein is a method of treating CD comprising administering a composition described herein to a subject in need thereof. In a further embodiment, provided herein is a method of treating pouchitis comprising administering a composition described herein to a subject in need thereof.

In various embodiments, a method comprises administering a composition described herein to treat UC in a subject in need thereof. UC is one form of IBD. It is a chronic disease of the colon, in which the lining of the colon becomes inflamed and develops tiny open sores, or ulcers, that produce pus and mucous. In some embodiments, methods described herein can ameliorate, reduce, or eliminate the inflammation and/or ulceration associated with UC. In some embodiments, methods described herein can ameliorate, reduce, or eliminate one or more symptoms associated with UC including but not limited to, abdominal discomfort or pain, frequent emptying of the colon, lose and urgent bowel movements, persistent diarrhea, bloody stool, loss of appetite, and weight loss. In some embodiments, methods described herein can reduce or prevent the delay in growth and development in children afflicted with UC.

In various embodiments, a method comprises administering a composition described herein to treat UC in a subject in need thereof. For example, a successful treatment of the subject can be measured using the indices below, e.g., the present methods cause a subject's activity score threshold to change from severe to moderate, mild, or remission; or cause a patient's score to change from moderate to mild or remission; or cause a patient's score to change from mild to remission:

| | Parameters | Scoring | Activity score thresholds | | | |
|---|---|---|---|---|---|---|
| Index | assessed | system | Remission | Mild | Moderate | Severe |
| Mayo score | Stool frequency | Cumulative | 0-2 | 3-5 | 6-10 | 11-12 |
| | Rectal bleeding | score | | | | |
| | Physician’s | | | | | |
| | global | | | | | |
| | assessment | | | | | |
| | Sigmoidoscopy | | | | | |
| UCDAI | Stool frequency | Cumulative | 0-2 | 3-8 | | 9-12 |
| | Rectal bleeding | score | | | | |
| | Physician’s | | | | | |
| | global | | | | | |
| | assessment | | | | | |
| | Sigmoidoscopy | | | | | |
| Rachmilewitz | Bowel | Cumulative | 0-4 | 5-10 | 11-17 | >17 |
| score (CAI) | movement | score | | | | |
| | frequency | | | | | |
| | Blood in stools | | | | | |
| | Physician’s | | | | | |
| | global | | | | | |
| | assessment | | | | | |
| | Abdominal | | | | | |
| | pain/cramps | | | | | |
| | Temperature | | | | | |
| | EIMs | | | | | |
| | Laboratory | | | | | |
| | findings (ESR, | | | | | |
| | hemoglobin) | | | | | |
| Powell-Tuck | Well-being | Cumulative | ≤3 | 4-10 | 11-14 | >14 |
| index (St | Abdominal pain | score | | | | |
| Mark’s index) | Bowel | | | | | |
| | movement | | | | | |
| | frequency | | | | | |
| | Stool | | | | | |
| | consistency | | | | | |
| | Bleeding | | | | | |
| | Anorexia | | | | | |
| | Nausea/vomiting | | | | | |
| | Abdominal | | | | | |
| | tenderness | | | | | |
| | Eye, joint, | | | | | |
| | mouth, or skin | | | | | |
| | complications | | | | | |
| | Temperature | | | | | |
| | Sigmoidoscopy | | | | | |
| SCCAI | Bowel | Cumulative | ≤2 | 3-20 | | |
| (Walmsley) | movement | score | <2.5 | | | |
| | frequency (day) | | | | | |
| | Bowel | | | | | |
| | movement | | | | | |
| | frequency | | | | | |
| | (night) | | | | | |
| | Urgency of | | | | | |
| | defecation | | | | | |
| | Blood in stool | | | | | |
| | Well-being | | | | | |
| | Extracolonic | | | | | |
| | features | | | | | |
| Lichtiger index | Diarrhea | Cumulative | ≤3 | 4-8 | 9-14 | >14 |
| | frequency | score | | | | |
| | Nocturnal | | | | | |
| | diarrhea | | | | | |
| | Visible blood | | | | | |
| | (% of | | | | | |
| | movements) | | | | | |
| | Fecal | | | | | |
| | incontinence | | | | | |
| | Abdominal | | | | | |
| | pain/cramping | | | | | |
| | Well-being | | | | | |
| | Abdominal | | | | | |
| | tenderness | | | | | |
| | Need for | | | | | |
| | antidiarrheal | | | | | |
| | medications | | | | | |
| Seo index | Bowel | Cumulative | <108 | <150 | 150-220 | >220 |
| | movement | score with | <120 | | | |
| | frequency | components | | | | |
| | ESR | Blood in stool given | | | | |
| | Hemoglobin | different | | | | |
| | Albumin | weightings + | | | | |
| | | (constant to | | | | |
| | | yield a mean | | | | |
| | | value as close | | | | |
| | | as possible to | | | | |
| | | Truelove-Witts criteria) | | | | |

In some embodiments, a method comprises administering a pharmaceutical composition described herein to treat IBS in a subject in need thereof. IBS is a common disorder that affects the colon and can cause cramping, abdominal pain, bloating, gas, diarrhea and constipation. IBS is classified based on the predominant symptom of diarrhea (IBS with predominant diarrhea, IBS-D), constipation (IBS with predominant constipation, IBS-C) or mixed symptoms (lBS with alternating constipation and diarrhea, lBS-A). Methods described herein can be effective in treating one or more of IBS-D, IBS-C, and/or lBS-A. In some embodiments, methods described herein (e.g., comprising administering a composition described herein) can reduce or eliminate one or more symptoms associated with one or more of IBS-D, IBS-C, and/or IBS-A.

In embodiments, a method comprises administering a pharmaceutical composition described herein to treat or prevent a disease/disorder associated with the presence of abnormal enteric microflora (e.g. intestinal dysbiosis) in a subject in need thereof. The disease/disorder can be selected from a gastro-intestinal disorder including irritable bowel syndrome or spastic colon, Functional Bowel Disease (FBD), including constipation predominant FBD, pain predominant FBD, upper abdominal FBD, Nonulcer Dyspepsia (NUD), gastro-esophageal reflux, inflammatory bowel disease including Crohn's disease, ulcerative colitis, indeterminate colitis, collagenous colitis, microscopic colitis, chronic Clostridium difficile infection, pseudomembranous colitis, mucous colitis, antibiotic associated colitis, idiopathic or simple constipation, diverticular disease, AIDS enteropathy, small bowel bacterial overgrowth, coeliac disease, polyposis coil, colonic polyps, chronic idiopathic pseudo obstructive syndrome, and toxic megacolon.

In embodiments, a method comprises administering a composition described herein to treat or prevent a disorder associated with a liver disorder in a subject in need thereof. Non-limiting examples of a liver disorder include primary biliary cirrhosis, Primary Sclerosing Cholangitis (PSC), fatty liver, and cryptogenic cirrhosis. In embodiments, such diseases/disorders are related to an intestinal dysbiosis of a subject.

In embodiments, a method comprises administering a composition described herein to treat or prevent a rheumatic disorder in a subject in need thereof. Non-limiting examples of a rheumatic disorder include rheumatoid arthritis, non-rheumatoid arthritis, non-rheumatoid factor positive arthritis, ankylosing spondylitis, Lyme disease, and Reiter's syndrome. In embodiments, such diseases/disorders are related to an intestinal dysbiosis of a subject.

In embodiments, a method comprises administering a composition described herein to treat or prevent an immune-mediated disorder in a subject in need thereof. Non-limiting examples of an immune-mediated disorder include glomerulonephritis, hemolytic uraemic syndrome, juvenile diabetes mellitus, mixed cryoglobulinaemia, polyarteritis, familial Mediterranean fever, amyloidosis, scleroderma, systemic lupus erythematosus, and Behçets syndrome. In embodiments, such diseases/disorders are related to an intestinal dysbiosis of a subject.

In embodiments, a method comprises administering a composition described herein to treat or prevent an autoimmune disorder in a subject in need thereof. Non-limiting examples of an autoimmune disorder include Acute Disseminated Encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, Antiphospholipid Syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, Autoimmune Inner Ear Disease (AIED), autoimmune myocarditis, autoimmune oophoritis, autoimmune pancreatitis, autoimmune retinopathy, Autoimmune Thrombocytopenic Purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, Chronic Inflammatory Demyelinating Polyneuropathy (CIDP), Chronic Recurrent Multifocal Ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, Granulomatosis With Polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, Mixed Connective Tissue Disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, Paroxysmal Nocturnal Hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, Primary Sclerosing Cholangitis (PSC), psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome, Subacute Bacterial Endocarditis (SBE), Susac's syndrome, sympathetic ophthalmia, Takayasu's arteritis, temporal arteritis/giant cell arteritis, Thrombocytopenic Purpura (TTP), Tolosa-Hunt syndrome, transverse myelitis, type 1 diabetes, asthma, ulcerative colitis, Undifferentiated Connective Tissue Disease (UCTD), uveitis, vasculitis, vesiculobullous dermatosis, vitiligo, and Wegener's granulomatosis. In embodiments, such disorders are related to an intestinal dysbiosis of a subject.

In embodiments, a method comprises administering a composition described herein to treat or prevent a neurological syndrome in a subject in need thereof. Non-limiting examples of a neurological syndrome include as chronic fatigue syndrome, migraine, multiple sclerosis, amyotrophic lateral sclerosis, myasthenia gravis, Gillain-Barre syndrome, Parkinson's disease, Alzheimer's disease, Chronic Inflammatory Demyelinating Polyneuropathy, and other degenerative disorders. In embodiments, such syndromes are related to an intestinal dysbiosis of a subject.

In embodiments, a method comprises administering a composition described herein to treat or prevent a psychiatric disorder in a subject in need thereof. Non-limiting examples of a psychiatric disorder include chronic depression, schizophrenia, psychotic disorders, manic depressive illness; regressive disorders including, Asperger's syndrome, Rett syndrome, Attention Deficit Hyperactivity Disorder (ADHD), and Attention Deficit Disorder (ADD); the regressive disorder; autism; Sudden Infant Death Syndrome (SIDS); anorexia nervosa. In embodiments, such diseases/disorders are related to an intestinal dysbiosis of a subject.

In embodiments, a method comprises administering a composition described herein to treat or prevent a dermatological condition in a subject in need thereof. Non-limiting examples of a dermatological condition include chronic urticaria, acne, dermatitis herpetiformis and vasculitis disorders. In embodiments, such diseases/disorders are related to an intestinal dysbiosis of a subject.

In embodiments, a method comprises administering a composition described herein to treat or prevent a cardiovascular and/or vascular disorder in a subject in need thereof. In embodiments, such diseases/disorders are related to an intestinal dysbiosis of a subject.

In embodiments, a method comprises administering a composition described herein to treat or prevent a bloodstream infection (BSI) in a subject in need thereof. Patients at risk for such BSIs include but are not limited to solid organ transplant patients; chronic kidney disease patients, e.g., on hemodialysis; and oncology patients. In embodiments, such BSIs are related to an intestinal dysbiosis of a subject.

In embodiments, a method comprises administering a composition described herein to treat or prevent a catheter or intravascular-line infection (e.g., central-line infection) in a subject in need thereof. In embodiments, such infections are related to an intestinal dysbiosis of a subject.

In embodiments, a method comprises administering a composition described herein to treat or prevent a skin or soft tissue infection in a subject in need thereof. In embodiments, such infections are related to an intestinal dysbiosis of a subject.

In embodiments, a method comprises administering a composition described herein to treat or prevent a surgical-site infection in a subject in need thereof. In embodiments, such infections are related to an intestinal dysbiosis of a subject.

In embodiments, a method comprises administering a composition described herein to treat or prevent a urinary tract infection (e.g., antibiotic-resistant urinary tract infections and catheter-associated urinary tract infections) in a subject in need thereof. In embodiments, such infections are related to an intestinal dysbiosis of a subject.

In embodiments, a method comprises administering a composition described herein to treat or prevent a wound infection in a subject in need thereof. In embodiments, such infections are related to an intestinal dysbiosis of a subject.

In embodiments, a method comprises administering a composition described herein to treat or prevent an infection in a subject in need thereof. Non-limiting examples of an infection include an antibiotic-resistant infection and an antibiotic-sensitive infection. In embodiments, such infections are related to an intestinal dysbiosis of a subject.

In embodiments, the pharmaceutical compositions and methods described herein can treat or prevent meningitis. In embodiments, the meningitis is related to an intestinal dysbiosis of a subject.

In embodiments, a method comprises administering a composition described herein to treat or prevent pneumonia, e.g., ventilator-associated pneumonia in a subject in need thereof. In embodiments, the pneumonia is related to an intestinal dysbiosis of a subject.

In embodiments, the compositions, formulations and methods described herein can be used in patient populations who are in an outpatient setting, hospitalized, and/or in long-term care facilities. Such patient populations are at risk for nosocomial infections. In embodiments, such infections are related to an intestinal dysbiosis of a subject.

In embodiments, a method comprises administering a composition described herein to treat or prevent Primary Sclerosing Cholangitis (PSC) in a subject in need thereof. For example, one or more bacterial isolates provided in a pharmaceutical composition administered to the subject can replace a dysbiotic gut microbiome with a healthy community, thereby, at least, reducing bile duct inflammation and/or improving liver function.

In embodiments, a method comprises administering a composition described herein to treat or prevent a diarrheal disease in a subject in need thereof. Non-limiting example s of a diarrheal disease include acute bloody diarrhea (e.g., dysentery), acute watery diarrhea (e.g., cholera), checkpoint inhibitor associated colitis, diarrhea due to food poisoning, persistent diarrhea, and traveler's diarrhea. In embodiments, the diarrhea is related to an intestinal dysbiosis of a subject.

In various embodiments, administration of a pharmaceutical composition described herein can reduce, ameliorate, or eliminate one or more symptom(s) associated with a herein-described condition, disease, or disorder. Exemplary symptoms include, but are not limited to, diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, and inflammation of the skin, eyes, joints, liver, and bile ducts. In embodiments, the symptom is related to an intestinal dysbiosis of a subject.

In some embodiments, a method comprises administering a composition described herein to treat or prevent an infection by pathogenic bacteria and/or inhibiting the growth or decreasing the number of pathogenic bacteria in the GI tract of a subject in need thereof. In an embodiment, the pathogenic bacteria is enterobacteria such as Salmonella. In various embodiments, a method comprises administering a composition described herein to mitigate or prevent the overgrowth of various coliforms in a patient's gut (including coliforms that are virulent and/or antibiotic resistant). Illustrative coliforms include Citrobacter, Enterobacter, Hafnia, Kelbsiella, and Escherichia. In some embodiments, the methods and compositions described herein prevent or diminish secondary infections with resistant organisms.

In still other embodiments, a method comprises administering a composition described herein to treat or prevent an infectious disease of the intestines in a subject in need thereof. Non-limiting examples of an infectious disease of the intestine include CDI and/or a CDAD, nosocomial infection, secondary emergent infection, amebiasis, intestinal tuberculosis, or parasitic disorder. In some embodiments, provided herein are methods for treating or preventing a CDI and/or a CDAD, comprising administering an effective amount of a pharmaceutical composition described herein to a subject or a patient need thereof. In various embodiments, the CDI or CDAD comprises one or more of: C. difficile diarrhea (CDD), C. difficile intestinal inflammatory disease, colitis, pseudomembranous colitis, fever, abdominal pain, dehydration and disturbances in electrolytes, megacolon, peritonitis, and perforation and/or rupture of the colon.

In various embodiments, a composition described herein is administered to a subject in need thereof to treat or prevent a disease or condition associated with GI dysbiosis in the context of initial onset or relapse/recurrence (e.g. due to continued or restarted antibiotic therapy). For example, in a subject that has previously suffered from a GI dysbiosis, the present pharmaceutical composition or formulation can be administered upon the first symptoms of recurrence in the subject. By way of non-limiting example, symptoms of recurrence include, in a mild case, about 5 to about 10 watery bowel movements per day, no significant fever, and only mild abdominal cramps while blood tests can show a mild rise in the white blood cell count up to about 15,000 (normal levels are up to about 10,000), and, in a severe case, more than about 12 watery stools per day, nausea, vomiting, high fever (e.g. about 102-104° F.), rectal bleeding, severe abdominal pain (e.g. with tenderness), abdominal distention, and a high white blood count (e.g. of about 15,000 to about 40,000).

In some embodiments, the methods described herein can be used to treat a subject or patient who is suffering from, or is susceptible to, a disease or condition associated with GI dysbiosis. For example, the subject can be undergoing or have undergone an initial and/or adjunctive therapy that renders the subject susceptible to a disease or condition associated with GI dysbiosis. In some embodiments, the subject is undergoing treatment, or has undergone treatment, with an antibiotic. For example, the subject can have taken an antibiotic during the past about 30 days and/or have an immune system that is weak (e.g. from a chronic illness). In another example, the patient can have recently been in the hospital, including in an intensive care unit. Accordingly, in some embodiments, a method comprises administering a composition described herein to treat or prevent a nosocomial infection and/or a secondary emergent infection and/or a hospital acquired infection (HAI) in a subject in need thereof.

In various embodiments, described herein are methods for treating antibiotic-induced adverse effects in the GI tract, comprising administering an effective amount of a microbial therapeutic (e.g., one or more bacterial isolates) to a subject in need thereof. In another embodiment, provided herein are methods for preventing an antibiotic-induced adverse effect in the GI tract, comprising administering an effective amount of a microbial therapeutic to a subject in need thereof.

In another aspect, a pharmaceutical composition or a plurality of pharmaceutical compositions, as disclosed herein, can be used in the manufacture of a medicament, e.g., for treating a herein-described condition, disease, or disorder in a subject in need thereof. In various embodiments, the bacterial isolates as described herein protect the intestinal microbiome from antibiotics-induced damage. In some embodiments, the methods described herein can treat or prevent an antibiotics-associated adverse effect including but not limited to diarrhea, nausea, vomiting, dysgeusia, colitis, and pseudomembranous colitis disease and/or symptoms. In an embodiment, methods described herein can be used to treat or prevent antibiotic-associated diarrhea (AAD).

Methods for measuring change and/or improvement in GI tract function can include, but are not limited to: endoscopy for direct examination of epithelium and mucosa; histological evaluation and/or tissue procurement for direct evaluation of structural changes and/or immune biomarkers; urine tests for assessment of permeability with non-absorbable sugars and LPS levels; stool tests for assessment of inflammation and/or microbiota changes (for example by PCR); and/or blood tests for assessment of specific markers, including CD4+ cell counts, Thl7 cell counts, and/or LPS levels.

In embodiments, the present disclosure provides a method for treating a disorder (e.g., C. difficile infection, autism spectrum disorder (ASD), inflammatory bowel disease, ulcerative colitis, Crohn's disease, or another indication listed herein) in a subject in need thereof, where the method comprises administering to the subject a pharmaceutically active dose of a pharmaceutical composition described herein. In embodiments, the present disclosure provides a method for treating a disorder (e.g., C. difficile infection, ASD, inflammatory bowel disease, ulcerative colitis, or Crohn's disease) in a subject in need thereof, where the method comprises administering daily to the subject a pharmaceutically active dose of a pharmaceutical composition described herein. In embodiments, a pharmaceutical composition is administered to a patient in need thereof at least once daily for at least two consecutive days. In embodiments, a pharmaceutical composition is administered at least once daily for at least 3, 4, 5, 6, 7, 8, 9, 10, 11, 12, 13, 14, or 15 consecutive days. In embodiments, a pharmaceutical composition is administered at least once daily for at least 1, 2, 3, 4, 5, 6, 7, 8, 9, 10, 11, or 12 consecutive weeks. In embodiments, a pharmaceutical composition is administered at least twice, three times, four times, or five times per week for at least 1, 2, 3, 4, 5, 6, 7, 8, 9, 10, 11, or 12 consecutive weeks. In embodiments, a pharmaceutical composition is administered at least once daily for at most 4, 5, 6, 7, 8, 9, 10, 11, 12, 13, 14, 15, 16, 17, 18, 19, or 20 consecutive days or weeks. In embodiments, a pharmaceutical composition is administered at least once daily for at most 1, 2, 3, 4, 5, 6, 7, 8, 9, 10, 11, or 12 consecutive weeks or months. In embodiments, a pharmaceutical composition is administered at least once for at least 1, 2, 3, 4, 5, 6, 7, 8, 9, 10, 11, or 12 consecutive months or years, chronically for a subject's entire life span, or an indefinite period of time.

In embodiments, a pharmaceutical composition is administered to a patient in need thereof at least twice daily for at least two consecutive days. In embodiments, a pharmaceutical composition is administered at least twice daily for at least 3, 4, 5, 6, 7, 8, 9, 10, 11, 12, 13, 14, or 15 consecutive days. In embodiments, a pharmaceutical composition is administered at least twice daily for at least 1, 2, 3, 4, 5, 6, 7, 8, 9, 10, 11, or 12 consecutive weeks. In embodiments, a pharmaceutical composition is administered at least twice daily for at most 4, 5, 6, 7, 8, 9, 10, 11, 12, 13, 14, 15, 16, 17, 18, 19, or 20 consecutive days or week. In embodiments, a pharmaceutical composition is administered at least twice daily for at most 1, 2, 3, 4, 5, 6, 7, 8, 9, 10, 11, or 12 consecutive weeks or months. In embodiments, a pharmaceutical composition is administered at least twice for at least 1, 2, 3, 4, 5, 6, 7, 8, 9, 10, 11, or 12 consecutive months or years, chronically for a subject's entire life span, or an indefinite period of time.

In embodiments, a pharmaceutical composition is administered to a patient in need thereof at least three times daily for at least two consecutive days. In embodiments, a pharmaceutical composition is administered at least three times daily for at least 3, 4, 5, 6, 7, 8, 9, 10, 11, 12, 13, 14, or 15 consecutive days. In embodiments, a pharmaceutical composition is administered at least three times daily for at least 1, 2, 3, 4, 5, 6, 7, 8, 9, 10, 11, or 12 consecutive weeks. In embodiments, a pharmaceutical composition is administered at least three times daily for at most 4, 5, 6, 7, 8, 9, 10, 11, 12, 13, 14, 15, 16, 17, 18, 19, or 20 consecutive days or weeks. In embodiments, a pharmaceutical composition is administered at least three times daily for at most 1, 2, 3, 4, 5, 6, 7, 8, 9, 10, 11, or 12 consecutive weeks or months. In embodiments, a pharmaceutical composition is administered at least three times for at least 1, 2, 3, 4, 5, 6, 7, 8, 9, 10, 11, or 12 consecutive months or years, chronically for a subject's entire life span, or an indefinite period of time.

In embodiments, the present disclosure provides a method for treating a disorder (e.g., C. difficile infection, ASD, inflammatory bowel disease, ulcerative colitis, or Crohn's disease) in a subject in need thereof, where the method comprises administering orally to the subject a pharmaceutically active dose of a pharmaceutical composition comprising one or more live, non-pathogenic, bacterial isolates described herein, where the dose is administered at a dosing schedule of at least once or twice daily for at least three consecutive days or weeks. In embodiments, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In embodiments, the present disclosure provides a method for treating a disorder (e.g., C. difficile infection, ASD, inflammatory bowel disease, ulcerative colitis, or Crohn's disease) in a subject in need thereof, where the method comprises a combination treatment or therapy.

For example, the method can comprise a double combination therapy, a triple combination therapy, or a quadruple combination therapy.

In yet another aspect, described herein is a plurality of pharmaceutical compositions, e.g., two or more pharmaceutical compositions, as disclosed herein, for use in the prevention or treatment a condition, disease, or disorder in a subject in need thereof. In embodiments, a first composition comprises one or more bacterial isolates having a first characteristic and a second composition comprises one or more bacterial isolates having a second characteristic. In embodiments, a first composition comprises one or more bacterial isolates having a first characteristic, a second composition comprises one or more bacterial isolates having a second characteristic, and a third composition comprises one or more bacterial isolates having a third characteristic. As used herein, a characteristic of a bacterial isolate can include, for example: an ability of a bacterial isolate to produce or enhance production of at least one short-chain fatty acid (SCFA) in a gut of a subject administered the composition (e.g., represented by bacterial isolates provided in Table 2); an ability of a bacterial isolate to modulate cytokine production in a eukaryotic cell (e.g., a host cell of a subject administered the composition) (e.g., represented by bacterial isolates provided in Table 3); a correspondence to a bacterial strain more highly abundant in a healthy subject relative to a patient with UC (e.g., the bacterial isolate comprises a 16S rRNA sequence having at least 97% identity to a 16S rRNA sequence of the bacterial strain) (e.g., represented by bacterial isolates provided in Table 4); and a combination thereof.

Described herein is a method for preventing or treating a condition, disease or disorder in a subject in need thereof, comprising administering to the subject an effective amount of one or more pharmaceutical compositions comprising one or more bacterial isolates disclosed herein. In an embodiment, the method comprises administering to the subject one or more bacterial isolates comprising a 16S rRNA sequence at least 95% identical to a 16S rRNA sequence of one or more of the bacterial isolates provided in Table 1. In an embodiment, the method comprises administering to the subject one or more bacterial isolates comprising a 16S rRNA sequence at least 95% identical to the 16S rRNA sequence of one or more of the bacterial isolates provided in any one of Table 2, Table 3, or Table 4.

In an embodiment, the method comprises administering to the subject a plurality of bacterial isolates comprising 16S rRNA sequences at least 95% identical to 16S rRNA sequences of each of the bacterial isolates provided in any one of Tables 7-21. In an embodiment, the method comprises administering to the subject one or more bacterial isolates comprising a 16S rRNA sequence at least 95% identical to the 16S rRNA sequence of one or more of the bacterial isolates provided in Table 22.

In embodiments, a plurality of bacterial isolates administered to a subject in a method described herein are administered in the same pharmaceutical composition. In embodiments, a plurality of bacterial isolates administered to a subject in a method described herein are administered in separate pharmaceutical compositions. For example, a method can comprise administering to a subject in need thereof an effective amount of a plurality of pharmaceutical compositions, e.g., two or more pharmaceutical compositions, as disclosed herein. The plurality of pharmaceutical compositions can be provided simultaneously or sequentially. Thus, if a subject is to be treated with, for example, three bacterial isolates, a first composition can comprise two of the bacterial isolates and the second composition can comprise the third bacterial isolate. In a different example, if a subject is to be treated with three bacterial isolates, a first composition can comprise the first bacterial isolate, the second composition can comprise the second bacterial isolate, and the third composition can comprise the third bacterial isolate.

In embodiments, disclosed herein is a method of treating a disorder, disease, or condition involving a dysbiosis of an intestinal microbiota in a subject in need thereof, the method comprising administering to the subject a pharmaceutical composition described herein, wherein the pharmaceutical composition comprises one or more bacterial isolates, wherein the one or more bacterial isolates are each administered at a particular dose (e.g., a particular number of cells or a particular number of live cells). In an embodiment, the method comprises administering one or more bacterial isolates, wherein the one or more bacterial isolates comprises one or more of O. splanchnicus, B. cellulosilyticus, A. shahii, A. muciniphila, R. faecis, F. prausnitzii, E. rectale, and S. variabile, wherein each of the one or more bacterial isolates is administered at a dosage of at least 10⁷ cells, between 10⁷ to 10⁸ cells, not more than 10⁸ cells, at least 10⁸ cells, between 10⁸ to 10⁹ cells, not more than 10⁹ cells, at least 10⁹ cells, between 10⁹ to 10¹⁰ cells, not more than 10¹⁰ cells, at least 10¹⁰ cells, between 10¹⁰ to 10¹¹ cells, not more than 10¹¹ cells, at least 10¹¹ cells, between 10¹¹ to 10¹² cells, not more than 10¹² cells, and at least 10¹² cells.

In another aspect, disclosed herein are a plurality of pharmaceutical compositions, e.g., two or more pharmaceutical compositions, as disclosed herein, for use in the prevention or treatment of a condition, disease, or disorder in a subject in need thereof. In embodiments, a first composition comprises one or more bacterial isolates described herein. In embodiments, a second composition comprises an uncultured fecal microbiota or a preparation of uncultured fecal bacteria (e.g., a substantially complete fecal microbiota purified from a stool sample). A subject can be treated with the first and second compositions in any order to treat or prevent a disorder. For example, in one embodiment a subject is treated with a composition comprising an uncultured fecal microbiota or a preparation of uncultured fecal bacteria, followed by a composition comprising one or more bacterial isolates. In another embodiment, a subject is treated with a composition comprising one or more bacterial isolates followed by a composition comprising an uncultured fecal microbiota or a preparation of uncultured fecal bacteria. In still other embodiments, a subject can be treated with a composition comprising one or more bacterial isolates and a composition comprising an uncultured fecal microbiota or a preparation of uncultured fecal bacteria simultaneously (for example, with a composition comprising both the bacterial isolate(s) and an uncultured fecal microbiota or a preparation of uncultured fecal bacteria, or with multiple compositions each comprising one of the bacterial isolate(s) or an uncultured fecal microbiota or a preparation of uncultured fecal bacteria).

In an embodiment, a method for treating or preventing a condition, disease or disorder of a subject comprises administration to the subject of (i) a pharmaceutical composition comprising one or more bacterial isolates; and (ii) an uncultured fecal microbiota or a preparation of uncultured fecal bacteria. For example, the one or more bacterial isolates can be administered before or after the uncultured fecal microbiota or a preparation of uncultured fecal bacteria, or at the same time (e.g., in different compositions or together in the same composition). In another embodiment, a method for treating a disorder of a subject comprises administration to the subject of: (i) a pharmaceutical composition comprising one or more bacterial isolates; and (ii) one or more antibiotics. For example, the one or more bacterial isolates can be administered before or after the uncultured fecal microbiota or a preparation of uncultured fecal bacteria, or at the same time (e.g., in different compositions or together in the same composition). Typically, the antibiotic is administered to the subject prior to administration of the one or more bacterial isolates, in order to purge the subject's intestine of harmful and/or pathogenic bacteria prior to replenishment of the gut with bacteria from the bacterial isolate(s). In an embodiment, a method for treating a disorder of a subject comprises administration to the subject of: (i) a pharmaceutical composition comprising one or more bacterial isolates; and (ii) a prebiotic. For example, the one or more bacterial isolates can be administered before or after the prebiotic, or at the same time (e.g., in different compositions or together in the same composition). For each of the above examples, it is further understood that any given component in a method of treatment can be administered multiple times. For example, an uncultured fecal microbiota or a preparation of uncultured fecal bacteria can be administered to the subject, followed by one or more bacterial isolates, followed by a second administration of the uncultured fecal microbiota or a preparation of uncultured fecal bacteria.

In an embodiment, a method for treating or preventing a condition, disease or disorder of a subject comprises administration to the subject of (i) a pharmaceutical composition comprising one or more bacterial isolates; (ii) an uncultured fecal microbiota or a preparation of uncultured fecal bacteria; and (iii) one or more antibiotics. The different components of (i)-(iii) can be administered to the subject in any order. For example, a subject can be administered one or more antibiotics, followed by an uncultured fecal microbiota or a preparation of uncultured fecal bacteria, followed by one or more bacterial isolates. In another example, the subject can be administered one or more antibiotics, followed by one or more bacterial isolates, followed by an uncultured fecal microbiota or a preparation of uncultured fecal bacteria. For each of the above examples, it is further understood that any given component in a method of treatment can be administered multiple times. For example, an antibiotic can be administered to the subject, followed by an uncultured fecal microbiota or a preparation of uncultured fecal bacteria, followed by one or more bacterial isolates, followed by a second administration of the uncultured fecal microbiota or a preparation of uncultured fecal bacteria.

In an embodiment, a method for treating or preventing a condition, disease or disorder of a subject comprises administration to the subject of (i) a pharmaceutical composition comprising one or more bacterial isolates; (ii) an uncultured fecal microbiota or a preparation of uncultured fecal bacteria; and (iii) one or more prebiotics. The different components of (i)-(iii) can be administered to the subject in any order. For example, a subject can be administered one or more prebiotics, followed by an uncultured fecal microbiota or a preparation of uncultured fecal bacteria, followed by one or more bacterial isolates. In another example, the subject can be administered one or more prebiotics, followed by one or more bacterial isolates, followed by an uncultured fecal microbiota or a preparation of uncultured fecal bacteria. In another example, the subject can be administered one or more prebiotics following administration of one or both of the one or more bacterial isolates and/or the uncultured fecal microbiota or a preparation of uncultured fecal bacteria. For each of the above examples, it is further understood that any given component in a method of treatment can be administered multiple times. For example, an uncultured fecal microbiota or a preparation of uncultured fecal bacteria can be administered to a subject, followed by a one or more bacterial isolates, followed by a prebiotic, followed by a second administration of the uncultured fecal microbiota or a preparation of uncultured fecal bacteria.

In an embodiment, a method for treating or preventing a condition, disease or disorder of a subject comprises administration to the subject of (i) a pharmaceutical composition comprising one or more bacterial isolates; (ii) an uncultured fecal microbiota or a preparation of uncultured fecal bacteria; (iii) one or more prebiotics; and (iv) one or more antibiotics. The different components of (i)-(iv) can be administered to the subject in any order. For example, a subject can be administered one or more antibiotics, followed by one or more prebiotics, followed by an uncultured fecal microbiota or a preparation of uncultured fecal bacteria, followed by one or more bacterial isolates. In another example, the subject can be administered one or more antibiotics, followed by one or more prebiotics, followed by one or more bacterial isolates, followed by an uncultured fecal microbiota or a preparation of uncultured fecal bacteria. In another example, the prebiotic can be administered after one or both of the one or more bacterial isolates and/or the uncultured fecal microbiota or a preparation of uncultured fecal bacteria. For each of the above examples, it is further understood that any given component in a method of treatment can be administered multiple times. For example, an antibiotic can be administered to a subject, followed by an uncultured fecal microbiota or a preparation of uncultured fecal bacteria, followed by one or more bacterial isolates, followed by a prebiotic, followed by a second administration of the uncultured fecal microbiota or a preparation of uncultured fecal bacteria.

In each of the above combination treatments, the duration of time between different treatments (e.g., between administration of an uncultured fecal microbiota or a preparation of uncultured fecal bacteria and one or more bacterial isolates) can be at least 1 hour, at least 2 hours, at least 6 hours, at least 12 hours, at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, or greater than 8 weeks.

In embodiments, the pharmaceutical composition is used in a method for treating a disorder related to an intestinal dysbiosis in a subject, e.g., a GI disorder.

### Kits

Described herein are kits comprising any herein-disclosed pharmaceutical composition and instructions for use. In embodiments, the kit further comprises an additional pharmaceutical composition comprising at least one prebiotic (e.g., wherein the additional pharmaceutical composition lacks a microbial therapeutic).

The instructions can describe, for example, dosing information. As examples, the frequency of administration and dose of a composition, e.g., the number of capsules of a pharmaceutical composition to be administered at a given time, and the number of times of administration per day/week). In embodiments in which the kit comprises more than one composition (e.g., a microbial therapeutic and an additional pharmaceutical composition lacking a microbial therapeutic), the instructions can describe the dosing of each composition. For example, one composition can be administered before another composition, e.g., sequential administration of the two pharmaceutical compositions separated by minutes, hours, days, weeks, months, or longer. Alternately, two compositions can be administered simultaneously.

### Methods of Manufacture

Disclosed herein is a method of manufacturing a pharmaceutical composition comprising a microbial cocktail, the method comprising combining two or more bacterial isolates to form the microbial cocktail.

Disclosed herein is a method of manufacturing a pharmaceutical composition that comprises one or more bacterial isolates, the method comprising selecting the one or more bacterial isolates based on at least one of the following characteristics: (i) an ability to produce a particular amount or level of one or more short-chain fatty acids (SCFAs) such as butyrate as measured in a functional assay (e.g., represented by bacterial isolates provided in Table 2); (ii) an ability to induce a particular amount or level of cytokine production or release by a eukaryotic cell incubated with the bacterial isolate in a functional assay (e.g., represented by bacterial isolates provided in Table 3); and/or (iii) a greater relative abundance of the bacterial isolate in a healthy human subject relative to a patient with an intestinal dysbiosis (e.g., ulcerative colitis (UC)), or a greater relative abundance of the bacterial isolate in a human subject in remission from an intestinal dysbiosis (e.g., UC) relative to a patient having the intestinal dysbiosis (e.g., represented by bacterial isolates provided in Table 4). The method can further comprise incorporating the one or more bacterial isolates into the pharmaceutical composition.

In embodiments, only one bacterial isolate comprising characteristic (i) is selected and incorporated into the pharmaceutical composition. In embodiments, only one bacterial isolate comprising characteristic (ii) is selected and incorporated into the pharmaceutical composition. In embodiments, only one bacterial isolate comprising characteristic (iii) is selected and incorporated into the pharmaceutical composition. In embodiments, the pharmaceutical composition comprises only one bacterial isolate.

Disclosed herein is a method of manufacturing a pharmaceutical composition that comprises a plurality of bacterial isolates, the method comprising selecting the plurality of bacterial isolates based on at least one of the following characteristics: (i) an ability of the bacterial isolate to produce one or more short-chain fatty acids (SCFAs) in a gut of a subject administered the pharmaceutical composition; (ii) an ability of the bacterial isolate to modulate cytokine production in a host cell of a subject administered the pharmaceutical composition; and/or (iii) a greater relative abundance of the bacterial isolate in a healthy human subject relative to a patient with an intestinal dysbiosis (e.g., ulcerative colitis (UC)), or a greater relative abundance of the bacterial isolate in a human subject in remission from an intestinal dysbiosis (e.g., UC) relative to a patient having the intestinal dysbiosis. The method can further comprise incorporating the plurality of bacterial isolates into a microbial cocktail, and incorporating the microbial cocktail into the pharmaceutical composition.

In embodiments, two or more bacterial isolates comprising characteristic (i) can be selected and incorporated into the microbial cocktail; two or more bacterial isolates comprising characteristic (ii) can be selected and incorporated into the microbial cocktail; and/or two or more bacterial isolates comprising characteristic; (iii) can be selected and incorporated into the microbial cocktail.

In embodiments, at least one bacterial isolate comprising characteristic (i) can be selected and incorporated into the microbial cocktail, and at least one bacterial isolate comprising characteristic (ii) can be selected and incorporated into the microbial cocktail. In embodiments, at least one bacterial isolate comprising characteristic (i) can be selected and incorporated into the microbial cocktail, and at least one bacterial isolate comprising characteristic (iii) can be selected and incorporated into the microbial cocktail. In embodiments, at least one bacterial isolate comprising characteristic (ii) can be selected and incorporated into the microbial cocktail, and at least one bacterial isolate comprising characteristic (iii) can be selected and incorporated into the microbial cocktail.

In embodiments, at least one bacterial isolate comprising characteristic (i) can be selected and incorporated into the microbial cocktail, and at least one bacterial isolate comprising characteristic (ii) can be selected and incorporated into the microbial cocktail, and at least one bacterial isolate comprising characteristic (iii) can be selected and incorporated into the microbial cocktail.

In embodiments, at least one bacterial isolate comprising characteristic (i) can be selected and incorporated into the pharmaceutical composition, at least one bacterial isolate comprising characteristic (ii) can be selected and incorporated into the microbial cocktail, and at least one bacterial isolate comprising characteristic (iii) can be selected and incorporated into the microbial cocktail.

In embodiments, a bacterial isolate comprising characteristic (i) is not selected. In embodiments, a bacterial isolate comprising characteristic (ii) is not selected. In embodiments, a bacterial isolate comprising characteristic (iii) is not selected.

In embodiments, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or greater than 20 bacterial isolates are selected based on characteristics (i) to (iii) and incorporated into a microbial cocktail.

In embodiments, all bacterial isolates incorporated into a microbial cocktail are selected based on at least one of characteristics (i) to (iii). In embodiments, all bacterial isolates incorporated into a microbial cocktail are selected based on at least two of characteristics (i) to (iii). In embodiments, all bacterial isolates incorporated into a microbial cocktail are selected based on all three of characteristics (i) to (iii). In embodiments, all bacterial isolates incorporated into a composition are selected based on characteristic (i). In embodiments, all bacterial isolates incorporated into a composition are selected based on only characteristic (i). In embodiments, all bacterial isolates incorporated into a composition are selected based on characteristic (ii). In embodiments, all bacterial isolates incorporated into a composition are selected based on only characteristic (ii). In embodiments, all bacterial isolates incorporated into a composition are selected based on characteristic (iii). In embodiments, all bacterial isolates incorporated into a composition are selected based on only characteristic (iii).

In embodiments, a bacterial isolate comprising Odoribacter sphlanchnicus is selected and incorporated into a pharmaceutical composition on the basis of the bacterial isolate exhibiting at least one of characteristic (i), characteristic (ii), and characteristic (iii). In embodiments, a bacterial isolate comprising Odoribacter sphlanchnicus is selected and incorporated into a pharmaceutical composition on the basis of the bacterial isolate exhibiting at least two of characteristics (i), characteristic (ii), and characteristic (iii). In embodiments, a bacterial isolate comprising Odoribacter sphlanchnicus is selected and incorporated into a pharmaceutical composition on the basis of the bacterial isolate exhibiting each of characteristic (i), characteristic (ii), and characteristic (iii).

In embodiments, a bacterial isolate comprising Faecalibacterium prausnitzii is selected and incorporated into a pharmaceutical composition on the basis of the bacterial isolate exhibiting at least one of characteristic (i), characteristic (ii) and characteristic (iii). In embodiments, a bacterial isolate comprising Faecalibacterium prausnitzii is selected and incorporated into a pharmaceutical composition on the basis of the bacterial isolate exhibiting at least two of characteristic (i), characteristic (ii) and characteristic (iii). In embodiments, a bacterial isolate comprising Faecalibacterium prausnitzii is selected and incorporated into a pharmaceutical composition on the basis of the bacterial isolate exhibiting each of characteristic (i), characteristic (ii) and characteristic (iii).

In embodiments, a bacterial isolate comprising Eubacterium rectale is selected and incorporated into a pharmaceutical composition on the basis of the bacterial isolate exhibiting at least one of characteristic (i) and characteristic (iii). In embodiments, a bacterial isolate comprising Eubacterium rectale is selected and incorporated into a pharmaceutical composition on the basis of the bacterial isolate exhibiting each of characteristic (i) and characteristic (ii).

In embodiments, a bacterial isolate comprising Roseburia faecis is selected and incorporated into a pharmaceutical composition on the basis of the bacterial isolate exhibiting characteristic (i).

In embodiments, a bacterial isolate comprising Akkermansia muciniphila is selected and incorporated into a pharmaceutical composition on the basis of the bacterial isolate exhibiting at least one of characteristic (ii) and characteristic (iii). In embodiments, a bacterial isolate comprising Akkermansia muciniphila is selected and incorporated into a pharmaceutical composition on the basis of the bacterial isolate exhibiting each of characteristic (ii) and characteristic (iii).

In embodiments, a bacterial isolate comprising Alistipes shahii is selected and incorporated into a pharmaceutical composition on the basis of the bacterial isolate exhibiting at least one of characteristic (ii) and characteristic (iii). In embodiments, a bacterial isolate comprising Alistipes shahii is selected and incorporated into a pharmaceutical composition on the basis of the bacterial isolate exhibiting each of characteristic (ii) and characteristic (iii).

In embodiments, a bacterial isolate comprising Bacteroides cellulosilyticus is selected and incorporated into a pharmaceutical composition on the basis of the bacterial isolate exhibiting characteristic (iii).

In embodiments, a bacterial isolate comprising Subdoligranulum variabile is selected and incorporated into a pharmaceutical composition on the basis of the bacterial isolate exhibiting at least one of characteristic (i), characteristic (ii) and characteristic (iii). In embodiments, a bacterial isolate comprising Subdoligranulum variabile is selected and incorporated into a pharmaceutical composition on the basis of the bacterial isolate exhibiting at least two of characteristics (i), characteristic (ii) and characteristic (iii). In embodiments, a bacterial isolate comprising Subdoligranulum variabile is selected and incorporated into a pharmaceutical composition on the basis of the bacterial isolate exhibiting each of characteristic (i), characteristic (ii) and characteristic (iii).

In embodiments, a bacterial isolate can be selected for inclusion in a pharmaceutical composition described herein based on its ability to produce a particular amount of an SCFA such as butyrate (i.e., based on characteristic (i)) as measured in a functional assay. For example, a series of bacterial isolates can be screened for an ability to produce a threshold concentration of an SCFA when incubated with an organic substrate capable of being converted to an SCFA during a period of time. In embodiments, the substrate can comprise at least one of an oligosaccharide (e.g., a fructooligosaccharide (FOS) or an xylooligosaccharide (XOS)), sunfiber/partially hydrolyzed guar gum (PHGG), or barley malt. The bacterial isolate can be incubated with the substrate for any period of time sufficient to allow conversion of the substrate to the SCFA, for example at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 35 hours, at least 40 hours, at least 44 hours, at least 48 hours, or greater than 48 hours.

In embodiments, a bacterial isolate can be selected for inclusion in a pharmaceutical composition described herein based on a production of SCFA (e.g., butyrate) over a duration of time (e.g., 24 hours) at a level of at least 5 mM, at least 10 mM, at least 15 mM, at least 20 mM, at least 25 mM, at least 30 mM, at least 35 mM, at least 40 mM, at least 45 mM at least 50 mM, at least 60 mM, at least 70 mM, at least 80 mM, at least 90 mM, at least 100 mM, or greater than 100 mM. In embodiments, a bacterial isolate selected for inclusion in a pharmaceutical composition is provided in Table 2, or comprises a 16S rRNA sequence at least 95% identical to a 16S rRNA sequence corresponding to a SEQ ID NO of a bacterial isolate provided in Table 2.

In embodiments, a bacterial isolate can be selected for inclusion in a pharmaceutical composition described herein based on its ability to modulate production and/or release of one or more cytokines by a eukaryotic cell incubated with the bacterial isolate (i.e., based on characteristic (ii)) as measured in a functional assay. For example, a series of bacterial isolates can be screened for an ability to modulate production and/or release of one or more cytokines from a eukaryotic cell incubated with each bacterial isolate. Examples of eukaryotic cells that can be used in the functional assay include an intestinal cell, an epithelial cell, an intestinal mucosal cell, an intestinal epithelial cell, an intestinal lamina propria cell, an endothelial cell, fibroblast, a stromal cell, a macrophage, a B lymphocyte, a T lymphocyte, a mast cell, and a peripheral blood mononuclear cell (PBMC). In embodiments, the eukaryotic cell can be a human cell.

The bacterial isolate can be incubated with the eukaryotic cell for any period of time sufficient to allow modulation of cytokine expression, for example at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 35 hours, at least 40 hours, at least 44 hours, at least 48 hours, or greater than 48 hours.

In embodiments, a bacterial isolate can be selected for inclusion in a pharmaceutical composition described herein based on the induction of IL-10 by a population of eukaryotic cells (e.g., population of PBMCs) incubated with the bacterial isolate during a period of time (e.g., 24 hours), as measured in a functional assay. For example, the bacterial isolate can induce IL-10 at a concentration of at least 1000 pg/ml, at least 1500 pg/ml, at least 2000 pg/ml, at least 2500 pg/ml, at least 3000 pg/ml, at least 3500 pg/ml, at least 4000 pg/ml, at least 5000 pg/ml, at least 6000 pg/ml, at least 7000 pg/ml, at least 8000 pg/ml, at least 9000 pg/ml, at least 10,000 pg/ml, or greater than 10,000 pg/ml. In embodiments, a bacterial isolate selected for inclusion in a pharmaceutical composition is provided in Table 3, or comprises a 16S rRNA sequence at least 95% identical to a 16S rRNA sequence corresponding to a SEQ ID NO of a bacterial isolate provided in Table 3.

In embodiments, a bacterial isolate can be selected for inclusion in a pharmaceutical composition described herein based on a limited production of GM-CSF by a population of eukaryotic cells (e.g., population of PBMCs) incubated with the bacterial isolate during a period of time (e.g., 24 hours), as measured in a functional assay. For example, the bacterial isolate can limit production of GM-CSF to a concentration of no more than 5 pg/ml, 10 pg/ml, 15 pg/ml, 20 pg/ml, 25 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, 80 pg/ml, 85 pg/ml, 90 pg/ml, 95 pg/ml, 100 pg/ml, 105 pg/ml, 110 pg/ml, 115 pg/ml, 120 pg/ml, 125 pg/ml, 130 pg/ml, 135 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, or 200 pg/ml. In embodiments, a bacterial isolate selected for inclusion in a pharmaceutical composition is provided in Table 3, or comprises a 16S rRNA sequence at least 95% identical to a 16S rRNA sequence corresponding to a SEQ ID NO of a bacterial isolate provided in Table 3.

In embodiments, a bacterial isolate can be selected for inclusion in a pharmaceutical composition described herein based on a limited production of IL-12 by a population of eukaryotic cells (e.g., population of PBMCs) incubated with the bacterial isolate during a period of time (e.g., 24 hours), as measured in a functional assay. For example, the bacterial isolate can limit production of IL-12 to a concentration of no more than 5 pg/ml, 10 pg/ml, 15 pg/ml, 20 pg/ml, 25 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, 80 pg/ml, 85 pg/ml, 90 pg/ml, 95 pg/ml, 100 pg/ml, 105 pg/ml, 110 pg/ml, 115 pg/ml, 120 pg/ml, 125 pg/ml, 130 pg/ml, 135 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, or 200 pg/ml. In embodiments, a bacterial isolate selected for inclusion in a pharmaceutical composition is provided in Table 3, or comprises a 16S rRNA sequence at least 95% identical to a 16S rRNA sequence corresponding to a SEQ ID NO of a bacterial isolate provided in Table 3.

In embodiments, a bacterial isolate can be selected for inclusion in a pharmaceutical composition described herein based on a limited production of IFN-gamma by a population of eukaryotic cells (e.g., population of PBMCs) incubated with the bacterial isolate during a period of time (e.g., 24 hours), as measured in a functional assay. For example, the bacterial isolate can limit production of IFN-gamma to a concentration of no more than 5 pg/ml, 10 pg/ml, 15 pg/ml, 20 pg/ml, 25 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, 80 pg/ml, 85 pg/ml, 90 pg/ml, 95 pg/ml, 100 pg/ml, 105 pg/ml, 110 pg/ml, 115 pg/ml, 120 pg/ml, 125 pg/ml, 130 pg/ml, 135 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, or 200 pg/ml. In embodiments, a bacterial isolate selected for inclusion in a pharmaceutical composition is provided in Table 3, or comprises a 16S rRNA sequence at least 95% identical to a 16S rRNA sequence corresponding to a SEQ ID NO of a bacterial isolate provided in Table 3.

In embodiments, a bacterial isolate can be selected for inclusion in a pharmaceutical composition described herein based on a limited production of TNF-alpha by a population of eukaryotic cells (e.g., population of PBMCs) incubated with the bacterial isolate during a period of time (e.g., 24 hours), as measured in a functional assay. For example, the bacterial isolate can limit production of TNF-alpha to a concentration of no more than 20 pg/ml, 30 pg/ml, 40 pg/ml, 50 pg/ml, 75 pg/ml, 100 pg/ml, 150 pg/ml, 200 pg/ml, 250 pg/ml, 300 pg/ml, 350 pg/ml, 400 pg/ml, 450 pg/ml, 500 pg/ml, 550 pg/ml, 600 pg/ml, 650 pg/ml, 700 pg/ml, 750 pg/ml, 800 pg/ml, 850 pg/ml, 900 pg/ml, 950 pg/ml, 1000 pg/ml, 1100 pg/ml, 1200 pg/ml, 1300 pg/ml, 1400 pg/ml, 1500 pg/ml, 1600 pg/ml, 1700 pg/ml, 1800 pg/ml, 1900 pg/ml, 2000 pg/ml, 2100 pg/ml, 2200 pg/ml, 2300 pg/ml, 2400 pg/ml, or 2500 pg/ml. In embodiments, a bacterial isolate selected for inclusion in a pharmaceutical composition is provided in Table 3, or comprises a 16S rRNA sequence at least 95% identical to a 16S rRNA sequence corresponding to a SEQ ID NO of a bacterial isolate provided in Table 3.

In embodiments, a bacterial isolate can be selected for inclusion in a pharmaceutical composition described herein based on a limited production of IL-23 by a population of eukaryotic cells (e.g., population of PBMCs) incubated with the bacterial isolate during a period of time (e.g., 24 hours), as measured in a functional assay. For example, the bacterial isolate can limit production of IL-23 to a concentration of no more than 5 pg/ml, 10 pg/ml, 15 pg/ml, 20 pg/ml, 25 pg/ml, 30 pg/ml, 35 pg/ml, 40 pg/ml, 45 pg/ml, 50 pg/ml, 55 pg/ml, 60 pg/ml, 65 pg/ml, 70 pg/ml, 75 pg/ml, 80 pg/ml, 85 pg/ml, 90 pg/ml, 95 pg/ml, 100 pg/ml, 105 pg/ml, 110 pg/ml, 115 pg/ml, 120 pg/ml, 125 pg/ml, 130 pg/ml, 135 pg/ml, 140 pg/ml, 145 pg/ml, 150 pg/ml, 155 pg/ml, 160 pg/ml, 165 pg/ml, 170 pg/ml, 175 pg/ml, 180 pg/ml, 185 pg/ml, 190 pg/ml, 195 pg/ml, 200 pg/ml, 250 pg/ml, or 300 pg/ml. In embodiments, a bacterial isolate selected for inclusion in a pharmaceutical composition is provided in Table 3, or comprises a 16S rRNA sequence at least 95% identical to a 16S rRNA sequence corresponding to a SEQ ID NO of a bacterial isolate provided in Table 3.

In embodiments, a bacterial isolate can be selected for inclusion in a pharmaceutical composition described herein based on an IL-10:IL-12 ratio produced by a population of eukaryotic cells (e.g., population of PBMCs) incubated with the bacterial isolate during a period of time (e.g., 24 hours), as measured in a functional assay. For example, the bacterial isolate can generate an IL-10:IL-12 ratio of at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 325, at least 350, at least 375, at least 400, at least 425, at least 450, at least 475, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, at least 2000, at least 2200, at least 2400, at least 2600, at least 2800, at least 3000, at least 3200, at least 3400, at least 3600, at least 3800, at least 4000, or greater than 4000. In an embodiment, at least one of the one or more bacterial isolates is provided in Table 3, or comprises a 16S rRNA sequence at least 95% identical to a SEQ ID NO of a bacterial isolate provided in Table 3.

In embodiments, a bacterial isolate can be selected for inclusion in a pharmaceutical composition described herein based on an IL-10:TNF-alpha ratio produced by a population of eukaryotic cells (e.g., population of PBMCs) incubated with the bacterial isolate during a period of time (e.g., 24 hours), as measured in a functional assay. For example, the bacterial isolate can generate an IL-10:TNF-alpha ratio of at least 0.5, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, at least 75, at least 80, at least 85, at least 90, at least 95, or at least 100. In embodiments, a bacterial isolate selected for inclusion in a pharmaceutical composition is provided in Table 3, or comprises a 16S rRNA sequence at least 95% identical to a 16S rRNA sequence corresponding to a SEQ ID NO of a bacterial isolate provided in Table 3.

In an embodiment, a method of manufacture comprises selecting one or more bacterial isolates comprising a 16S rRNA sequence that is at least 95% identical to the 16S rRNA sequence of one or more of the bacterial isolates provided in Table 1, and incorporating the one or more bacterial isolates into a pharmaceutical composition. In an embodiment, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or greater than 20 bacterial isolates comprising a 16S rRNA sequence that is at least 95% identical to the 16S rRNA sequence of one or more of the bacterial isolates provided in Table 1 are selected and incorporated into a microbial cocktail.

In an embodiment, a method of manufacture comprises selecting one or more bacterial isolates comprising a 16S rRNA sequence that is at least 95% identical to the 16S rRNA sequence of one or more of the bacterial isolates provided in Tables 2-4, and incorporating the one or more bacterial isolates into a pharmaceutical composition.

In an embodiment, a method of manufacture comprises selecting bacterial isolates comprising 16S rRNA sequences at least 95% identical to the 16S rRNA sequences of all of the bacterial isolates provided in one of Tables 7-21, incorporating the bacterial isolates into a microbial cocktail, and incorporating the microbial cocktail into a pharmaceutical composition.

In embodiments, one or more bacterial isolates can be rationally selected for inclusion in a pharmaceutical composition described herein based on one or more desired traits or characteristics exhibited or possessed by the isolate. Non-limiting examples of a trait possessed or exhibited by a bacterial isolate that can form the basis of rational selection can include: a coding or non-coding DNA sequence; an RNA sequence; a polypeptide sequence; production or lack of production of a particular RNA or protein product; activity or inactivity of a metabolic pathway; production or lack of production of a particular metabolic product; production or lack of production of a particular ligand or set of ligands; ability to induce or inhibit production of a compound or pathway in a target cell (e.g., eukaryotic cell), association of the presence or abundance of the strain with the existence or severity of a condition or indication (e.g. in a human patient); and the particular taxonomic classification of the strain, including genus or species.

In embodiments, a method of manufacture comprises selecting a dosage of a bacterial isolate to be incorporated into a pharmaceutical composition, and incorporating the bacterial isolate into the pharmaceutical composition at the selected dosage. In an embodiment, selecting a dosage of a bacterial isolate comprises determining a threshold or minimum dosage required for engraftment of the bacterial isolate in the intestine of a subject administered the bacterial isolate, and selecting a dosage of the bacterial isolate that is above the threshold or minimum dosage.

### EMBODIMENTS

Embodiment 1: A pharmaceutical composition comprising a cocktail of bacterial isolates, wherein at least one of the bacterial isolates comprises a 16S rRNA sequence that is at least 95% identical to a 16S rRNA sequence of a bacterial isolate provided in Table 1, and wherein the at least two bacterial isolates are isolated from stool samples of different donors.

Embodiment 2: The pharmaceutical composition of embodiment 1, wherein at least two of the bacterial isolates comprise a 16S rRNA sequence that is at least 95% identical to a 16S rRNA sequence of a bacterial isolate provided in Table 1.

Embodiment 3: The pharmaceutical composition of embodiment 1 or embodiment 2, wherein at least three of the bacterial isolates comprise a 16S rRNA sequence that is at least 95% identical to a 16S rRNA sequence of a bacterial isolate provided in Table 1.

Embodiment 4: The pharmaceutical composition of any one of embodiments 1 to 3, wherein at least four of the bacterial isolates comprise a 16S rRNA sequence that is at least 95% identical to a 16S rRNA sequence of a bacterial isolate provided in Table 1.

Embodiment 5: The pharmaceutical composition of any one of embodiments 1 to 4, wherein at least five of the bacterial isolates comprise a 16S rRNA sequence that is at least 95% identical to a 16S rRNA sequence of a bacterial isolate provided in Table 1.

Embodiment 6: The pharmaceutical composition of any one of embodiments 1 to 5, wherein at least six of the bacterial isolates comprise a 16S rRNA sequence that is at least 95% identical to a 16S rRNA sequence of a bacterial isolate provided in Table 1.

Embodiment 7: The pharmaceutical composition of any one of embodiments 1 to 6, wherein at least seven of the bacterial isolates comprise a 16S rRNA sequence that is at least 95% identical to a 16S rRNA sequence of a bacterial isolate provided in Table 1.

Embodiment 8: The pharmaceutical composition of any one of embodiments 1 to 7, wherein at least eight of the bacterial isolates comprise a 16S rRNA sequence that is at least 95% identical to a 16S rRNA sequence of a bacterial isolate provided in Table 1.

Embodiment 9: The pharmaceutical composition of any one of embodiments 1 to 8, wherein at least nine of the bacterial isolates comprise a 16S rRNA sequence that is at least 95% identical to a 16S rRNA sequence of a bacterial isolate provided in Table 1.

Embodiment 10: The pharmaceutical composition of any one of embodiments 1 to 9, wherein at least ten of the bacterial isolates comprise a 16S rRNA sequence that is at least 95% identical to a 16S rRNA sequence of a bacterial isolate provided in Table 1.

Embodiment 11: A pharmaceutical composition comprising a plurality of bacterial isolates, wherein the plurality of bacterial isolates comprises at least two bacterial isolates selected from the group consisting of Eubacterium rectale, Odoribacter splanchnicus and Subdoligranulum variabile, wherein the at least two bacterial isolates are isolated from stool samples of different donors.

Embodiment 12: The pharmaceutical composition of embodiment 11, wherein the Subdoligranulum variabile comprises a 16S rRNA sequence that is at least 95% identical to at least one of SEQ ID NO: 22 or SEQ ID NO: 23.

Embodiment 13: The pharmaceutical composition of embodiment 11 or embodiment 12, wherein the Odoribacter comprises a 16S rRNA sequence that is at least 95% identical to SEQ ID NO: 2.

Embodiment 14: The pharmaceutical composition of any one of embodiments 11 to 13, wherein the Eubacterium rectale comprises a 16S rRNA sequence that is at least 95% identical to SEQ ID NO: 8.

Embodiment 15: The pharmaceutical composition of any one of embodiments 11 to 13, wherein the at least two bacterial isolates do not include Eubacterium rectale.

Embodiment 16: The pharmaceutical composition of any one of embodiment 11, embodiment 12, embodiment 13, or embodiment 14, wherein the at least two bacterial isolates do not include Odoribacter splanchnicus.

Embodiment 17: The pharmaceutical composition of any one of embodiment 11, embodiment 13, or embodiment 14, wherein the at least two bacterial isolates do not include Subdoligranulum variabile.

Embodiment 18: The pharmaceutical composition of any one of embodiments 11 to 14 or embodiment 17, wherein the at least two bacterial isolates comprise Odoribacter splanchnicus and Eubacterium rectale.

Embodiment 19: The pharmaceutical composition of embodiment 18, wherein the plurality of bacterial isolates further comprises at least one of Bacteroides cellulosilyticus, Faecalibacterium prausnitzii, Alistipes shahii, and Blautia obeum.

Embodiment 20: The pharmaceutical composition of embodiment 19, wherein the plurality of bacterial isolates further comprises each of Bacteroides cellulosilyticus, Faecalibacterium prausnitzii, Alistipes shahii, and Blautia obeum.

Embodiment 21: The pharmaceutical composition of embodiment 19 or embodiment 20, wherein the Bacteroides cellulosilyticus comprises a 16S rRNA sequence that is at least 95% identical to SEQ ID NO: 14.

Embodiment 22: The pharmaceutical composition of any one of embodiments 19 to 21, wherein the Faecalibacterium prausnitzii comprises a 16S rRNA sequence that is at least 95% identical to at least one of SEQ ID NO: 1 or SEQ ID NO: 7.

Embodiment 23: The pharmaceutical composition of any one of embodiments 19 to 22, wherein the Alistipes shahii comprises a 16S rRNA sequence that is at least 95% identical to SEQ ID NO: 18.

Embodiment 24: The pharmaceutical composition of any one of embodiments 19 to 23, wherein the Blautia obeum comprises a 16S rRNA sequence that is at least 95% identical to SEQ ID NO: 9.

Embodiment 25: The pharmaceutical composition of embodiment 18, wherein the plurality of bacterial isolates further comprises at least one of Bacteroides cellulosilyticus, Faecalibacterium prausnitzii, Alistipes shahii, Anaerostipes hadrus, Roseburia faecis, and Blautia obeum.

Embodiment 26: The pharmaceutical composition of embodiment 25, wherein the plurality of bacterial isolates further comprises each of Bacteroides cellulosilyticus, Faecalibacterium prausnitzii, Alistipes shahii, Anaerostipes hadrus, and Roseburia faecis

Embodiment 27: The pharmaceutical composition of embodiment 25 or embodiment 26, wherein the Bacteroides cellulosilyticus comprises a 16S rRNA sequence that is at least 95% identical to SEQ ID NO: 14.

Embodiment 28: The pharmaceutical composition of any one of embodiments 25 to 27, wherein the Faecalibacterium prausnitzii comprises a 16S rRNA sequence that is at least 95% identical to at least one of SEQ ID NO: 1 or SEQ ID NO: 7.

Embodiment 29: The pharmaceutical composition of any one of embodiments 25 to 28, wherein the Alistipes shahii comprises a 16S rRNA sequence that is at least 95% identical to SEQ ID NO: 18.

Embodiment 30: The pharmaceutical composition of any one of embodiments 25 to 29, wherein the Anaerostipes hadrus comprises a 16S rRNA sequence that is at least 95% identical to SEQ ID NO: 3.

Embodiment 31: The pharmaceutical composition of any one of embodiments 25 to 30, wherein the Roseburia faecis comprises a 16S rRNA sequence that is at least 95% identical to SEQ ID NO: 19.

Embodiment 32: The pharmaceutical composition of embodiment 25, wherein the plurality of bacterial isolates does not comprise at least one of Blautia obeum and Anaerostipes hadrus.

Embodiment 33: The pharmaceutical composition of embodiment 32, wherein the plurality of bacterial isolates does not comprise Blautia obeum.

Embodiment 34: The pharmaceutical composition of embodiment 18, wherein the plurality of bacterial isolates further comprises each of Bacteroides cellulosilyticus, Faecalibacterium prausnitzii, Alistipes shahii, Blautia obeum, and Roseburia faecis.

Embodiment 35: The pharmaceutical composition of embodiment 34, wherein the Bacteroides cellulosilyticus comprises a 16S rRNA sequence that is at least 95% identical to SEQ ID NO: 14.

Embodiment 36: The pharmaceutical composition of embodiment 34, wherein the Bacteroides cellulosilyticus comprises a 16S rRNA sequence that is at least 97% identical to SEQ ID NO: 14.

Embodiment 37: The pharmaceutical composition of any one of embodiments 34 to 36, wherein the Faecalibacterium prausnitzii comprises a 16S rRNA sequence that is at least 95% identical to at least one of SEQ ID NO: 1 or SEQ ID NO: 7.

Embodiment 38: The pharmaceutical composition of any one of embodiments 34 to 37, wherein the Alistipes shahii comprises a 16S rRNA sequence that is at least 95% identical to SEQ ID NO: 18.

Embodiment 39: The pharmaceutical composition of any one of embodiments 34 to 38, wherein the Blautia obeum comprises a 16S rRNA sequence that is at least 95% identical to SEQ ID NO: 9.

Embodiment 40: The pharmaceutical composition of any one of embodiments 34 to 39, wherein the Roseburia faecis comprises a 16S rRNA sequence that is at least 95% identical to SEQ ID NO: 19.

Embodiment 41: The pharmaceutical composition of embodiment 18, wherein the plurality of bacterial isolates further comprises at least one of Bacteroides cellulosilyticus, Alistipes shahii, and Roseburia faecis.

Embodiment 42: The pharmaceutical composition of embodiment 41, wherein the plurality of bacterial isolates further comprises each of Bacteroides cellulosilyticus, Alistipes shahii, and Roseburia faecis.

Embodiment 43: The pharmaceutical composition of embodiment 41 or embodiment 42, wherein the Bacteroides cellulosilyticus comprises a 16S rRNA sequence that is at least 95% identical to SEQ ID NO: 14.

Embodiment 44: The pharmaceutical composition of embodiment 41 or embodiment 42, wherein the Bacteroides cellulosilyticus comprises a 16S rRNA sequence that is at least 97% identical to SEQ ID NO: 14.

Embodiment 45: The pharmaceutical composition of any one of embodiments 41 to 44, wherein the Roseburia faecis comprises a 16S rRNA sequence that is at least 95% identical to SEQ ID NO: 19.

Embodiment 46: The pharmaceutical composition of any one of embodiments 41 to 45, wherein the Alistipes shahii comprises a 16S rRNA sequence that is at least 95% identical to SEQ ID NO: 18.

Embodiment 47: The pharmaceutical composition of embodiment 18, wherein the plurality of bacterial isolates further comprises at least one of Faecalibacterium prausnitzii, Roseburia faecis, and Anaerostipes hadrus.

Embodiment 48: The pharmaceutical composition of embodiment 47, wherein the plurality of bacterial isolates further comprises each of Faecalibacterium prausnitzii, Roseburia faecis, and Anaerostipes hadrus.

Embodiment 49: The pharmaceutical composition of embodiment 47 or embodiment 48, wherein the Faecalibacterium prausnitzii comprises a 16S rRNA sequence that is at least 95% identical to at least one of SEQ ID NO: 1 or SEQ ID NO: 7.

Embodiment 50: The pharmaceutical composition of embodiment 47 or embodiment 48, wherein the Faecalibacterium prausnitzii comprises a 16S rRNA sequence that is at least 97% identical to at least one of SEQ ID NO: 1 or SEQ ID NO: 7.

Embodiment 51: The pharmaceutical composition of any one of embodiment 47 to 50, wherein the Roseburia faecis comprises a 16S rRNA sequence that is at least 95% identical to SEQ ID NO: 19.

Embodiment 52: The pharmaceutical composition of any one of embodiment 47 to 51, wherein theAnaerostipes hadrus comprises a 16S rRNA sequence that is at least 95% identical to SEQ ID NO: 3.

Embodiment 53: The pharmaceutical composition of embodiment 18, wherein the plurality of bacterial isolates further comprises at least one of Bacteroides cellulosilyticus, Blautia obeum, and Alistipes shahii.

Embodiment 54: The pharmaceutical composition of embodiment 53, wherein the plurality of bacterial isolates further comprises each of Bacteroides cellulosilyticus, Blautia obeum, and Alistipes shahii.

Embodiment 55: The pharmaceutical composition of embodiment 53 or embodiment 54, wherein the Bacteroides cellulosilyticus comprises a 16S rRNA sequence that is at least 95% identical to SEQ ID NO: 14.

Embodiment 56: The pharmaceutical composition of embodiment 53 or embodiment 54, wherein the Bacteroides cellulosilyticus comprises a 16S rRNA sequence that is at least 97% identical to SEQ ID NO: 14.

Embodiment 57: The pharmaceutical composition of any one of embodiments 53 to 56, wherein the Blautia obeum comprises a 16S rRNA sequence that is at least 95% identical to SEQ ID NO: 9.

Embodiment 58: The pharmaceutical composition of any one of embodiments 53 to 57, wherein the Alistipes shahii comprises a 16S rRNA sequence that is at least 95% identical to SEQ ID NO: 18.

Embodiment 59: The pharmaceutical composition of embodiment 18, wherein the plurality of bacterial isolates further comprises at least one of Faecalibacterium prausnitzii, Alistipes shahii, and Roseburia faecis.

Embodiment 60: The pharmaceutical composition of embodiment 59, wherein the plurality of bacterial isolates further comprises each of Faecalibacterium prausnitzii, Alistipes shahii, and Roseburia faecis.

Embodiment 61: The pharmaceutical composition of embodiment 59 or embodiment 60, wherein the Faecalibacterium prausnitzii comprises a 16S rRNA sequence that is at least 95% identical to at least one of SEQ ID NO: 1 or SEQ ID NO: 7.

Embodiment 62: The pharmaceutical composition of embodiment 59 or embodiment 60, wherein the Faecalibacterium prausnitzii comprises a 16S rRNA sequence that is at least 97% identical to at least one of SEQ ID NO: 1 or SEQ ID NO: 7.

Embodiment 63: The pharmaceutical composition of any one of embodiments 59 to 62, wherein the Alistipes shahii comprises a 16S rRNA sequence that is at least 95% identical to SEQ ID NO: 18.

Embodiment 64: The pharmaceutical composition of any one of embodiments 59 to 63, wherein the Roseburia faecis comprises a 16S rRNA sequence that is at least 95% identical to SEQ ID NO: 19.

Embodiment 65: The pharmaceutical composition of embodiment 18, wherein the plurality of bacterial isolates further comprises at least one of Faecalibacterium prausnitzii, Blautia obeum, and Roseburia faecis.

Embodiment 66: The pharmaceutical composition of embodiment 65, wherein the plurality of bacterial isolates further comprises each of Faecalibacterium prausnitzii, Blautia obeum, and Roseburia faecis.

Embodiment 67: The pharmaceutical composition of embodiment 65 or embodiment 66, wherein the Faecalibacterium prausnitzii comprises a 16S rRNA sequence that is at least 95% identical to at least one of SEQ ID NO: 1 or SEQ ID NO: 7.

Embodiment 68: The pharmaceutical composition of embodiment 65 or embodiment 66, wherein the Faecalibacterium prausnitzii comprises a 16S rRNA sequence that is at least 97% identical to at least one of SEQ ID NO: 1 or SEQ ID NO: 7.

Embodiment 69: The pharmaceutical composition of any one of embodiments 65 to 68, wherein the Blautia obeum comprises a 16S rRNA sequence that is at least 95% identical to SEQ ID NO: 9.

Embodiment 70: The pharmaceutical composition of any one of embodiments 65 to 69, wherein the Roseburia faecis comprises a 16S rRNA sequence that is at least 95% identical to SEQ ID NO: 19.

Embodiment 71: The pharmaceutical composition of any one of embodiments 65 to 69, wherein the Roseburia faecis comprises a 16S rRNA sequence that is at least 97% identical to SEQ ID NO: 19.

Embodiment 72: The pharmaceutical composition of embodiment 11, wherein the at least two bacterial isolates comprise Eubacterium rectale and Subdoligranulum variabile.

Embodiment 73: The pharmaceutical composition of embodiment 72, wherein the plurality of bacterial isolates further comprises at least one of Faecalibacterium prausnitzii, Coprococcus comes, Anaerostipes hadrus, and Roseburia faecis.

Embodiment 74: The pharmaceutical composition of embodiment 72 or embodiment 73, wherein the plurality of bacterial isolates further comprises each of Faecalibacterium prausnitzii, Coprococcus comes, Anaerostipes hadrus, and Roseburia faecis.

Embodiment 75: The pharmaceutical composition of any one of embodiments 72 to 74, wherein the Faecalibacterium prausnitzii comprises a 16S rRNA sequence that is at least 95% identical to at least one of SEQ ID NO: 1 or SEQ ID NO: 7.

Embodiment 76: The pharmaceutical composition of any one of embodiments 72 to 75, wherein the Anaerostipes hadrus comprises a 16S rRNA sequence that is at least 95% identical to SEQ ID NO: 3.

Embodiment 77: The pharmaceutical composition of any one of embodiments 72 to 76, wherein the Roseburia faecis comprises a 16S rRNA sequence that is at least 95% identical to SEQ ID NO: 19.

Embodiment 78: The pharmaceutical composition of any one of embodiments 72 to 77, wherein the Coprococcus comes comprises a 16S rRNA sequence that is at least 95% identical to SEQ ID NO: 17.

Embodiment 79: The pharmaceutical composition of any one of embodiments 1 to 18, wherein the plurality of bacterial strains does not include at least one of Faecalibacterium prausnitzii, Roseburia faecis, Bacteroides cellulosilyticus, Alistipes shahii, or Blautia obeum.

Embodiment 80: A pharmaceutical composition comprising a plurality of bacterial isolates, wherein the plurality of bacterial isolates comprises Roseburia faecis and Bacteroides cellulosilyticus, and at least one of Faecalibacterium prausnitzii and Alistipes shahii.

Embodiment 81: The pharmaceutical composition of embodiment 80, wherein the plurality of bacterial isolates further comprises at least one of Eubacterium rectale, Anaerostipes hadrus, and Blautia obeum.

Embodiment 82: The pharmaceutical composition of embodiment 80 or embodiment 81, wherein the plurality of bacterial isolates comprises each of Roseburia faecis, Bacteroides cellulosilyticus, Faecalibacterium prausnitzii, Alistipes shahii, Eubacterium rectale, and Anaerostipes hadrus.

Embodiment 83: The pharmaceutical composition of any one of embodiments 80 to 82, wherein the Roseburia faecis comprises a 16S rRNA sequence that is at least 95% identical to SEQ ID NO: 19.

Embodiment 84: The pharmaceutical composition of any one of embodiments 80 to 83, wherein the Bacteroides cellulosilyticus comprises a 16S rRNA sequence that is at least 95% identical to SEQ ID NO: 14.

Embodiment 85: The pharmaceutical composition of any one of embodiments 80 to 84, wherein the Faecalibacterium prausnitzii comprises a 16S rRNA sequence that is at least 95% identical to at least one of SEQ ID NO: 1 or SEQ ID NO: 7.

Embodiment 86: The pharmaceutical composition of any one of embodiments 80 to 85, wherein the Alistipes shahii comprises a 16S rRNA sequence that is at least 95% identical to SEQ ID NO: 18.

Embodiment 87: The pharmaceutical composition of any one of embodiments 80 to 86, wherein the Eubacterium rectale comprises a 16S rRNA sequence that is at least 95% identical to SEQ ID NO: 8.

Embodiment 88: The pharmaceutical composition of any one of embodiments 80 to 87, wherein the Anaerostipes hadrus comprises a 16S rRNA sequence that is at least 95% identical to SEQ ID NO: 3.

Embodiment 89: The pharmaceutical composition of any one of embodiments 80 to 89, wherein the plurality of bacterial isolates does not comprise at least one of Anaerostipes hadrus and Blautia obeum.

Embodiment 90: The pharmaceutical composition of embodiment 80, wherein the plurality of bacterial isolates comprises each of Roseburia faecis, Bacteroides cellulosilyticus, Faecalibacterium prausnitzii, Alistipes shahii, and further comprises Eubacterium rectale and Blautia obeum.

Embodiment 91: The pharmaceutical composition of embodiment 90, wherein the Roseburia faecis comprises a 16S rRNA sequence that is at least 95% identical to SEQ ID NO: 19.

Embodiment 92: The pharmaceutical composition of embodiment 90 or embodiment 91, wherein the Bacteroides cellulosilyticus comprises a 16S rRNA sequence that is at least 95% identical to SEQ ID NO: 14.

Embodiment 93: The pharmaceutical composition of any one of embodiments 90 to 92, wherein the Faecalibacterium prausnitzii comprises a 16S rRNA sequence that is at least 95% identical to at least one of SEQ ID NO: 1 or SEQ ID NO: 7.

Embodiment 94: The pharmaceutical composition of any one of embodiments 90 to 93, wherein the Alistipes shahii comprises a 16S rRNA sequence that is at least 95% identical to SEQ ID NO: 18.

Embodiment 95: The pharmaceutical composition of any one of embodiments 90 to 94, wherein the Eubacterium rectale comprises a 16S rRNA sequence that is at least 95% identical to SEQ ID NO: 8.

Embodiment 96: The pharmaceutical composition of any one of embodiments 90 to 95, wherein the Blautia obeum comprises a 16S rRNA sequence that is at least 95% identical to SEQ ID NO: 9.

Embodiment 97: The pharmaceutical composition of any one of embodiment 1 to 96, wherein one or more of the plurality of bacterial isolates secretes a short-chain fatty acid (SCFA) in an intestine of a subject administered the composition.

Embodiment 98: The pharmaceutical composition of embodiment 97, wherein the SCFA is selected from the group consisting of acetic acid, butyric acid, caproic acid, formic acid, heptanoic acid, isobutyric acid, isocaproic acid, isovaleric acid, propionic acid, valeric acid, and a combination thereof.

Embodiment 99: The pharmaceutical composition of embodiment 97 or embodiment 98, wherein the SCFA is butyric acid.

Embodiment 100: The pharmaceutical composition of embodiment 99, wherein a level of the butyric acid produced by the one or more bacterial isolates over a period of 24 hours is at least 5 mM.

Embodiment 101: The pharmaceutical composition of embodiment 99, wherein a level of the butyric acid produced by the one or more bacterial isolates over a period of 24 hours is at least 10 mM.

Embodiment 102: The pharmaceutical composition of embodiment 99, wherein a level of the butyric acid produced by the one or more bacterial isolates over a period of 24 hours is at least 15 mM.

Embodiment 103: The pharmaceutical composition of embodiment 99, wherein a level of the butyric acid produced by the one or more bacterial isolates over a period of 24 hours is at least 20 mM.

Embodiment 104: The pharmaceutical composition of embodiment 99, wherein a level of the butyric acid produced by the one or more bacterial isolates over a period of 24 hours is at least 25 mM.

Embodiment 105: The pharmaceutical composition of embodiment 99, wherein a level of the butyric acid produced by the one or more bacterial isolates over a period of 24 hours is at least 30 mM.

Embodiment 106: The pharmaceutical composition of embodiment 99, wherein a level of the butyric acid produced by the one or more bacterial isolates over a period of 24 hours is at least 35 mM.

Embodiment 107: The pharmaceutical composition of embodiment 99, wherein a level of the butyric acid produced by the one or more bacterial isolates over a period of 24 hours is at least 40 mM.

Embodiment 108: The pharmaceutical composition of embodiment 99, wherein the one or more bacterial isolates producing at least one SCFA comprises a 16S rRNA sequence that is at least 95% identical to a 16S rRNA sequence of a bacterial isolate provided in Table 2.

Embodiment 109: A pharmaceutical composition comprising a plurality of bacterial isolates, wherein the plurality of bacterial isolates comprises a bacterial isolate comprising Parabacteroides merdae and at least one of Alistipes finegoldii and Alistipes onderdonkii.

Embodiment 110: The pharmaceutical composition of embodiment 109, wherein the plurality of bacterial isolates comprises both Alistipes finegoldii and Alistipes onderdonkii.

Embodiment 111: The pharmaceutical composition of embodiment 110, wherein the plurality of bacterial isolates further comprises at least one of Akkermansia muciniphila, Dorea longicatena, Blautia obeum, Blautia sp., Bacteroides uniformis or Bacteroides vulgatus.

Embodiment 112: The pharmaceutical composition of embodiment 111, wherein the plurality of bacterial isolates comprises each of Akkermansia muciniphila, Dorea longicatena, Blautia sp., Bacteroides uniformis and Bacteroides vulgatus.

Embodiment 113: The pharmaceutical composition of embodiment 111 or embodiment 112, wherein the Parabacteroides merdae comprises a 16S rRNA sequence that is at least 95% identical to SEQ ID NO: 5.

Embodiment 114: The pharmaceutical composition of any one of embodiments 111 to 113, wherein the Akkermansia muciniphila comprises a 16S rRNA sequence that is at least 95% identical to SEQ ID NO: 20.

Embodiment 115: The pharmaceutical composition of any one of embodiments 111 to 114, wherein the Alistipes finegoldii comprises a 16S rRNA sequence that is at least 95% identical to SEQ ID NO: 15.

Embodiment 116: The pharmaceutical composition of any one of embodiments 111 to 115, wherein the Dorea longicatena comprises a 16S rRNA sequence that is at least 95% identical to SEQ ID NO: 6.

Embodiment 117: The pharmaceutical composition of any one of embodiments 111 to 116, wherein the Alistipes onderdonkii comprises a 16S rRNA sequence that is at least 95% identical to SEQ ID NO: 4.

Embodiment 118: The pharmaceutical composition of any one of embodiments 111 to 117, wherein the Blautia sp. comprises a 16S rRNA sequence that is at least 95% identical to SEQ ID NO: 34.

Embodiment 119: The pharmaceutical composition of any one of embodiments 111 to 118, wherein the Bacteroides uniformis comprises a 16S rRNA sequence that is at least 95% identical to at least one of SEQ ID NO: 11 and SEQ ID NO: 16.

Embodiment 120: The pharmaceutical composition of any one of embodiments 111 to 119, wherein the Bacteroides vulgatus comprises a 16S rRNA sequence that is at least 95% identical to SEQ ID NO: 12.

Embodiment 121: The pharmaceutical composition of any one of embodiments 111 to 120, wherein the plurality of bacterial isolates does not include one ofAlistipesfinegoldii and Alistipes onderdonkii.

Embodiment 122: The pharmaceutical composition of embodiment 121, wherein the plurality of bacterial isolates does not include Alistipes finegoldii.

Embodiment 123: The pharmaceutical composition of embodiment 109, wherein the plurality of bacterial isolates comprises Alistipes finegoldii and further comprises Akkermansia muciniphila, Dorea longicatena, Blautia obeum, Blautia sp., Bacteroides uniformis and Bacteroides vulgatus.

Embodiment 124: The pharmaceutical composition of embodiment 123, wherein the Parabacteroides merdae comprises a 16S rRNA sequence that is at least 95% identical to SEQ ID NO: 5.

Embodiment 125: The pharmaceutical composition of embodiment 123 or embodiment 124, wherein the Akkermansia muciniphila comprises a 16S rRNA sequence that is at least 95% identical to SEQ ID NO: 20.

Embodiment 126: The pharmaceutical composition of any one of embodiments 123 to 125, wherein the Alistipes finegoldii comprises a 16S rRNA sequence that is at least 95% identical to SEQ ID NO: 15.

Embodiment 127: The pharmaceutical composition of any one of embodiments 123 to 126, wherein the Dorea longicatena comprises a 16S rRNA sequence that is at least 95% identical to SEQ ID NO: 6.

Embodiment 128: The pharmaceutical composition of any one of embodiments 123 to 127, wherein the Blautia obeum comprises a 16S rRNA sequence that is at least 95% identical to SEQ ID NO: 9.

Embodiment 129: The pharmaceutical composition of any one of embodiments 123 to 128, wherein the Blautia sp. comprises a 16S rRNA sequence that is at least 95% identical to SEQ ID NO: 34.

Embodiment 130: The pharmaceutical composition of any one of embodiments 123 to 129, wherein the Bacteroides uniformis comprises a 16S rRNA sequence that is at least 95% identical to at least one of SEQ ID NO: 11 and SEQ ID NO: 16.

Embodiment 131: The pharmaceutical composition of any one of embodiments 123 to 130, wherein the Bacteroides vulgatus comprises a 16S rRNA sequence that is at least 95% identical to SEQ ID NO: 12.

Embodiment 132: A pharmaceutical composition comprising a plurality of bacterial isolates, wherein the plurality of bacterial isolates comprises Alistipes finegoldii and at least one of Bacteroides uniformis and Dorea longicatena.

Embodiment 133: The pharmaceutical composition of embodiment 132, wherein the plurality of bacterial isolates further comprises at least one of Akkermansia muciniphila, Bacteroides vulgatus, and Blautia sp.

Embodiment 134: The pharmaceutical composition of embodiment 133, wherein the plurality of bacterial isolates comprises each of Bacteroides uniformis, Dorea longicatena, Akkermansia muciniphila, Bacteroides vulgatus, and Blautia sp.

Embodiment 135: The pharmaceutical composition of embodiment 133 or embodiment 134, wherein the Alistipes finegoldii comprises a 16S rRNA sequence that is at least 95% identical to SEQ ID NO: 15.

Embodiment 136: The pharmaceutical composition of any one of embodiments 133 to 135, wherein the Bacteroides uniformis comprises a 16S rRNA sequence that is at least 95% identical to SEQ ID NO: 11.

Embodiment 137: The pharmaceutical composition of any one of embodiments 133 to 136, wherein the Dorea longicatena comprises a 16S rRNA sequence that is at least 95% identical to SEQ ID NO: 6.

Embodiment 138: The pharmaceutical composition of any one of embodiments 133 to 137, wherein the Akkermansia muciniphila comprises a 16S rRNA sequence that is at least 95% identical to SEQ ID NO: 20.

Embodiment 139: The pharmaceutical composition of any one of embodiments 133 to 138, wherein the Bacteroides vulgatus comprises a 16S rRNA sequence that is at least 95% identical to SEQ ID NO: 12.

Embodiment 140: The pharmaceutical composition of any one of embodiments 133 to 139, wherein the Blautia sp. comprises a 16S rRNA sequence that is at least 95% identical to SEQ ID NO: 34.

Embodiment 141: The pharmaceutical composition of any one of embodiments 133 to 140, wherein the plurality of bacterial isolates does not comprise one of Bacteroides uniformis or Dorea longicatena.

Embodiment 142: The pharmaceutical composition of any one of embodiments 1 to 141, wherein the plurality of bacterial isolates comprises at least one bacterial isolate provided in Table 3.

Embodiment 143: The pharmaceutical composition of embodiment 142, wherein the at least one bacterial isolate modulates cytokine production or release by a eukaryotic cell.

Embodiment 144: The pharmaceutical composition of embodiment 143, wherein the at least one bacterial isolate decreases production or release of a pro-inflammatory cytokine by the eukaryotic cell.

Embodiment 145: The pharmaceutical composition of embodiment 144, wherein the pro-inflammatory cytokine is selected from the group consisting of: IFNγ, IL-12p70, IL-1 (e.g., IL-1α, IL-1β), IL-6, IL-8, IL-12, IL-17, IL-18, IL-23, MCP1, MIP1α, MIP1β, TNFα, TNF-γ, and a combination thereof.

Embodiment 146: The pharmaceutical composition of any one of embodiments 143 to 145, wherein the at least one bacterial isolate increases production or release of an anti-inflammatory cytokine by the eukaryotic cell.

Embodiment 147: The pharmaceutical composition of embodiment 146, wherein the anti-inflammatory cytokine is selected from the group consisting of: IL-10, IL-13, IL-4, IL-5, TGF-β, and a combination thereof.

Embodiment 148: The pharmaceutical composition of embodiment 147, wherein the anti-inflammatory cytokine is IL-10.

Embodiment 149: The pharmaceutical composition of embodiment 148, wherein the at least one bacterial isolate induces the eukaryotic cell to produce or release at least 500 pg/ml of IL-10.

Embodiment 150: The pharmaceutical composition of embodiment 148, wherein the at least one bacterial isolate induces the eukaryotic cell to produce or release at least 1000 pg/ml of IL-10.

Embodiment 151: The pharmaceutical composition of embodiment 148, wherein the at least one bacterial isolate induces the eukaryotic cell to produce or release at least 1500 pg/ml of IL-10.

Embodiment 152: The pharmaceutical composition of embodiment 148, wherein the at least one bacterial isolate induces the eukaryotic cell to produce or release at least 2000 pg/ml of IL-10.

Embodiment 153: The pharmaceutical composition of embodiment 148, wherein the at least one bacterial isolate induces the eukaryotic cell to produce or release at least 2500 pg/ml of IL-10.

Embodiment 154: The pharmaceutical composition of embodiment 148, wherein the at least one bacterial isolate induces the eukaryotic cell to produce or release at least 3000 pg/ml of IL-10.

Embodiment 155: The pharmaceutical composition of any one of embodiments 142 to 154, wherein the eukaryotic cell is a cultured cell.

Embodiment 156: The pharmaceutical composition of embodiment 155, wherein the cultured cell is a peripheral blood mononuclear cell (PBMC).

Embodiment 157: The pharmaceutical composition of any one of embodiments 142 to 168, wherein the eukaryotic cell is a cell of a subject administered the composition.

Embodiment 158: The pharmaceutical composition of embodiment 157, wherein the cell of the subject is selected from the group consisting of: an epithelial cell, an intestinal lamina propria cell, an endothelial cell, a fibroblast, a stromal cell, a macrophage, a B lymphocyte, a T lymphocyte, a mast cell, and a peripheral blood mononuclear cell (PBMC).

Embodiment 159: The pharmaceutical composition of embodiment 158, wherein the cell of the subject is an epithelial cell and the epithelial cell is an intestinal epithelial cell.

Embodiment 160: The pharmaceutical composition of any one of embodiments 1 to 159, wherein the pharmaceutical composition is formulated as a capsule for oral administration.

Embodiment 161: The pharmaceutical composition of embodiment 160, wherein the capsule comprises a delayed-release coating.

Embodiment 162: The pharmaceutical composition of embodiment 160 or embodiment 161, wherein the capsule comprises a hydrophobic coating.

Embodiment 163: The pharmaceutical composition of any one of embodiments 160 to 162, wherein the pharmaceutical composition is formulated for delivery of the cocktail to the intestine.

Embodiment 164: The pharmaceutical composition of embodiment 163, wherein the pharmaceutical composition is formulated for delivery of the cocktail to the small intestine.

Embodiment 165: The pharmaceutical composition of embodiment 163 or embodiment 164, wherein the composition is formulated for delivery of the cocktail to the large intestine.

Embodiment 166: The pharmaceutical composition of any one of embodiments 1 to 165, wherein the cocktail is lyophilized.

Embodiment 167: The pharmaceutical composition of any one of embodiments 1 to 166, wherein the pharmaceutical composition further comprises at least one of a pharmaceutically acceptable antioxidant, cryoprotectant, lyoprotectant, binder, disintegrant, excipient, filler, preservative, acid suppressant, antacid, H2 antagonist, and/or proton pump inhibitor.

Embodiment 168: A method of treating or preventing a disorder related to an intestinal dysbiosis in a subject in need thereof, wherein the method comprises administering to the subject a pharmaceutical composition of any one of embodiments 1 to 167.

Embodiment 169: The pharmaceutical composition of embodiment 168, wherein the disorder is selected from the group consisting of inflammatory bowel disease (IBD), irritable bowel syndrome (IBS), C. difficile infection (CDI), C. difficile-associated disease (CDAD), an antibiotic-induced adverse effect, and a combination thereof.

Embodiment 170: A method of treating or preventing irritable bowel syndrome in a subject in need thereof, comprising administering to the subject a plurality of bacterial isolates, wherein one of the bacterial isolates comprises Bacteroides cellulosilyticus, and wherein at least two of the plurality of bacterial isolates are isolated from stool samples of different donors.

Embodiment 171: The method of embodiment 170, wherein the Bacteroides cellulosilyticus comprises a 16S rRNA sequence that is at least 95% identical to SEQ ID NO: 26.

Embodiment 172: The method of embodiment 170 or embodiment 171, wherein the plurality of bacterial isolates further comprises Odoribacter splanchnicus.

Embodiment 173: A method of manufacturing a pharmaceutical composition containing a cocktail of bacterial isolates, the method comprising selecting a first bacterial isolate based on a level of a short-chain fatty acid (SCFA) produced by the first bacterial isolate; selecting a second bacterial isolate based on a relative abundance of a corresponding bacterial strain in a healthy human subject versus a subject having inflammatory bowel disease, wherein the second bacterial isolate comprises a 16S rRNA sequence at least 97% identical to a 16S rRNA sequence of the corresponding bacterial strain; and combining the first and second bacterial isolates to produce the cocktail of bacterial isolates.

Embodiment 174: The method of embodiment 173, wherein the first bacterial isolate comprises a 16S rRNA sequence at least 95% identical to a 16S rRNA sequence of a bacterial isolate provided in Table 2.

Embodiment 175: The method of embodiment 173 or embodiment 174, wherein the second bacterial isolate comprises a 16S rRNA sequence at least 95% identical to a 16S rRNA sequence of a bacterial isolate provided in Table 4.

Embodiment 176: The method of any one of embodiments 173 to 175, wherein the method further comprises selecting a third bacterial isolate based on a modulation of a level of a cytokine by the bacterial isolate.

Embodiment 177: The method of embodiment 176, wherein the third bacterial isolate comprises a 16S rRNA sequence at least 95% identical to a 16S rRNA sequence of a bacterial isolate provided in Table 3.

Embodiment 178: A pharmaceutical composition, comprising: a first human-derived bacterial isolate which induces at least one of an IL-10:IL-12 ratio of at least 50 or an IL-10:TNF-alpha ratio of at least 1 when incubated with a population of eukaryotic cells in a first functional assay; and a second human-derived bacterial isolate which produces a short chain fatty acid (SCFA) at a concentration of at least 10 mM as measured by a second functional assay; wherein the first and second bacterial isolates are capable of engrafting into the intestine of a subject following administration of the pharmaceutical composition to the subject.

Embodiment 179: The pharmaceutical composition of embodiment 178, wherein the IL-10:IL-12 ratio is at least 100.

Embodiment 180: The pharmaceutical composition of embodiment 178, wherein the IL-10:IL-12 ratio is at least 500.

Embodiment 181: The pharmaceutical composition of embodiment 178, wherein the IL-10:IL-12 ratio is at least 1000.

Embodiment 182: The pharmaceutical composition of embodiment 178, wherein the IL-10:IL-12 ratio is at least 2000.

Embodiment 183: The pharmaceutical composition of embodiment 178, wherein the IL-10:TNF-alpha ratio is at least 2.

Embodiment 184: The pharmaceutical composition of embodiment 178, wherein the IL-10:TNF-alpha ratio is at least 5.

Embodiment 185: The pharmaceutical composition of embodiment 178, wherein the IL-10:TNF-alpha ratio is at least 10.

Embodiment 186: The pharmaceutical composition of embodiment 178, wherein the IL-10:TNF-alpha ratio is at least 20.

Embodiment 187: The pharmaceutical composition of any one of embodiments 179 to 186, wherein the population of eukaryotic cells comprises a population of PBMCs.

Embodiment 188: The pharmaceutical composition of any one of embodiments 179 to 187, wherein the first human-derived bacterial isolate is incubated with the population of eukaryotic cells for about 24 hours.

Embodiment 189: The pharmaceutical composition of any one of embodiments 178 to 188, wherein the SCFA is butyrate.

Embodiment 190: The pharmaceutical composition of any one of embodiments 178 to 189, wherein the SCFA is produced at a concentration of at least 20 mM.

Embodiment 191: The pharmaceutical composition of any one of embodiments 178 to 190, wherein the SCFA is produced at a concentration of at least 25 mM.

Embodiment 192: The pharmaceutical composition of any one of embodiments 178 to 191, wherein the SCFA is produced at a concentration of at least 30 mM.

Embodiment 193: The pharmaceutical composition of any one of embodiments 178 to 192, wherein the SCFA is produced at a concentration of at least 35 mM.

Embodiment 194: The pharmaceutical composition of any one of embodiments 178 to 193, wherein the second functional assay comprises incubating the second bacterial isolate with a substrate.

Embodiment 195: The pharmaceutical composition of embodiment 194, wherein the substrate comprises at least one of an oligosaccharide, sunfiber, or barley malt.

Embodiment 196: The pharmaceutical composition of embodiment 195, wherein the oligosaccharide comprises at least one of a fructooligosaccharide (FOS) and an xylooligosaccharide (XOS).

Embodiment 197: The pharmaceutical composition of embodiment 196, wherein the substrate comprises both of a fructooligosaccharide (FOS) and an xylooligosaccharide (XOS).

Embodiment 198: The pharmaceutical composition of any one of embodiments 178 to 196, wherein the second bacterial isolate comprises a 16S rRNA sequence at least 95% identical to a 16S rRNA sequence of a bacterial isolate provided in Table 2.

Embodiment 199: The pharmaceutical composition of any one of embodiments 178 to 198, wherein the second bacterial isolate comprises Anaerostipes sp.

Embodiment 200: The pharmaceutical composition of embodiment 199, wherein the Anaerostipes sp. is Anaerostipes hadrus.

Embodiment 201: The pharmaceutical composition of any one of embodiments 178 to 200, wherein the second bacterial isolate comprises a 16S rRNA sequence at least 95% identical to the sequence corresponding to SEQ ID NO: 3.

Embodiment 202: The pharmaceutical composition of any one of embodiments 178 to 201, wherein the second bacterial isolate comprises Roseburia sp.

Embodiment 203: The pharmaceutical composition of embodiment 202, wherein the Roseburia sp. is Roseburia faecis.

Embodiment 204: The pharmaceutical composition of any one of embodiments 178 to 203, wherein the second bacterial isolate comprises a 16S rRNA sequence at least 95% identical to the sequence corresponding to SEQ ID NO: 19.

Embodiment 205: The pharmaceutical composition of any one of embodiments 178 to 204, wherein the second bacterial isolate comprises Eubacterium sp.

Embodiment 206: The pharmaceutical composition of embodiment 205, wherein the Eubacterium sp. is Eubacterium rectale.

Embodiment 207: The pharmaceutical composition of any one of embodiments 178 to 206, wherein the second bacterial isolate comprises a 16S rRNA sequence at least 95% identical to the sequence corresponding to SEQ ID NO: 8.

Embodiment 208: The pharmaceutical composition of any one of embodiments 178 to 207, wherein the second bacterial isolate comprises Coprococcus sp.

Embodiment 209: The pharmaceutical composition of embodiment 208, wherein the Coprococcus sp. is Coprococcus comes.

Embodiment 210: The pharmaceutical composition of any one of embodiments 178 to 209, wherein the second bacterial isolate comprises a 16S rRNA sequence at least 95% identical to the sequence corresponding to SEQ ID NO: 17.

Embodiment 211: The pharmaceutical composition of any one of embodiments 178 to 196, wherein the first bacterial isolate comprises a 16S sequence at least 95% identical to a 16S rRNA sequence of a bacterial isolate provided in Table 3.

Embodiment 212: The method of any one of embodiments 173 to 177, wherein the SCFA is butyrate.

Embodiment 213: The method of embodiment 198, wherein the first bacterial isolate is selected based on production of a level of butyrate of at least 20 mM.

Embodiment 214: A method of treating inflammatory bowel disease comprising administering the pharmaceutical composition of any one of embodiments 178 to 199 to a subject in need thereof.

Embodiment 215: The method of embodiment 200, wherein a dosage of at least one of the first bacterial isolate and second bacterial isolate is less than 10¹⁰ cells/ml.

Embodiment 216: A pharmaceutical composition comprising a plurality of bacterial isolates, wherein the plurality of bacterial isolates comprises Bacteroides cellulosilyticus and at least one of Odoribacter splanchnicus, Roseburia faecis, Faecalibacterium prausnitzii, Akkermansia muciniphila, Alistipes shahii, Subdoligranulum variabile, and Eubacterium rectale, wherein at least two of the plurality of bacterial isolates are isolated from stool samples of different donors.

Embodiment 217: The pharmaceutical composition of embodiment 216, wherein the plurality of bacterial isolates comprises Odoribacter splanchnicus.

Embodiment 218: The pharmaceutical composition of embodiment 217, wherein the plurality of bacterial isolates comprises Faecalibacterium prausnitzii.

Embodiment 219: The pharmaceutical composition of embodiment 218, wherein the plurality of bacterial isolates comprises two different bacterial isolates that are each members of the species Faecalibacterium prausnitzii.

Embodiment 220: The pharmaceutical composition of embodiment 218, wherein the plurality of bacterial isolates comprises Subdoligranulum variabile.

Embodiment 221: The pharmaceutical composition of embodiment 219, wherein the plurality of bacterial isolates comprises Roseburia faecis, Akkermansia muciniphila, Alistipes shahii, and Eubacterium rectale.

Embodiment 222: The pharmaceutical composition of embodiment 220, wherein the plurality of bacterial isolates comprises Roseburia faecis, Akkermansia muciniphila, Alistipes shahii, and Eubacterium rectale.

Embodiment 223: A pharmaceutical composition comprising a plurality of bacterial isolates, wherein the plurality of bacterial isolates comprises two different bacterial isolates that are each members of the species Faecalibacterium prausnitzii.

Embodiment 224: The composition of embodiment 223, wherein the two different bacterial isolates are isolated from stool samples of different human donors.

Embodiment 225: The composition of embodiment 223, wherein the plurality of bacterial isolates further comprises at least one of Bacteroides cellulosilyticus, Odoribacter splanchnicus, Roseburia faecis, Akkermansia muciniphila, Alistipes shahii, and Eubacterium rectale.

Embodiment 226: The composition of embodiment 223, wherein the plurality of bacterial isolates further comprises each of Bacteroides cellulosilyticus, Odoribacter splanchnicus, Roseburia faecis, Akkermansia muciniphila, Alistipes shahii, and Eubacterium rectale.

Embodiment 227: A method of treating or preventing irritable bowel syndrome in a subject in need thereof, comprising administering to the subject a plurality of bacterial isolates, wherein one of the bacterial isolates comprises Bacteroides cellulosilyticus, and wherein at least two of the plurality of bacterial isolates are administered to the subject in different pharmaceutical compositions.

Embodiment 228: The method of embodiment 227, wherein the plurality of bacterial isolates further comprises at least one of Faecalibacterium prausnitzii, Odoribacter splanchnicus, Roseburia faecis, Akkermansia muciniphila, Alistipes shahii, Subdoligranulum variabile and Eubacterium rectale.

Embodiment 229: The method of embodiment 227, wherein the plurality of bacterial isolates further comprises each of Faecalibacterium prausnitzii, Odoribacter splanchnicus, Roseburia faecis, Akkermansia muciniphila, Alistipes shahii, Subdoligranulum variabile and Eubacterium rectale.

Embodiment 230: The method of embodiment 227, wherein the plurality of bacterial isolates further comprises each of Odoribacter splanchnicus, Roseburia faecis, Akkermansia muciniphila, Alistipes shahii, Eubacterium rectale, and two different bacterial isolates that are each members of the species Faecalibacterium prausnitzii.

Embodiment 231: A method of manufacture, the method comprising culturing Bacteroides cellulosilyticus as a pure culture; and lyophilizing bacteria from the pure culture of B. cellulosilyticus to produce a B. cellulosilyticus lyophilate.

Embodiment 232: The method of embodiment 231, further comprising combining the B. cellulosilyticus lyophilate with a second lyophilate, wherein the second lyophilate is produced by lyophilizing bacteria from a pure culture of at least one of Faecalibacterium prausnitzii, Odoribacter splanchnicus, Roseburia faecis, Akkermansia muciniphila, Alistipes shahii, Subdoligranulum variabile and Eubacterium rectale.

Embodiment 233: A pharmaceutical composition comprising a plurality of bacterial isolates, wherein an amount of cells of a first bacterial isolate of the plurality of bacterial isolates is at least 1% greater than an amount of cells of a second bacterial isolate of the plurality of bacterial isolates, wherein the plurality of bacterial isolates are selected from the group consisting of: Bacteroides cellulosilyticus, Faecalibacterium prausnitzii, Subdogranulum variabile, Eubacterium rectale, Odoribacter splanchnicus, Alistipes shahii, and Akkermansia muciniphila.

Embodiment 234: The pharmaceutical composition of embodiment 233, wherein the amount of cells of the first bacterial isolate is at least 10% greater than the amount of cells of the second bacterial isolate.

Embodiment 235: The pharmaceutical composition of embodiment 233 or 234, wherein the first bacterial isolate comprises one of Eubacterium rectale and Faecalibacterium prausnitzii.

Embodiment 236: The pharmaceutical composition of embodiment 233 or 234, wherein the second bacterial isolate comprises one of Alistipes shahii, and Akkermansia muciniphila.

Embodiment 237: The pharmaceutical composition of embodiment 233 or 234, wherein each of the first and second bacterial isolates comprises Faecalibacterium prausnitzii.

Embodiment 238: The pharmaceutical composition of embodiment 237, wherein the first bacterial isolate comprises a 16S rRNA sequence that is at least 95% identical to SEQ ID NO: 7.

Embodiment 239: The pharmaceutical composition of embodiment 237 or 238, wherein the second bacterial isolate comprises a 16S rRNA sequence that is at least 95% identical to SEQ ID NO: 1.

Embodiment 240: A pharmaceutical composition comprising a plurality of bacterial isolates, wherein an amount of cells of a first bacterial isolate of the plurality of bacterial isolates is at least 1% greater than an amount of cells of a second bacterial isolate of the plurality of bacterial isolates, wherein each of the first and second bacterial isolates comprises Faecalibacterium prausnitzii.

Embodiment 241: The pharmaceutical composition of embodiment 240, wherein the amount of cells of the first bacterial isolate is at least 10% greater than the amount of cells of the second bacterial isolate.

Embodiment 242: The pharmaceutical composition of embodiment 240 or 241, wherein the first bacterial isolate comprises a 16S rRNA sequence that is at least 95% identical to SEQ ID NO: 7.

Embodiment 243: The pharmaceutical composition of any one of embodiments 240 to 242, wherein the second bacterial isolate comprises a 16S rRNA sequence that is at least 95% identical to SEQ ID NO: 1.

Embodiment 244: A pharmaceutical composition comprising a bacterial isolate, wherein the bacterial isolate comprises a 16S rRNA sequence that is at least 97% identical to a 16S rRNA sequence of a bacterial strain that is either (i) enriched in a group of healthy subjects over a group of patients with ulcerative colitis (UC) and/or (ii) correlated with clinical remission of one or more UC symptoms in a group of patients following treatment of each patient of the group of patients with a fecal microbiota transplant, wherein a cross-sectional combined p-value of the bacterial strain is less than 1×10⁻¹⁰.

Embodiment 245: The pharmaceutical composition of embodiment 244, wherein the bacterial isolate comprises a 16S rRNA sequence that is at least 99% identical to a 16S rRNA sequence of the bacterial strain.

Embodiment 246: The pharmaceutical composition of embodiment 244 or 245, wherein the bacterial isolate comprises at least one of Odoribacter splanchnicus, Eubacterium rectale, Bacteroides cellulosilyticus and Alistipes shahii.

Embodiment 247: The pharmaceutical composition of any one of embodiments 244 to 246, wherein the cross-sectional combined p-value of the bacterial strain is less than 1×10⁻¹⁴.

Embodiment 248: The pharmaceutical composition of any one of embodiments 244 to 247, wherein the bacterial isolate comprises at least one of Odoribacter splanchnicus and Alistipes shahii.

Embodiment 249: The pharmaceutical composition of any one of embodiments 244 to 248, wherein the cross-sectional combined p-value of the bacterial strain is less than 1×10⁻²⁰.

Embodiment 250: The pharmaceutical composition of any one of embodiments 244 to 249, wherein the bacterial isolate comprises Alistipes shahii.

Embodiment 251: The pharmaceutical composition of embodiment 250, wherein the Alistipes shahii comprises a 16S rRNA sequence that is at least 97% identical to SEQ ID NO: 18.

Embodiment 252: A method of treating inflammatory bowel disease comprising administering the pharmaceutical composition of any one of embodiments 216 to 252 to a subject in need thereof.

Embodiment 253: A method of treating or preventing inflammatory bowel disease (IBD) in a subject in need thereof, the method comprising administering to the subject a pharmaceutical composition comprising a plurality of bacterial isolates, wherein the plurality of bacterial isolates comprises Bacteroides cellulosilyticus, and at least one of Faecalibacterium prausnitzii, Odoribacter splanchnicus, Roseburia faecis, Akkermansia muciniphila, Alistipes shahii, Subdoligranulum variabile and Eubacterium rectale, wherein at least two of the plurality of bacterial isolates are isolated from stool samples of different human donors.

Embodiment 254: The method of embodiment 253, wherein the Bacteroides cellulosilyticus comprises a 16S ribosomal ribonucleic acid (rRNA) sequence that has at least about 95%, or at least about 97%, or at least about 98%, or at least about 99% sequence identity with the nucleotide sequence of SEQ ID NO: 14 and/or 26.

Embodiment 255: The method of embodiment 253 or embodiment 254, wherein the plurality of bacterial isolates comprises two of Faecalibacterium prausnitzii, Odoribacter splanchnicus, Roseburia faecis, Akkermansia muciniphila, Alistipes shahii, Subdoligranulum variabile and Eubacterium rectale.

Embodiment 256: The method of any one of embodiments 253-255, wherein the plurality of bacterial isolates comprises three of Faecalibacterium prausnitzii, Odoribacter splanchnicus, Roseburia faecis, Akkermansia muciniphila, Alistipes shahii, Subdoligranulum variabile and Eubacterium rectale.

Embodiment 257: The method of any one of embodiments 253-256, wherein the plurality of bacterial isolates comprises four of Faecalibacterium prausnitzii, Odoribacter splanchnicus, Roseburia faecis, Akkermansia muciniphila, Alistipes shahii, Subdoligranulum variabile and Eubacterium rectale.

Embodiment 258: The method of any one of embodiments 253-257, wherein the plurality of bacterial isolates comprises five of Faecalibacterium prausnitzii, Odoribacter splanchnicus, Roseburia faecis, Akkermansia muciniphila, Alistipes shahii, Subdoligranulum variabile and Eubacterium rectale.

Embodiment 259: The method of any one of embodiments 253-258, wherein the plurality of bacterial isolates comprises six of Faecalibacterium prausnitzii, Odoribacter splanchnicus, Roseburia faecis, Akkermansia muciniphila, Alistipes shahii, Subdoligranulum variabile and Eubacterium rectale.

Embodiment 260: The method of any one of embodiments 253-259, wherein the plurality of bacterial isolates comprises each of Faecalibacterium prausnitzii, Odoribacter splanchnicus, Roseburia faecis, Akkermansia muciniphila, Alistipes shahii, Subdoligranulum variabile and Eubacterium rectale.

Embodiment 261: The method of any one of embodiments 253-260, wherein the plurality of bacterial isolates comprises a 16S rRNA sequence that has at least about 95%, or at least about 97%, or at least about 98%, or at least about 99% sequence identity with a nucleotide sequence selected from SEQ ID NOs: 1, 2, 7, 18, 19, 20, 22, and 23.

Embodiment 262: The method of any one of embodiments 253-261, wherein the plurality of bacterial isolates comprises: (a) a 16S rRNA sequence that are at least about 95%, or at least about 97%, or at least about 98%, or at least about 99% sequence identity with nucleotide sequence of SEQ ID NO: 1 and/or 7; (b) a 16S rRNA sequence that are at least about 95%, or at least about 97%, or at least about 98%, or at least about 99% sequence identity with nucleotide sequence of SEQ ID NO: 2; (c) a 16S rRNA sequence that are at least about 95%, or at least about 97%, or at least about 98%, or at least about 99% sequence identity with nucleotide sequence of SEQ ID NO: 19; (d) a 16S rRNA sequence that are at least about 95%, or at least about 97%, or at least about 98%, or at least about 99% sequence identity with nucleotide sequence of SEQ ID NO: 20; (e) a 16S rRNA sequence that are at least about 95%, or at least about 97%, or at least about 98%, or at least about 99% sequence identity with nucleotide sequence of SEQ ID NO: 18; (f) a 16S rRNA sequence that are at least about 95%, or at least about 97%, or at least about 98%, or at least about 99% sequence identity with nucleotide sequence of SEQ ID NO: 22 and/or 23; and/or (g) a 16S rRNA sequence that are at least about 95%, or at least about 97%, or at least about 98%, or at least about 99% sequence identity with nucleotide sequence of SEQ ID NO: 8.

Embodiment 263: The method of any one of embodiments 253-262, wherein the plurality of bacterial isolates comprises at least two bacterial isolates comprising Faecalibacterium prausnitzii, wherein the at least two bacterial isolates comprise different 16S rRNA sequences.

Embodiment 264: The method of embodiment 263, wherein at least two bacterial isolates comprising Faecalibacterium prausnitzii comprise 16S rRNA sequences that are at least about 95%, or at least about 97%, or at least about 98%, or at least about 99% identity sequence identity with nucleotide sequences of SEQ ID NO: 1 and SEQ ID NO: 7.

Embodiment 265: The method of any one of embodiments 253-264, wherein the plurality of bacterial isolates further comprises at least one of Anaerostipes hadrus, Subdoligranulum variabile, Bacteroides uniformis.

Embodiment 266: The method of embodiment 265, wherein the plurality of bacterial isolates further comprises at least one 16S rRNA sequence that has at least about 95%, or at least about 97%, or at least about 98%, or at least about 99% sequence identity with a nucleotide sequence selected from SEQ ID NOs: 3, 11, 16, 22, and 23.

Embodiment 267: The method of any one of embodiments 253-266, wherein the plurality of bacterial isolates comprises lyophilized bacteria.

Embodiment 268: The method of any one of embodiments 253-267, wherein the plurality of bacterial isolates does not include Escherichia coli.

Embodiment 269: The method of any one of embodiments 253-268, wherein the pharmaceutical composition is not a stool sample, or a minimally processed version thereof.

Embodiment 270: A pharmaceutical composition comprising a plurality of bacterial isolates, wherein the plurality of bacterial isolates comprise Bacteroides cellulosilyticus, and at least one of, Faecalibacterium prausnitzii, Odoribacter splanchnicus, Roseburia faecis, Akkermansia muciniphila, Alistipes shahii, Subdoligranulum variabile and Eubacterium rectale, wherein at least two of the plurality of bacterial isolates are isolated from stool samples of different human donors.

Embodiment 271: The pharmaceutical composition of embodiment 270, wherein the Bacteroides cellulosilyticus comprises a 16S ribosomal ribonucleic acid (rRNA) sequence that has at least about 95%, or at least about 97%, or at least about 98%, or at least about 99% sequence identity with the nucleotide sequence of SEQ ID NO: 14 and/or 26.

Embodiment 272: The pharmaceutical composition of embodiment 270 or embodiment 271, wherein the plurality of bacterial isolates comprises two of Faecalibacterium prausnitzii, Odoribacter splanchnicus, Roseburia faecis, Akkermansia muciniphila, Alistipes shahii, Subdoligranulum variabile and Eubacterium rectale.

Embodiment 273: The pharmaceutical composition of any one of embodiments 270-272, wherein the plurality of bacterial isolates comprises three of Faecalibacterium prausnitzii, Odoribacter splanchnicus, Roseburia faecis, Akkermansia muciniphila, Alistipes shahii, Subdoligranulum variabile and Eubacterium rectale.

Embodiment 274: The pharmaceutical composition of any one of embodiments 270-273, wherein the plurality of bacterial isolates comprises four of Faecalibacterium prausnitzii, Odoribacter splanchnicus, Roseburia faecis, Akkermansia muciniphila, Alistipes shahii, Subdoligranulum variabile and Eubacterium rectale.

Embodiment 275: The pharmaceutical composition of any one of embodiments 270-274, wherein the plurality of bacterial isolates comprises five of Faecalibacterium prausnitzii, Odoribacter splanchnicus, Roseburia faecis, Akkermansia muciniphila, Alistipes shahii, Subdoligranulum variabile and Eubacterium rectale.

Embodiment 276: The pharmaceutical composition of any one of embodiments 270-275, wherein the plurality of bacterial isolates comprises six of Faecalibacterium prausnitzii, Odoribacter splanchnicus, Roseburia faecis, Akkermansia muciniphila, Alistipes shahii, Subdoligranulum variabile and Eubacterium rectale.

Embodiment 277: The pharmaceutical composition of any one of embodiments 270-276, wherein the plurality of bacterial isolates comprises each of Faecalibacterium prausnitzii, Odoribacter splanchnicus, Roseburia faecis, Akkermansia muciniphila, Alistipes shahii, Subdoligranulum variabile and Eubacterium rectale.

Embodiment 278: The pharmaceutical composition of any one of embodiments 270-277, wherein the plurality of bacterial isolates comprises a 16S rRNA sequence that has at least about 95%, or at least about 97%, or at least about 98%, or at least about 99% sequence identity with a nucleotide sequence selected from SEQ ID NOs: 1, 2, 7, 18, 19, 20, 22, and 23.

Embodiment 279: The pharmaceutical composition of any one of embodiments 270-278, wherein the plurality of bacterial isolates comprises at least two bacterial isolates comprising Faecalibacterium prausnitzii, wherein the at least two bacterial isolates comprise different 16S rRNA sequences.

Embodiment 280: The pharmaceutical composition of any one of embodiments 270-279, wherein at least two bacterial isolates comprising Faecalibacterium prausnitzii comprise 16S rRNA sequences that are at least about 95%, or at least about 97%, or at least about 98%, or at least about 99% identity sequence identity with nucleotide sequences of SEQ ID NO: 1 and SEQ ID NO: 7.

Embodiment 281: The pharmaceutical composition of any one of embodiments 270-280, wherein the plurality of bacterial isolates further comprises at least one of Anaerostipes hadrus, Subdoligranulum variabile, Bacteroides uniformis.

Embodiment 282: The pharmaceutical composition of embodiment 281, wherein the plurality of bacterial isolates further comprises at least one 16S rRNA sequence that has at least about 95%, or at least about 97%, or at least about 98%, or at least about 99% sequence identity with a nucleotide sequence selected from SEQ ID NOs: 3, 11, 16, 22, and 23.

Embodiment 283: A pharmaceutical composition comprising a microbial cocktail, wherein the microbial cocktail comprises at least two bacterial isolates from the species Faecalibacterium prausnitzii, wherein the at least two bacterial isolates are isolated from stool samples of different human donors.

Embodiment 284: The pharmaceutical composition of embodiment 283, wherein the two bacterial isolates from the species Faecalibacterium prausnitzii comprise a 16S rRNA sequence that are at least about 95%, or at least about 97%, or at least about 98%, or at least about 99% sequence identity with nucleotide sequence of SEQ ID NO: 1 and/or 7

Embodiment 285: The pharmaceutical composition of embodiment 283 or embodiment 284, wherein the two bacterial isolates from the species Faecalibacterium prausnitzii comprise different 16S rRNA sequences.

Embodiment 286: The pharmaceutical composition of any one of embodiments 283-285, wherein two bacterial isolates from the species Faecalibacterium prausnitzii comprise 16S rRNA sequences that are at least about 95%, or at least about 97%, or at least about 98%, or at least about 99% identity sequence identity with nucleotide sequences of SEQ ID NO: 1 and SEQ ID NO: 7.

Embodiment 287: The pharmaceutical composition of any one of embodiments 283-286, wherein the microbial cocktail further comprises at least one of Bacteroides cellulosilyticus, Odoribacter splanchnicus, Roseburia faecis, Faecalibacterium prausnitzii, Akkermansia muciniphila, Alistipes shahii, Subdoligranulum variabile, and Eubacterium rectale.

Embodiment 288: The pharmaceutical composition of embodiment 287, wherein the microbial cocktail comprises at least two, or at least three, or at least four or at least five, or at least six, or at least seven, or each of Bacteroides cellulosilyticus, Odoribacter splanchnicus, Roseburia faecis, Faecalibacterium prausnitzii, Akkermansia muciniphila, Alistipes shahii, Subdoligranulum variabile, and Eubacterium rectale.

Embodiment 289: The pharmaceutical composition of embodiment 287 or embodiment 288, wherein the microbial cocktail comprises a 16S rRNA sequence that has at least about 95%, or at least about 97%, or at least about 98%, or at least about 99% sequence identity with a nucleotide sequence selected from SEQ ID NOs: 1, 2, 7, 8, 14, 18, 19, 20, 22, 23, and 26.

Embodiment 290: A pharmaceutical composition comprising a microbial cocktail, wherein the microbial cocktail comprises (i) at least two bacterial isolates from the species Faecalibacterium prausnitzii; and (ii) at least one bacterial isolate selected from Bacteroides cellulosilyticus, Odoribacter splanchnicus, Roseburia faecis, Akkermansia muciniphila, Alistipes shahii, Subdoligranulum variabile and Eubacterium rectale.

Embodiment 291: The pharmaceutical composition of embodiment 290, wherein the at least two bacterial isolates from the species Faecalibacterium prausnitzii comprise a 16S rRNA sequence that are at least about 95%, or at least about 97%, or at least about 98%, or at least about 99% sequence identity with nucleotide sequence of SEQ ID NO: 1 and/or 7

Embodiment 292: The pharmaceutical composition of embodiment 290 or embodiment 291, wherein the at least two bacterial isolates from the species Faecalibacterium prausnitzii comprise different 16S rRNA sequences.

Embodiment 293: The pharmaceutical composition of any one of embodiments 290-292, wherein at least two bacterial isolates from the species Faecalibacterium prausnitzii comprise 16S rRNA sequences that are at least about 95%, or at least about 97%, or at least about 98%, or at least about 99% identity sequence identity with nucleotide sequences of SEQ ID NO: 1 and SEQ ID NO: 7.

Embodiment 294: The pharmaceutical composition of any one of embodiments 290-293, wherein the microbial cocktail comprises at least two, or at least three, or at least four or at least five, or at least six, or each of Bacteroides cellulosilyticus, Odoribacter splanchnicus, Roseburia faecis, Akkermansia muciniphila, Alistipes shahii, Subdoligranulum variabile and Eubacterium rectale.

Embodiment 295: The pharmaceutical composition of any one of embodiments 290-294, wherein the microbial cocktail comprises a 16S rRNA sequence that has at least about 95%, or at least about 97%, or at least about 98%, or at least about 99% sequence identity with a nucleotide sequence selected from SEQ ID NOs: 2, 8, 14, 18, 19, 20, 22, 23, and 26.

Embodiment 296: A pharmaceutical composition comprising a plurality of bacterial isolates, wherein an amount of cells of a first bacterial isolate of the plurality of bacterial isolates is at least 1% greater than an amount of cells of a second bacterial isolate of the plurality of bacterial isolates, wherein the plurality of bacterial isolates are selected from Bacteroides cellulosilyticus, Faecalibacterium prausnitzii, Roseburia faecis, Eubacterium rectale, Odoribacter splanchnicus, Alistipes shahii, and Akkermansia muciniphila.

Embodiment 297: The pharmaceutical composition of embodiment 296, wherein the microbial cocktail comprises a 16S rRNA sequence that has at least about 95%, or at least about 97%, or at least about 98%, or at least about 99% sequence identity with a nucleotide sequence selected from SEQ ID NOs: 1, 2, 7, 8, 14, 18, 19, 20, and 26.

Embodiment 298: The pharmaceutical composition of embodiment 296 or embodiment 297, wherein the amount of cells of the first bacterial isolate is at least 10% greater than the amount of cells of the second bacterial isolate.

Embodiment 299: The pharmaceutical composition of any one of embodiments 296-298, wherein the first bacterial isolate comprises one of Eubacterium rectale and Faecalibacterium prausnitzii, and the second bacterial isolate comprises one of A. shahii, Bacteroides cellulosilyticus, and Akkermansia muciniphila.

Embodiment 300: The pharmaceutical composition of any one of embodiments 270-299, wherein the plurality of bacterial isolates comprises lyophilized bacteria.

Embodiment 301: The pharmaceutical composition of any one of embodiments 270-300, wherein the plurality of bacterial isolates does not include Escherichia coli.

Embodiment 302: The pharmaceutical composition of any one of embodiments 270-301, wherein the pharmaceutical composition is not a stool sample, or a minimally processed version thereof.

Embodiment 303: A method of preparing a bacterial isolate, the method comprising: (i) providing bacterial isolates from fecal samples from (a) healthy subjects, (b) patients with ulcerative colitis (UC), and (c) patients having clinical remission of one or more UC symptoms following treatment of each patient of the group of patients with a fecal microbiota transplant, (ii) analyzing 16S rRNA sequences from the bacterial isolates, and (iii) preparing the bacterial isolate that comprises a 16S rRNA sequence that is at least 97% identical to a 16S rRNA sequence of a bacterial strain that is (a) enriched in a group of healthy subjects over a group of patients with ulcerative colitis (UC); and/or (b) correlated with clinical remission of one or more UC symptoms in a group of patients following treatment of each patient of the group of patients with a fecal microbiota transplant, wherein a cross-sectional combined p-value of the bacterial strain is less than 1×10⁻¹⁴.

Embodiment 304: The method of embodiment 303, wherein the bacterial isolate comprises a 16S rRNA sequence that is at least 99% identical to a 16S rRNA sequence of the bacterial strain.

Embodiment 305: The method of embodiment 303 or embodiment 304, wherein the bacterial isolate comprises at least one of Odoribacter splanchnicus and Alistipes shahii.

Embodiment 306: The method of any one of embodiments 303-305, wherein the microbial cocktail comprises a 16S rRNA sequence that has at least about 95%, or at least about 97%, or at least about 98%, or at least about 99% sequence identity with a nucleotide sequence of SEQ ID NO: 2 and SEQ ID NO: 18.

Embodiment 307: The method of any one of embodiments 303-306, wherein the first bacterial strain enriched in a group of healthy subjects over a group of patients with ulcerative colitis (UC) is selected from Faecalibacterium prausnitzii, Odoribacter splanchnicus, Anaerostipes hadrus, Alistipes onderdonkii, Faecalibacterium prausnitzii, Eubacterium rectale, Blautia obeum, Bacteroides uniformis, Bacteroides vulgatus, Bacteroides cellulosilyticus, Alistipes finegoldii, Alistipes shahii, Akkermansia muciniphila, Phascolarctobacterium faecium, Subdoligranulum variabile, Subdoligranulum variabile, Blautia sp., Alistipes putredinis, Alistipes putredinis, Alistipes putredinis, and any two or more thereof.

Embodiment 308: The method of any one of embodiments 303-307, wherein the first bacterial strain enriched in a group of healthy subjects over a group of patients with ulcerative colitis (UC) comprises a 16S rRNA sequence that has at least about 95%, or at least about 97%, or at least about 98%, or at least about 99% sequence identity with a nucleotide sequence selected from SEQ ID NOs: 1, 2, 3, 4, 7, 8, 9, 11, 12, 14, 15, 16, 18, 20, 21, 22, 23, 34, 35, 36, and 37.

Embodiment 309: The method of any one of embodiments 303-308, wherein the first bacterial strain correlated with clinical remission of one or more UC symptoms in a group of patients following treatment of each patient of the group of patients with a fecal microbiota transplant comprises: Alistipes finegoldii, but does not comprise Alistipes shahii or Alistipes putredinis, or Alistipes putredinis, but does not comprise Alistipes shahii or Alistipes finegoldii.

Embodiment 310: The method of any one of embodiments 303-309, wherein the first bacterial strain correlated with clinical remission of one or more UC symptoms in a group of patients following treatment of each patient of the group of patients with a fecal microbiota transplant comprises: a 16S rRNA sequence that has at least about 95%, or at least about 97%, or at least about 98%, or at least about 99% sequence identity with a nucleotide sequence of SEQ ID NO: 15, but not a nucleotide sequence selected from SEQ ID NO: 18, 35, 36 or 37; or a 16S rRNA sequence that has at least about 95%, or at least about 97%, or at least about 98%, or at least about 99% sequence identity with the a nucleotide sequence selected from SEQ ID NOs: 35-37, but not a nucleotide sequence selected from SEQ ID NO: 15 and 18.

Embodiment 311: The method of any one of embodiments 303-310, wherein the method further comprises selecting a third bacterial isolate comprising: (iv) providing human-derived bacterial isolates from fecal samples; (v) performing a first functional assay comprising: (a) contacting a population of eukaryotic cells with the human-derived bacterial isolates, (b) measuring a level of a cytokine in the population of eukaryotic cells, and (c) identifying the bacterial isolates as capable of modulation of the level of the cytokine in the population of eukaryotic cells.

Embodiment 312: The method of embodiment 311, wherein the population of eukaryotic cells comprises a population of PBMCs.

Embodiment 313: The method of embodiment 311 or embodiment 312, wherein the cytokine is selected from IL-10, GM-CSF, IFN-gamma, TNF-alpha, IL-23, and IL-12.

Embodiment 314: The method of any one of embodiments 311-313, wherein the third bacterial isolate is selected from Faecalibacterium prausnitzii, Odoribacter splanchnicus, Anaerostipes hadrus, Alistipes shahii, Akkermansia muciniphila, Subdoligranulum variabile, Bacteroides uniformis, and any two or more thereof.

Embodiment 315: The method of any one of embodiments 311-314, wherein the third bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 11, 16, 18, 20, 22, and 23.

Embodiment 316: A method of preparing a pharmaceutical composition comprising a microbial cocktail, the method comprising: (i) providing human-derived bacterial isolates from fecal samples; (ii) performing a first functional assay comprising: (a) contacting a population of eukaryotic cells with the human-derived bacterial isolates, (b) measuring an IL-10:IL-12 ratio and an IL-10:TNF-alpha ratio, and (c) identifying a first bacterial isolate capable of inducing an IL-10:IL-12 ratio of at least about 50 or an IL-10:TNF-alpha ratio of at least about 1 when incubated with a population of eukaryotic cells, (iii) performing a second functional assay comprising: (a) incubating the human-derived bacterial isolates with media, (b) measuring a short chain fatty acid (SCFA) in the media, and (c) identifying a second bacterial isolate capable of the SCFA at a concentration of at least about 10 mM, and (iv) preparing the microbial cocktail comprising: (a) the first bacterial isolate capable of inducing an IL-10:IL-12 ratio of at least about 50 or an IL-10:TNF-alpha ratio of at least about 1 when incubated with a population of eukaryotic cells in the first functional assay, and (b) the second bacterial isolate capable of producing a short chain fatty acid (SCFA) at a concentration of at least about 10 mM as measured by the second functional assay; wherein the first and second bacterial isolates engraft in the intestine of a subject following administration of the pharmaceutical composition to the subject.

Embodiment 317: The method of embodiment 316, wherein the first bacterial isolate is selected from Faecalibacterium prausnitzii, Odoribacter splanchnicus, Anaerostipes hadrus, Bacteroides uniformis, Bacteroides vulgatus, Coprococcus comes, Alistipes shahii, Akkermansia muciniphila, Subdoligranulum variabile, and any two or more thereof.

Embodiment 318: The method of embodiment 316 or embodiment 317, wherein the first bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 11, 12, 16, 17, 18, 20, 22 and 23.

Embodiment 319: The method of any one of embodiments 316-318, wherein the population of eukaryotic cells comprises a population of PBMCs.

Embodiment 320: The method of any one of embodiments 316-319, wherein the SCFA is selected from acetic acid, butyric acid, caproic acid, formic acid, heptanoic acid, isobutyric acid, isocaproic acid, isovaleric acid, propionic acid, valeric acid, and any two or more thereof.

Embodiment 321: The method of any one of embodiments 316-320, wherein the SCFA is butyric acid.

Embodiment 322: The method of any one of embodiments 316-321, wherein the second bacterial isolate is selected from Odoribacter splanchnicus, Eubacterium rectale, Coprococcus comes, Faecalibacterium prausnitzii, Roseburia faecis, Anaerostipes hadrus, Subdogranulum variabile, and any two or more thereof.

Embodiment 323: The method of any one of embodiments 316-322, wherein the second bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 8, 17, 19, 22, and 23.

Embodiment 324: A method of treating or preventing Inflammatory Bowel Disease (IBD) in a subject in need thereof, comprising administering to the subject a plurality of bacterial isolates, wherein one of the bacterial isolates comprises Bacteroides cellulosilyticus, and wherein at least two of the plurality of bacterial isolates are isolated from stool samples of different donors.

Embodiment 325: The method of embodiment 324, wherein the plurality of bacterial isolates further comprises at least one of Faecalibacterium prausnitzii, Odoribacter splanchnicus, Roseburia faecis, Akkermansia muciniphila, Alistipes shahii, and Eubacterium rectale.

Embodiment 326: The method of embodiment 325, wherein the plurality of bacterial isolates comprises at least two, or at least three, or at least four or at least five, or each of Faecalibacterium prausnitzii, Odoribacter splanchnicus, Roseburia faecis, Akkermansia muciniphila, Alistipes shahii, and Eubacterium rectale.

Embodiment 327: The method of embodiment 325 or embodiment 326, wherein the plurality of bacterial isolates comprises a 16S rRNA sequence that has at least about 95%, or at least about 97%, or at least about 98%, or at least about 99% sequence identity with a nucleotide sequence selected from SEQ ID NOs: 1, 2, 7, 8, 18, 19, and 20.

Embodiment 328: A method of treating or preventing Inflammatory Bowel Disease (IBD) in a subject in need thereof, comprising administering to the subject a first pharmaceutical composition comprising a first bacterial isolate comprising Bacteroides cellulosilyticus, and administering to the subject a second pharmaceutical composition comprising a second bacterial isolate.

Embodiment 329: The method of embodiment 328, wherein at least one of the first and/or second bacterial isolates are lyophilized.

Embodiment 330: The method of embodiment 328 or embodiment 329, wherein the first pharmaceutical composition comprises a 16S rRNA sequence that has at least about 95%, or at least about 97%, or at least about 98%, or at least about 99% sequence identity with a nucleotide sequence of SEQ ID NO: 14 and/or 26.

Embodiment 331: The method of any one of embodiments 328-330, wherein the second pharmaceutical composition comprises at least one of Faecalibacterium prausnitzii, Odoribacter splanchnicus, Roseburia faecis, Akkermansia muciniphila, Alistipes shahii, and Eubacterium rectale.

Embodiment 332: The method of embodiment 331, wherein the plurality of bacterial isolates comprises at least two, or at least three, or at least four or at least five, or each of Faecalibacterium prausnitzii, Odoribacter splanchnicus, Roseburia faecis, Akkermansia muciniphila, Alistipes shahii, and Eubacterium rectale.

Embodiment 333: The method of any one of embodiments 328-332, wherein the second pharmaceutical composition comprises a 16S rRNA sequence that has at least about 95%, or at least about 97%, or at least about 98%, or at least about 99% sequence identity with a nucleotide sequence selected from SEQ ID NOs: 1, 2, 7, 8, 18, 19, and 20.

Embodiment 334: A method of producing a composition, comprising culturing Bacteroides cellulosilyticus as a pure culture; and lyophilizing bacteria from the B. cellulosilyticus pure culture to produce a B. cellulosilyticus lyophilate.

Embodiment 335: The method of embodiment 334, wherein the Bacteroides cellulosilyticus comprises a 16S rRNA sequence that has at least about 95%, or at least about 97%, or at least about 98%, or at least about 99% sequence identity with a nucleotide sequence of SEQ ID NO: 14 and/or 26.

Embodiment 336: The method of embodiment 334 or embodiment 335, further comprising mixing the B. cellulosilyticus lyophilate with a second lyophilate, wherein the second lyophilate comprises at least one bacterial isolate selected from Faecalibacterium prausnitzii, Odoribacter splanchnicus, Roseburia faecis, Akkermansia muciniphila, Alistipes shahii, Subdoligranulum variabile, Eubacterium rectale.

Embodiment 337: The method of embodiment 336, wherein the second lyophilate comprises at least two, or at least three, or at least four or at least five, or at least six, or each of bacterial isolate selected from Faecalibacterium prausnitzii, Odoribacter splanchnicus, Roseburia faecis, Akkermansia muciniphila, Alistipes shahii, Subdoligranulum variabile, Eubacterium rectale.

Embodiment 338: The method of any one of embodiments 330-337, wherein the second lyophilate comprises a 16S rRNA sequence that has at least about 95%, or at least about 97%, or at least about 98%, or at least about 99% sequence identity with a nucleotide sequence selected from SEQ ID NOs: 1, 2, 7, 8, 18, 19, 20, 22 and 23.

### EXAMPLES

The examples herein are provided to illustrate advantages and benefits of the present technology and to further assist a person of ordinary skill in the art with preparing or using compositions of the present technology. The examples herein are also presented in order to more fully illustrate the preferred aspects of the present technology. The examples should in no way be construed as limiting the scope of the present disclosure, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1: Isolating and Growing Bacterial Isolates

Stool samples from candidate human donors was collected and screened for the presence of pathogens. Six donors whose stool samples did not contain pathogens were selected. Donor stool was then collected in Whirl-Pak® filter bags, suspended in buffer (12.5% glycerol and 0.9% PBS, pH 7.4), and homogenized. Filtrate was diluted ˜10³× in PBS containing 0.05% cysteine and either plated on solid Isolation Media (IM; see below for each strain) or inoculated into IM broth, and grown under anaerobic conditions. Bacterial isolates were purified from plates by picking bacterial colonies and from IM broth by diluting to extinction. Once purified, each bacterial isolate was grown in IM broth under anaerobic conditions for further characterization, and aliquots frozen at −80° C. in 20% glycerol to make stocks.

Isolation Media (IM) for Strains

The following IM was used to culture strains used in this study. In each case, agar was omitted for cultures grown in broth.

1. Complex Gut Medium (CGM)

CGM was used to culture bacteria from the following genus and species:
Alistipes onderdonkii (e.g., a bacterial isolate having SEQ ID NO: 4, which was isolated from stool of a disease-screened and healthy human donor (“Donor E”));
Bacteroides uniformis (e.g., a bacterial isolate having SEQ ID NO: 11 or SEQ ID NO:16; which were each isolated from stool of a disease-screened and healthy human donor (respectively “Donor K” and “Donor E”)).

Formulation (Per Liter):

| Tryptone | 2 | g |
|---|---|---|
| Peptone from casein | 2 | g |
| Yeast extract | 1 | g |
| D-glucose | 0.4 | g |
| L-cysteine | 0.5 | g |
| D-(+)-Cellobiose | 1 | g |
| D-(+)-Maltose | 1 | g |
| D-(−) Fructose | 1 | g |
| Meat extract | 5 | g |
| MgSO₄·7H₂O | 0.0025 | g |
| NaHCO₃ | 0.4 | g |
| NaCl₂ | 0.08 | g |
| Phosphate buffer | 100 | mM |
| CaCl₂ | 0.8% | |
| Vitamin K (menadione) | 5.8 | mM |
| FeSO₄ | 1.44 | mM |
| Histidine hematin | 0.1% | |
| Tween80 | 0.05% | |
| ATCC Vitamin mix | 1% | |
| ATCC Trace Mineral mix | 1% | |
| Acetic acid | 30 | mM |
| Isovaleric acid | 1 | mM |
| Propionic acid | 8 | mM |
| Butyric acid | 4 | mM |
| Resazurin | 4 | mM |

| | |
|---|---|
| Agar | 1.5% |
| Final pH 7.0 ± 0.2 | |

2. Minimal Mucin Media (Min Muc)

Minimal mucin media was used to culture bacteria from the following genus and species:
Dorea longicatena (e.g., a bacterial isolate having SEQ ID NO: 6, which was isolated from stool of a disease-screened and healthy human donor (“Donor F”)).

Formulation (Per Liter):

| | | |
|---|---|---|
| 5× Minimum salts (see below) | 11.28 | g |
| Agar | 12 | g |
| Mucin from porcine stomach Type II | 2.5 | g |
| Final pH 6.8 ± 0.2 | | |

5× Minimum salts (see below) 11.28 g

Formulation (Per Liter):

| | | |
|---|---|---|
| Disodium phosphate | 33.9 | g |
| Potassium phosphate | 15 | g |
| Sodium chloride | 2.5 | g |
| Ammonium chloride | 5 | g |

3. Veggie Brain Heart Infusion Agar with Supplements (vBHI3)

vBHI3 media was used to culture bacteria from the following genus and species:
Odoribacter splanchnicus (e.g., a bacterial isolate having SEQ ID NO: 2, which was isolated from stool of a disease-screened and healthy human donor (“Donor G”));
Bacteroides cellulosilyticus (e.g., a bacterial isolate having SEQ ID NO: 14, which was isolated from stool of a disease-screened and healthy human donor (“Donor G”));
Blautia obeum (e.g., a bacterial isolate having SEQ ID NO: 9, which was isolated from stool of a disease-screened and healthy human donor (“Donor G”));
Blautia sp., (e.g., a bacterial isolate having SEQ ID NO: 34, which was isolated from stool of a disease-screened and healthy human donor (“Donor H”);
Parabacteroides merdae (e.g., a bacterial isolate having SEQ ID NO: 5, which was isolated from stool of a disease-screened and healthy human donor (“Donor (“Donor H”)).

Formulation (Per Liter):

| | | |
|---|---|---|
| HiVeg ™ Peptone No. 3 (HiMedia ® RM005V) | 10 | g |
| HiVeg ™ Special Infusion (HiMedia ® RM188V) | 7.5 | g |
| HiVeg ™ Infusion (HiMedia ® RM191V) | 10 | g |
| Dextrose | 2 | g |
| Sodium Chloride | 5 | g |
| Disodium phosphate | 2.5 | g |
| Agar | 12 | g |
| L-cysteine | 0.005 | g |
| Hemin | 0.05 | mg |
| Vitamin K | 0.0025 | mg |
| Final pH 7.4 ± 0.2 | | |

4. Veggie Brain Heart Infusion agar with supplements and sodium taurocholate (vBHI3+NaTau)

vBHI3+NaTau media was used to culture bacteria from the following genus and species:
Anaerostipes hadrus (e.g., a bacterial isolate having SEQ ID NO: 3, which was isolated from stool of a disease-screened and healthy human donor (“Donor G”));

Formulation (Per Liter):

Use recipe for Veggie Brain Heart Infusion agar with supplements (vBHI3)

Plus Sodium taurocholate 1 g

Final pH 7.4±0.2

5. 0.2× Veggie Brain Heart Infusion agar with supplements and mucin (vBHI3+mucin)

vBHI3+mucin media was used to culture bacteria from the following genus and species:
Alistipes finegoldii (e.g., a bacterial isolate having SEQ ID NO: 15, which was isolated from stool of a disease-screened and healthy human donor (“Donor E”)).

Formulation (Per Liter):

| | | |
|---|---|---|
| HiVeg ™ Peptone No. 3 (HiMedia ® RM005V) | 2 | g |
| HiVeg ™ Special Infusion (HiMedia ® RM188V) | 1.5 | g |
| HiVeg ™ Infusion (HiMedia ® RM191V) | 2 | g |
| Dextrose | 0.4 | g |
| Sodium Chloride | 1 | g |
| Disodium phosphate | 0.5 | g |
| Agar | 12 | g |
| L-cysteine | 0.005 | g |
| Hemin | 0.05 | mg |
| Vitamin K | 0.0025 | mg |
| Mucin from porcine stomach Type II | 2.5 | g |
| Final pH 7.4 ± 0.2 | | |

6. Veggie Reinforced Clostridial Medium with rumen (vRCM+rumen)

vRCM+rumen media was used to culture bacteria from the following genus and species:
Coprococcus comes (e.g., a bacterial isolate having SEQ ID NO: 17, which was isolated from stool of a disease-screened and healthy human donor (“Donor J”)).

Formulation (Per Liter):

| Differential Reinforced Clostridial HiVeg broth | 29 | g |
|---|---|---|
| (see below) | | |
| Noble agar | 12 | g |
| Rumen fluid | 30% | |
| L-cysteine | 0.005 | g |
| Final pH 7.2 ± 0.2 | | |

Differential Reinforced Clostridial HiVeg™ broth (HiMedia MV549) (used in vRCM+rumen media)

Formulation (Per Liter).

| | | |
|---|---|---|
| HiVeg ™ peptone | 10 | g |
| HiVeg ™ extract | 10 | g |
| Yeast extract | 1.5 | g |
| Starch | 1 | g |
| Sodium acetate | 5 | g |
| Glucose | 1 | g |
| L-cysteine hydrochloride | 0.5 | g |

7. Wilkins-Chalgren Anaerobe agar with cooked meat (WCA+CM)

WCA+CM media was used to culture bacteria from the following genus and species:
Eubacterium rectale (e.g., a bacterial isolate having SEQ ID NO: 8, which was isolated from stool of a disease-screened and healthy human donor (“Donor F”)).

Formulation (Per Liter):

| | | |
|---|---|---|
| Wilkins-Chalgren Anaerobe broth | 33 | g |
| (see below) | | |
| Noble agar | 12 | g |
| Ground cooked meat granules | 10 | g |
| L-cysteine | 0.005 | g |
| Hemin | 0.05 | mg |
| Vitamin K | 0.0025 | mg |
| Final pH 7 ± 0.2 | | |

Wilkins-Chalgren Anaerobe broth (Oxoid CM0643) (used in WCA+CM media)

Formulation (Per Liter):

| | | |
|---|---|---|
| Tryptone | 10 | g |
| Gelatin peptone | 10 | g |
| Yeast extract | 5 | g |
| Glucose | 1 | g |
| Sodium chloride | 5 | g |
| L-Arginine | 1 | g |
| Sodium pyruvate | 1 | g |
| Menadione | 0.0005 | g |
| Haemin | 0.005 | g |
| Final pH 7.1 ± 0.2 | | |

8. Yeast Casitone Fatty Acids Agar with Carbohydrates (YCFAC)

YCFAC media was used to culture bacteria from the following genus and species:
Faecalibacterium prausnitzii (e.g., a bacterial isolate having SEQ ID NO: 1 or SEQ ID NO: 7, which were each isolated from stool of a disease-screened and healthy human donor (respectively “Donor E” and “Donor F”);
Alistipes shahii (e.g., a bacterial isolate having SEQ ID NO: 18, which was isolated from stool of a disease-screened and healthy human donor (“Donor E”);
Bacteroides uniformis (e.g., a bacterial isolate having SEQ ID NO: 11 or SEQ ID NO:16; which were each isolated from stool of a disease-screened and healthy human donor (respectively “Donor K” and “Donor E”)).
Bacteroides vulgatus (e.g., a bacterial isolate having SEQ ID NO: 12, which was isolated from stool of a disease-screened and healthy human donor (“Donor K”);
Roseburia faecis (e.g., a bacterial isolate having SEQ ID NO: 19, which was isolated from stool of a disease-screened and healthy human donor (“Donor E”);

Media was pre-purchased from Anaerobe Systems (Cat #AS-675)

Formulation (Per Liter):

| | | |
|---|---|---|
| Casitone | 10.00 | g |
| Yeast Extract | 2.50 | g |
| Sodium Bicarbonate | 4.00 | g |
| Glucose | 2.00 | g |
| Cellobiose | 2.00 | g |
| Maltose | 2.00 | g |
| Potassium Phosphate Monobasic | 0.45 | g |
| Potassium Phosphate Dibasic | 0.45 | g |
| Sodium Chloride | 0.90 | g |
| Ammonium Sulfate | 0.90 | g |
| Magnesium Sulfate Heptahydrate | 0.09 | g |
| Calcium Chloride | 0.09 | g |
| Hemin (0.1% solution) | 10.00 | mL |
| Vitamin Mix | 10.00 | mL |
| L-Cysteine (25.0% solution) | 4.00 | mL |
| Resazurin (0.025% solution) | 4.00 | mL |
| Volatile Fatty Acid Solution | 2.90 | mL |
| Agar | 15.00 | g |
| Final pH 6.8 ± 0.3 | | |

9. HiVeg™ Brucella Blood Agar (vBBA)

vBBA media was used to culture bacteria from the following genus and species:
Phascolarctobacterium faecium (e.g., a bacterial isolate having SEQ ID NO: 21, which was isolated from stool of a disease-screened and healthy human donor (“Donor K”).

Media can be purchased as a dehydrated mix (Brucella HiVeg Agar Base; HiMedia MV074), followed by addition of L-cysteine and horse serum.

Formulation (Per Liter):

| | | |
|---|---|---|
| HiVeg hydrolysate | 10 | g |
| HiVeg peptone | 10 | g |
| Yeast extract | 2 | g |
| Dextrose | 1 | g |
| Sodium chloride | 5 | g |
| Sodium bisulphite | 0.1 | g |
| Agar | 15 | g |
| Horse serum | 50 | mL |
| L-cysteine | 0.005 | g |
| Final pH 7.0 ± 0.2 | | |

10. Minimal Media+Purified Mucin

Minimal media+purified mucin was used to culture bacteria from the following genus and species:
Akkermansia muciniphila (e.g., a bacterial isolate having SEQ ID NO: 20, which was isolated from stool of a disease-screened and healthy human donor (“Donor F”).

Formulation (Per Liter):

| | | |
|---|---|---|
| Monopotassium phosphate | 0.4 | g |
| Disodium phosphate | 0.53 | g |
| Ammonium chloride | 0.3 | g |
| Sodium chloride | 0.3 | g |
| Magnesium chloride | 0.047 | g |
| Calcium chloride | 0.11 | g |
| Sodium bicarbonate | 4 | g |
| Sodium sulfite | 0.005 | g |
| L-cysteine | 0.005 | g |
| ATCC Trace Mineral solution | 10 | mL |
| ATCC Vitamin solution | 10 | mL |
| Ethanol-purified Type III mucin | 2.5 | g |

11. M10+rumen fluid+mucin

M10+rumen fluid+mucin was used to culture bacteria from the following genus and species:
Subdoligranulum variabile (e.g., a bacterial isolate having SEQ ID NO: 22, which was isolated from stool of a disease-screened and healthy human donor (“Donor F”).
Alistipes putredinis (e.g., a bacterial isolate having SEQ ID NO: 35, which was isolated from stool of a disease-screened and healthy human donor (“Donor #”)

Formulation (Per Liter):

| | | |
|---|---|---|
| Potassium phosphate monobasic | 1.3 | mM |
| Sodium chloride | 0.76 | mM |
| Potassium phosphate dibasic | 1.7 | mM |
| ATCC Trace Mineral Solution | 10 | mL |
| Sodium bicarbonate | 1 | g |
| Clarified rumen fluid | 300 | mL |
| Type II mucin | 5 | g |
| ATCC Vitamin solution | 10 | mL |
| Acetic acid | 170 | μL |
| Propionic acid | 60 | μL |
| Butyric acid | 40 | μL |
| Isobutyric acid | 10 | μL |
| n-valeric acid | 10 | μL |
| Isovaleric acid | 10 | μL |
| DL-α-methylbutyric acid | 10 | μL |
| L-cysteine | 0.005 | g |
| Hemin | 0.05 | mg |
| Vitamin K | 0.0025 | mg |
| Final pH 6.8 ± 0.2 | | |

### Example 2: Bacterial Isolates that Produce a Short-Chain Fatty Acid (SCFA)

A functional assay was developed for screening bacterial isolates for production of butyrate. Bacterial isolates were isolated from stool samples of healthy human donors and grown in liquid culture as described above. Cells were pelleted by centrifugation and resuspended in deep well plates in 150 μL of fresh culture media and 150 μL of assay buffer (0.1M sodium phosphate buffer (pH 7) containing 2 grams/200 mL sodium phosphate buffer of each of fructooligosaccharides (FOS), xylooligosaccharides (XOS), sunfiber/partially hydrolyzed guar gum (PHGG) and barley malt). Plates were incubated at 37° C. in an anaerobic chamber for 6 hours or 24 hours on a plate shaker at 200 rpm in order to allow fermentation of carbohydrates to butyrate. Following incubation, cells were pelleted and 100 μl of supernatant was transferred to a fresh vial for SCFA extraction.

100 μl of supernatant was mixed with 10 μl of 50% sulfuric acid and 500 μl of diethyl ether containing 5 mM 2-methylpentanoic acid in a 2 mL glass vial. Samples were centrifuged at 3,220 g for 10 minutes to obtain a clear phase boundary. SCFA levels were measured using a Gas Chromatograph (GC) with a Flame Ionization Detector (FID) using the following parameters:
Injection: A 1 μL sample injected at a sample depth of 8 mm with a fast plunger speed and four sample washes.
Inlet: Split mode at 225° C. with a 20:1 split ratio.
Carrier Gas: Helium
Oven: Isothermal temperature program at 140° C. for 5 min.
Column: Nitroterephthalic-acid modified/polyethylene glycol (PEG) capillary column that is 0.25 mm in diameter, approximately 30 m long with a 0.25 μm film thickness. Column kept at a constant flow of 6.0 mL/min.
Flame Ionization Detector (FID): Temperature set at 225° C. Flow rate for hydrogen is 30.0 mL/min. Flow rate for air is 400.0 mL/min. Makeup flow of helium is 20.0 mL/min.

Levels of butyrate were determined as net concentrations (t24-t0, and t6-t0) and as a percent of total SCFAs produced, on a per carbon basis.

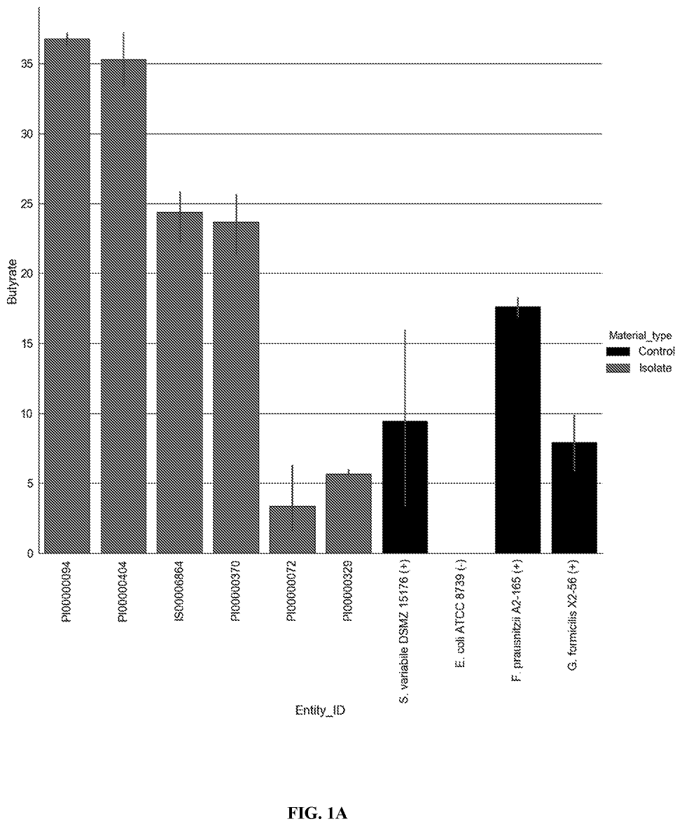

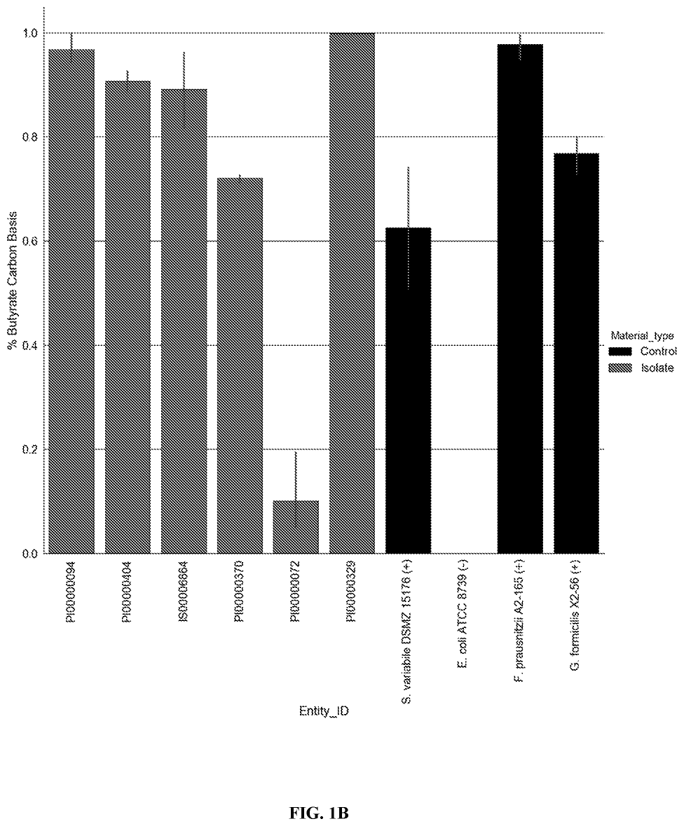

FIG. 1A shows butyrate concentration (mM) produced by bacterial isolates after 24 hours incubation in substrate buffer. For these same bacterial isolates, FIG. 1B shows the % butyrate produced of total SCFA production per carbon after 24 hours incubation in substrate buffer. Taxonomic information for each isolate, as well as SEQ ID NOs corresponding to each 16S rRNA sequence, can be found in Table 2. Also shown is data for positive control strains DSM 15176 (Subdoligranulum variabile), DSM 17677 (Faecalibacterium prausnitzii A2-165), and ATCC 27749 (Gemmiger formicilis X2-56). The E. coli ATCC 8739 strain, which does not secrete butyrate, was used as a negative control. The two S. variabile strains listed in Table 2 (IS00007359 and IS00007357) are also predicted to produce butyrate based on their taxonomic similarity to strain DSM 15176.

The ability of these bacterial isolates to produce butyrate (e.g., in the gut of a subject administered one or more of the bacterial isolates) makes them excellent candidates for inclusion in a pharmaceutical composition (e.g., microbial cocktail) described herein for the treatment of an intestinal dysbiosis (e.g. IBD or ulcerative colitis) in a subject in need thereof.

### Example 3: Bacterial Isolates that Modulate Cytokine Production

Functional assays were developed for screening bacterial isolates for modulation of human immune cells, e.g., by influencing specific cytokine production in human peripheral blood mononuclear cells (PBMC).

A screen was performed to identify bacterial strains that induce production of anti-inflammatory cytokines while not inducing (or possibly inhibiting) production of pro-inflammatory cytokines from host cells. Bacterial isolates were co-cultured with human PBMCs and the levels of six cytokines measured. The six cytokines were: IL-10, a significant anti-inflammatory cytokine; GM-CSF, which evidence suggests has protective effects on gut immunology; and IL-12p70, IFN-gamma, TNF-alpha, and IL-23 and which are believed to have pro-inflammatory effects.

Bacterial isolates were cultured in liquid culture as described above and cells were washed and re-suspended at 1×10¹ CFU/mL (optionally, stored at −80° C. until the later co-culture step). Human PBMCs were seeded into 24-well plates and incubated in 5% CO₂ overnight. Bacterial isolates were added to the PBMCs at a multiplicity of infection (MOI) of 0.4×, 2×, or 10× and co-cultured for 24 hours. The co-culture supernatants were then collected and cytokine levels (of IL-10, IL-12, IFN-gamma, GM-CSF, TNF-alpha, and IL-23) were quantified by Luminex.

Tables 24 and 24a shows the results of the assay for isolates assayed using PBMCs from three human donors (results in pg/ml). Also shown is data for positive control strain DSM 17677 (Faecalibacterium prausnitzii A2-165) and negative controls (E. coli ATCC 8739, PBMCs alone, and cell culture medium alone). Each of the tested isolates induced an anti-inflammatory profile, characterized by high levels of IL-10 production and/or low levels of IL-12, TNF-alpha, IFN-gamma, and/or IL-23, as shown by the ratios of IL-10/IL-12 and IL-10/TNF-alpha in Tables 24 and 24a. The bacterial isolates identified as beneficial in immunomodulation in the PBMC assay are each identified above in Table 3, along with the Sequence Identifier (SEQ ID NO) for their respective 16S rRNA sequences.

The ability of these bacterial isolates to induce an anti-inflammatory cytokine profile from host cells (e.g., in the gut of a subject administered one or more of the bacterial isolates) makes them excellent candidates forinclusion in a pharmaceutical composition (e.g., microbial cocktail) described herein for the treatment of an intestinal dysbiosis (e.g. IBD or ulcerative colitis) in a subject in need thereof.

TABLE 24

| | | | GM- | IL- | IFN- | TNF- | | IL-10/ | IL-10/ |
|---|---|---|---|---|---|---|---|---|---|
| ID Number | Taxonomy | IL-10 | CSF | 12p70 | gamma | alpha | IL-23 | IL12 | TNF-alpha |
| PI00000072 | O. splanchnicus | 3149 | 29.32 | 14.50 | 13.28 | 364.1 | 34.15 | 217.3 | 8.65 |
| PI00000395 | A. shahii | 2885 | 49.93 | 41.39 | 13.65 | 693.3 | 110.6 | 69.70 | 4.16 |
| PI00000094 | A. hadrus | 3453 | 72.88 | 8.54 | 12.55 | 523.8 | 114.9 | 404.5 | 6.59 |
| PI00000137 | B. uniformis | 2811 | 91.25 | 21.38 | 6.62 | 1225 | 226.3 | 131.5 | 2.29 |
| PI00000352 | B. uniformis | 3032 | 64.37 | 92.60 | 29.75 | 2680 | 309.3 | 32.74 | 1.13 |
| PI00000138 | B. vulgatus | 2751 | 62.21 | 57.46 | 21.72 | 1680 | 135.0 | 47.88 | 1.64 |
| PI00000370 | C. comes | 3639 | 117.3 | 69.71 | 24.28 | 2713 | 180.2 | 52.20 | 1.34 |
| PI00000329 | F. prausnitzii | 1672 | 10.73 | 0.63 | 1.72 | 45.23 | 7.74 | 2637 | 36.96 |
| IS00006632 | F. prausnitzii | 2760 | 45.88 | 7.85 | 3.99 | 565.8 | 71.72 | 351.8 | 4.88 |
| DSM 17677 | F. prausnitzii | 3949 | 46.47 | 23.08 | 6.24 | 2398 | 73.16 | 171.1 | 1.65 |
| ATCC 8739 | E. coli | 3585 | 479.0 | 351.57 | 361.42 | 27867 | 1999 | 10.20 | 0.13 |
| | PBMC only | 0.85 | 13.38 | 1.66 | 0.50 | 2.49 | 3.72 | 0.51 | 0.34 |
| | cell culture | 0.05 | 0.50 | 0.04 | 0.16 | 0.40 | 0.07 | 1.23 | 0.13 |
| | medium only | | | | | | | | |

TABLE 24a

| | | | GM- | IL- | IFN- | TNF- | | IL-10/ | IL-10/ |
|---|---|---|---|---|---|---|---|---|---|
| ID Number | Taxonomy | IL-10 | CSF | 12p70 | gamma | alpha | IL-23 | IL12 | TNF-alpha |
| IS00007180 | A. muciniphila | 2136 | 44.53 | 33.12 | 12.86 | 511.2 | 79.19 | 64.49 | 4.18 |
| IS00007359 | S. variabile | 2948 | 66.14 | 51.30 | 14.70 | 971.8 | 157.5 | 57.48 | 3.03 |
| IS00007357 | S. variabile | 1788 | 26.46 | 3.25 | 2.14 | 136.7 | 1.29 | 550.4 | 13.08 |
| DSM 17677 | F. prausnazii | 1790 | 64.04 | 20.14 | 13.58 | 1249 | 117 | 88.89 | 1.43 |
| ATCC 8739 | E. coil | 2561 | 180.2 | 1371 | 487 | 10386 | 4113 | 1.87 | 0.25 |
| | PBMC only | 2.41 | 3.34 | 1.66 | 3.60 | 0.53 | 5.04 | 1.45 | 4.52 |

### Example 4: Reduction of Inflammatory Markers

The anti-inflammatory activity of a microbial cocktail comprising bacterial isolates described herein was tested. The PBMC assay described in Example 3 was modified by co-incubating the PBMCs with viable E. coli to induce an inflammatory response, thus generating a sensitized platform incorporating the physiologically and disease (e.g., IBD) relevant PBMC cell type for determining the capacity of a bacterial consortium to reduce inflammation.

Bacterial isolates were cultured in liquid culture as described above and cells were washed and re-suspended at 1×10⁹ CFU/mL (optionally, stored at −80° C. until the later co-culture step). Human PBMCs were seeded into 24-well plates and incubated in 5% C02 overnight. For consortia assays each individual bacterial isolate was added to the PBMCs at a multiplicity of infection (MOI) of 0.4 and co-cultured for 24 hours. The MOI of E. coli was kept at 0.4 in all experiments. The co-culture supernatants were then collected and cytokine levels (of IL-10, IL-12, IFN-gamma, TNF-alpha, and IL-23) were quantified by Luminex.

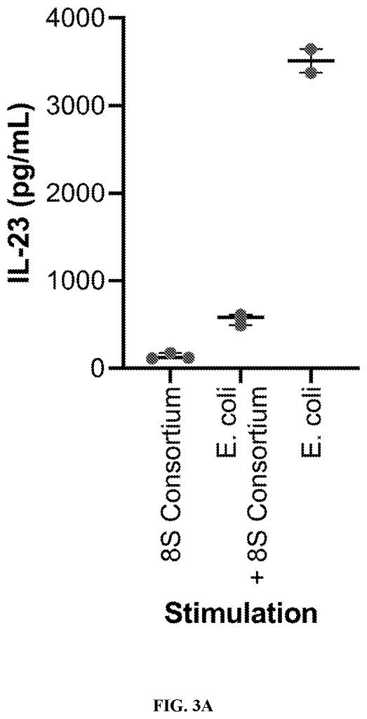

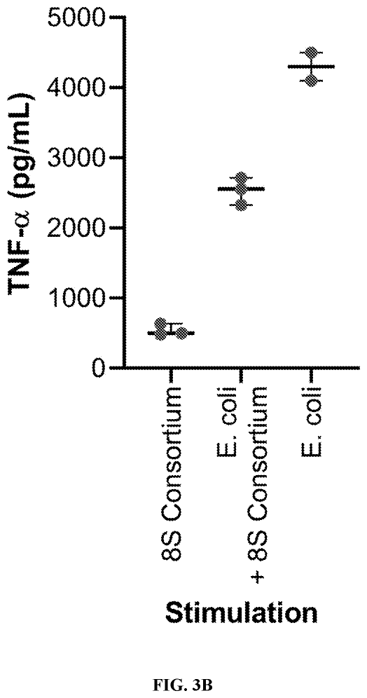

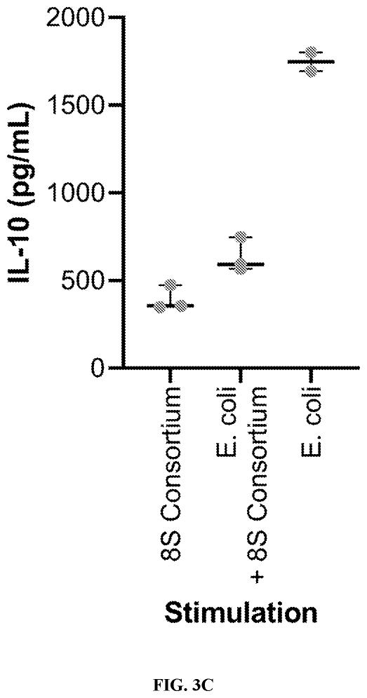

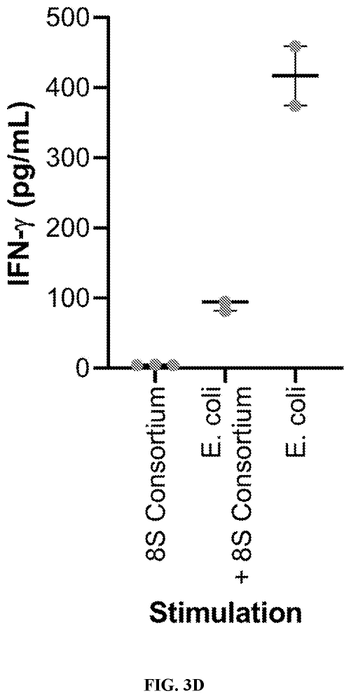

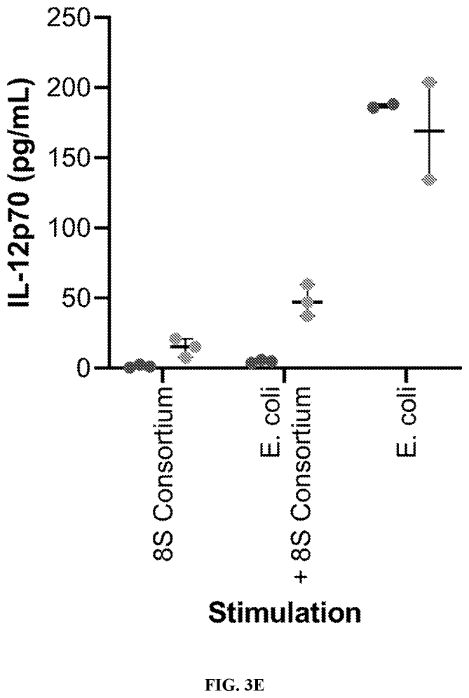

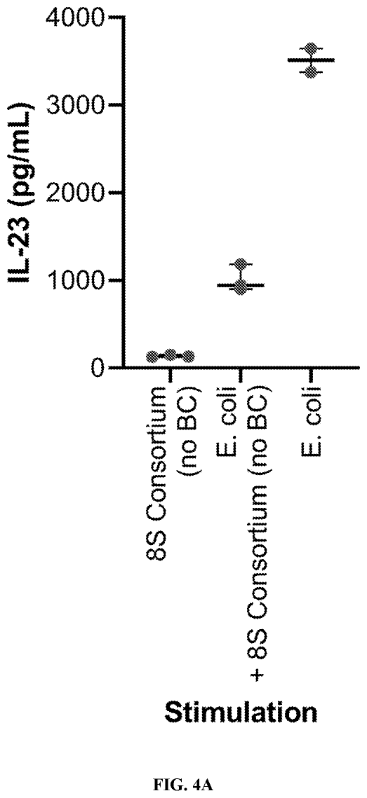

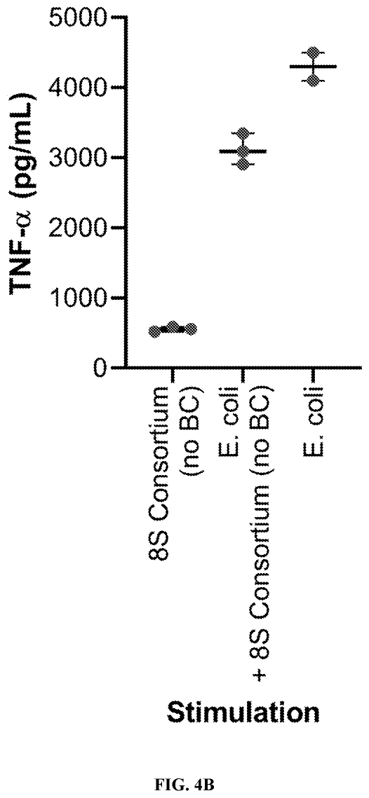

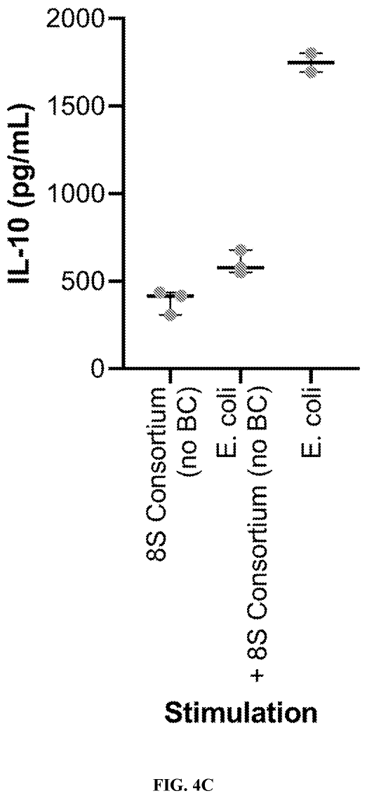

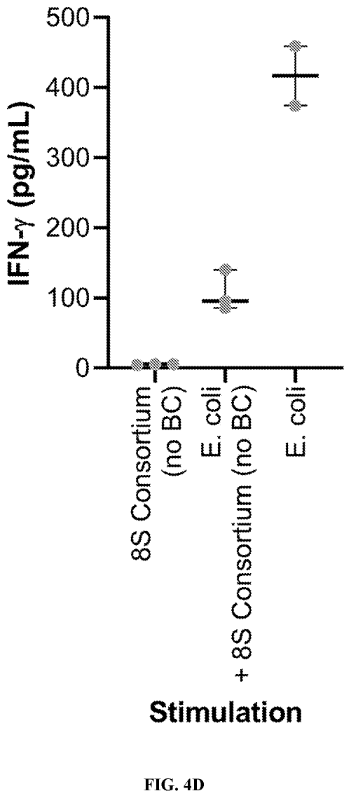

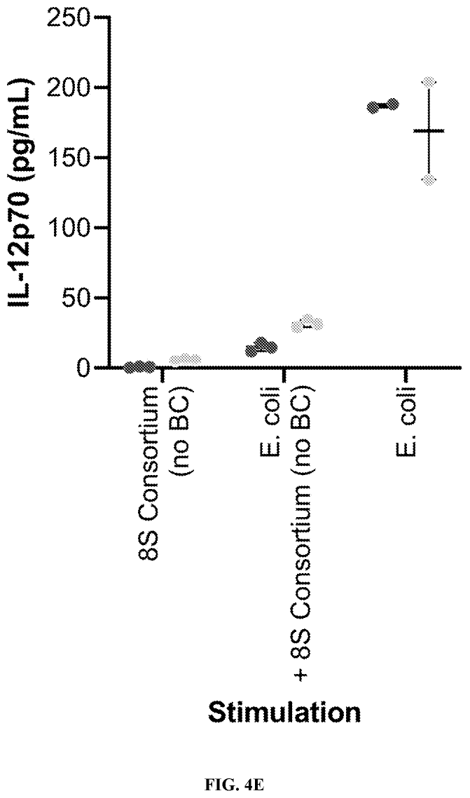

FIG. 3A-E and FIG. 4A-E shows the anti-inflammatory effects of incubation of the PBMC/E. coli mixture with 8-strain and 7-strain microbial cocktails, respectively. The 8-strain microbial cocktail consisted of Odoribacter splanchnicus (PI00000072), two isolates of Faecalibacterium prausnitzii (IS00006632 and PI00000329) Akkermansia muciniphila (IS00007180), Bacteroides cellulosilyticus (PI00000316), Eubacterium rectale, (IS00006864) Alistipes shahii (PI00000395) and Roseburia faecis (PI00000404). The 7-strain microbial cocktail included the same strains, except B. cellulosilyticus was removed. Incubation with each microbial cocktail reduced the anti-inflammatory effect of treatment with E. coli, as shown by reduced levels of IL-23 (FIGS. 3A and 4A), TNF-α (FIGS. 3B and 4B), IL-10 (FIGS. 3C and 4C), IFN-γ (FIGS. 3D and 4D) and IL12-p70 (FIGS. 3E and 4E).

### Example 5: Bacterial Isolates Enriched in Healthy Subjects

A mechanism agnostic empirical approach was developed to identify species associated with reducing the dysbiosis associated with Ulcerative Colitis (UC). 16S ribosomal DNA (rDNA) and shotgun metagenomic sequences were incorporated from interventional (FMT), cross-sectional and time series datasets to develop predictive features associated with either a healthy status or clinical response to FMT. These features were used to rank and select bacterial phylogenetic clades for (i) enrichment in healthy subjects over patients diagnosed with UC; and/or (ii) association/correlation with clinical remission or response of UC symptoms in UC patients following FMT treatment. Clades were ranked based on a “Cross-sectional combined p-value” which compares the presence and abundances of bacterial strains between healthy subjects and patients with UC. The lower the value, the more likely the organisms in the clade are having an effect on the treatment, inhibition or prevention of UC based on: (i) depletion of the strain in UC patients and/or (ii) high abundance of the strain in healthy subjects. Isolated bacterial strains were then selected by 16S rDNA similarity to the ranked phylogenetic clades or by ranking their 16S rDNA directly according to the aforementioned criteria.

Twenty-three bacterial strains were identified as enriched in healthy subjects relative to UC patients. To identify bacterial isolates corresponding to the twenty-three bacterial strains, microbiota extracted from stool samples of donors were screened for bacteria having at least 97% identity to the 16S rRNA sequence of each identified bacterial strain. Table 25 shows these isolates' taxonomic information, sequence identifier (SEQ ID NO) for its 16S rRNA sequence, and cross-sectional combined p-value.

TABLE 25

| | | SEQ ID NO for 16S | Cross sectional |
|---|---|---|---|
| Isolate Latin Name | ID Number | rRNA Sequence | combined p-value |
| Faecalibacterium prausnitzii | PI00000329 | 1 | 8.0E−3 |
| Odoribacter splanchnicus | PI00000072 | 2 | 1.31E−15 |
| Anaerostipes hadrus | PI00000094 | 3 | 1.37E−04 |
| Alistipes onderdonkii | IS00004389 | 4 | 1.35E−17 |
| Parabacteroides merdae | IS00006167 | 5 | 1.30E−07 |
| Dorea longicatena | IS00006618 | 6 | 2.00E−05 |
| Faecalibacterium prausnitzii | IS00006632 | 7 | 0.123420709 |
| Eubacterium rectale | IS00006864 | 8 | 2.69E−11 |
| Blautia obeum | PI00000053 | 9 | 3.91E−05 |
| Bacteroides uniformis | PI00000137 | 11 | 5.38E−06 |
| Bacteroides vulgatus | PI00000138 | 12 | 0.001351122 |
| Bacteroides cellulosilyticus | PI00000316 | 14 | 4.04E−12 |
| Alistipes finegoldii | PI00000340 | 15 | 1.13E−18 |
| Bacteroides uniformis | PI00000352 | 16 | 3.66E−06 |
| Alistipes shahii | PI00000395 | 18 | 6.30E−21 |
| Akkermansia mucimphila | IS00007180 | 20 | 7.0E−8 |
| Phascolarctobacterium faecium | PI00000289 | 21 | 7.0E−9 |
| Subdoligranulum variabile | IS00007359 | 22 | 1.0E−5 |
| Subdoligranulum variabile | IS00007357 | 23 | 1.0E−3 |
| Blautia sp. | IS00002788 | 34 | 2.0E−10 |
| Alisapes putredinis | IS00008139 | 35 | 1.0E−10 |
| Alistipes putredinis | IS00008142 | 36 | 1.0E−10 |
| Alistipes putredinis | IS00008177 | 37 | 1.0E−10 |

An additional ten bacterial isolates having a 16S rRNA sequence at least 95% identical to a bacterial isolate of Table 25 were also characterized. Table 26 lists each isolate's identification number, the ID number of the related bacterial isolate from Table 25, the percent identity of the isolate's 16S rRNA sequence to the related isolate in Table 25, the cross-sectional combined p value of the isolate, and the isolate's sequence identifier (SEQ ID NO). These data demonstrate that the functionality of the bacterial isolates identified in Table 25 in relieving one or more symptoms of UC is exemplary only and is also possessed by a larger group of related isolates (e.g., sharing at least 95% 16S rRNA sequence identity with the bacterial isolates of Table 25).

TABLE 26

| | Related to ID | % identity of 16S | Cross sectional | SEQ ID NO. |
|---|---|---|---|---|
| | Number of | to ID Number of | combined | for 16S rRNA |
| ID Number | Table 25 | Table 25 | p-value | Sequence |
| PI00000070 | PI00000072 | 98.6 | 1.31E−15 | 24 |
| PI00000092 | PI00000094 | 98.1 | 1.17E−05 | 25 |
| PI00000339 | IS00004389 | 96.6 | 1.13E−18 | 26 |
| PI00000327 | IS00006167 | 99.4 | 1.13E−07 | 27 |
| PI00000056 | PI00000053 | 96.2 | 3.91E−05 | 28 |
| PI00000152 | PI00000138 | 98.7 | 0.001351122 | 29 |
| PI00000043 | PI00000316 | 99.2 | 2.71E−11 | 30 |
| IS00003009 | PI00000340 | 97.6 | 2.15E−11 | 31 |
| PI00000052 | PI00000352 | 96.6 | 2.85E−11 | 32 |
| PI00000330 | PI00000395 | 95.3 | 1.13E−18 | 33 |

### Example 6: Engraftment of Bacterial Isolates

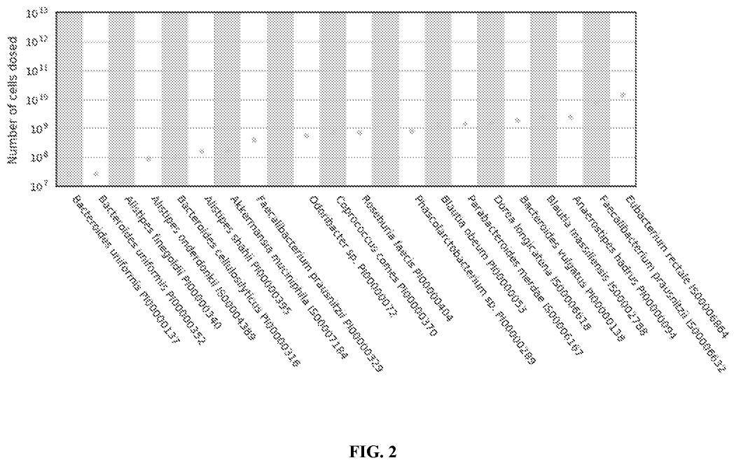

FIG. 2 shows the relationship between the dosage of various bacterial strains corresponding by 16S rRNA sequence to bacterial isolates and engraftment of the strains in the gut of a subject following administration to the subject of a substantially complete fecal microbiota (i.e., a preparation of uncultured fecal bacteria) containing the strains.

For each bacterial isolate corresponding to a given strain in the fecal microbiota, number of cells dosed to a patient was calculated by the following formula: frequency of bacteria in the administered composition having a 16S sequence matching the 16S sequence of the bacterial isolate×9×10¹⁰ (approximate number of cells per gram of the administered composition)×cumulative dose of the composition administered to a patient (in grams).

Engraftment of bacteria was measured for each unique 16S sequence by comparing relative abundances in the administered composition, the patient's baseline community, and the patient's post-treatment community. Positive engraftment was called for cases where the abundance in the patient's baseline was <20% of the administered material, and the post-treatment abundance increased at least 5-fold.

Results show that the minimum dose to achieve engraftment for most bacterial isolates screened (with the exception of the E. rectale isolate IS00006864) is below 10¹⁰ cells.

### Example 7: Engraftment of Bacterial Isolates

To assess the engraftment of bacterial isolates in the intestine of a subject following administration of a microbial cocktail, germ free C57BL/6 mice were inoculated via gavage with the 8-strain consortium described in Example 4 grown under anaerobic conditions. Fecal pellets were collected at 7, 14, 21 and 28 days and DNA was isolated for 16srRNA gene sequencing. Relative abundance of strains was determined based on 16S rRNA gene abundance.

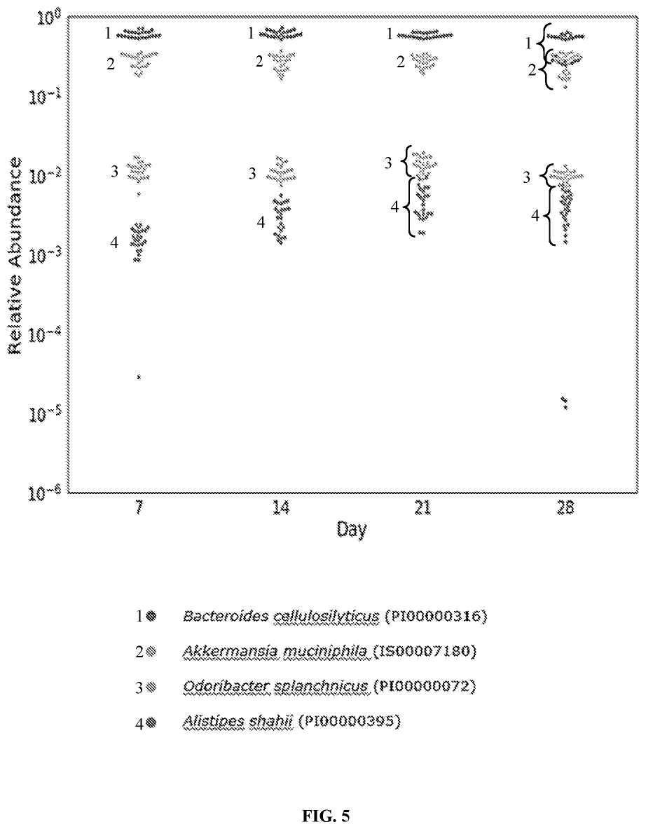

FIG. 5 shows the engraftment of four bacterial isolates (Bacteroides cellulosilyticus, Akkermansia muciniphila, Odoribacter sphlanchnicus, and Alistipes shahii) in the intestine of the germ free mice following administration of the microbial cocktail comprising the isolates.

### Example 8: Production of a Therapeutic Agent

Bacterial isolates of any one of Tables 7-22 are independently cultured and mixed together before administration. Isolates are independently grown anaerobically in supportive media, e.g. at 37° C., pH 7, as shown in Example 1. After each isolate reaches a sufficient biomass, it is optionally preserved for banking by adding 15% glycerol and then frozen at −80° C. in 1 ml cryotubes.

Each bacterial isolate is then cultivated to a concentration of about 10¹⁰ CFU/mL, then concentrated 20-fold by tangential flow microfiltration; the spent medium is exchanged by diafiltering with a preservative medium consisting of 2% gelatin, 100 mM trehalose, and 10 mM sodium phosphate buffer, or other suitable preservative medium. The suspension is freeze-dried to a powder and titrated.

After drying, the powder is blended with microcrystalline cellulose and magnesium stearate and formulated into a 250 mg gelatin capsule containing 10 mg of lyophilized powder (10⁸ to 10¹¹ bacteria), 160 mg microcrystalline cellulose, 77.5 mg gelatin, and 2.5 mg magnesium stearate.

### Example 9: Treatment of Ulcerative Colitis

A patient with a diagnosis of Ulcerative Colitis (UC) by endoscopy and histology consistent with diagnosis is treated with a pharmaceutical composition disclosed herein.

Efficacy is monitored as described in Travis, et al. Aliment Pharmacol Ther. 2011; 34(2):113-124 (which is incorporated by reference in its entirety), including one or more of the following indices (for each index, the associated publication is incorporated by reference in its entirety):

### Clinical:

Truelove and Witts (Truelove S C, Witts L J. Cortisone in ulcerative colitis. Final report on a therapeutic trial. BMJ 1955; 2: 1041-8),
Powel-Tuck Index (St. Mark's Index; Powell-Tuck J, et al. Correlations between defined sigmoidoscopic appearances and other measures of disease activity in ulcerative colitis. Dig DisSci 1982; 27: 533-7),
Clinical Activity Index (CAI; Rachmilewitz D. Coated mesalazine (5-aminosalicylic acid) versus sulphasalazine in the treatment of active ulcerative colitis: a randomised trial. BMJ 1989; 298: 82-6),
Lichtiger Index (S. Lichtiger, D. H. Present Preliminary report: cyclosporin in treatment of severe active ulcerative colitis Lancet, 336 (1990), pp. 16-19),
Seo Index (Seo M, et al. An index of disease activity in patients with ulcerative colitis. Am J Gastroenterol 1992; 87: 971-6),
Physician's Global Assessment (Hanauer S et al., Mesalamine capsules for treatment of active ulcerative colitis: results of a controlled trial Am J Gastroenterol. 1993 August; 88(8):1188-97),
Investigator's Global Evaluation (Hanauer S B et al., Budesonide enema for the treatment of active, distal ulcerative colitis and proctitis: a dose-ranging study. U.S. Budesonide enema study group Gastroenterology. 1998 September; 115(3):525-32).
Simple Clinical Colitis Activity Index (Walmsley R S, et al., A simple clinical colitis activity index. Gut 1998; 43: 29-32),
Improvement Based on Individual Symptom Scores (Levine et al., A randomized, double blind, dose-response comparison of balsalazide (6.75 g), balsalazide (2.25 g), and mesalamine (2.4 g) in the treatment of active, mild-to-moderate ulcerative colitis Am J Gastroenterol. 2002 June; 97(6):1398-407), and
Patient-defined remission (Higgins P, et al. Is endoscopy necessary for the measurement of disease activity in ulcerative colitis. Am J Gastroenterol 2005; 100: 355-61).

### Endoscopic:

Truelove Witts Sigmoidoscopic Assessment (Seo M, et al. Evaluation of disease activity in patients with moderately active ulcerative colitis: comparisons between a new activity index and Truelove and Witts classification. Am J Gastroenterol 1995; 90: 1759-63),
Baron Score (Baron J H, et al. Variation between observers in describing mucosal appearances in proctocolitis. BMJ 1964; 1: 89-92),
Endoscopic Index (Rachmilewitz D. Coated mesalazine (5-aminosalicylic acid) versus sulphasalazine in the treatment of active ulcerative colitis: a randomised trial. BMJ 1989; 298: 82-6),
Sigmoidoscopic Index (Hanauer S et al., Mesalamine capsules for treatment of active ulcerative colitis: results of a controlled trial Am J Gastroenterol. 1993 August; 88(8):1188-97),
Sigmoidoscopic Inflammation Grade Score (Lemann M et al., Comparison of budesonide and 5-aminosalicylic acid enemas in active distal ulcerative colitis Aliment Pharmacol Ther. 1995 October; 9(5):557-62.),
Mayo Score Flexible Proctosigmoidoscopy Assessment (Schroeder K W, et al. Coated oral 5-aminosalicylic acid therapy for mildly to moderately active ulcerative colitis. A randomized study. N Engl J Med 1987; 317: 1625-9), and
Modified Baron Score (Rutgeerts P, et al. Infliximab for induction and maintenance therapy for ulcerative colitis. N Engl J Med 2005; 353: 2462-76).

### Clinical and Endoscopic:

Mayo Clinic Score (Schroeder K W, et al. Coated oral 5-aminosalicylic acid therapy for mildly to moderately active ulcerative colitis. A randomized study. N Engl J Med 1987; 317: 1625-9) and
Sutherland Index (Sutherland L R, et al. 5-Aminosalicylic acid enema in the treatment of distal ulcerative colitis, proctosigmoiditis, and proctitis. Gastroenterology 1987; 92: 1894-8).

### Quality of Life:

Rating form of IBD Patient Concerns (Drossman D A et al. Health status and health care use in persons with inflammatory bowel disease. A national sample. Dig Dis Sci. 1991 December; 36(12):1746-55),
Inflammatory Bowel Disease Questionnaire (Irvine E J, et al. Quality of life: a valid and reliable measure of outcome for clinical trials in inflammatory bowel disease. Gastroenterology. 1194; 106:287-96), and
Short Form-36 (Ware J E et al. The MOS 36-item short-form health survey (SF-36). I. Conceptual framework and item selection Med Care. 1992 June; 30(6):473-83).

### Histological:

Riley Index (Riley S A, et al. Microscopic activity in ulcerative colitis: what does it mean? Gut 1991; 32: 174-8),
Gebboes Index (Geboes K et al. A reproducible grading scale for histological assessment of inflammation in ulcerative colitis Gut. 2000 September; 47(3):404-9), and
Chicago Index (Rubin D T, et al. Increased degree of histological inflammation predicts colectomy and hospitalization in patients with ulcerative colitis. Gastroenterology 2007; 132(Suppl. 1): A-19 (Abstract 103)).

### Example 12: In Vivo Testing of a Therapeutic Agent in Mice

A composition or various compositions comprising the eight isolates of any one of Tables 13, 14, 15, 15a and 15b are provided. Alternatively, a therapeutic agent, e.g., without limitation, made via the methods of Example 8, comprising the eight isolates of any one of Tables 13, 14, 15, 15a and 15b are provided.

A suitable murine model for UC is selected and provided. Illustrative models include chemical models (such as DSS, TNBS, oxazolone, acetic acid, and sulfhydryl inhibitors) and transgenic/knock out models (such as TCRα^{-/-}, WASP^{-/-}, Mdr1a^{-/-}, IL-2^{-/-}, Gui2^{-/-}, IL-7 Tg, TRUC, TGFβRIIDN, and C3H/HeJBir). See World J Gastroenterol. 2007 Nov. 14; 13(42): 5581-5593 and Drug Des Devel Ther. 2013; 7: 1341-1357 (2013), the entire contents of which are incorporated by reference.

Taking DDS as anon-limiting example, oral administration of 3-5% DSS in drinking water to mice is undertaken. For instance, one cycle of 3%-5% DSS administration for 5-7 d, followed by regular water, results in extensive injury with complete crypt depletion (e.g. basal crypt) and relatively slow regeneration of colonic epithelium. Animals which develop UC-like symptoms are treated with the composition or various compositions comprising the nine isolates of any one of Tables 36-43 or a therapeutic agent (alternatively, the bacterial compositions can be administered concurrently with the DSS) and comparison is made to determine therapeutic effect (e.g. comparing one or more parameters, such as measuring clinical scores of colitis, including weight change, diarrhea, colorectal bleeding and survival; overall disease severity may be assessed by a clinical scoring system, and a disease activity index (DAI) score may be calculated for each animal). Alternative measures include endoscopy, histological analyses, microbiota analysis by 16S rRNA sequencing, and the like. A more detailed protocol is found in Curr Protoc Immunol. 2014 Feb. 4; 104: Unit-15.25, the entire contents of which are incorporated by reference. The present Example allows for variations in the detailed protocol is found in Curr Protoc Immunol. 2014 Feb. 4; 104: Unit-15.25 as necessary or desired to determine the presence or absence of a therapeutic effect.

### Example 13: Treatment of UC

A composition or various compositions comprising the eight isolates of any one of Tables 13, 14, 15, 15a and 15b is provided. Alternatively, a therapeutic agent, e.g., without limitation, made via the methods of Example 8, comprising the eight isolates of any one of Tables 13, 14, 15, 15a and 15b is provided. A study of a UC as described in Example 9 is undertaken with such compositions as the therapeutic agent.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior invention.

As used herein, all headings are simply for organization and are not intended to limit the disclosure in any manner. The content of any individual section may be equally applicable to all sections.

1. A method of preparing a therapeutic bacterial isolate, the method comprising:
(i) providing a preparation of bacteria from fecal material from
(a) a group of healthy subjects,
(b) a group of patients with a gastrointestinal disease or disorder, and
(c) a group of patients having clinical remission of one or more symptoms of the gastrointestinal disease or disorder following treatment of each patient of the group of patients with a fecal microbiota transplant,
(ii) analyzing 16S rRNA sequences from the preparation of bacteria, and
(iii) isolating a bacterial isolate from stool of a human donor that comprises a 16S rRNA sequence that is at least 97% identical to a 16S rRNA sequence of a bacterial strain from the preparation of bacteria that is
(a) enriched in a group of healthy subjects over a group of patients with the gastrointestinal disease or disorder; and/or
(b) correlated with clinical remission of one or more symptoms of the gastrointestinal disease or disorder in a group of patients following treatment of each patient of the group of patients with the fecal microbiota transplant,
wherein a cross-sectional combined p-value of the bacterial isolate is less than 1×10⁻¹⁴.

2. The method of claim 1, wherein the bacterial strain enriched in a group of healthy subjects over a group of patients with the gastrointestinal disease or disorder is selected from Faecalibacterium prausnitzii, Odoribacter splanchnicus, Anaerostipes hadrus, Alistipes onderdonkii, Faecalibacterium prausnitzii, Eubacterium rectale, Blautia obeum, Bacteroides uniformis, Bacteroides vulgatus, Bacteroides cellulosilyticus, Alistipes finegoldii, Alistipes shahii, Akkermansia muciniphila, Phascolarctobacterium faecium, Subdoligranulum variabile, Subdoligranulum variabile, Blautia sp., Alistipes putredinis, Alistipes putredinis, Alistipes putredinis, and any two or more thereof.

3. The method of claim 1 or claim 2, wherein the bacterial strain enriched in a group of healthy subjects over a group of patients with the gastrointestinal disease or disorder comprises a 16S rRNA sequence that has at least about 98%, or at least about 99% sequence identity with a nucleotide sequence selected from SEQ ID NOs: 1, 2, 3, 4, 7, 8, 9, 11, 12, 14, 15, 16, 18, 20, 21, 22, 23, 34, 35, 36, and 37.

4. The method of any one of claims 1-3, wherein the bacterial isolate comprises at least one of Odoribacter splanchnicus and Alistipes shahii.

5. The method of any one of claims 1-4, wherein the bacterial isolate comprises a 16S rRNA sequence that has at least about 98%, or at least about 99% sequence identity with a nucleotide sequence of SEQ ID NO: 2 and SEQ ID NO: 18.

6. The method of any one of claims 1-5, wherein the bacterial isolate comprises a 16S rRNA sequence that is at least 99% identical to a 16S rRNA sequence of the bacterial strain.

7. The method of any one of claims 1-6, wherein the bacterial strain correlated with clinical remission of one or more symptoms of the gastrointestinal disease or disorder in a group of patients following treatment of each patient of the group of patients with the fecal microbiota transplant comprises:
Alistipes finegoldii, but does not comprise Alistipes shahii or Alistipes putredinis, or
Alistipes putredinis, but does not comprise Alistipes shahii or Alistipes finegoldii.

8. The method of any one of claims 1-7, wherein the bacterial strain correlated with clinical remission of one or more symptoms of the gastrointestinal disease or disorder in a group of patients following treatment of each patient of the group of patients with the fecal microbiota transplant comprises:
a 16S rRNA sequence that has at least about 95%, or at least about 97%, or at least about 98%, or at least about 99% sequence identity with a nucleotide sequence of SEQ ID NO: 15, but not a nucleotide sequence selected from SEQ ID NO: 18, 35, 36 or 37; or
a 16S rRNA sequence that has at least about 95%, or at least about 97%, or at least about 98%, or at least about 99% sequence identity with the nucleotide sequence selected from SEQ ID NOs: 35-37, but not a nucleotide sequence selected from SEQ ID NO: 15 and 18.

9. The method of any one of claims 1-8, wherein the method further comprises isolating a second therapeutic bacterial isolate by:
(iv) providing multiple bacterial isolates from human fecal samples; and
(v) performing a functional assay comprising:
(a) contacting a population of eukaryotic cells with the human-derived bacterial isolates,
(b) measuring a level of a cytokine in the population of eukaryotic cells, and
(c) selecting the bacterial isolate capable of modulation of the level of the cytokine in the population of eukaryotic cells.

10. The method of claim 9, wherein the population of eukaryotic cells comprises a population of PBMCs.

11. The method of claim 9 or claim 10, wherein the cytokine is selected from IL-10, GM-CSF, IFN-gamma, TNF-alpha, IL-23, and IL-12.

12. The method of any one of claims 9-11, wherein the second bacterial isolate is selected from Faecalibacterium prausnitzii, Odoribacter splanchnicus, Anaerostipes hadrus, Alistipes shahii, Akkermansia muciniphila, Subdoligranulum variabile, Bacteroides uniformis, and any two or more thereof.

13. The method of any one of claims 9-12, wherein the second bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 11, 16, 18, 20, 22, and 23.

14. A method of preparing a microbial cocktail comprising a first and second bacterial isolate, the method comprising:
(i) isolating multiple bacterial isolates from human stool;
(ii) performing a first functional assay comprising:
(a) contacting a population of eukaryotic cells with the bacterial isolates,
(b) measuring an IL-10:IL-12 ratio and an IL-10:TNF-alpha ratio, and
(c) identifying as the first bacterial isolate a bacterial isolate capable of inducing an IL-10:IL-12 ratio of at least about 50:1 or an IL-10:TNF-alpha ratio of at least about 1:1 when incubated with a population of eukaryotic cells,
(iii) performing a second functional assay comprising:
(a) measuring a level of a short chain fatty acid (SCFA) produced by the bacterial isolates, and
(b) identifying as the second bacterial isolate a bacterial isolate that produces the SCFA at a concentration of at least about 10 mM, and
(iv) combining the first and second bacterial isolates to produce the microbial cocktail.

15. The method of claim 14, wherein the first bacterial isolate is selected from Faecalibacterium prausnitzii, Odoribacter splanchnicus, Anaerostipes hadrus, Bacteroides uniformis, Bacteroides vulgatus, Coprococcus comes, Alistipes shahii, Akkermansia muciniphila, Subdoligranulum variabile, and any two or more thereof.

16. The method of claim 14 or claim 15, wherein the first bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 11, 12, 16, 17, 18, 20, 22 and 23.

17. The method of any one of claims 14-16, wherein the population of eukaryotic cells comprises a population of PBMCs.

18. The method of any one of claims 14-17, wherein the SCFA is selected from acetic acid, butyric acid, caproic acid, formic acid, heptanoic acid, isobutyric acid, isocaproic acid, isovaleric acid, propionic acid, valeric acid, and any two or more thereof.

19. The method of claim 18, wherein the SCFA is butyric acid.

20. The method of any one of claims 14-19, wherein the second bacterial isolate is selected from Odoribacter splanchnicus, Eubacterium rectale, Coprococcus comes, Faecalibacterium prausnitzii, Roseburia faecis, Anaerostipes hadrus, Subdogranulum variabile, and any two or more thereof.

21. The method of any one of claims 14-20, wherein the second bacterial isolate comprises a 16S rRNA sequence that has at least about 95%, or at least about 97%, or at least about 98%, or at least about 99% sequence identity with a nucleotide sequence selected from SEQ ID NOs: 1, 2, 3, 7, 8, 17, 19, 22, and 23.

22. The method of any one of claims 14-21, further comprising isolating a third bacterial isolate for incorporation into the microbial cocktail by:
(i) analyzing 16S rRNA sequences from preparations of bacteria from fecal material of:
(a) a group of healthy subjects,
(b) a group of patients with a gastrointestinal disease or disorder, and
(c) a group of patients having clinical remission of one or more symptoms of the gastrointestinal disease or disorder following treatment of each patient of the group of patients with a fecal microbiota transplant, and
(ii) isolating as the third bacterial isolate a bacterial isolate from stool of a human donor that comprises a 16S rRNA sequence that is at least 97% identical to a 16S rRNA sequence of a bacterial strain from the preparations of bacteria that is
(a) enriched in a group of healthy subjects over a group of patients with the gastrointestinal disease or disorder; and/or
(b) correlated with clinical remission of one or more symptoms of the gastrointestinal disease or disorder in a group of patients following treatment of each patient of the group of patients with the fecal microbiota transplant.

23. The method of claim 22, wherein the third bacterial isolate comprises a 16S rRNA sequence that is at least 99% identical to a 16S rRNA sequence of the bacterial strain.

24. The method of any one of claims 1-13, wherein the gastrointestinal disease or disorder is selected from inflammatory bowel disease (IBD), irritable bowel syndrome (IBS), C. difficile infection (CDI), C. difficile-associated disease (CDAD), and an antibiotic-induced adverse effect.

25. The method of claim 24, wherein the IBD is selected from one or more of ulcerative colitis (UC), Crohn's disease (CD), and pouchitis.

26. The method of any one of claims 14-23, wherein the gastrointestinal disease or disorder is selected from is selected from inflammatory bowel disease (IBD), irritable bowel syndrome (IBS), C. difficile infection (CDI), C. difficile-associated disease (CDAD), and an antibiotic-induced adverse effect.

27. The method of claim 26, wherein the IBD is selected from one or more of ulcerative colitis (UC), Crohn's disease (CD), and pouchitis.


SEQUENCE LISTING (35 sequences)

SEQ ID NO: 1 - DNA, 1509 bp, Artificial Sequence
  Synthetic Polynucleotide
tataaagagtttgatcctggctcaggacgaacgctggcggcgcgcctaacacatgcaagtcgaacgagcgagagagagcttgctttctcgagcgagtggcgaacgggtgagtaacgcgtgaggaacctgcctcaaagagggggacaacagttggaaacgactgctaataccgcataagcccacggctcggcatcgagcagagggaaaaggagcaatccgctttgagatggcctcgcgtccgattagctagttggtgaggtaacggcccaccaaggcaacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttggggaagataatgacggtacccaacaaggaagtgacggctaactacgtgccagcagccgcggtaaaacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacaccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcccttgacagacatagaaatatgtaatctcttcggagcaaggagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgtggatcagcatgccacggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtaggagaacctgcggctggatcacctccttt

SEQ ID NO: 2 - DNA, 1381 bp, Artificial Sequence
  Synthetic Polynucleotide
ggggtatagcccatggaaacgtggattaacaccccatagtacttttatcctgcatgggatgtgagttaaatgttcaaggtatcggatgggcatgcgtcctattagttagttggcggggtaacagcccaccaagacgatgataggtaggggttctgagaggaaggtcccccacattggaactgagacacggtccaaactcctacgggaggcagcagtgaggaatattggtcaatggacgtaagtctgaaccagccaagtcgcgtgagggaagactgccctatgggttgtaaacctcttttataagggaagaataagttctacgtgtagaatgatgcctgtaccttatgaataagcatcggctaactccgtgccagcagccgcggtaatacggaggatgcgagcgttatccggatttattgggtttaaagggtgcgtaggcggtttattaagttagtggttaaatatttgagctaaactcaattgtgccattaatactggtaaactggagtacagacgaggtaggcggaataagttaagtagcggtgaaatgcatagatataacttagaactccgatagcgaaggcagcttaccagactgtaactgacgctgatgcacgagagcgtgggtagcgaacaggattagataccctggtagtccacgccgtaaacgatgctcactggttctgtgcgatatattgtacgggattaagcgaaagtattaagtgagccacctggggagtacgtcggcaacgatgaaactcaaaggaattgacgggggcccgcacaagcggaggaacatgtggtttaattcgatgatacgcgaggaaccttacctgggtttaaatgggaaatgccgtatttggaaacagatattctcttcggagcgtttttcaaggtgctgcatggttgtcgtcagctcgtgccgtgaggtgtcgggttaagtcccataacgagcgcaacccttaccgttagttgctagcatgtaatgatgagcactctaacgggactgccaccgtaaggtgagaggaaggcggggatgacgtcaaatcagcacggcccttacacccagggctacacacgtgttacaatggccggtacagagggccgctaccaggtgactggatgccaatctcaaaagccggtcgtagttcggattggagtctgtaacccgactccatgaagttggattcgctagtaatcgcgcatcagccatggcgcggtgaatacgttcccgggccttgtacacaccgcccgtcaagccatggaagccgggggtgcctgaagtccgtaaccgcgaggatcggcctagggcaaaactggtaactggggctaagtcgtaacaaggtagccgtaccggaaggtgcggctggaacacctcctt

SEQ ID NO: 3 - DNA, 1299 bp, Artificial Sequence
  Synthetic Polynucleotide
tggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactagagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacttttccttcgggacaggagtgacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctccttt

SEQ ID NO: 4 - DNA, 800 bp, Artificial Sequence
  Synthetic Polynucleotide
tgtgtatcgccgacaacgagttatcatcgtttactgcgtggactaccagggtatctaatcctgtttgctccccacgctttcgtgcctcaacgtcagatatagtttggtaagctgccttcgcaatcggtgttctgtatgatctctaagcatttcaccgctacaccatacattccgcctaccgcaactactctctagtctaacagtattagaggcagttccggagttaagccccgggatttcacctctaacttatcaaaccgcctacgcaccctttaaacccaataaatccggataacgcttgaatcctccgtattaccgcggctgctggcacggagttagccgatccttattcgtacgatactttcagtcacctacacgtaggtgagtttaccctcgtacaaaagcagtttacaactcatagagccgtcttcctgcacgcggcatggctggttcagacttgcgtccattgaccaatattcctcactgctgcctcccgtaggagtctgatccgtgtctcagtaccagtgtggggggttaacctctcagtccccctatgtatcgttgccttggtgagccgttacctcaccaactagctaatacaacgcatgtccatctataaccaccggagttttcaacccggagagatgcctctccgaatattatggggaattagtaccaatttctcagtgttatgcccctgttataggtaggttacatacgcgttacgcacccgtgcgccggtcgccggcaattgaagcaagcttcaatcccgatgcccctcgacttgcatgtgttaggcc

SEQ ID NO: 5 - DNA, 1527 bp, Artificial Sequence
  Synthetic Polynucleotide
cgaagagtttgatcctggctcaggatgaacgctagcgacaggcttaacacatgcaagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactctttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtagccgtaccggaaggtgcggctggaacacctccttt

SEQ ID NO: 6 - DNA, 1529 bp, Artificial Sequence
  Synthetic Polynucleotide
aacgagagtttgatcctggctcaggatgaacgctggcggcgtgcttaacacatgcaagtcgagcgaagcactttggaaagattcttcggatgatttcctttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagtttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagagtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctccttt

SEQ ID NO: 7 - DNA, 1509 bp, Artificial Sequence
  Synthetic Polynucleotide
tataaagagtttgatcctggctcaggacgaacgctggcggcgcgcctaacacatgcaagtcgaacgagagagaaggagcttgcttcttcgatcgagtggcgaacgggtgagtaacgcgtgaggaacctgcctcaaagagggggacaacagttggaaacgactgctaataccgcataagcccacgggtcggcatcgaccagagggaaaaggagcaatccgctttgagatggcctcgcgtccgattagctagttggtgaggtaatggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacaccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacggacatagaaatatgtctttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgtggatcagcatgccacggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtaggagaacctgcggctggatcacctccttt

SEQ ID NO: 8 - DNA, 1529 bp, Artificial Sequence
  Synthetic Polynucleotide
atgagagtttgatcctggctcaggatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacagcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggtttggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagctgtcgaaggcaggctcgataactggggtgaagtcgtaacaaggtagccgtatcggaaggtgcggctggatcacctccttt

SEQ ID NO: 9 - DNA, 1118 bp, Artificial Sequence
  Synthetic Polynucleotide
aacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgttccttaaccggaactttccttcgggacaggggagacaggtggtgcatggttgtcgtcagctcgtgtcgtgagatgttgggttaagtcccgcaacgagcgcaacccctatccccagtagccagcagtccggctgggcactctgaggagactgccagggataacctggaggaaggcggggatgacgtcaaatcatcatgccccttatgatttgggctacacacgtgctacaatggcgtaaacaaagggaagcaagcctgcgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaac

SEQ ID NO: 10 - DNA, 1523 bp, Artificial Sequence
  Synthetic Polynucleotide
atgaagagtttgatcctggctcaggatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtggaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaactggtgattggggctaagtcgtaacaaggtagccgtaccggaaggtgcggctggaacacctcctt

SEQ ID NO: 11 - DNA, 1319 bp, Artificial Sequence
  Synthetic Polynucleotide
tgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttataaaggaataaagtcgggtatgtatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtttcgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctt

SEQ ID NO: 12 - DNA, 1519 bp, Artificial Sequence
  Synthetic Polynucleotide
atgaagagtttgatcctggctcaggatgaacgctagctacaggcttaacacatgcaagtcgaggggcagcatgacctagcaataggttgatggcgaccggcgcacgggtgagtaacacgtatccaacctaccggttattccgggatagcctttcgaaagaaagattaataccggatagtataacgagaaggcatctttttgttattaaagaatttcgataaccgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgagccacgtgtggctttttgtatgtaccatacgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatctgaataatttggaaacagattagccgcaaggcagatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacagcgatgtgatgctaatcccaaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtccgtaaccgtaaggagcggcctagggtaaaactggtaattggggctaagtcgtaacaaggtagccgtaccggaaggtgcggctggaacacctcctt

SEQ ID NO: 13 - DNA, 1521 bp, Artificial Sequence
  Synthetic Polynucleotide
atggagagtttgatcctggctcaggatgaacgctagcggcaggcctaacacatgcaagtcgaggggcatcacgaggtagcaatactttggtggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacgggaggcagcagtgaggaatattggtcaatggacgcaagtctgaaccagccatgccgcgtgcaggaagacggctctatgagttgtaaactgcttttgtacgagggtaaactcacctacgtgtaggtgactgaaagtatcgtacgaataaggatcggctaactccgtgccagcagccgcggtaatacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttccctttggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggtcgctactccgtgaggagatgccaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtaactggggctaagtcgtaacaaggtagccgtaccggaaggtgcggctggaacacctccttt

SEQ ID NO: 14 - DNA, 1523 bp, Artificial Sequence
  Synthetic Polynucleotide
atgaagagtttgatcctggctcaggatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaactggtgattggggctaagtcgtaacaaggtagccgtaccggaaggtgcggctggaacacctcctt

SEQ ID NO: 15 - DNA, 1527 bp, Artificial Sequence
  Synthetic Polynucleotide
acgagagtttgatcctggctcaggatgaacgctggcggcgtgcttaacacatgcaagtcgaacgaagcacctggatttgattcttcggatgaagatccttgtgactgagtggcggacgggtgagtaacgcgtgggtaacctgcctcatacagggggataacagttagaaatgactgctaataccgcataagaccacagggtcgcatgacctggtgggaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcatgtaatgatgccttttcttcggaacaccggtgacaggtggtgcatggttgtcgtcagctcgtgtcgtgagatgttgggttaagtcccgcaacgagcgcaacccttatcttcagtagccagcattttaggtgggcactctggagagactgccagggataacctggaggaaggtggggatgacgtcaaatcatcatgccccttatgagcagggctacacacgtgctacaatggcgtaaacaaagggaagcaaacctgtgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgactcaaccgtaaggagagagctgccgaaggtgggaccgataactggggtgaagtcgtaacaaggtagccgtatcggaaggtgcggctggatcacctccttt

SEQ ID NO: 16 - DNA, 1521 bp, Artificial Sequence
  Synthetic Polynucleotide
atggagagtttgatcctggctcaggatgaacgctagcggcaggcctaacacatgcaagtcgaggggcagcacggtgtagcaatacactggtggcgaccggcgcacgggtgcgtaacgcgtatgcaacctacccataacagggggataacactgagaaattggtactaataccccataacatcaggaccggcatcggttctggttgaaaactccggtggttatggatgggcatgcgttgtattagctggttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtacgagagtaaacgctcttacgtgtaagagcctgaaagtatcgtacgaataaggatcggctaactccgtgccagcagccgcggtaatacggaggatccaagcgttatccggatttattgggtttaaagggtgcgtaggcggtttgataagttagaggtgaaataccggtgcttaacaccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacggtcggcggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtagccgtaccggaaggtgcggctggaacacctccttt

SEQ ID NO: 17 - DNA, 1530 bp, Artificial Sequence
  Synthetic Polynucleotide
atgagagtttgatcctggctcaggatgaacgctggcggcgtgcttaacacatgcaagtcgaacgaagcactctatttgattttcttcggaaatgaagattttgtgactgagtggcggacgggtgagtaacgcgtgggtaacctgcctcatacagggggataacagttggaaacgactgctaataccgcataagcgcacaggatcacatgatccggtgtgaaaaactccggtggtatgagatggacccgcgtctgattagccagttggcagggtaacggcctaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccgatgacagggtatgtaatgtactttctcttcggagcatcggtgacaggtggtgcatggttgtcgtcagctcgtgtcgtgagatgttgggttaagtcccgcaacgagcgcaacccctgtccttagtagccagcggttcggccgggcactctagggagactgccagggataacctggaggaaggcggggatgacgtcaaatcatcatgccccttatgacttgggctacacacgtgctacaatggcgtaaacaaagggaagcggagccgtgaggccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttggaaatgcccgaagtcagtgacccaaccgcaaggagggagctgccgaaggcaggttcgataactggggtgaagtcgtaacaaggtagccgtatcggaaggtgcggctggatcacctccttt

SEQ ID NO: 18 - DNA, 1509 bp, Artificial Sequence
  Synthetic Polynucleotide
atggagagtttgattctggctcagaacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctccttt

SEQ ID NO: 19 - DNA, 1557 bp, Artificial Sequence
  Synthetic Polynucleotide
attggagagtttgatcctggctcaggacgaacgctggcggcgtgcctaacacatgcaagtcgaacggagaattttatttcggtagaattcttagtggcgaacgggtgagtaacgcgtaggcaacctgccctttagacggggacaacattccgaaaggagtgctaataccggatgtgatcatcgtgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagatttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagtaatcgcaggtcagcatactgcggtgaatacgttcccgggccttgtacacaccgcccgtcacaccacgaaagttggtaacacccgaagccggtgaggtaacctataaggagccagccgtctaaggtggggccgatgattggggtgaagtcgtaacaaggtagccgtatcggaaggtgcggctggatcacctccttt

SEQ ID NO: 20 - DNA, 809 bp, Artificial Sequence
  Synthetic Polynucleotide
  misc_feature (631)..(631): n is a, c, g, or t
ctgagggggtcaatcccccaacacctagtaatcatcgtttacggcatggactaccagggtatctaatcctgtttgctacccatgctttcgagcctcagcgtcagttggtgcccagtagaccgccttcgccactggtgttcctcccgatatctacgcattccaccgctacaccgggaattccatctacctctgcactactcaagaaaaacagttttgaaagcaattcatgggttgagcccatggttttcacttccaacttgtcttcccgcctgcgctccctttacacccagtaattccggacaacgcttgcaccctacgttttaccgcggctgctggcacgtagttagccggtgctttcttgttaggtaccgtcattatcgtccctaacgacaggagtttacaatccgaaaaccttcttcctccacgcggcgtcgctgcatcagggtttcccccattgtgcaatatcccccactgctgcctcccgtaggagtctgggccgtgtctcagtcccaatgtggccgttcaacctctcagtccggctaccaatcgtcgccttggtgggccgttacctcaccaactagctaattggacgcgagtccatcctaaagcgaataaatccttttccctcagnaccatgcggtaccgtgggcttatgcggtattagcagtcgtttccaactgttgtcccccactctagggcaggttactcacgcgttactcacccgttcgccactaagcttaaaaagcaagctctctaagctccgttcgacttgcatgtgttaggcgcgccgccagcgttcgtcctgagcc

SEQ ID NO: 21 - DNA, 902 bp, Artificial Sequence
  Synthetic Polynucleotide
  misc_feature (796)..(796): n is a, c, g, or t
  misc_feature (825)..(825): n is a, c, g, or t
  misc_feature (838)..(838): n is a, c, g, or t
  misc_feature (841)..(841): n is a, c, g, or t
  misc_feature (881)..(881): n is a, c, g, or t
tgctttctcgagcgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaaggcaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtaaacgatgattactaggtgttggaggattgaccccttcagtgccgcagttaacacaataagtaatccacctggggagtacgaccgcaanggttgaaactcaaaggaattgacgggggnccgcacaagcagnggngtatgtggtttaattcgacgcaacgcgaagaaccttaccnagtcttgacatcctgcgacgg

SEQ ID NO: 22 - DNA, 1000 bp, Artificial Sequence
  Synthetic Polynucleotide
ataccttgatggcgaccggcgcacgggtgagtaacgcgtatgcaacctgcctgataccggggtatagcccatggaaacgtggattaacaccccatagtacttttatcctgcatgggatgtgagttaaatgttcaaggtatcggatgggcatgcgtcctattagttagttggcggggtaacagcccaccaagacgatgataggtaggggttctgagaggaaggtcccccacattggaactgagacacggtccaaacttctacgggaggcagcagtgaggaatattggtcaatggacgtaagtctgaaccagccaagtcgcgtgagggaagactgccctatgggttgtaaacctcttttataagggaagaataagttctacgtgtagaatgatgcctgtaccttatgaataagcatcggctaactccgtgccagcagccgcggtaatacggaggatgcgagcgttatccggatttattgggtttaaagggtgcgtaggcggtttattaagttagtggttaaatatttgagctaaactcaattgtgccattaatactggtaaactggagtacagacgaggtaggcggaataagttaagtagcggtgaaatgcatagatataacttagaactccgatagcgaaggcagcttaccagactgtaactgacgctgatgcacgagagcgtgggtagcgaacaggattagataccctggtagtccacgccgtaaacgatgctcactggttctgtgcgatatattgtacgggattaagcgaaagtattaagtgagccacctggggagtacgtcggcaacgatgaaactcaaaggaattgacggggcccgcacaagcggaggaacatgtggtttaattcgatgatacgcgaggaaccttacctgggtttaaatgggaatgtcgtatttgggaacagatattctcttcgagcgtttttcaaggtgctgcatggttgtcgtcagctcgtgcggtgaggggtcggtagtccata

SEQ ID NO: 23 - DNA, 982 bp, Artificial Sequence
  Synthetic Polynucleotide
ttttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttattcgaagcaacgcgaagaaccttacctggtcttgacatccttctgaccggtccttaaccggactttccttcgggacagagtgacaaggtggtgcatgtgtcgtcagctcgtgtctga

SEQ ID NO: 24 - DNA, 387 bp, Artificial Sequence
  Synthetic Polynucleotide
agcaatactttgggtggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacgggaggcagcagtgaggaatattggtcaatggacgcaagtctgaaccagccaggccgcgtgcaggaagacggctctatgagttgtaaactgcttttgtacgagggtaaactcacctacgtg

SEQ ID NO: 25 - DNA, 843 bp, Artificial Sequence
  Synthetic Polynucleotide
attgtatcgcaaactcctagtaatcatcgtttactgcgtggactaccagggtatctaatcctgtttgatccccacgctttcgtgcttcagtgtcagttatggtttagtaagctgccttcgcaatcggagttctgcgtgatatctatgcatttcaccgctacaccacgcattccgcctacctcaaacacactcaagtaacccagtttcaacggcaattttatggttgagccacaaactttcaccgctgacttaaatcaccacctacgcaccctttaaacccaataaatccggataacgctcgcatcctccgtattaccgcggctgctggcacggagttagccgatgcttattcatagggtacatacaaaaaaggacacgtcctccactttattcccctataaaagaagtttacaaaccatagatccttcttccttcacgcgacttggctggttcagcctctcggccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttaacaaatattcccatgcgggacccctgttttatggggtattaatccgactttcgccgggctatccccctctgataggtaagttgcatacgcgttactcacccgtgcgccggtcgccatcaatctgtattgctacaaatcatgctgcccctcgacttgcatgtgttaagcctgtcgctagcgttcatcctgagccagattcaaactctataa

SEQ ID NO: 26 - DNA, 939 bp, Artificial Sequence
  Synthetic Polynucleotide
gaagcttcggcagatttggccggtttctagtggcggacgggtgagtaacgcgtgggtaacctgccttatacagggggataacaaccagaaatggttgctaataccgcataagcgcacaggaccgcatggtccagtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctgggtagtccacgccgtaaacgatgaatactaggtgttgggggagcaaagctcttcggtgccgccgcaaacgcattaagtattccacctggggagtacgttcgcaagaatgaaactcaaaggatttgacggggacccgcacaagcgtggagcatgtgtttattcgaagcaaccggaagaacttaccaggcttgacatcctctgacgttcgtaacggaactttcttcggacaggaaa

SEQ ID NO: 27 - DNA, 956 bp, Artificial Sequence
  Synthetic Polynucleotide
tgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttataaaggaataaagtcgggtatgtatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaactcaaaggaattgacggggcccgcacaagcggagaacatgtggtttaattcgatgatacgcgaggaccttacccggcttaaattgcagatgaattacgtgaagccgtaagccgcaagcatctgtgaagtgctgcat

SEQ ID NO: 28 - DNA, 980 bp, Artificial Sequence
  Synthetic Polynucleotide
taggttgatggcgaccggcgcacgggtgagtaacacgtatccaacctaccggttattccgggatagcctttcgaaagaaagattaataccggatagtataacgagaaggcatctttttgttattaaagaatttcgataaccgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgagccacgtgtggctttttgtatgtaccatacgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcgagaacatgtggtttaattcgatgatacgcgagaaccttaccgggcttaaattgcatctgaataatttggaaacagattagccgcaagcagatgtgaaggtgctgcatggtgtcgtcagctcgtgcgtgaggt

SEQ ID NO: 29 - DNA, 867 bp, Artificial Sequence
  Synthetic Polynucleotide
tgcttcaattgccggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacgggaggcagcagtgaggaatattggtcaatggacgcaagtctgaaccagccatgccgcgtgcaggaagacggctctatgagttgtaaactgcttttgtacgagggtaaactcacctacgtgtaggtgactgaaagtatcgtacgaataaggatcggctaactccgtgccagcagccgcggtaatacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaaacaggattagataccctggtagtccacgcagtaaacgatgataactcgttgtcggcgatacacagtcggtgactaagcgaaagcgataagttatccacctggggagtacgttcgcaagaatgaacttaaaggaattgacggggcccgcacaagcggagaactgtggttatt

SEQ ID NO: 30 - DNA, 261 bp, Artificial Sequence
  Synthetic Polynucleotide
ttgctaagtttgatggcgaccggcgcacgggtgagtaacacgtatccaacctgccgatgactcggggatagcctttcgaaagaaagattaatacccgatggcatatttcttccgcatggtagaattattaaagaatttcggtcatcgatggggatgcgttccattaggttgttggcggggtaacggcccaccaagccttcgatggataggggttctgagaggaaggtcccccacattggaactgagccaggatcccaccat

SEQ ID NO: 31 - DNA, 832 bp, Artificial Sequence
  Synthetic Polynucleotide
gtgtatcgccgacaacgagttatcatcgtttactgcgtggactaccagggtatctaatcctgtttgctccccacgctttcgtgcctcaacgtcagatatagtttggtaagctgccttcgcaatcggtgttctgtatgatctctaagcatttcaccgctacaccatacattccgcctaccgcaactactctctagtctaacagtattagaggcagttccggagttaagccccgggatttcacctctaacttatcaaaccgcctacgcaccctttaaacccaataaatccggataacgcttgaatcctccgtattaccgcggctgctggcacggagttagccgatccttattcgtacgatactttcagtcacctacacgtaggtgagtttaccctcgtacaaaagcagtttacaactcatagagccgtcttcctgcacgcggcatggctggttcagacttgcgtccattgaccaatattcctcactgctgcctcccgtaggagtctggtccgtgtctcagtaccagtgtggggggttaacctctcagtccccctatgtatcgttgccttggtgagccgttacctcaccaactagctaatacaacgcatgtccatctataaccaccggagttttcaacccggagagatgcctctccgaatattatggggaattagtaccaatttctcagtgttatgcccctgttataggtaggttacatacgcgttacgcacccgtgcgccggtcgccaccaaagtattgctacctcgtgatgcccctcgacttgcatgtgttaggcctgccgctagcgttcatcctgagccagattcaaactct

SEQ ID NO: 32 - DNA, 1527 bp, Artificial Sequence
  Synthetic Polynucleotide
tcagagagtttgatcctggctcaggatgaacgctggcggcgtgcttaacacatgcaagtcgaacgggaaaccttttattgaagcttcggcagatttagattgtttctagtggcggacgggtgagtaacgcgtgggtaacctgccttatacagggggataacagtcagaaatgactgctaataccgcataagcgcacagagctgcatggctcggtgtgaaaaactccggtggtataagatggacccgcgttggattagctagttggtgaggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggatggacaagtctgatgtgaaaggctggggctcaaccccgggactgcattggaaactgcccgtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgcggtaaacgatgaatgctaggtgtcggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaagtcttgacatccttctgaccggaacttaaccgttccttcccttcggggcagaagtgacaggtggtgcatggttgtcgtcagctcgtgtcgtgagatgttgggttaagtcccgcaacgagcgcaacccctatcctcagtagccagcatataaggtgggcactctggggagactgccagggataacctggaggaaggcggggatgacgtcaaatcatcatgccccttatgatttgggctacacacgtgctacaatggcgtaaacaaagggaagcgaacctgcgagggtgggcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaaccgcaaggaaggagctgccgaaggcgggaccgatgactggggtgaagtcgtaacaaggtagccgtatcggaaggtgcggctggatcacctccttt

SEQ ID NO: 33 - DNA, 1055 bp, Artificial Sequence
  Synthetic Polynucleotide
  misc_feature (868)..(868): n is a, c, g, or t
  misc_feature (917)..(917): n is a, c, g, or t
  misc_feature (925)..(925): n is a, c, g, or t
  misc_feature (984)..(986): n is a, c, g, or t
  misc_feature (1002)..(1002): n is a, c, g, or t
  misc_feature (1015)..(1015): n is a, c, g, or t
  misc_feature (1020)..(1020): n is a, c, g, or t
  misc_feature (1049)..(1049): n is a, c, g, or t
gaggggcatgccatgatggagtagtaatatctatggtggcagaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggccncgcacaagcggaggaacatgtgggtttaattcgatgatacgcgaggaanccttaccncgggcttgaaagttagcgacgattcttgaaagaggatttcccttcggggcgcgaaactnnngctgcatggttgtcgncagctcgtgccgngaggngtcgggttaagtcccataacgagcgcacnccctac

SEQ ID NO: 34 - DNA, 988 bp, Artificial Sequence
  Synthetic Polynucleotide
  misc_feature (5)..(5): n is a, c, g, or t
acacntgcagtcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttccctttcggggcgcgaaact

SEQ ID NO: 35 - DNA, 1029 bp, Artificial Sequence
  Synthetic Polynucleotide
  misc_feature (5)..(5): n is a, c, g, or t
  misc_feature (989)..(991): n is a, c, g, or t
acacntggaagtcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttcccttcggggcgcgaaactnnngctgcatggtgtcgtcagctcgtgccgtgaggtgtcgg